(19) United States
(12) Patent Application Publication
(10) Pub. No.: US 2026/0284220 A1
(43) Pub. Date: Sep. 24, 2026

(54) Macrocyclic Compound and Preparation Method and Use Thereof

(71) Applicant: SICHUAN KELUN-BIOTECH BIOPHARMACEUTICAL CO., LTD., Chengdu, Sichuan (CN)

(72) Inventors: Qiang TIAN, Chengdu, Sichuan (CN); Yitao ZHANG, Chengdu, Sichuan (CN); Dong LONG, Chengdu, Sichuan (CN); Jian YE, Chengdu, Sichuan (CN); Qin ZHOU, Chengdu, Sichuan (CN); Hu LONG, Chengdu, Sichuan (CN); Yu YANG, Chengdu, Sichuan (CN); Xiaoxi YUAN, Chengdu, Sichuan (CN); Hongmei SONG, Chengdu, Sichuan (CN); Junyou GE, Chengdu, Sichuan (CN)

(21) Appl. No.: 19/469,982

(22) PCT Filed: May 10, 2024
(86) PCT No.: PCT/CN2024/092442

§ 371 Date: Sep. 26, 2025

(30) Foreign Application Priority Data
May 12, 2023 (CN) 202310538792.7
Oct. 13, 2023 (CN) 202311330295.4

(51) Int. Cl. A61K 47/68; A61P 35/00; C07K 16/32

(52) U.S. Cl. CPC A61K 47/6855; A61K 47/6803; A61K 47/6889; A61P 35/00; C07K 16/32

(57) ABSTRACT

Provided are a macrocyclic compound and a preparation method and a use thereof, which has a structure shown in the formula M′-L-E-D. The compound can be used to prepare an antibody-drug conjugate. The conjugate has a relatively good drug-antibody conjugation ratio and has an excellent targeted killing effect on solid tumors such as gastric cancer, breast cancer, lung cancer, and urothelial carcinoma.

The present application is based on and claims priority to the Chinese application with application number CN 202310538792.7 (filed on May 12, 2023), and the Chinese application with application number CN 202311330295.4 (filed on Oct. 13, 2023). All the contents of the Chinese applications are hereby incorporated in their entirety into the present application.

### TECHNICAL FIELD

The present application relates to the field of medicine, and specifically to a macrocyclic compound as well as preparation method and use thereof.

### BACKGROUND ART

Antibody-drug conjugate (ADC) is typically composed of a monoclonal antibody, a biologically active molecule and a linker. The antibody recognizes a specific target on the surface of tumor cells, directs the ADC to the tumor microenvironment and cancer cell surfaces, and allows the ADC to enter the cancer cells through endocytosis; the bioactive molecule is covalently coupled to the antibody via the linker; then the bioactive molecule is released in the cancer cells, and kills the cancer cells by effects such as damaging the DNA of the cancer cells or inhibiting the tubulin of the cancer cells, while minimizing damage to normal tissue cells.

With the widespread clinical application of the above-mentioned ADC drugs in the treatment tumors, safety issues including hepatotoxicity, interstitial pneumonia, neurotoxicity and hematotoxicity and drug resistance issues have gradually emerged (Pharmacology & Therapeutics 2019, 200, 110-125; Breast Cancer Research and Treatment 2020, 183, 23-39; JAMA Oncol. 2021, 7, 1873-1881; Drug Deliv. 2022, 29, 1335-1344; Cancers 2023, 15, 1130; Cancers 2023, 15, 1278.).

As an important component of antibody-drug conjugates, the structure of the drug-linker moiety is closely related to the overall efficacy and safety of ADC drugs. Therefore, the development of novel drug-linker structures is of great significance for the development of antibody-drug conjugates with good efficacy and safety.

### CONTENTS OF THE PRESENT INVENTION

The present application relates to a macrocyclic compound having a structure represented by the general formula D-E-L-M′. The macrocyclic compound can be used to prepare an antibody-drug conjugate, and the conjugate has a good, targeted killing effect on tumors.

### Compound

In one aspect, the present application provides a compound or pharmaceutically acceptable salt thereof, which has a structure represented by the formula D-E-L-M′, wherein:
M′ represents -M-Lg, wherein Lg is a leaving group for a nucleophilic substitution reaction, and M is a structural fragment that connects to a targeting moiety;
L represents a structural fragment connecting M and E;
E represents a structural fragment connecting L and D;
D represents a cytotoxic drug fragment.

In some embodiments, M is selected from the group consisting of the following substituted or unsubstituted structural fragments:

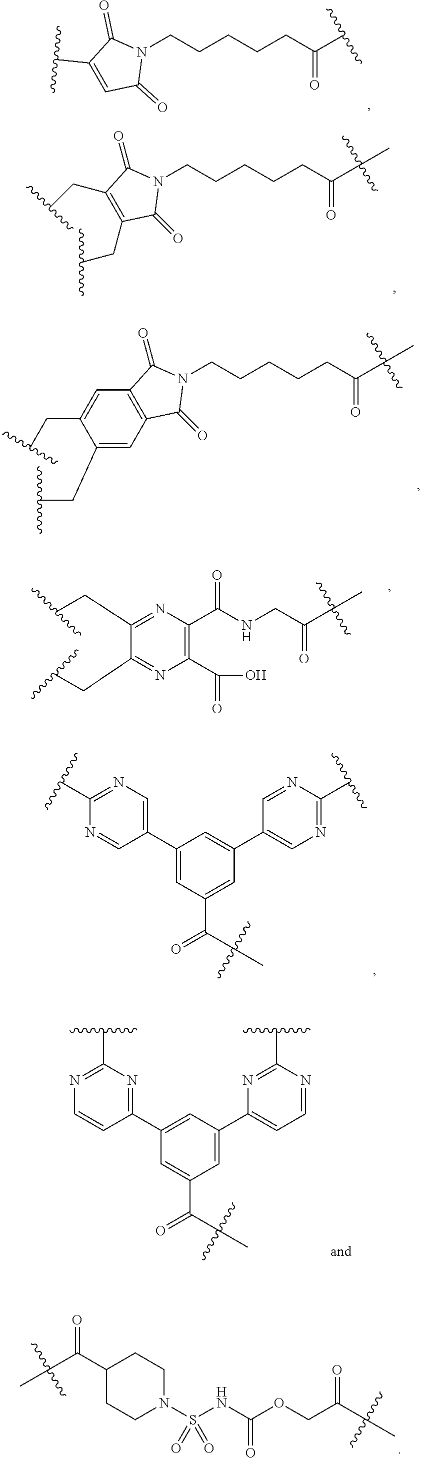

In some embodiments, M is selected from the group consisting of the following substituted or unsubstituted structural fragments:

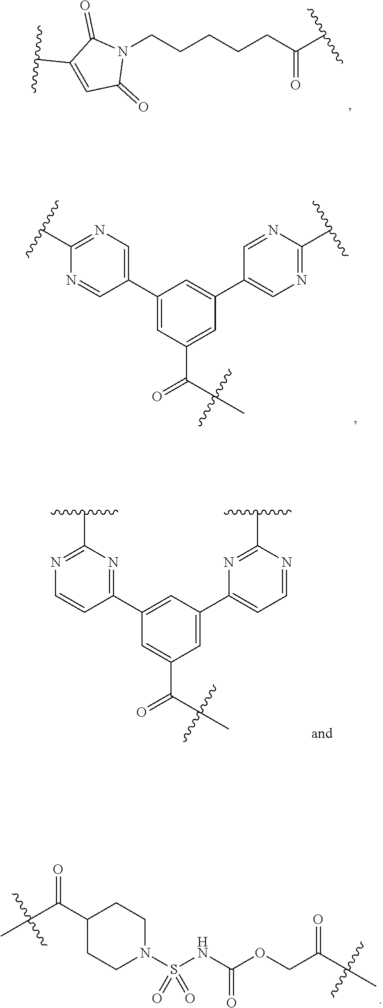 [SMILES: *C(=O)CCCCCN1C(=O)C=C(*)C1=O ; *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the group consisting of the following substituted or unsubstituted structural fragments:

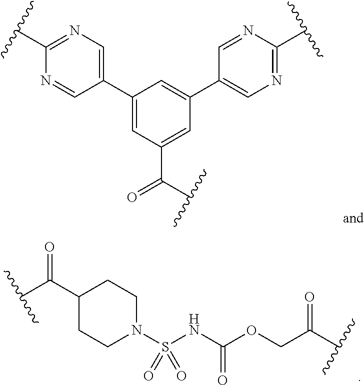 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

In some embodiments, M is selected from the group consisting of the following substituted or unsubstituted structural fragments:

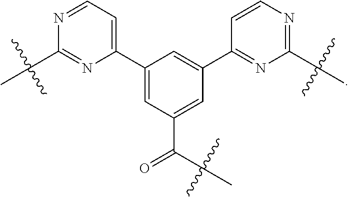

In some embodiments, M is selected from the group consisting of the following substituted or unsubstituted structural fragments:

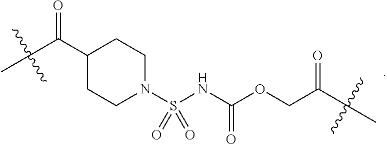 [SMILES: *C(=O)COC(=O)NS(=O)(=O)N1CCC(C(*)=O)CC1]

In some embodiments, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, hydroxyl (—OH), mercapto (—SH), amino (—NH₂), nitro, azido, cyano, alkenyl, alkynyl, and alkynyl-containing structural fragment, wherein the halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, alkenyl, alkynyl, and alkynyl-containing structural fragment are optionally substituted with one or more suitable substituents.

In some embodiments, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁0.6 alkylsulfoxide group, halogenated phenoxy, hydroxyl (—OH), mercapto (—SH), amino (—NH₂), nitro, azido, cyano, alkenyl, alkynyl, and alkynyl-containing structural fragment.

In some embodiments, Lg is selected from the group consisting of halogen, substituted or unsubstituted C₁₋₆ alkylsulfonyl (C₁₋₆ alkyl-SO₂—), halogenated phenoxy, hydroxyl (—OH), mercapto (—SH), or amino (—NH₂).

In some embodiments, Lg is selected from the group consisting of halogen, substituted or unsubstituted methylsulfonyl, halogenated phenoxy, hydroxyl (—OH), mercapto (—SH), or amino (—NH₂).

In some embodiments, Lg is selected from the group consisting of methylsulfonyl or pentafluorophenoxy.

In some embodiments, L is selected from the group consisting of the substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), and short peptide composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly (GGFG, SEQ ID NO: 41), Gly-Gly-Val-Ala (GGVA, SEQ ID NO: 42), Gly-Phe-Leu-Gly (GFLG, SEQ ID NO: 43), Glu-Ala-Ala-Ala (EAAA, SEQ ID NO: 44), Gly-Gly-Gly-Gly-Gly (GGGGG, SEQ ID NO: 45), Ala-Ala-Glu),

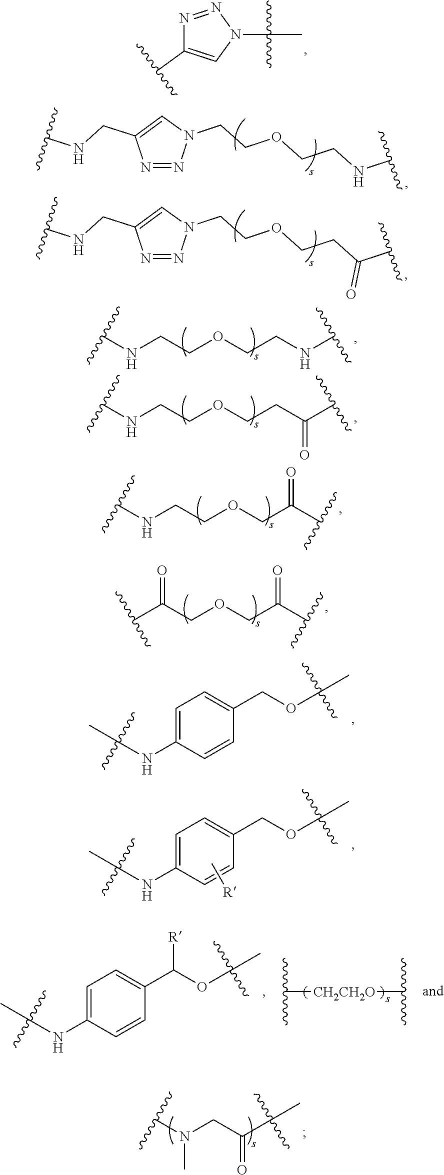

wherein R′ is composed of one or more of the following groups, including but not limited to hydrogen, C₁₋₆ alkyl, C₁₋₆ alkylene, amino, hydroxyl, carboxyl, acyl, —O—, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, polyethylene glycol fragment containing 1 to 10 EO units (i.e. —(CH₂CH₂O)₁₋₁₀—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-a-propionyl), or NOTA (1,4,7-triazacyclononane-N,N′,N″-triacetic acid residue), wherein r is an integer selected from 1 to 20 (e.g., an integer of 1 to 15, such as 1 to 12, 3 to 12, 1 to 10, 1 to 8, 3 to 8, 1 to 6, 1 to 4, 1 to 2, such as 1, 2, 3, 4, 5, 6, 7, 8, 9, 10, 11, 12, 13, 14, 15, 16, 17, 18, 19 or 20); s is an integer selected from 1 to 20 (e.g., an integer of 1 to 15, such as an integer of 1 to 12, 3 to 12, such as 1, 2, 3, 4, 5, 6, 7, 8, 9, 10, 11, 12, 13, 14, 15, 16, 17, 18, 19 or 20).

In some embodiments, s is denoted as n.

In some embodiments, “—NOTA” refers to

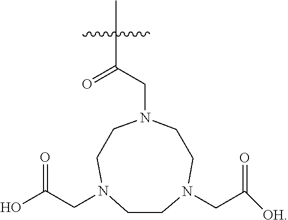 [SMILES: *C(=O)CN1CCN(CC(=O)O)CCN(CC(=O)O)CC1]

In some embodiments, “-DOTA” refers to

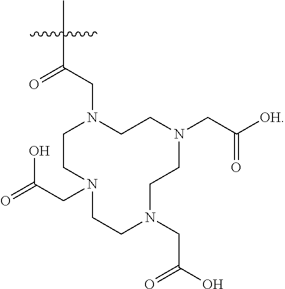 [SMILES: *C(=O)CN1CCN(CC(=O)O)CCN(CC(=O)O)CCN(CC(=O)O)CC1]

In some embodiments, “-DOTAGA” refers to

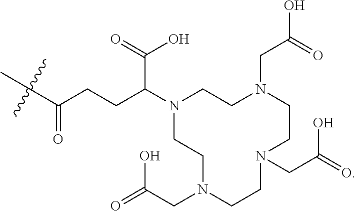 [SMILES: *C(=O)CCC(C(=O)O)N1CCN(CC(=O)O)CCN(CC(=O)O)CCN(CC(=O)O)CC1]

In some embodiments, L is selected from the group consisting of the substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), and short peptide composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

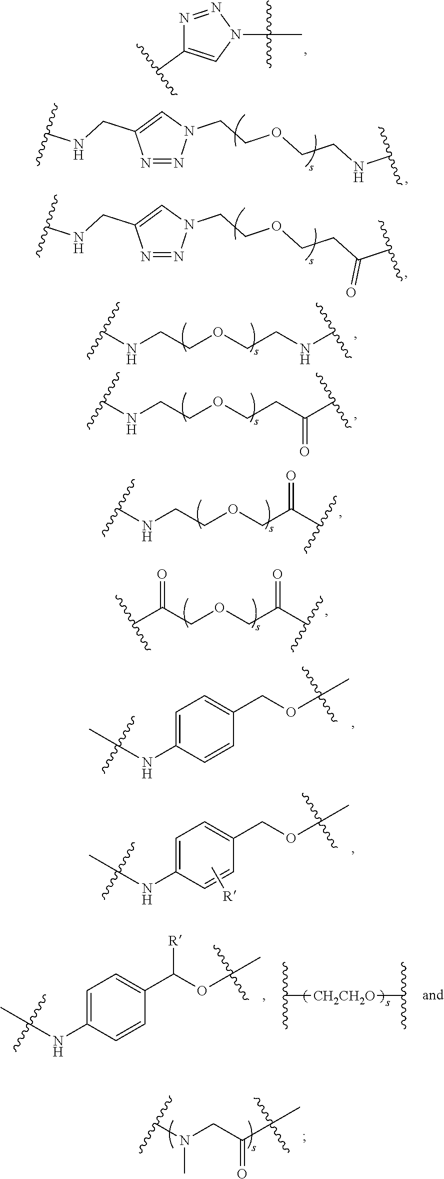

wherein R′ represents hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, or polyethylene glycol fragment containing 1 to 10 EO units (i.e. —(CH₂CH₂O)ᵣ—C₁₋₆ alkyl), wherein r is an integer selected from 1 to 20; s is an integer selected from 1 to 20.

In some embodiments, L is selected from the group consisting of the substituted or unsubstituted structural fragments consisting of one or more of the following groups: C₁₋₆ alkylene, carbonyl, —NH—, Ala-Ala, Ala-Lys, Ala-Pro, Gly-Glu, Gly-Gly, Phe-Lys, Val-Ala, Val-Cit, Val-Lys, Ala-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

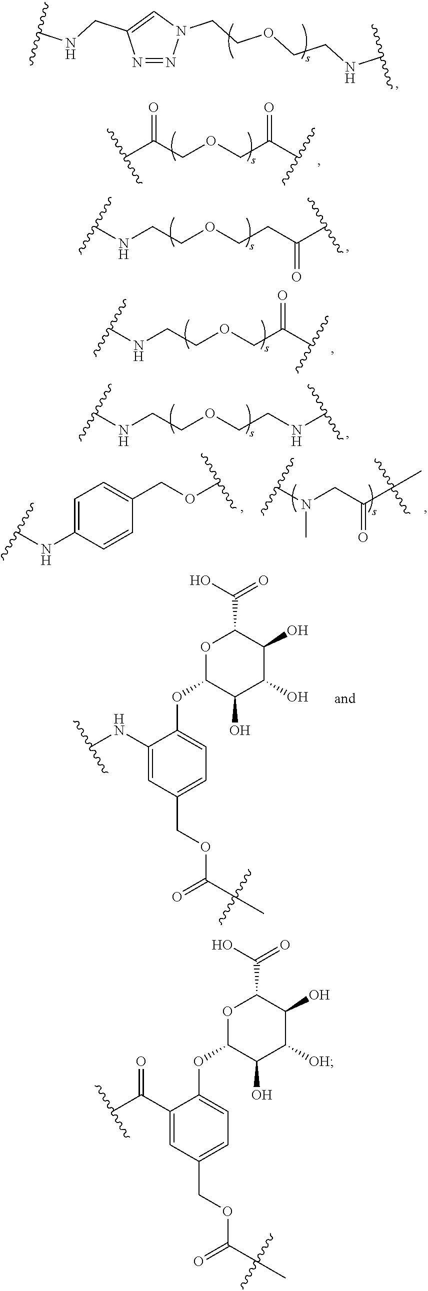

wherein s is an integer selected from 1 to 20.

In some embodiments, L is selected from the group consisting of the substituted or unsubstituted structural fragments composed of one or more of the following groups:

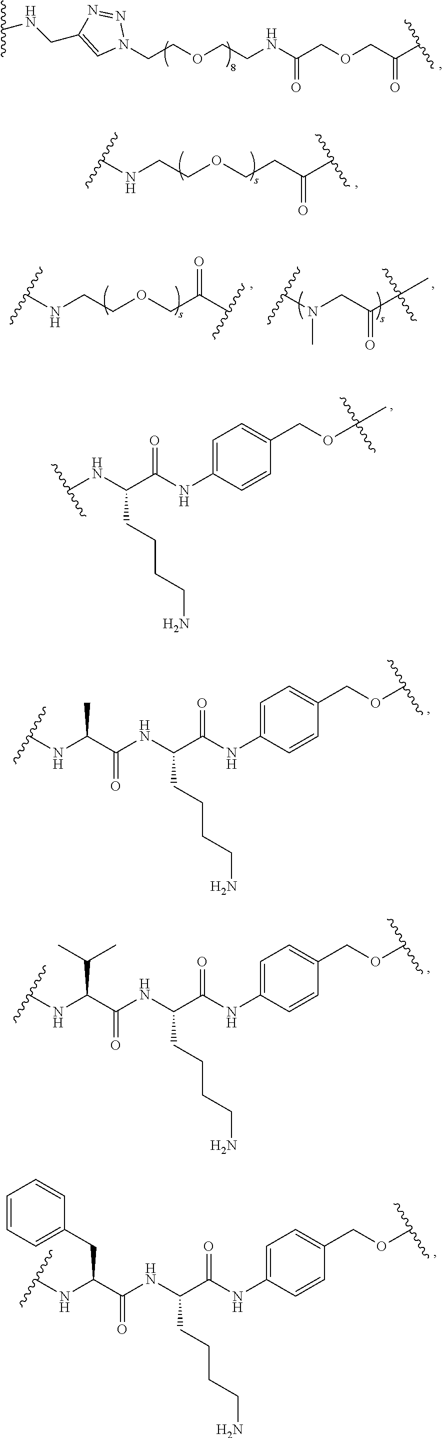

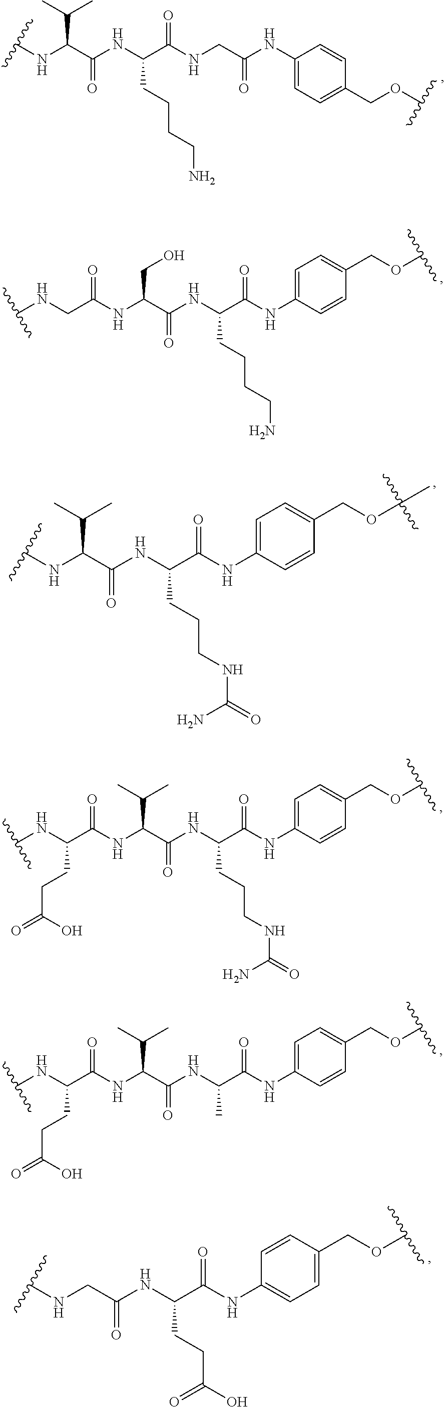 [SMILES: *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C ; *NCC(=O)N[C@@H](CCC(=O)O)C(=O)Nc1ccc(CO*)cc1]

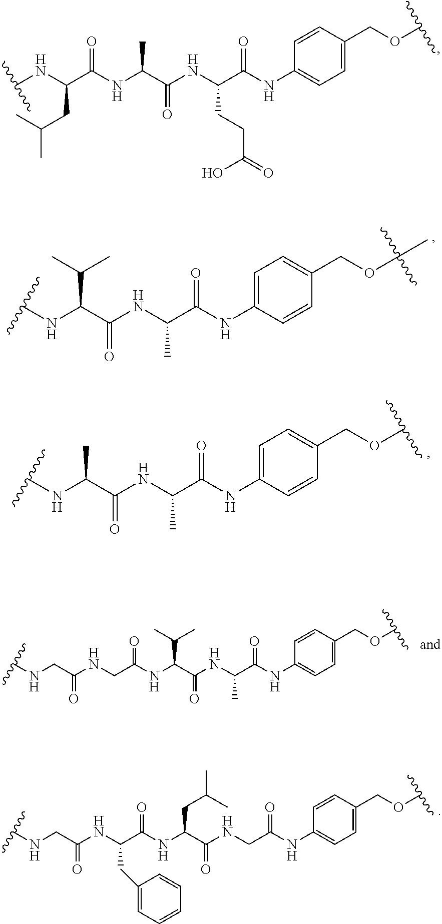 [SMILES: *N[C@H](CC(C)C)C(=O)N[C@@H](C)C(=O)N[C@@H](CCC(=O)O)C(=O)Nc1ccc(CO*)cc1 ; *N[C@H](C(=O)N[C@@H](C)C(=O)Nc1ccc(CO*)cc1)C(C)C ; *N[C@@H](C)C(=O)N[C@@H](C)C(=O)Nc1ccc(CO*)cc1 ; *NCC(=O)NCC(=O)N[C@H](C(=O)N[C@@H](C)C(=O)Nc1ccc(CO*)cc1)C(C)C ; *NCC(=O)N[C@@H](Cc1ccccc1)C(=O)N[C@@H](CC(C)C)C(=O)NCC(=O)Nc1ccc(CO*)cc1]

In some embodiments, L is selected from the group consisting of the substituted or unsubstituted structural fragments composed of one or more of the following groups:

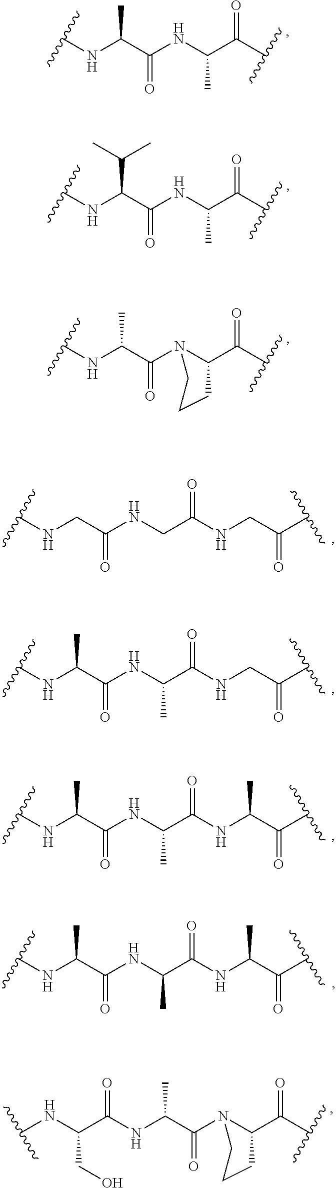

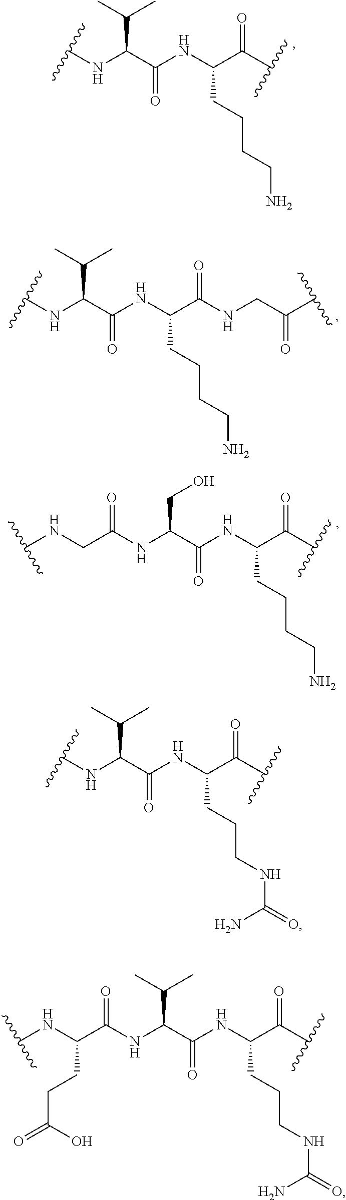

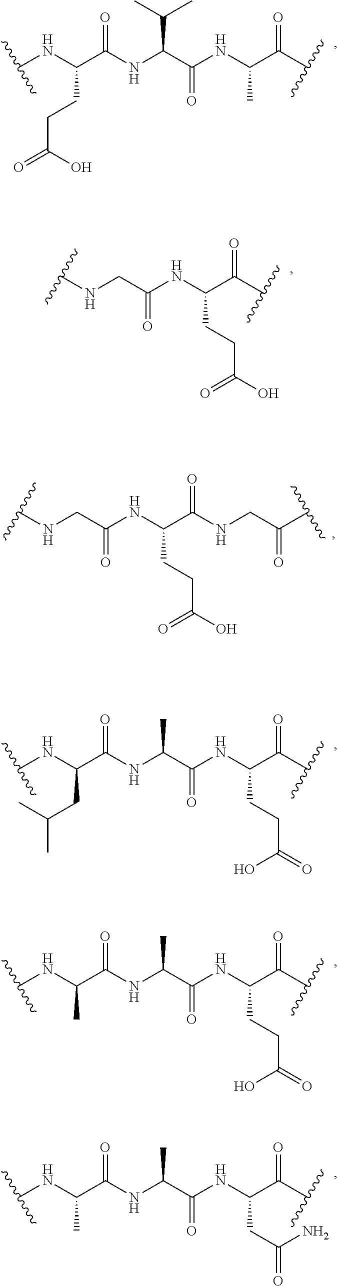 [SMILES: *N[C@@H](CCC(=O)O)C(=O)N[C@H](C(=O)N[C@@H](C)C(*)=O)C(C)C ; *NCC(=O)N[C@@H](CCC(=O)O)C(*)=O ; *NCC(=O)N[C@@H](CCC(=O)O)C(=O)NCC(*)=O ; *N[C@H](CC(C)C)C(=O)N[C@@H](C)C(=O)N[C@@H](CCC(=O)O)C(*)=O ; *N[C@H](C)C(=O)N[C@@H](C)C(=O)N[C@@H](CCC(=O)O)C(*)=O ; *N[C@@H](C)C(=O)N[C@@H](C)C(=O)N[C@@H](CC(N)=O)C(*)=O]

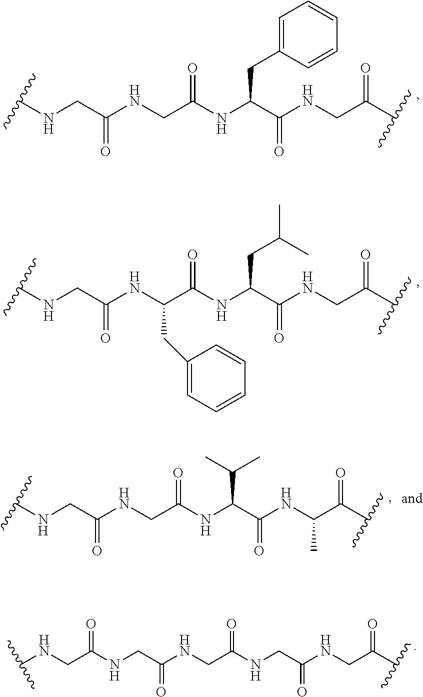

In some embodiments, L is selected from the group consisting of the substituted or unsubstituted structural fragments composed of one or more of the following groups:

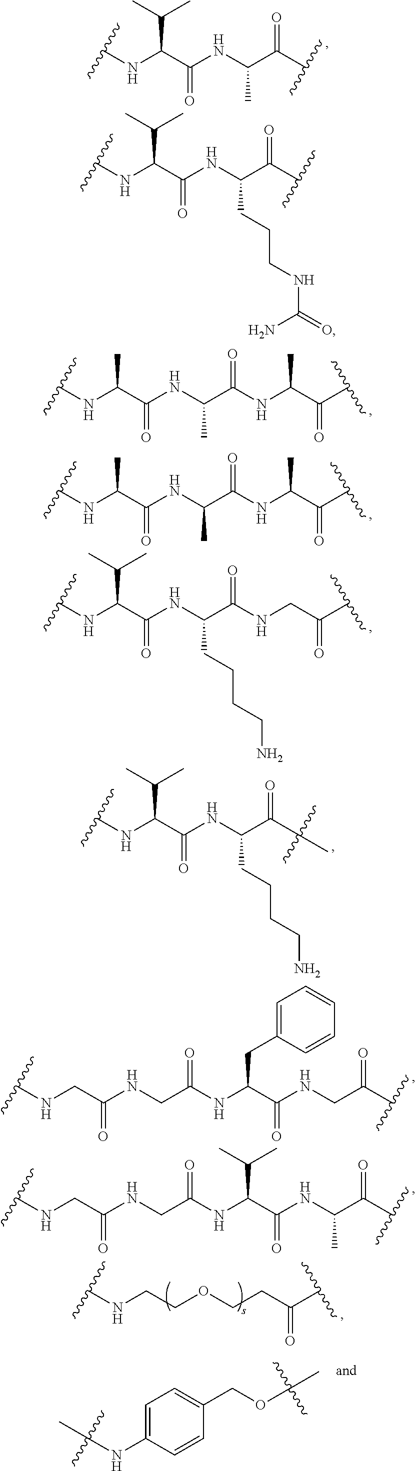

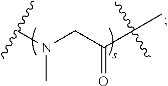

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments, L is selected from the group consisting of the substituted or unsubstituted structural fragments composed of one or more of the following groups:

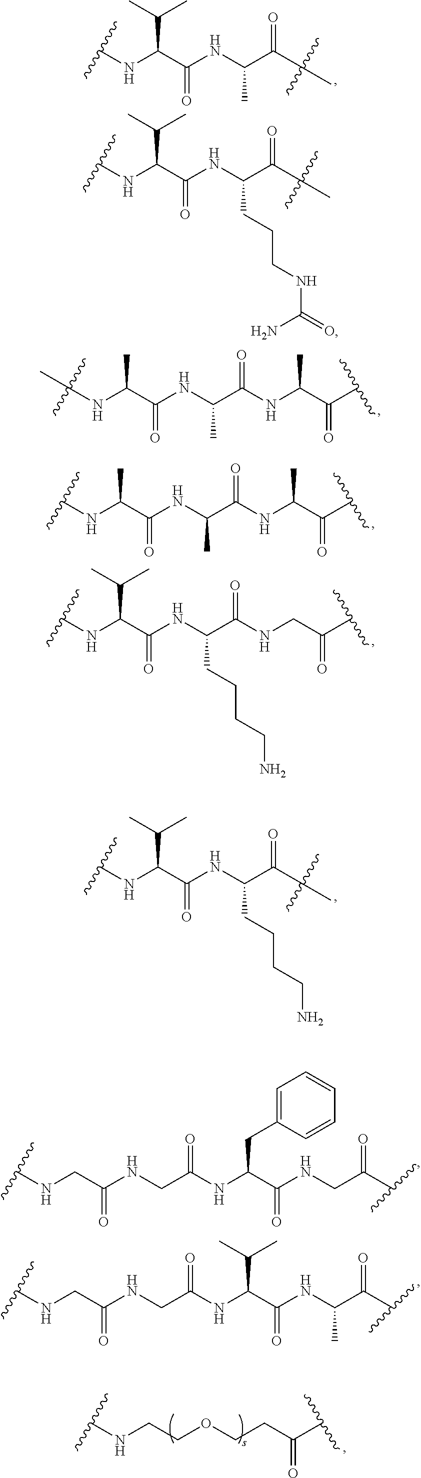

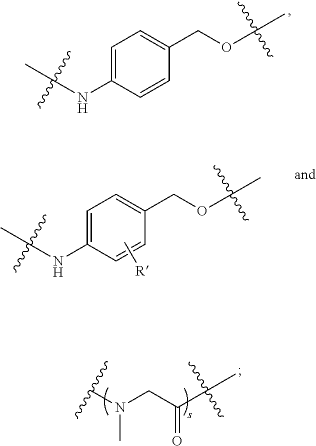

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15; R′ is composed of one or more of the following groups: C₁₋₆ alkyl, C₁₋₆ alkylene, amino, acyl, —O—, DOTA (1,4,7,10-tetraazacyclododecane-1,4,7,10-tetraacetic acid residue).

In some embodiments, L is selected from the group consisting of the substituted or unsubstituted structural fragments composed of one or more of the following groups:

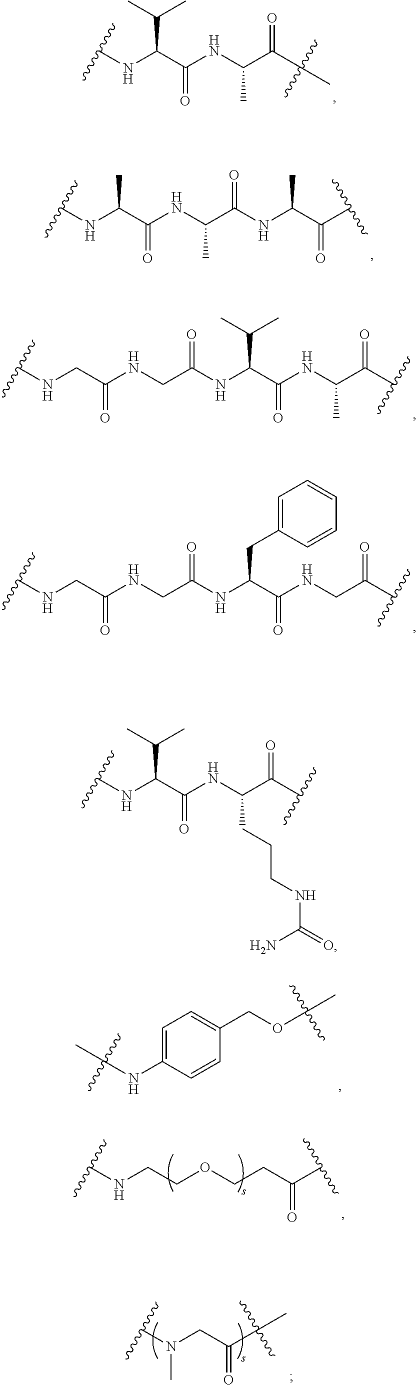

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments, L is selected from the group consisting of the substituted or unsubstituted structural fragments composed of one or more of the following group:

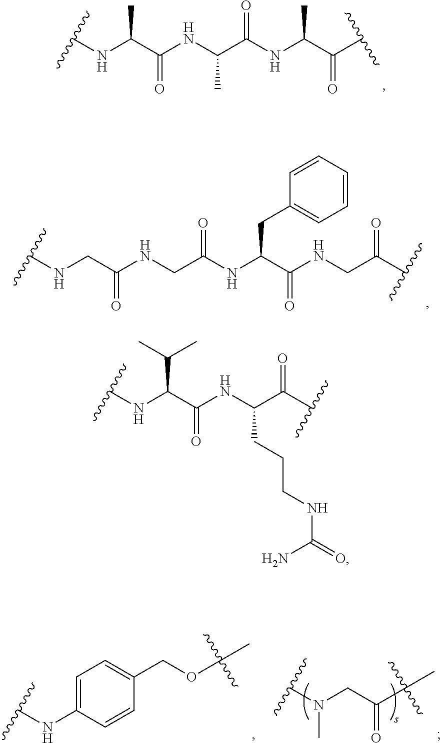

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments, L is selected from the group consisting of the substituted or unsubstituted structural fragments composed of one or more of the following group:

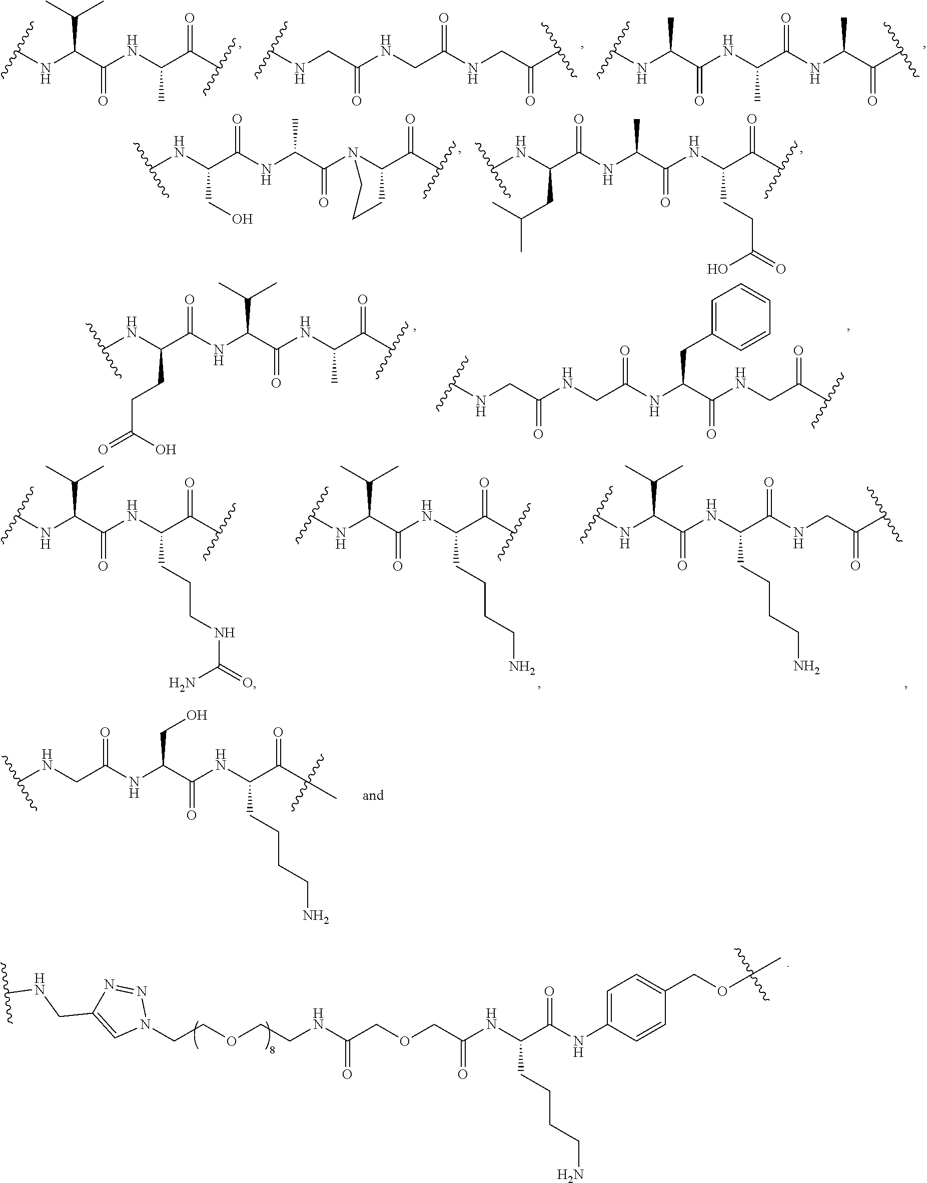

In some embodiments, L is selected from the group consisting of the following substituted or unsubstituted structural fragments:

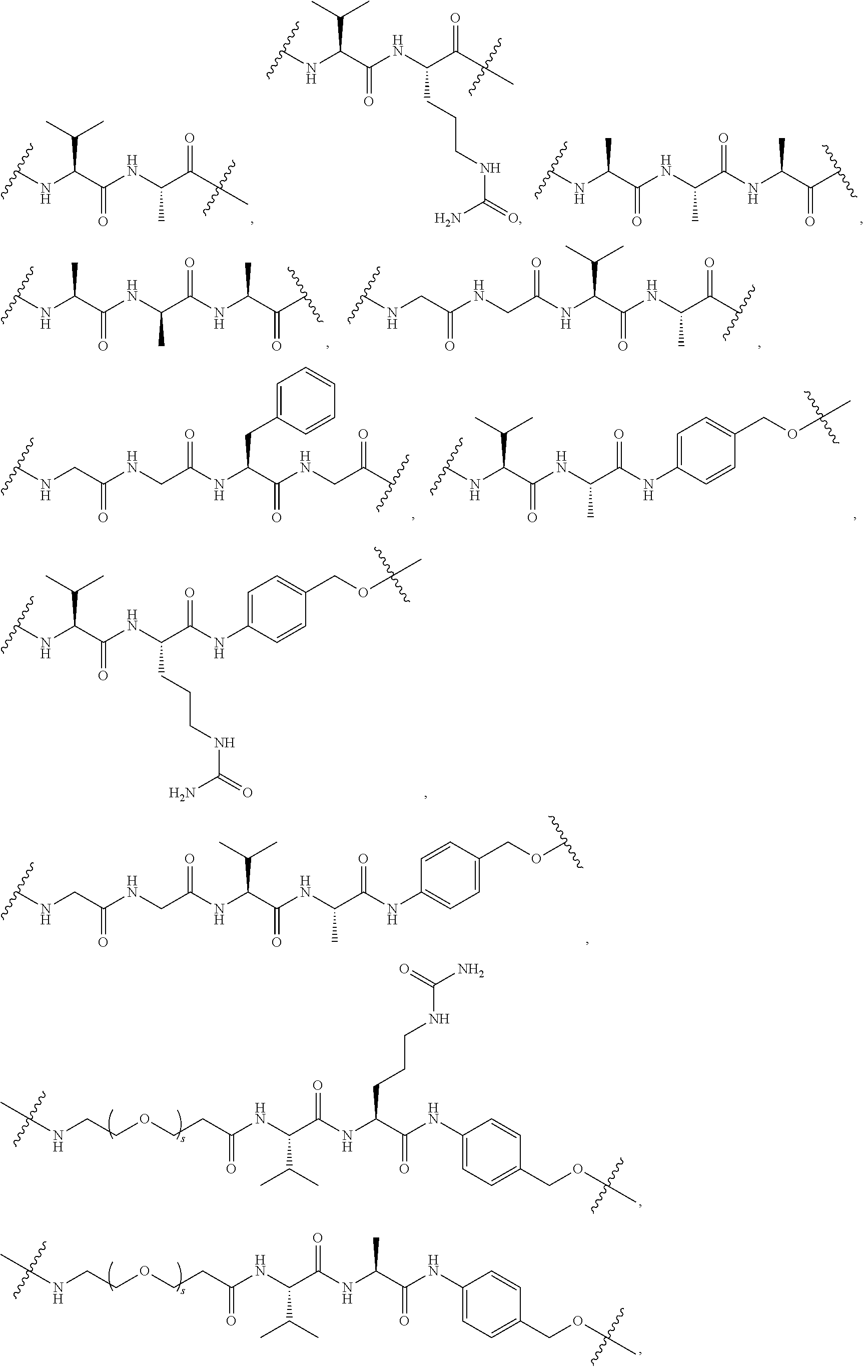

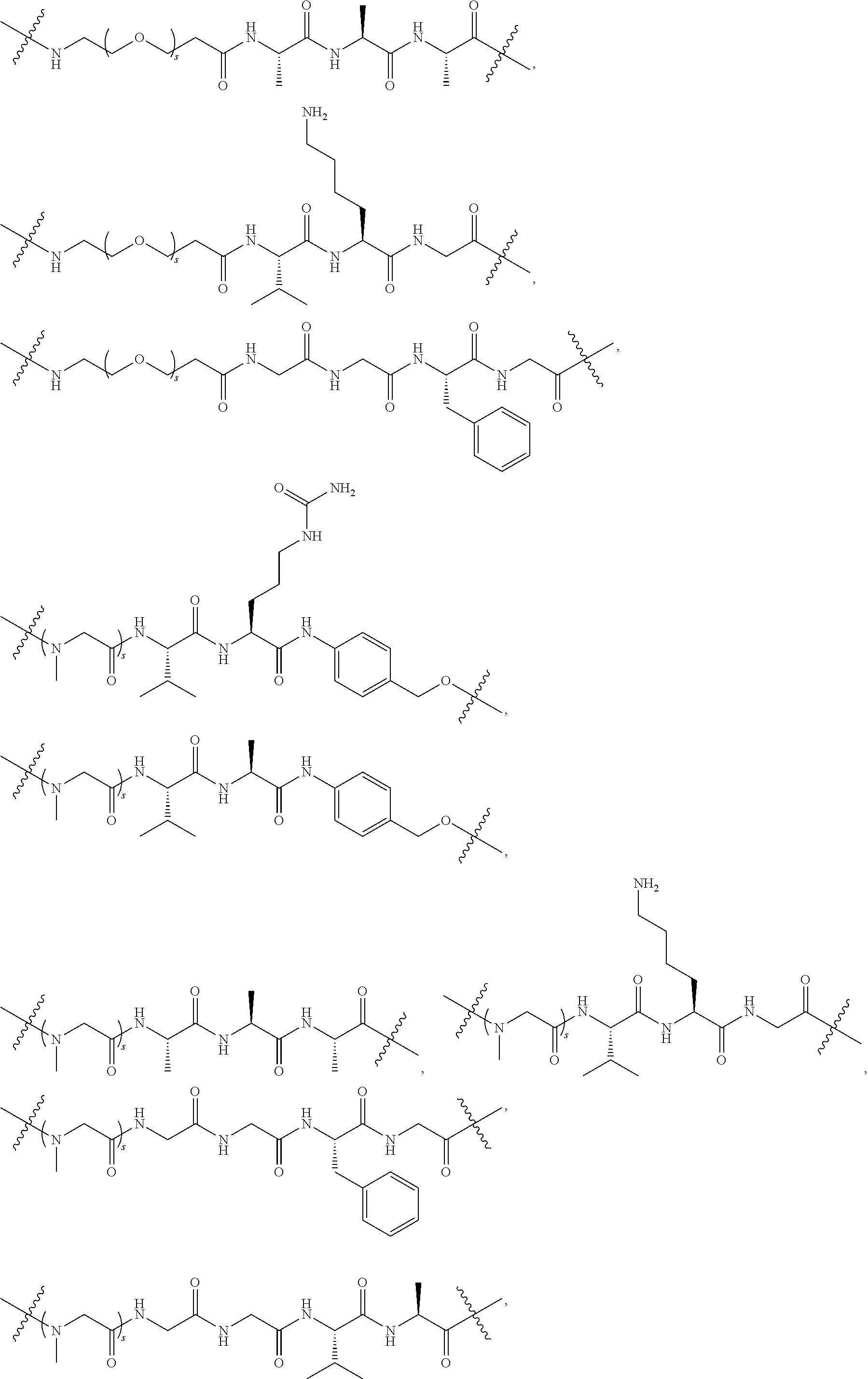

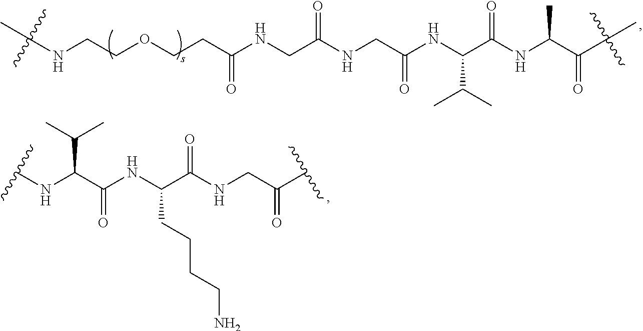

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10,11, 12, 13, 14 or 15.

In some embodiments, L is selected from the group consisting of the following substituted or unsubstituted structural fragments:

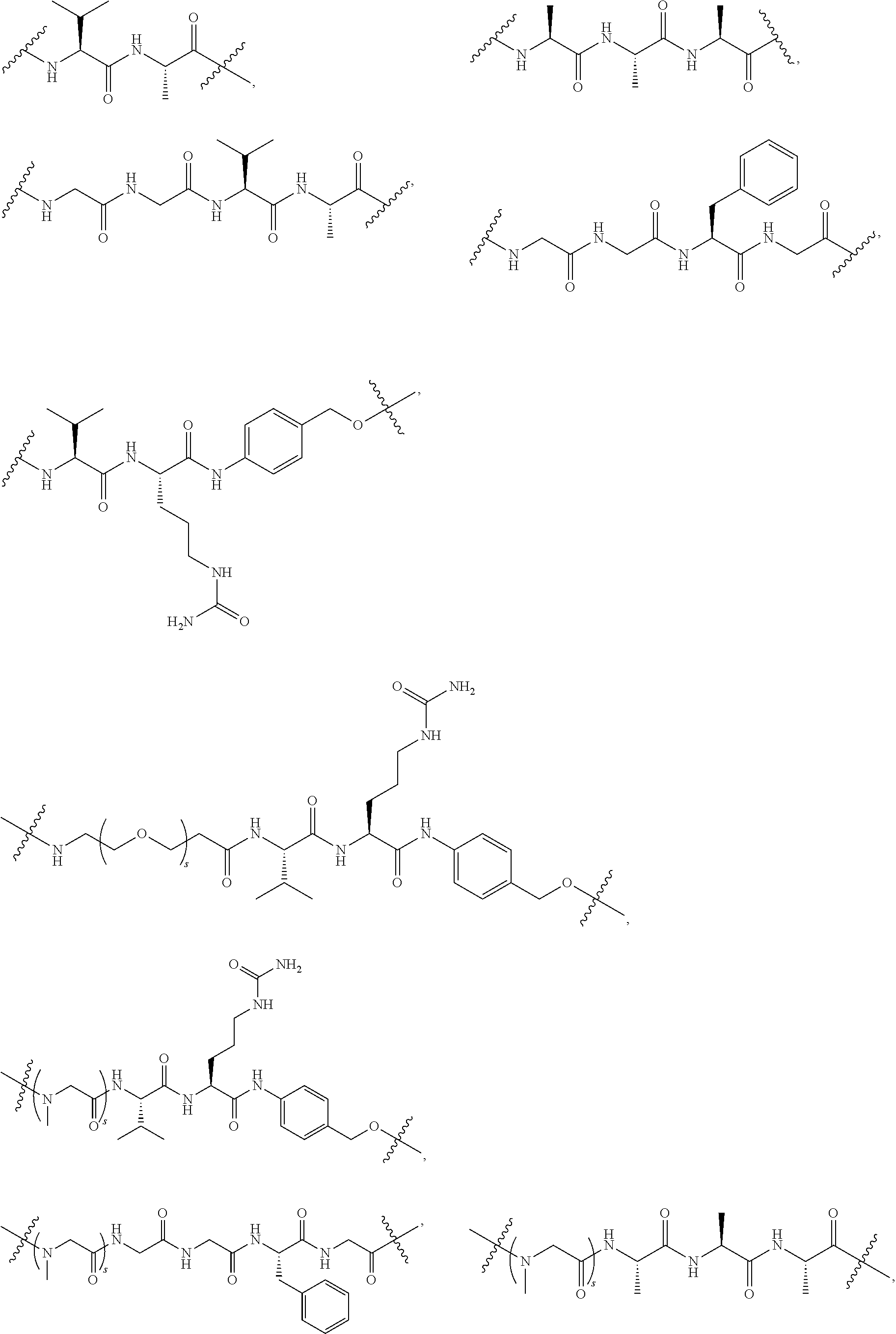

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments, L is selected from the group consisting of the following substituted or unsubstituted structural fragments:

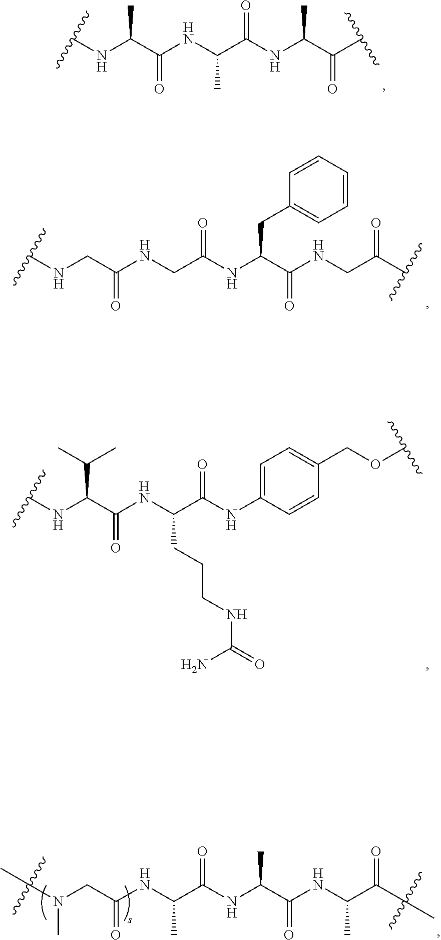

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments, E is a single bond, substituted or unsubstituted —NH—CH₂—, or selected from the group consisting of the following substituted or unsubstituted structural fragments:

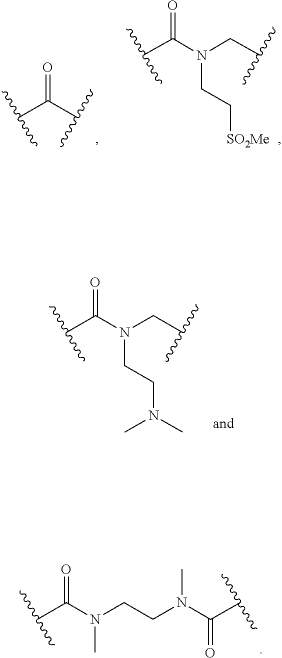

In some embodiments, E is a single bond, substituted or unsubstituted —NH—CH₂— or

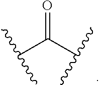 [SMILES: *C(*)=O]

In some embodiments, E is a substituted or unsubstituted —NH—CH₂— or

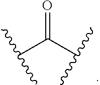 [SMILES: *C(*)=O]

In some embodiments, E is a single bond or

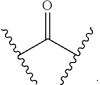 [SMILES: *C(*)=O]

In some embodiments,

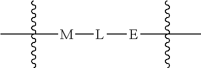

is selected from the group consisting of the following substituted or unsubstituted structures:

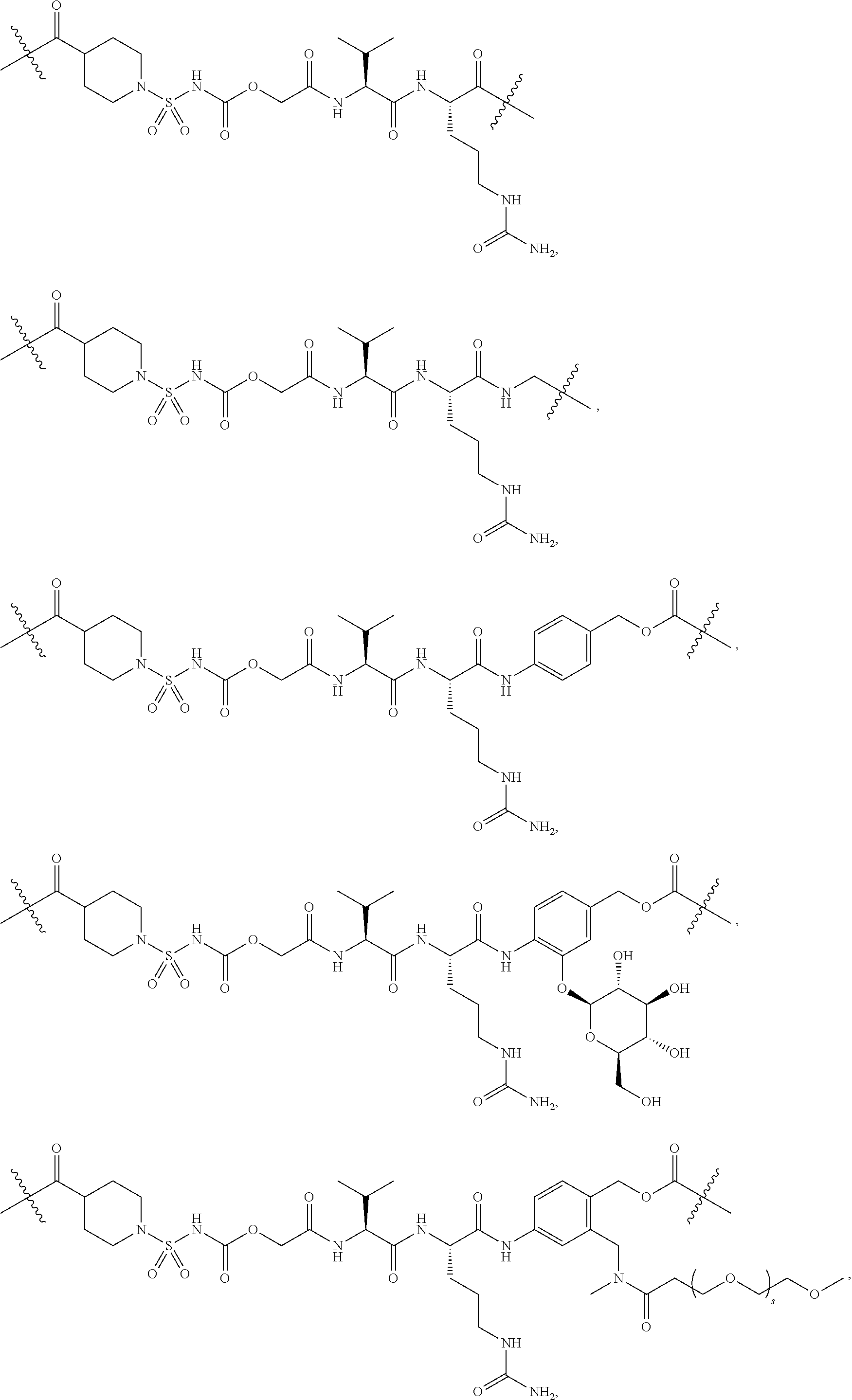

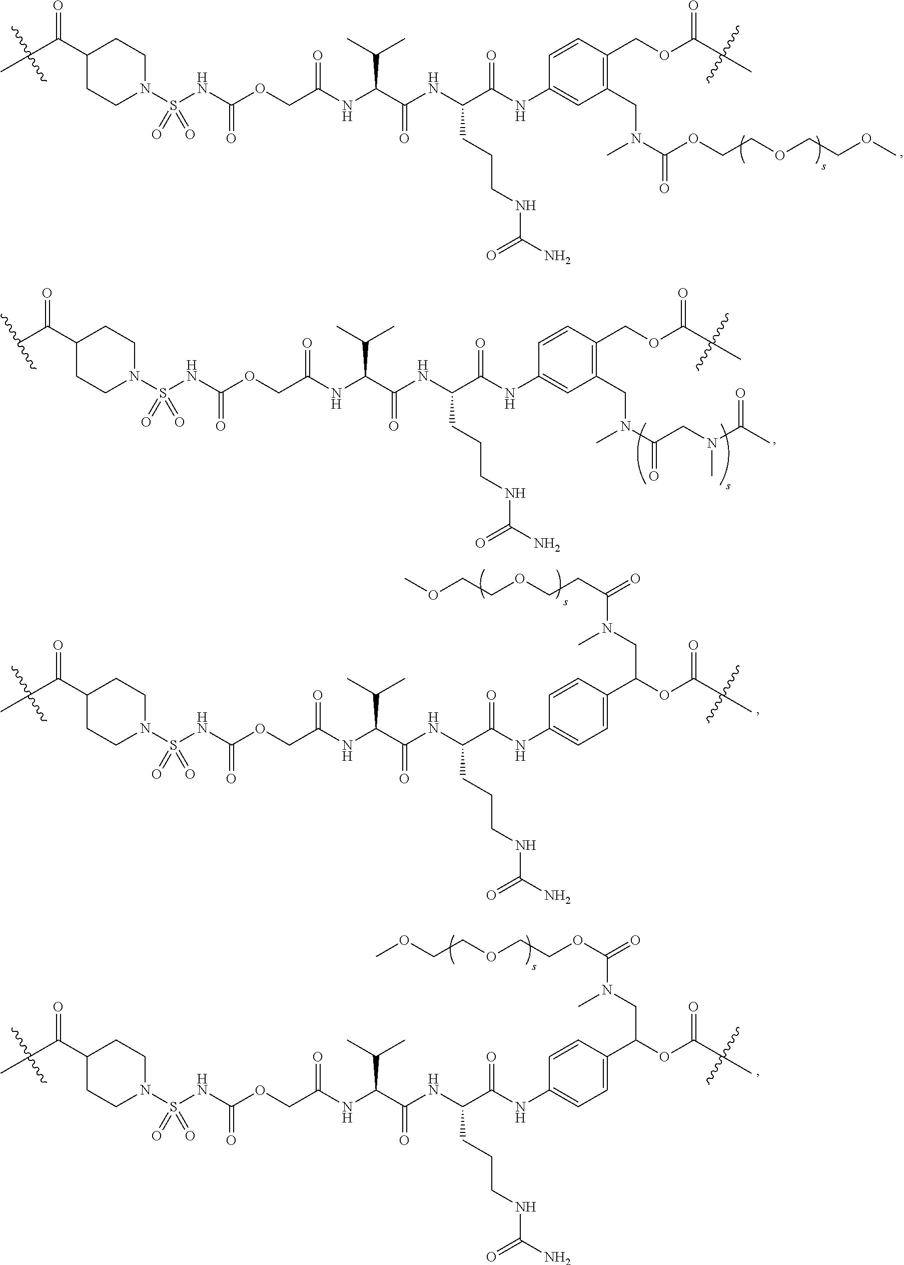

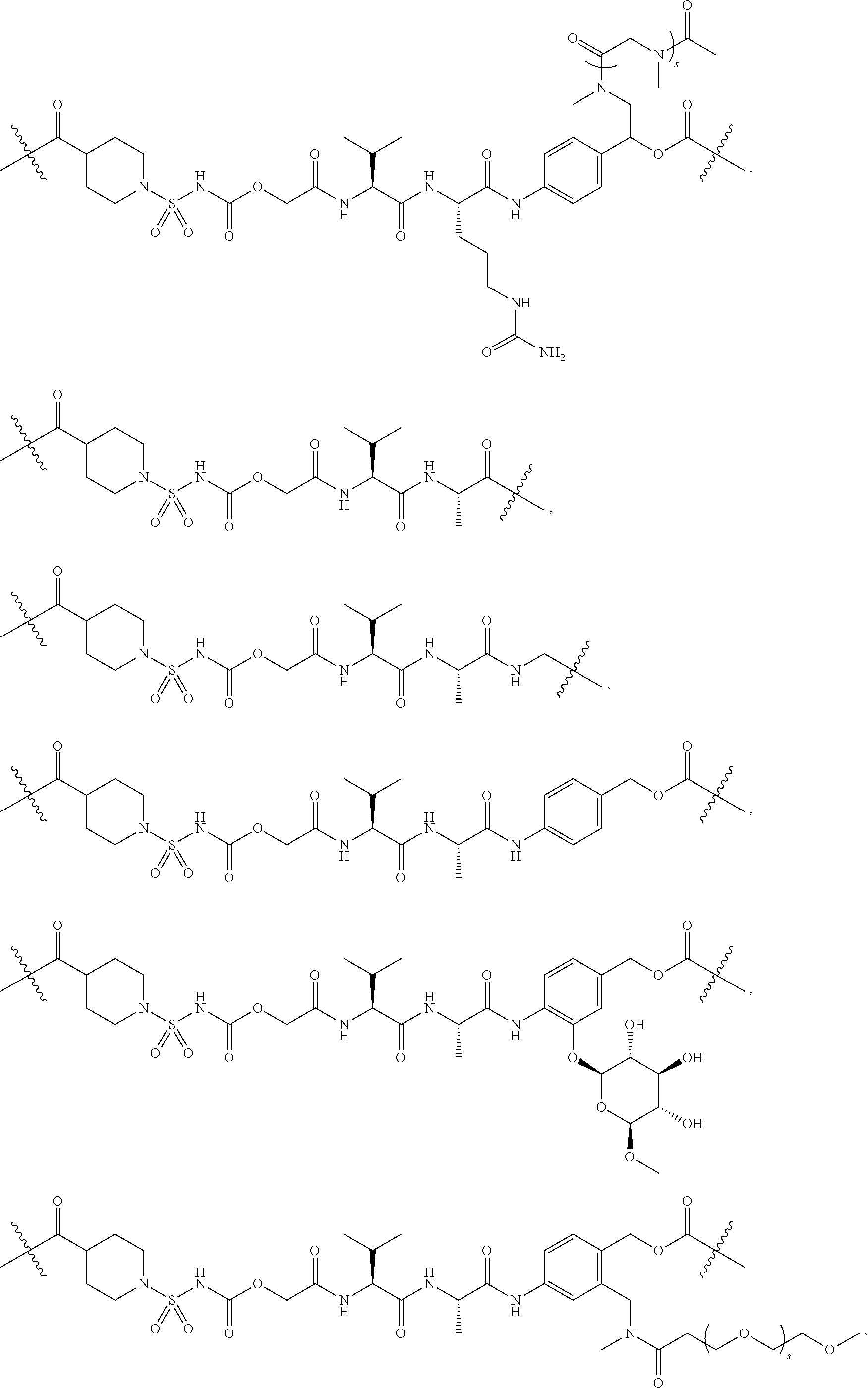

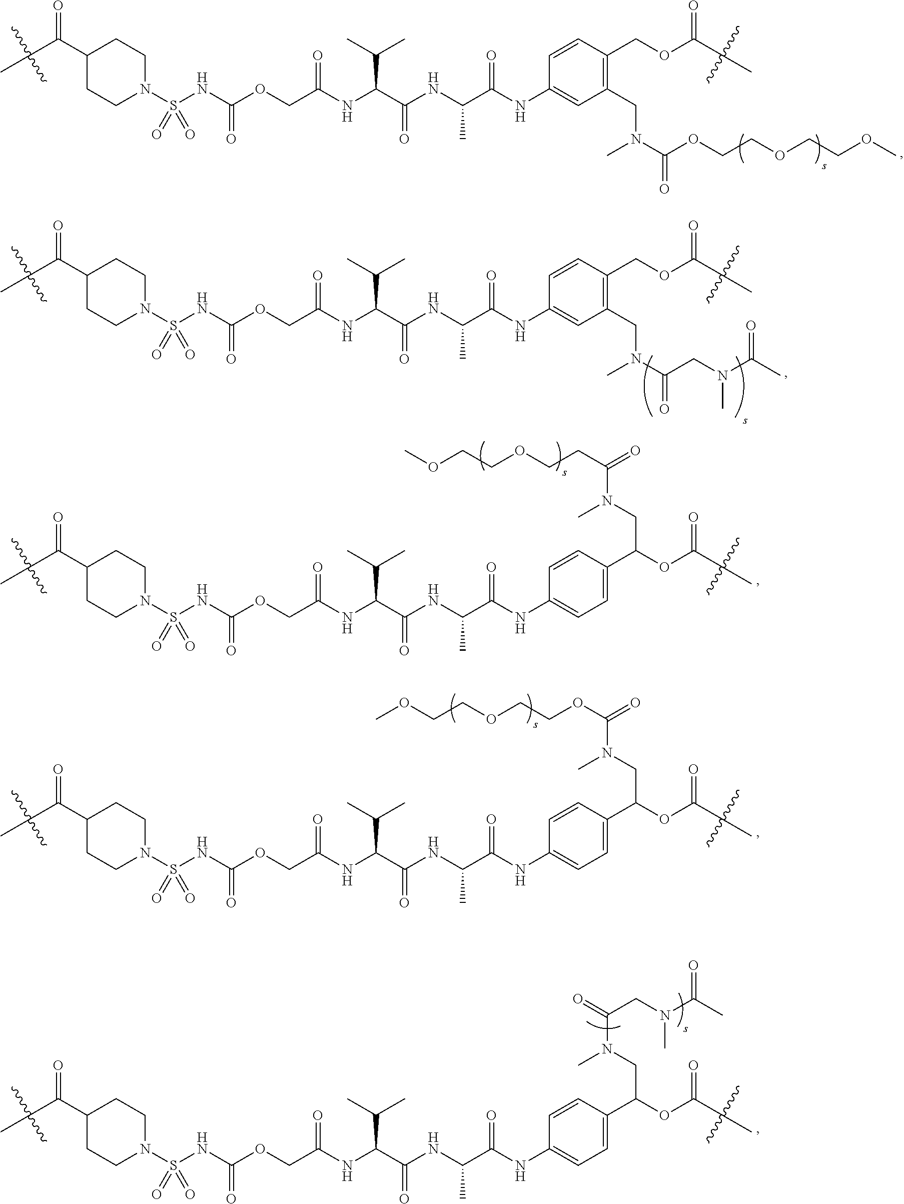

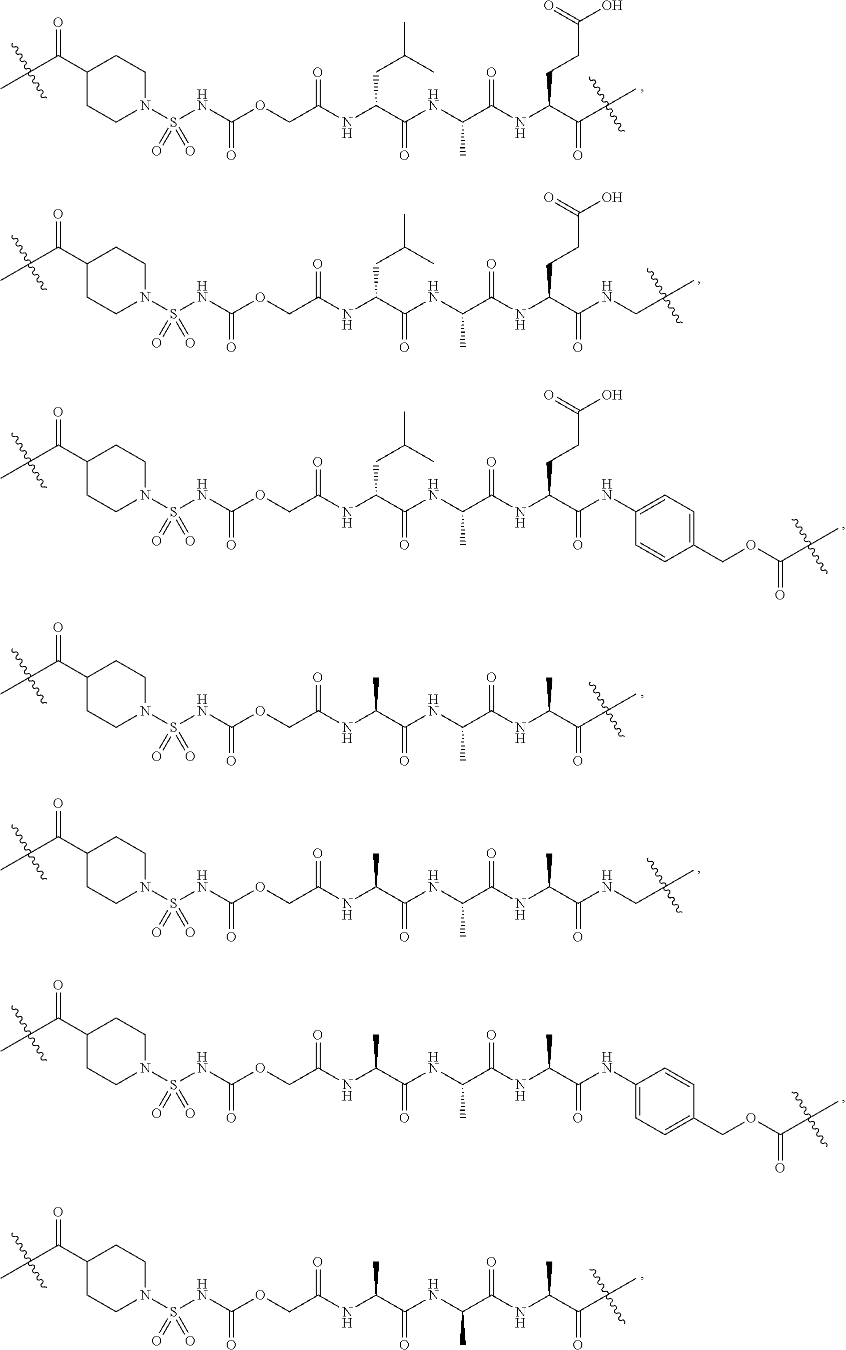

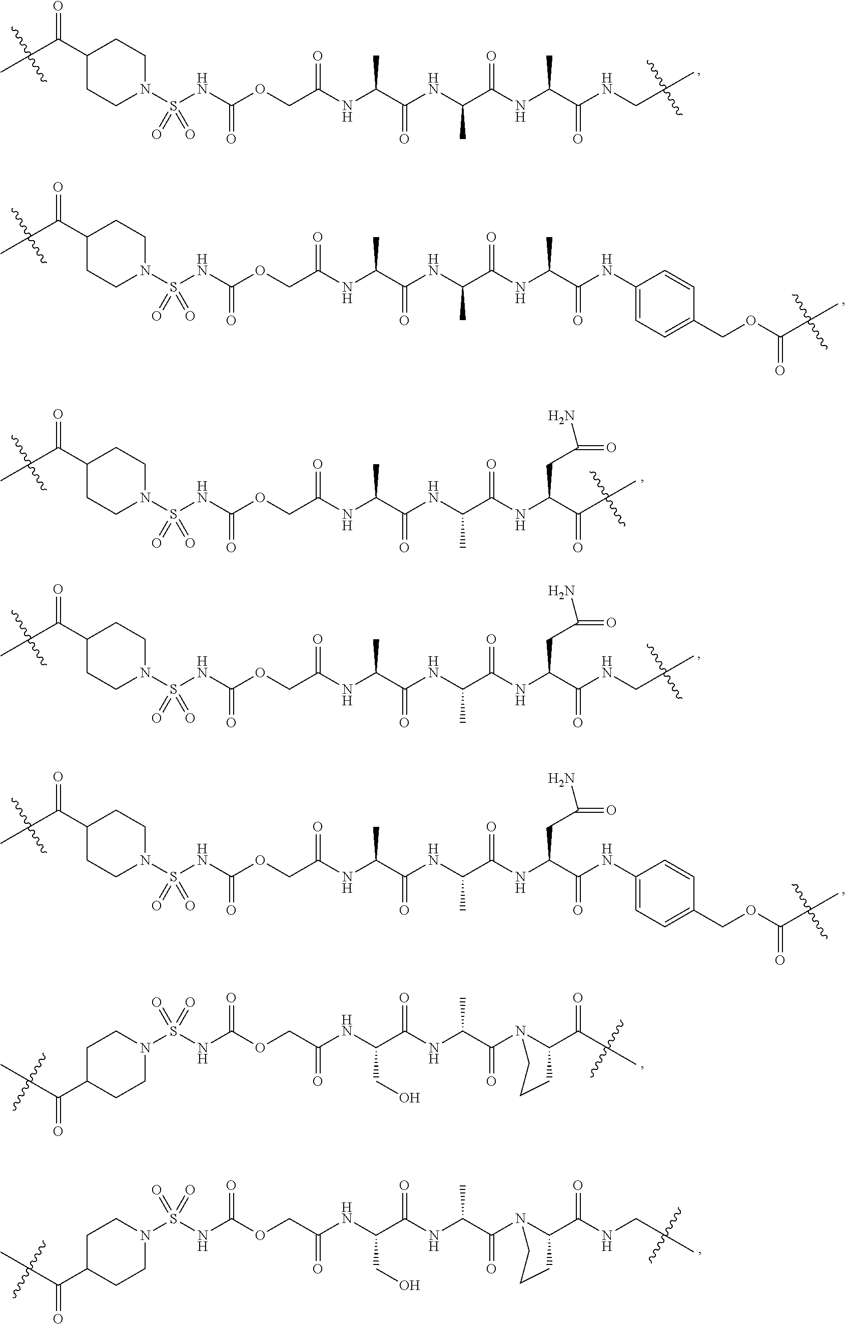

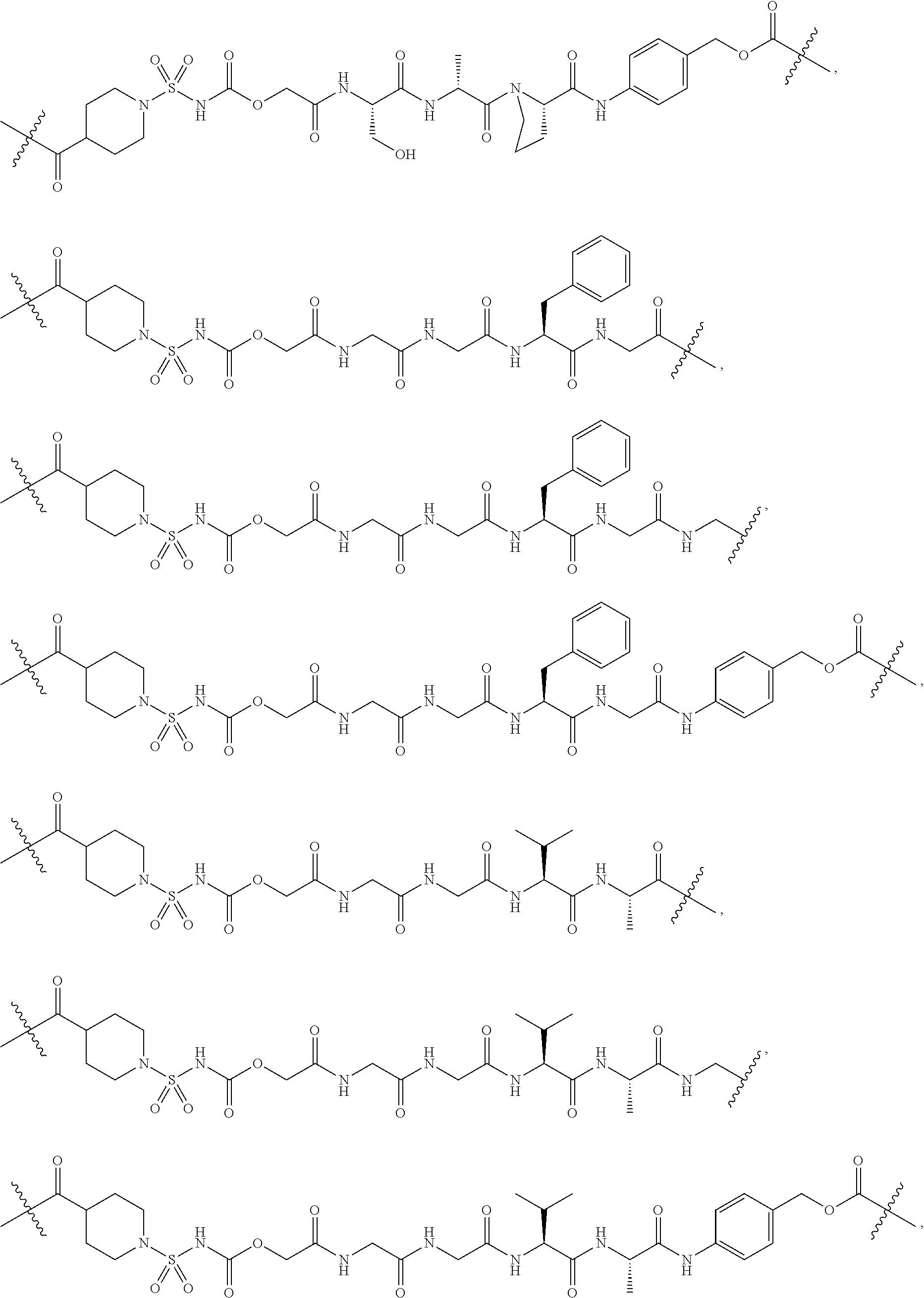

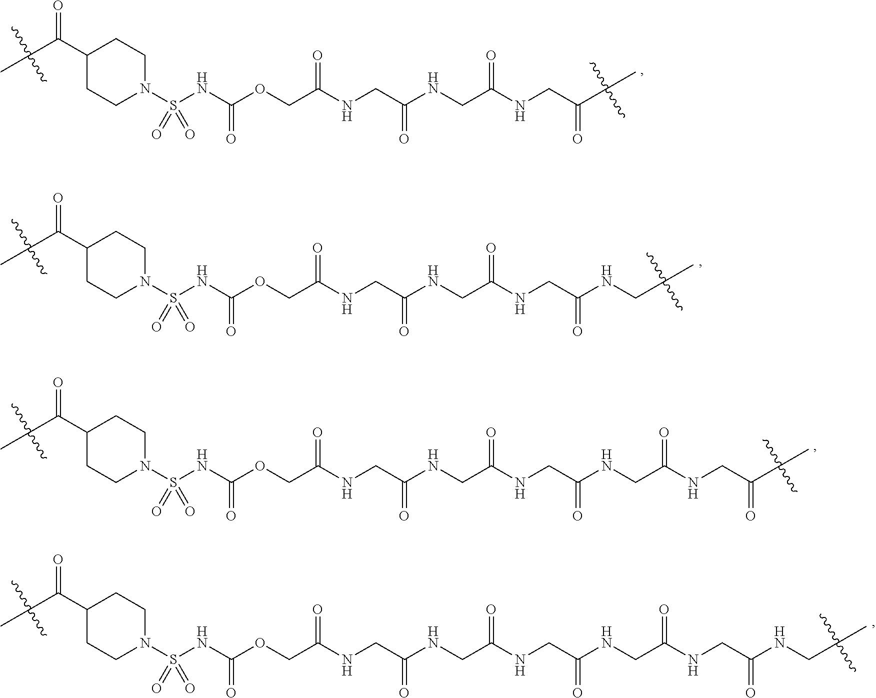 [SMILES: *C(=O)CNC(=O)CNC(=O)CNC(=O)COC(=O)NS(=O)(=O)N1CCC(C(*)=O)CC1 ; *CNC(=O)CNC(=O)CNC(=O)CNC(=O)COC(=O)NS(=O)(=O)N1CCC(C(*)=O)CC1 ; *C(=O)CNC(=O)CNC(=O)CNC(=O)CNC(=O)CNC(=O)COC(=O)NS(=O)(=O)N1CCC(C(*)=O)CC1 ; *CNC(=O)CNC(=O)CNC(=O)CNC(=O)CNC(=O)CNC(=O)COC(=O)NS(=O)(=O)N1CCC(C(*)=O)CC1]

s is an integer selected from 1 to 20.

In some embodiments,

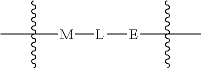

is selected from the group consisting of the following substituted or unsubstituted structures:

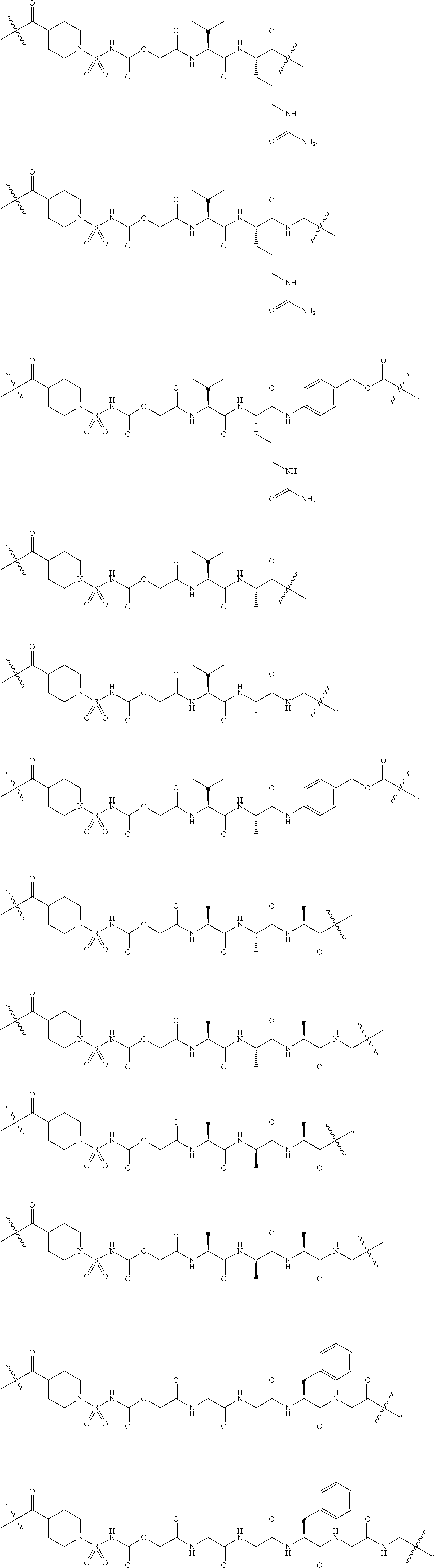

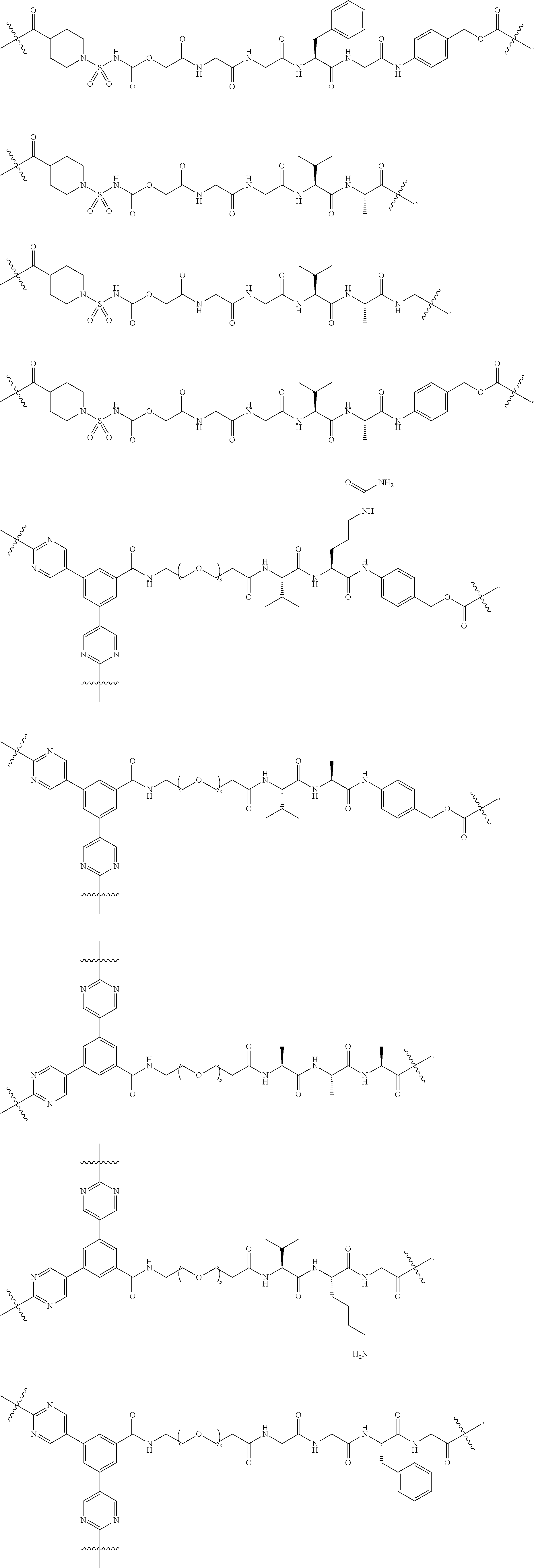

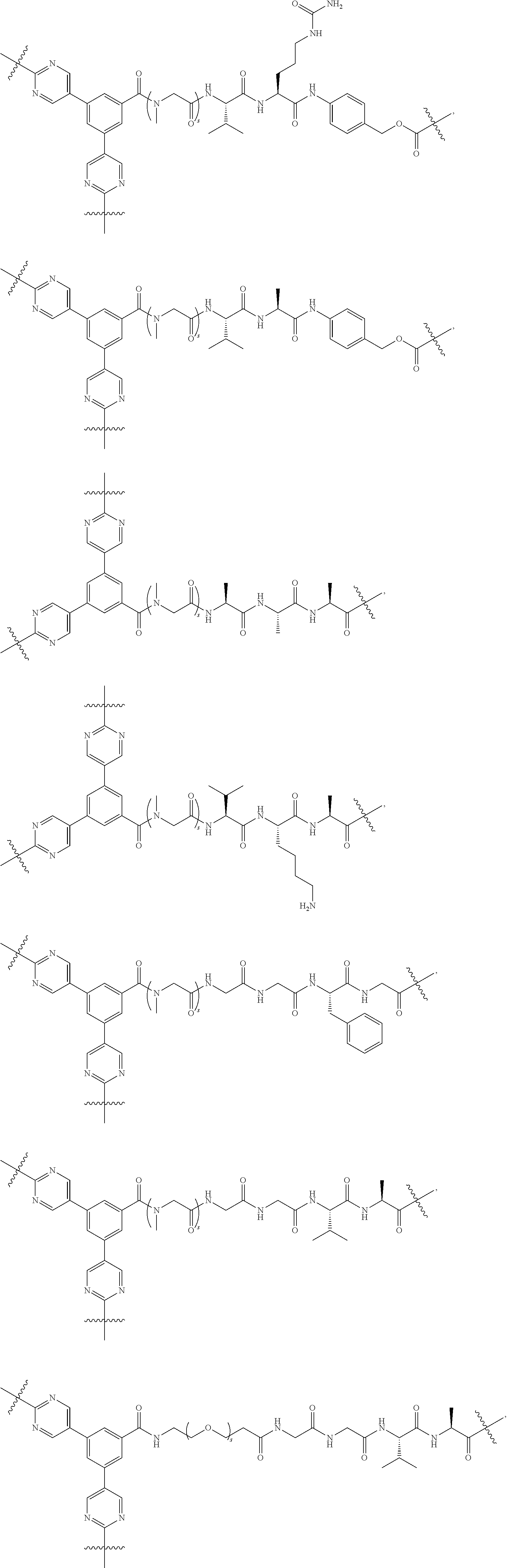

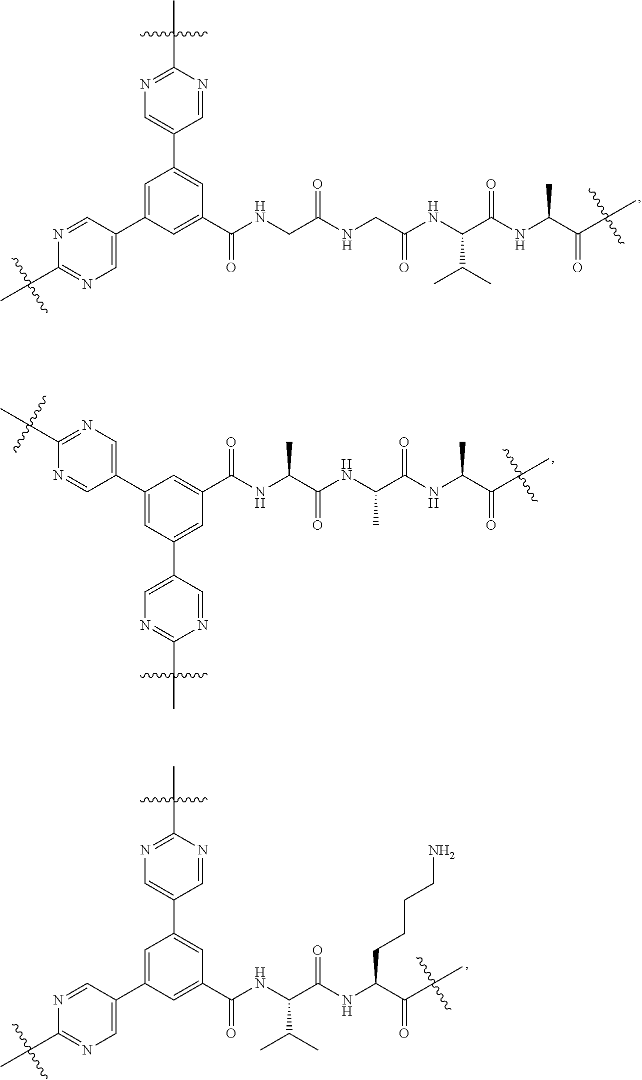 [SMILES: *C(=O)[C@H](C)NC(=O)[C@@H](NC(=O)CNC(=O)CNC(=O)c1cc(-c2cnc(*)nc2)cc(-c2cnc(*)nc2)c1)C(C)C ; *C(=O)[C@H](C)NC(=O)[C@H](C)NC(=O)[C@H](C)NC(=O)c1cc(-c2cnc(*)nc2)cc(-c2cnc(*)nc2)c1 ; *C(=O)[C@H](CCCCN)NC(=O)[C@@H](NC(=O)c1cc(-c2cnc(*)nc2)cc(-c2cnc(*)nc2)c1)C(C)C]

s is an integer selected from 1 to 20.

In some embodiments,

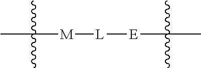

is selected from the group consisting of the following substituted or unsubstituted structures:

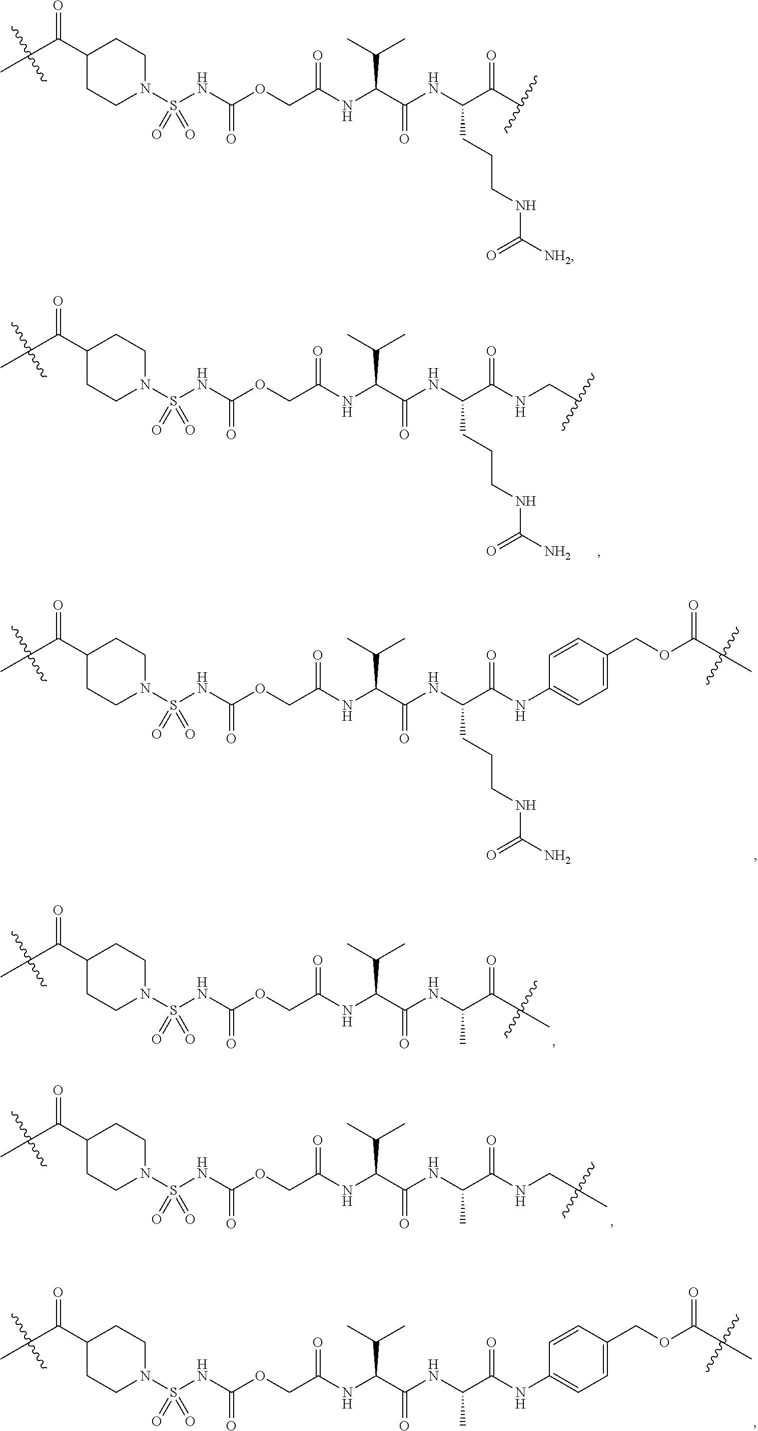

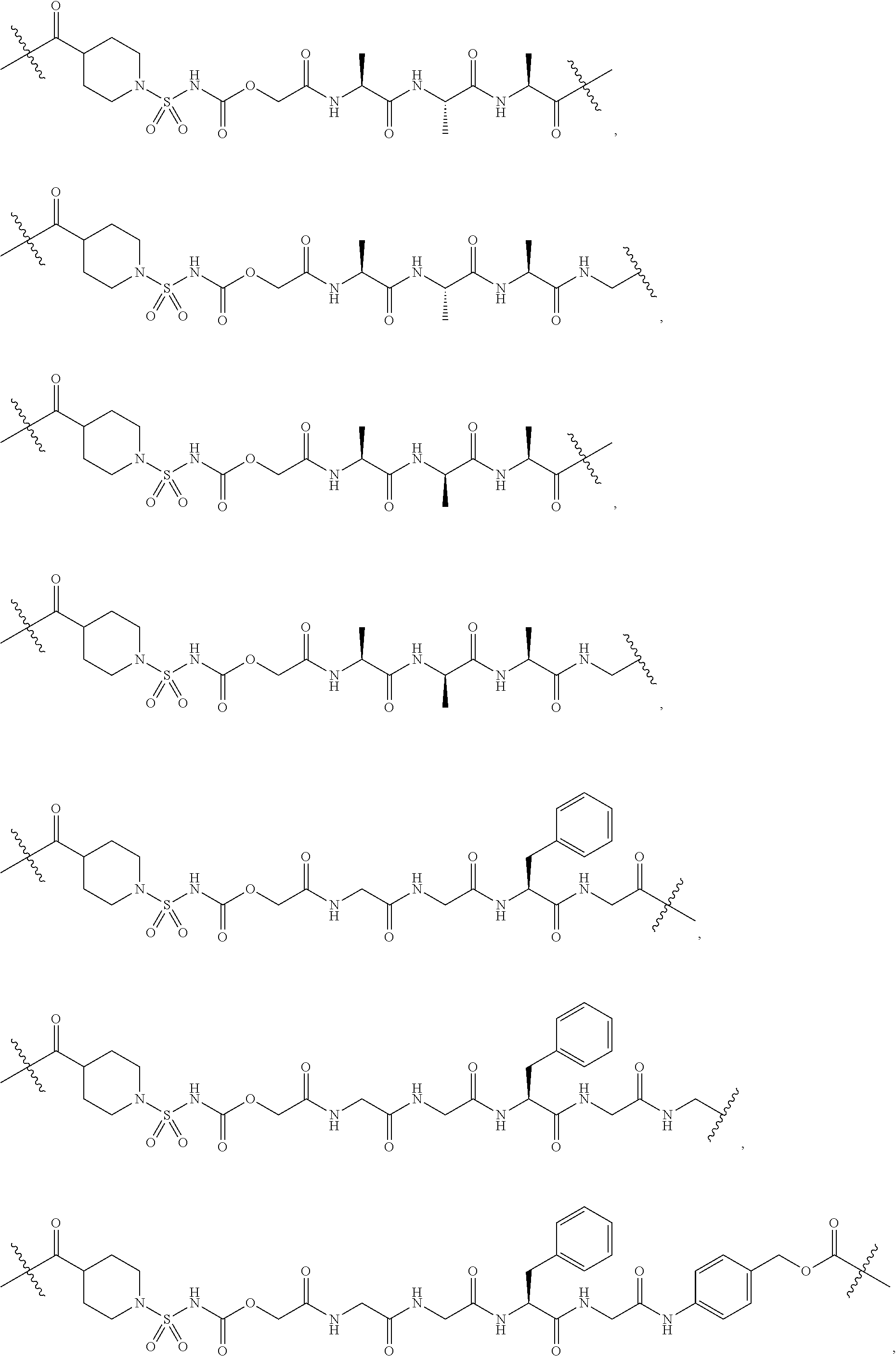

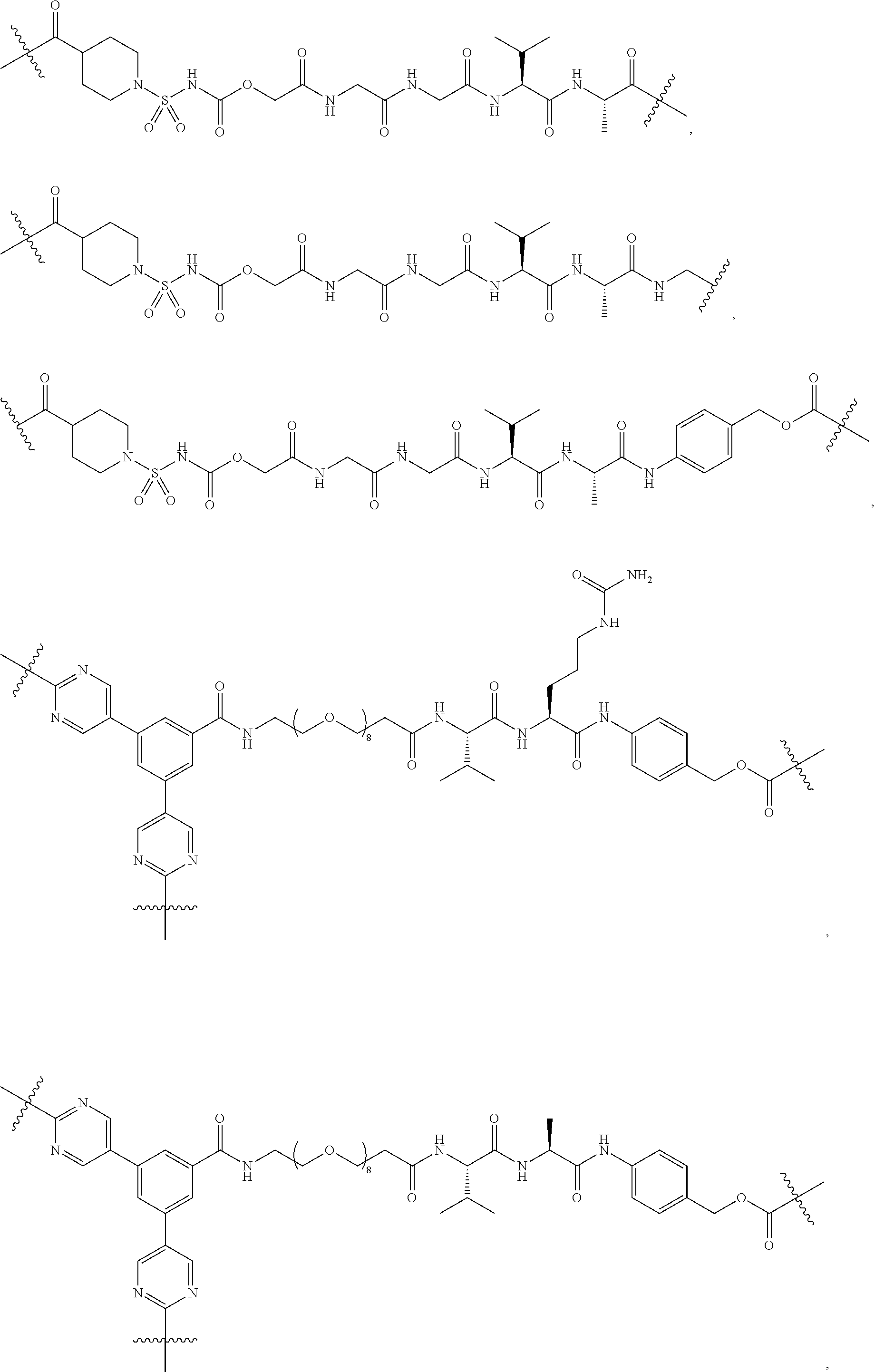

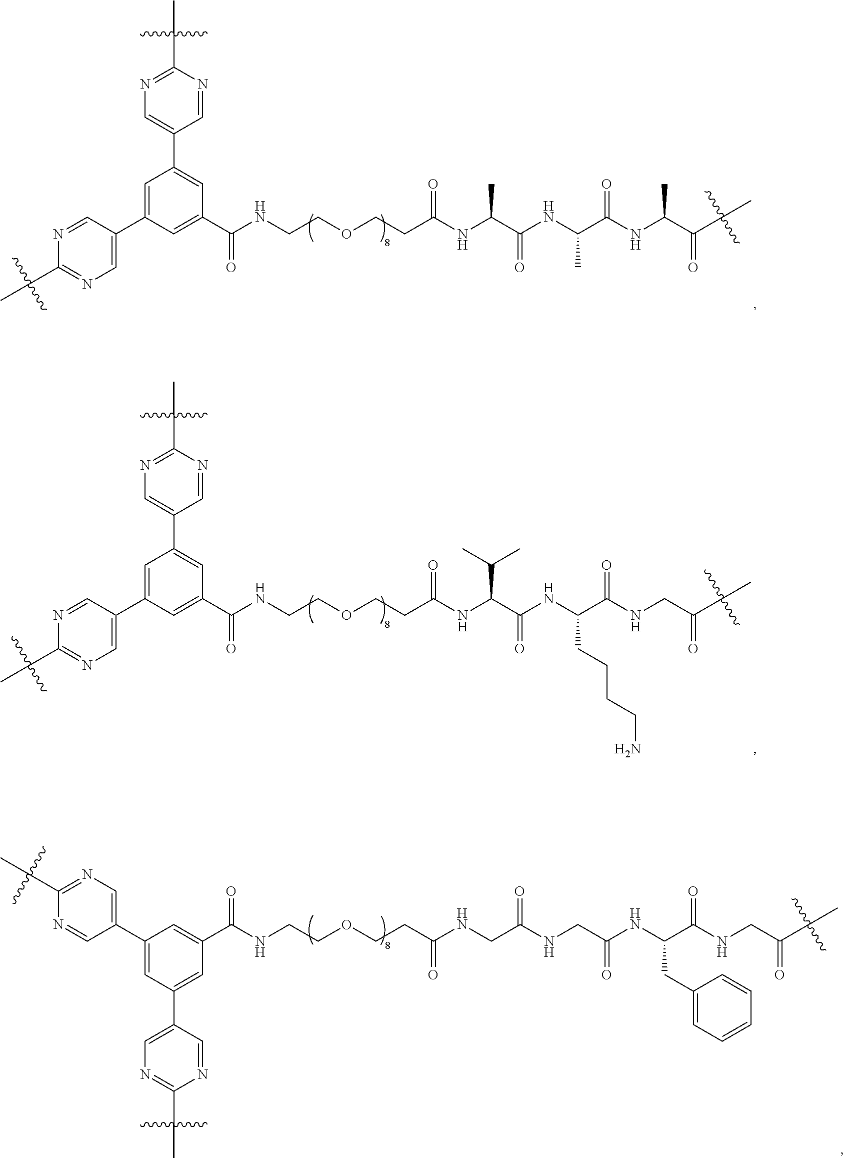

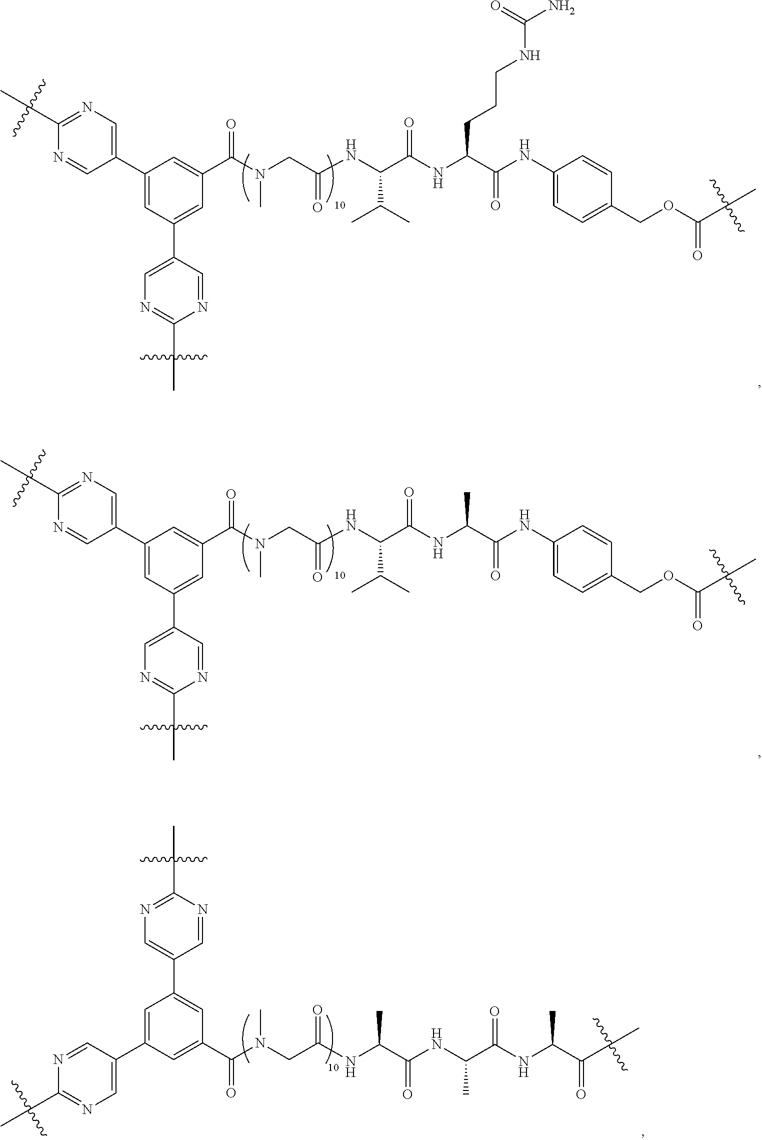

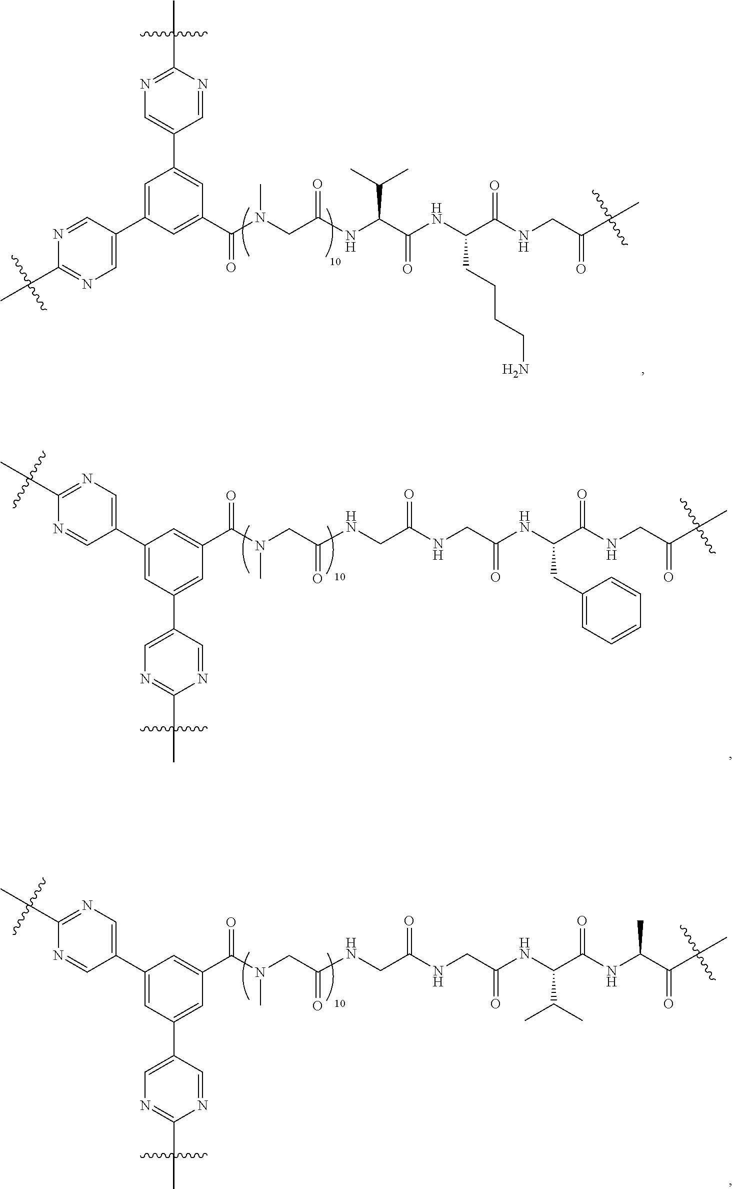

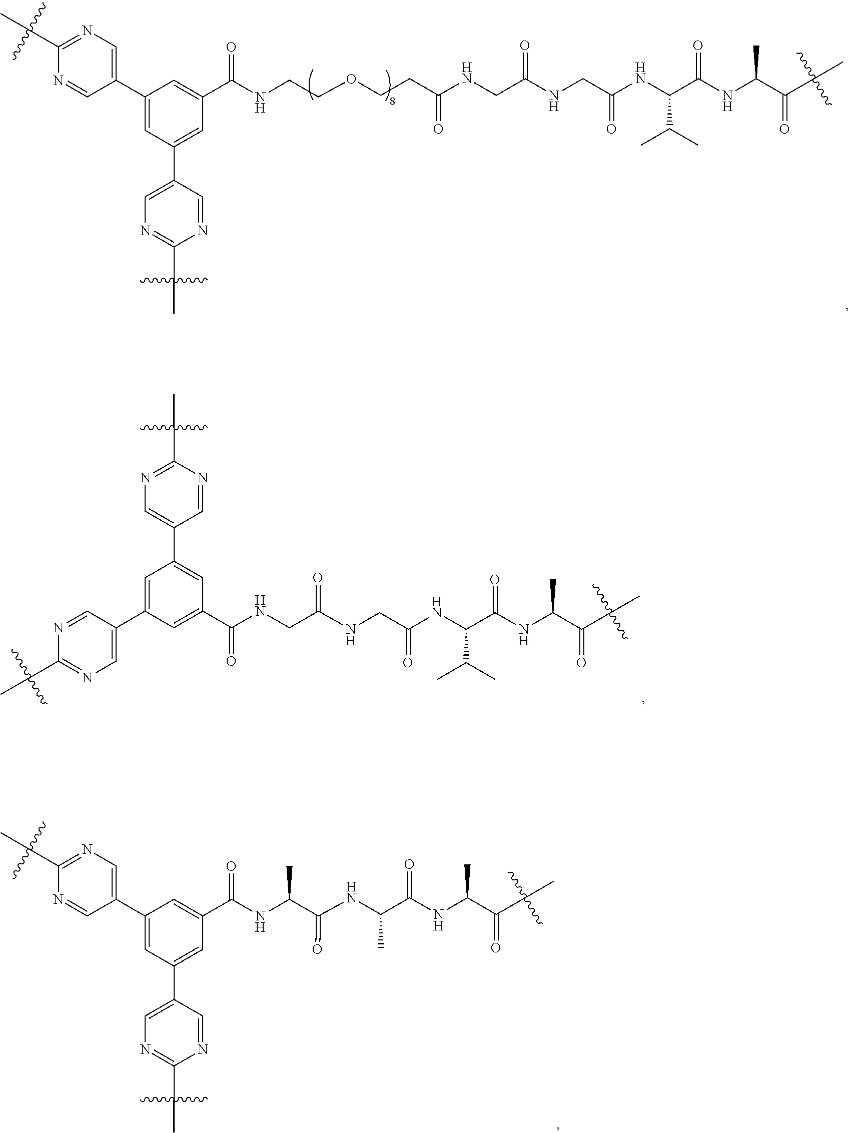

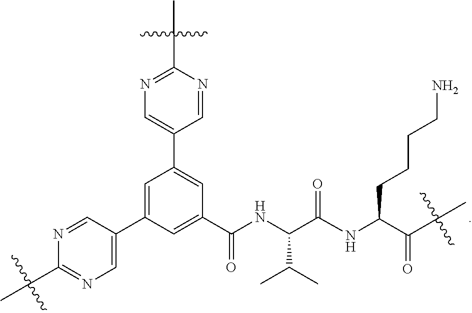 [SMILES: *C(=O)[C@H](CCCCN)NC(=O)[C@@H](NC(=O)c1cc(-c2cnc(*)nc2)cc(-c2cnc(*)nc2)c1)C(C)C]

In some embodiments,

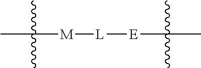

is selected from the following substituted or unsubstituted structures:

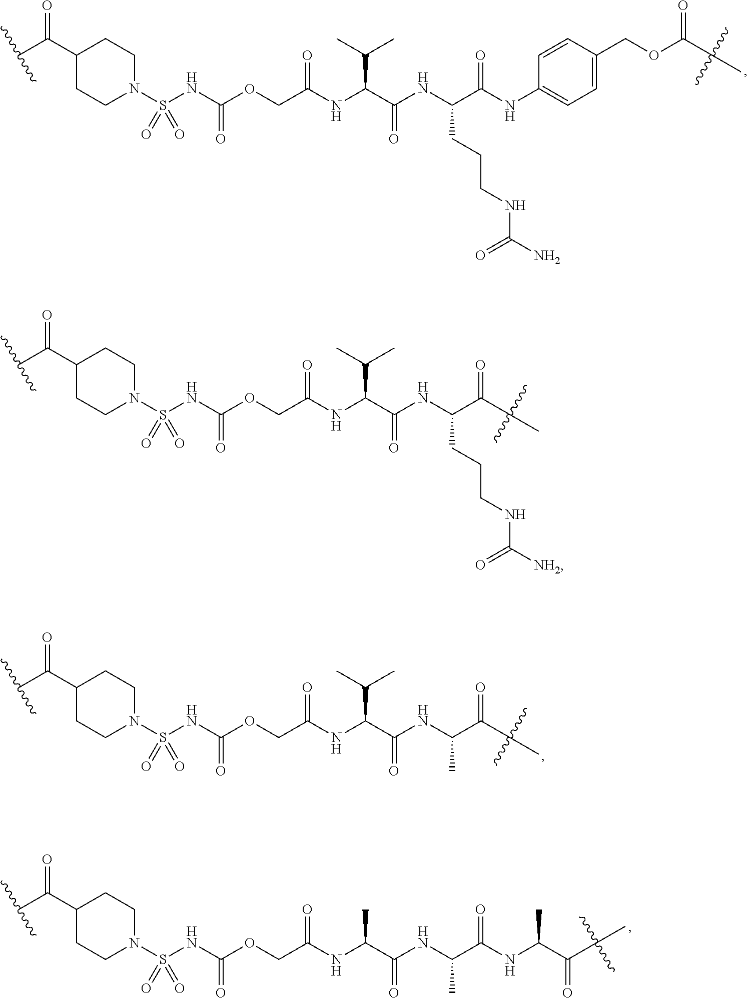

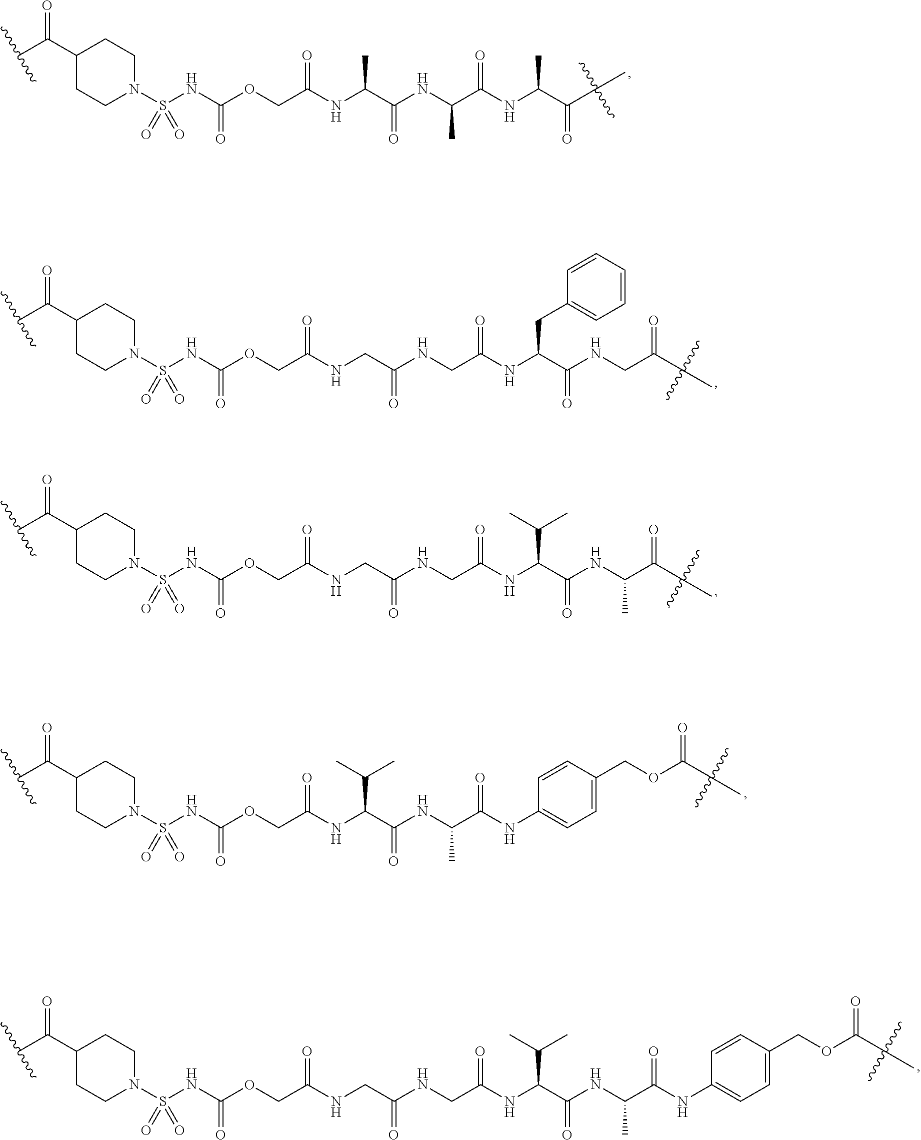

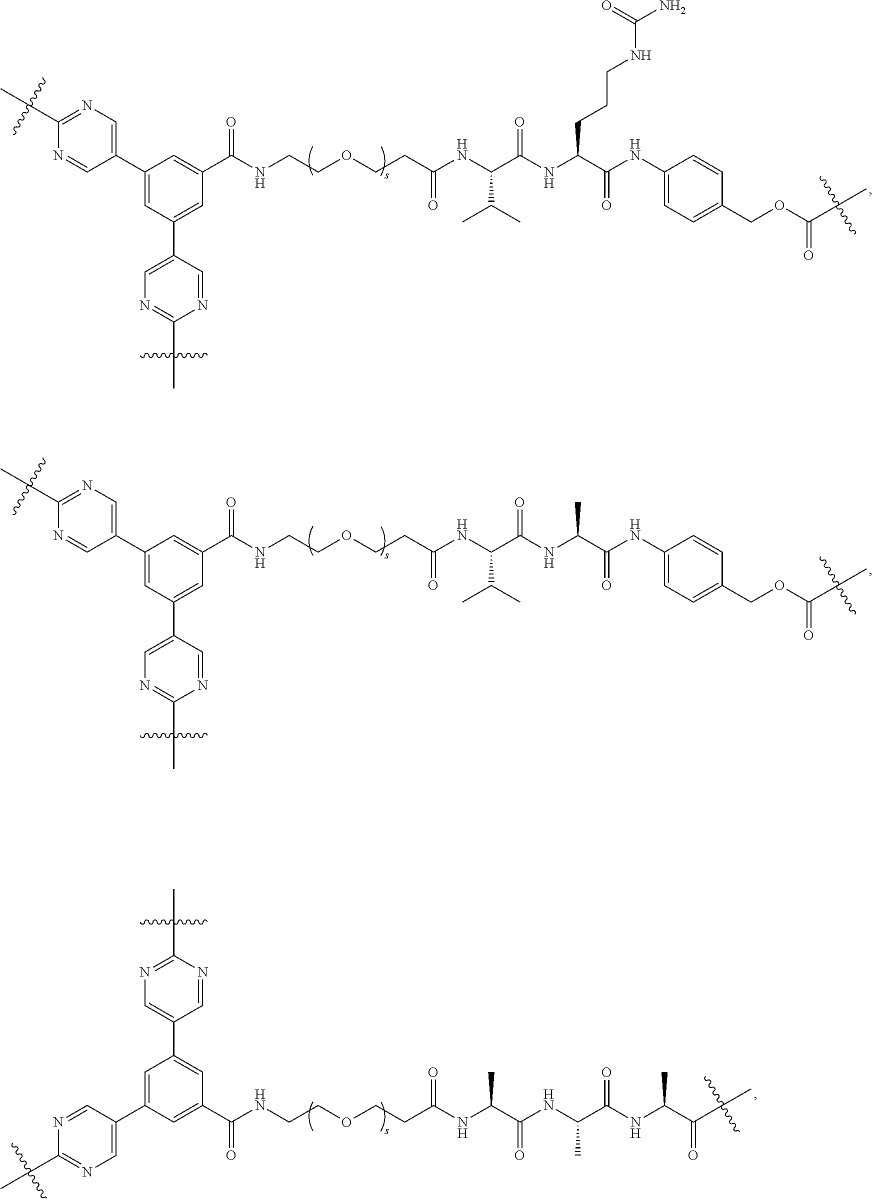

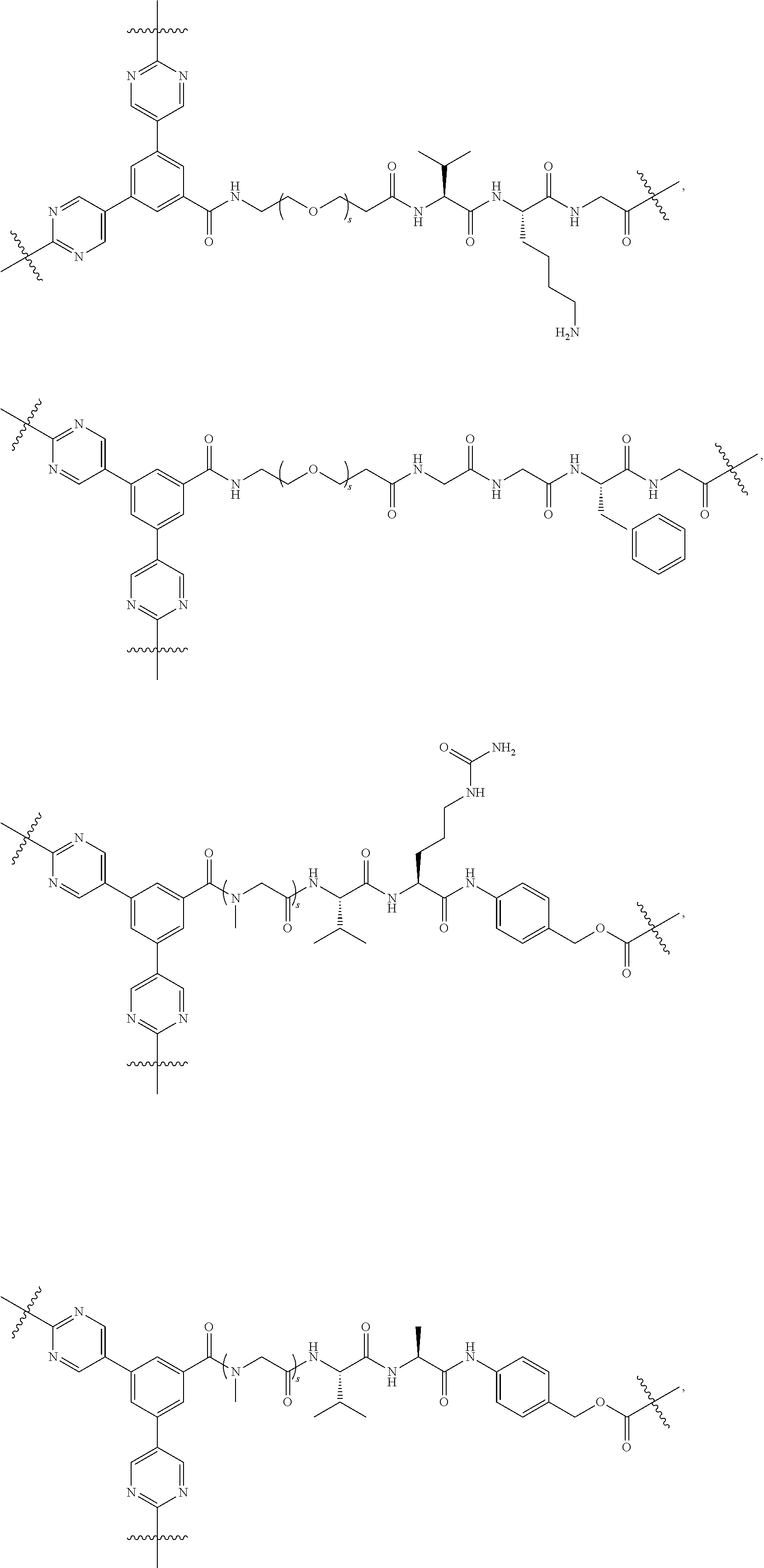

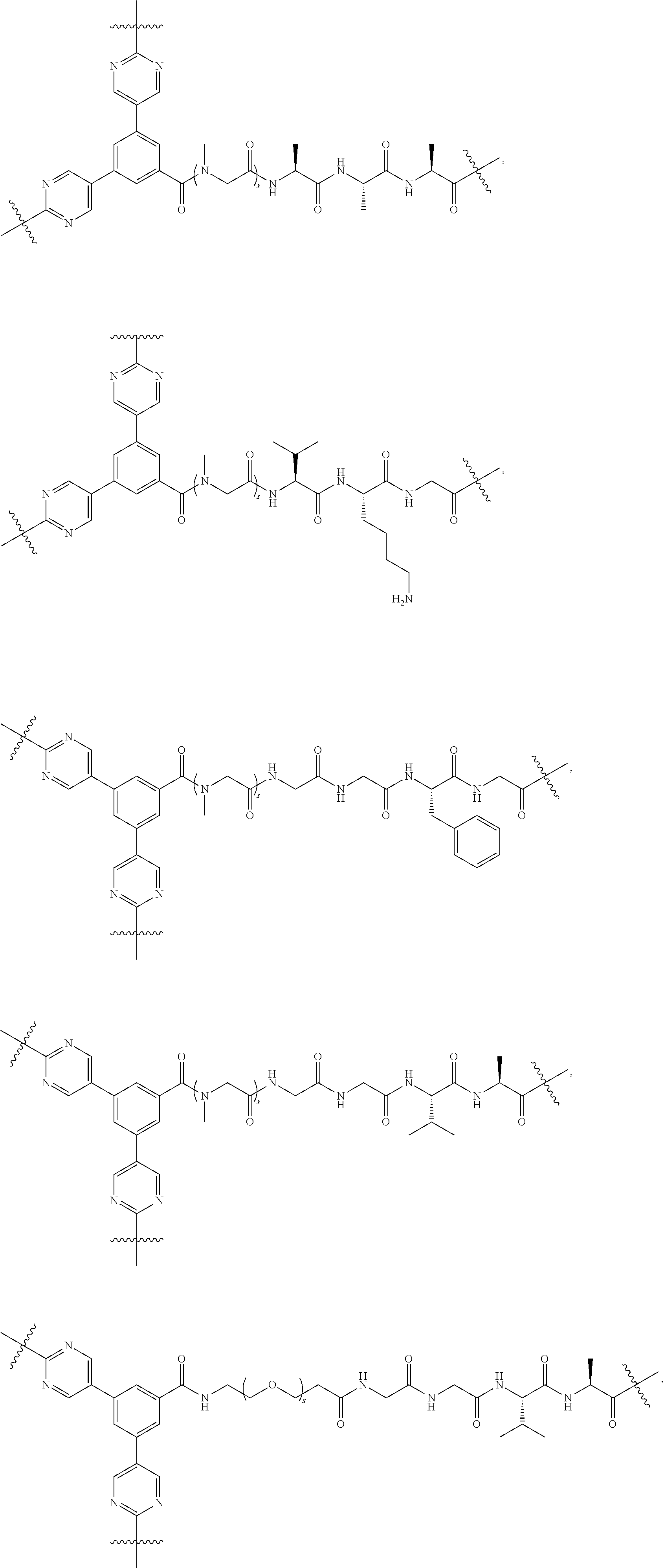

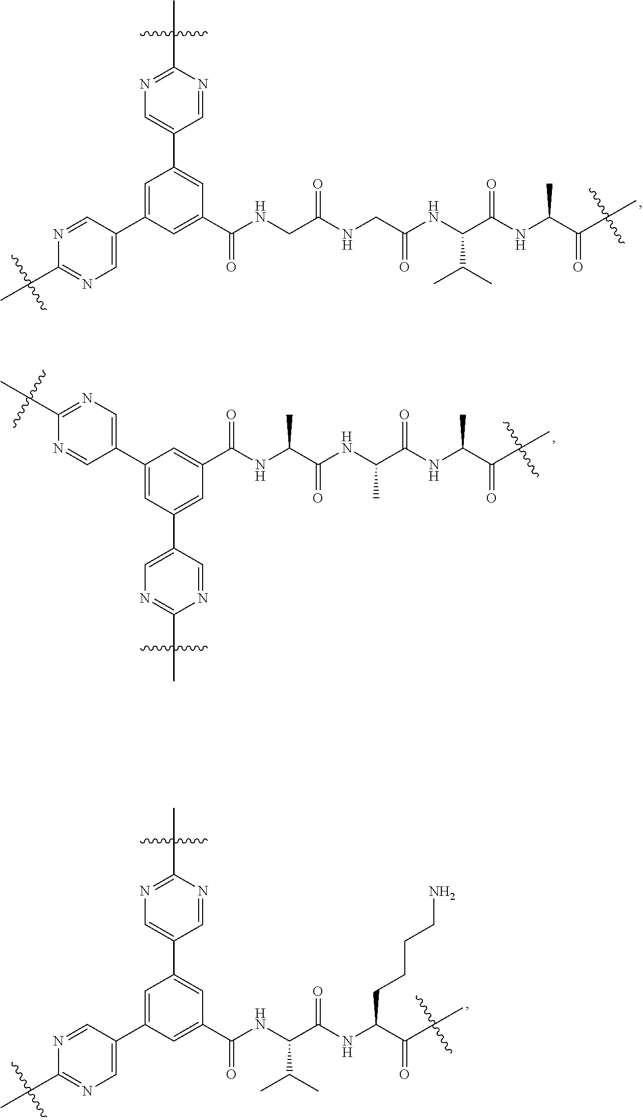

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments,

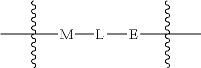

is selected from the group consisting of the following substituted or unsubstituted structures:

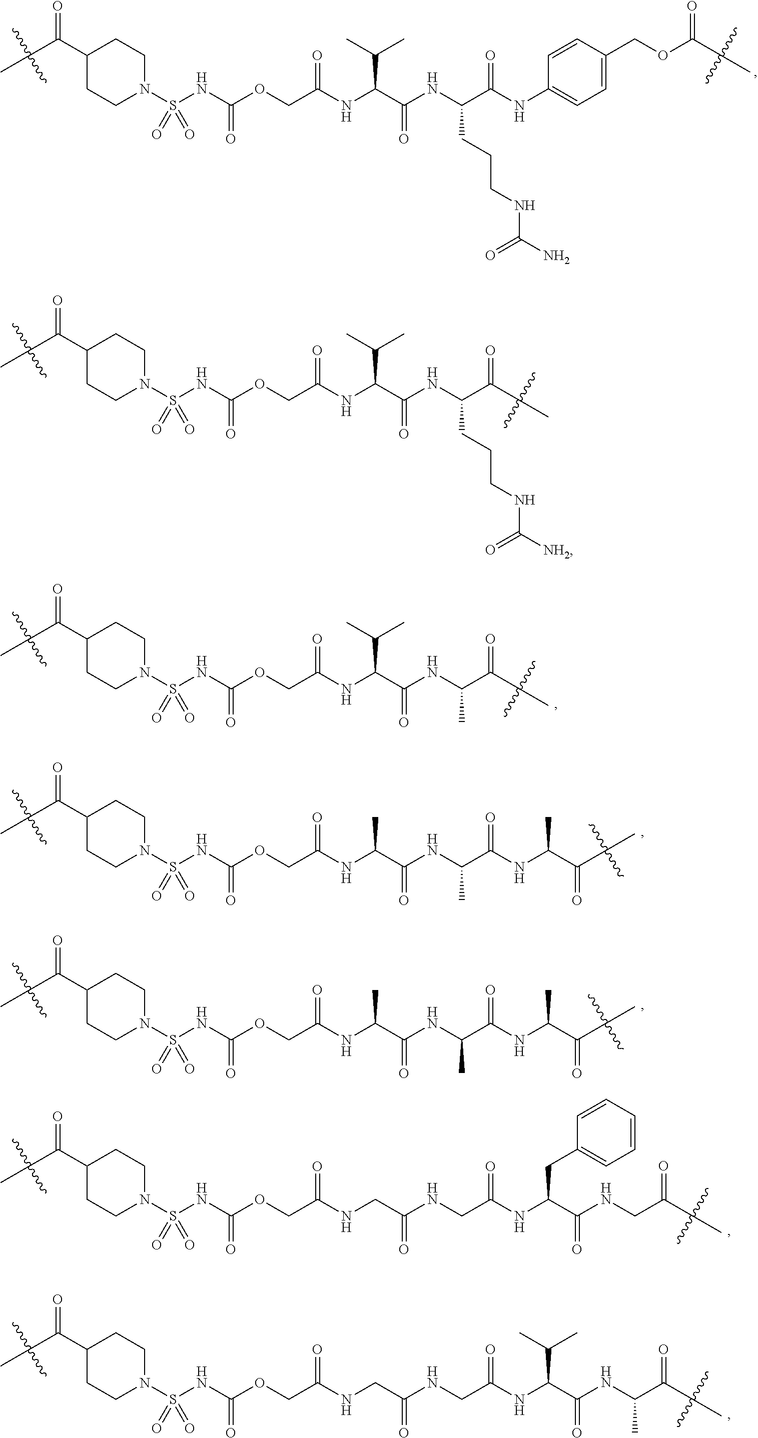

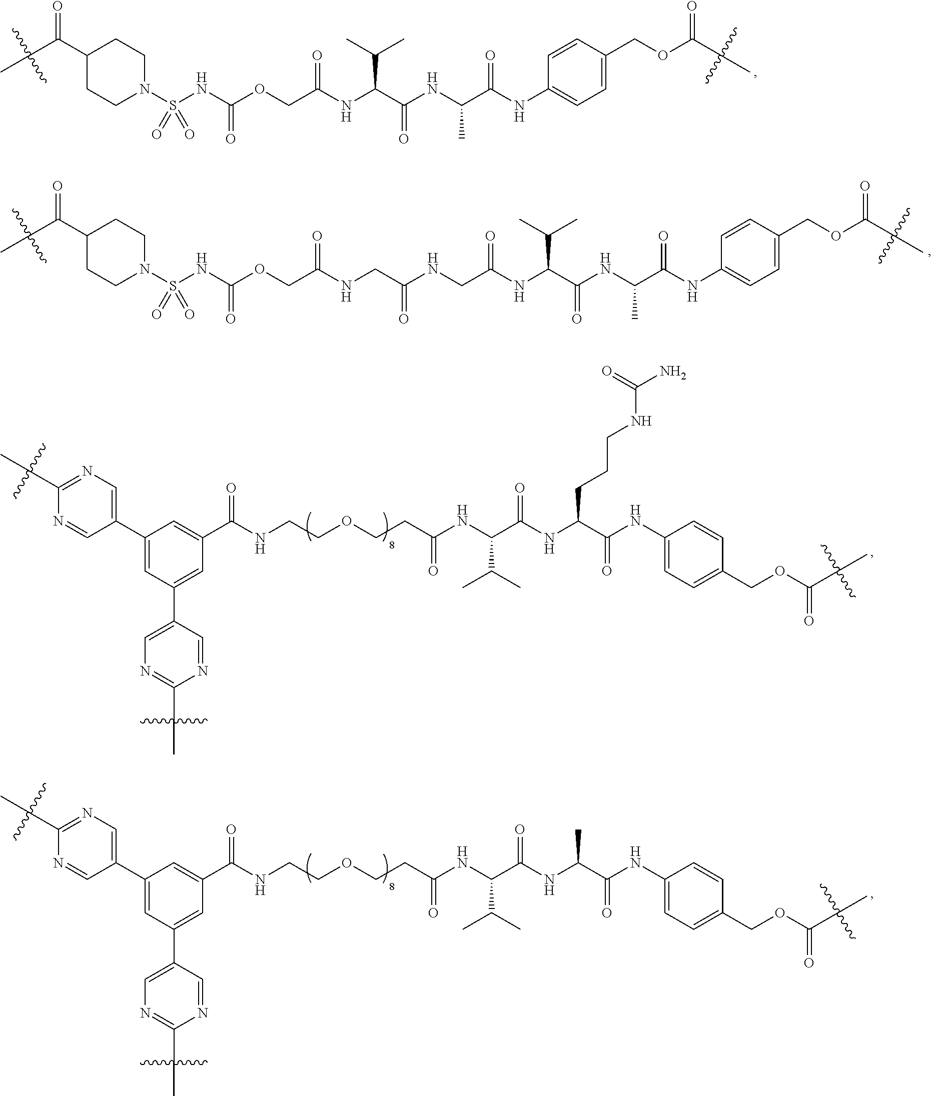

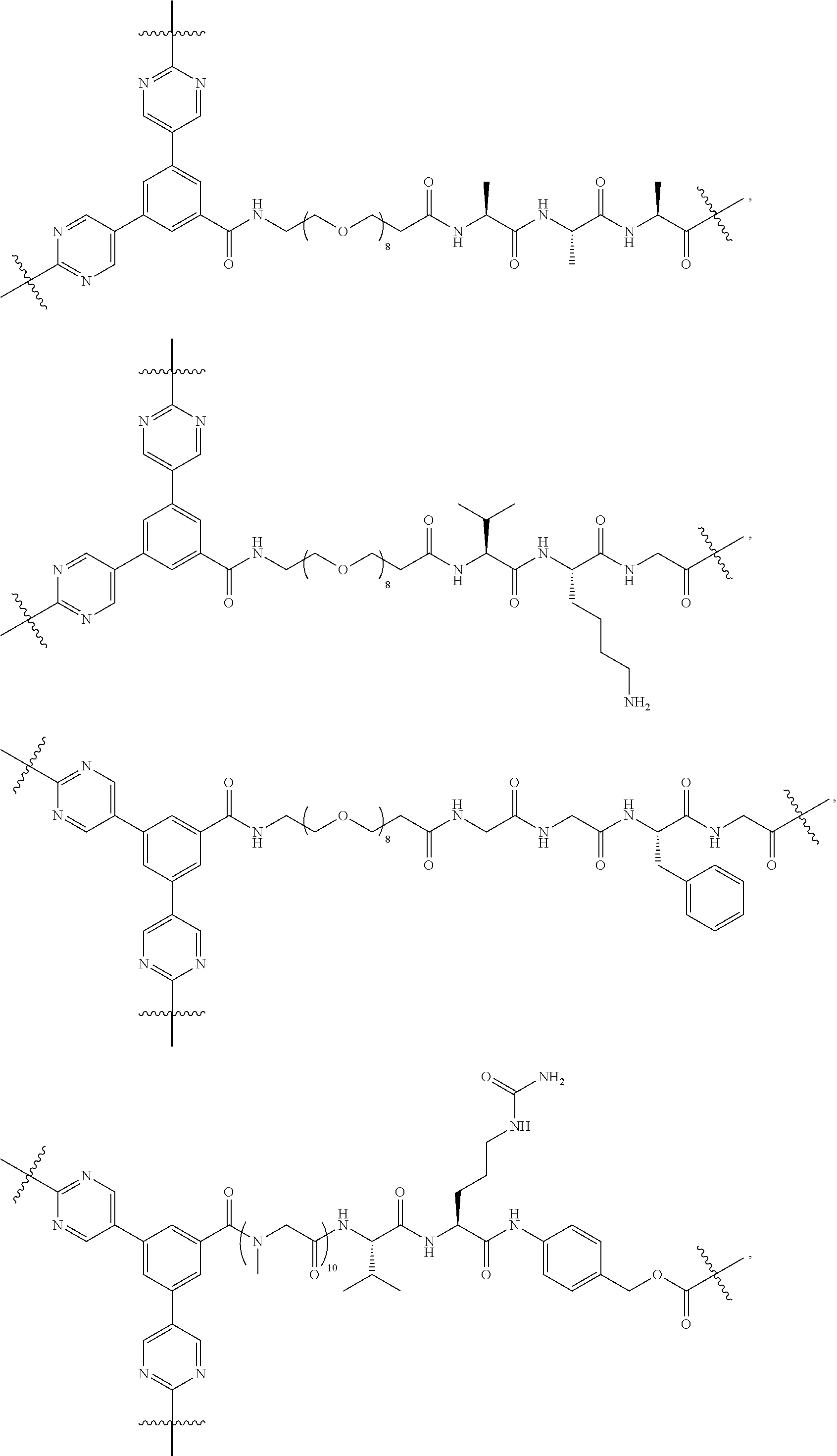

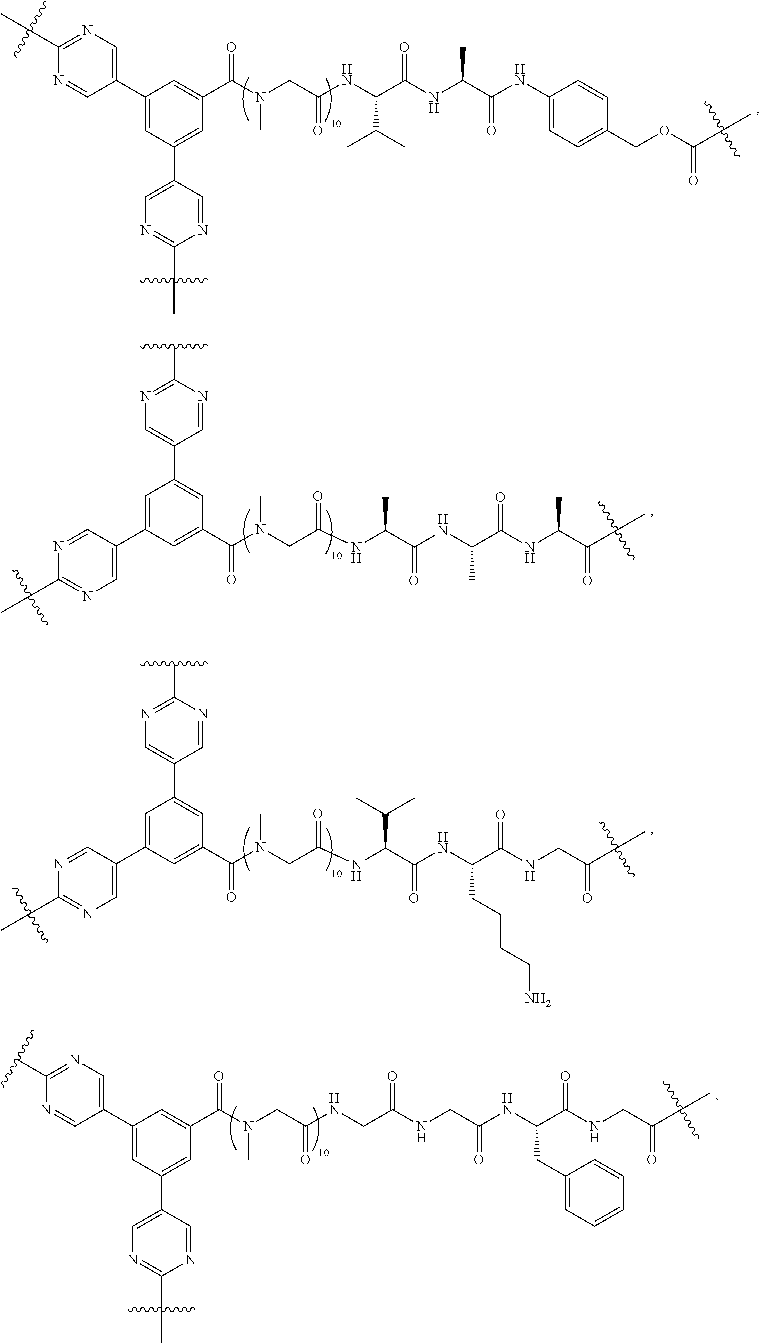

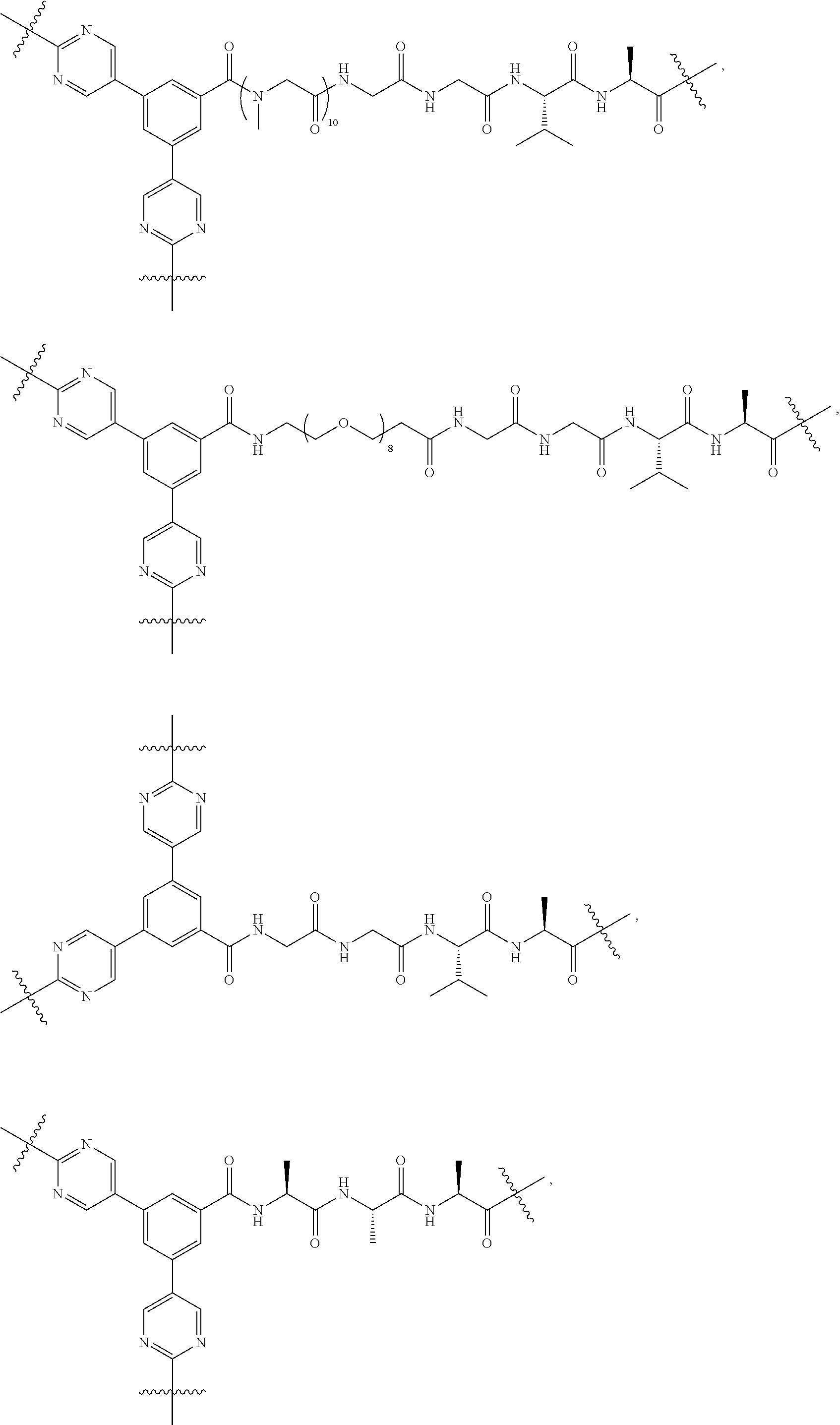

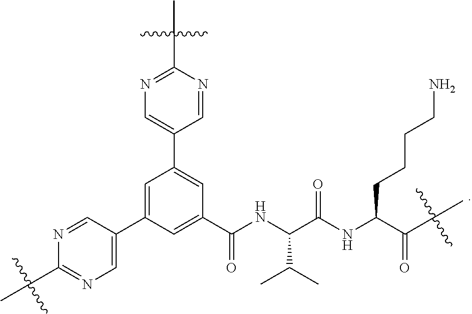 [SMILES: *C(=O)[C@H](CCCCN)NC(=O)[C@@H](NC(=O)c1cc(-c2cnc(*)nc2)cc(-c2cnc(*)nc2)c1)C(C)C]

In some embodiments,

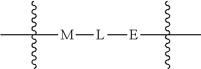

is selected from the group consisting of the following substituted or unsubstituted structures:

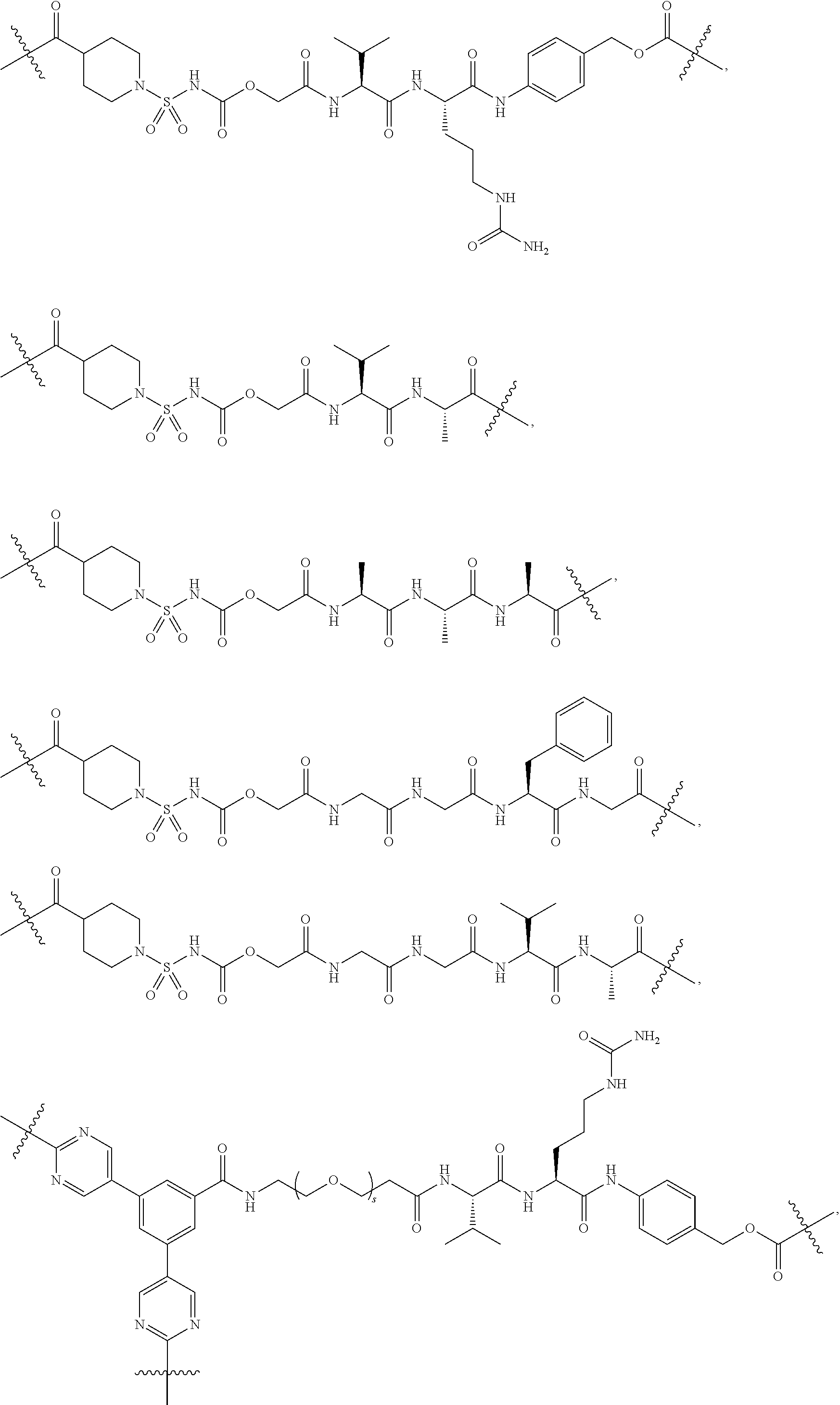

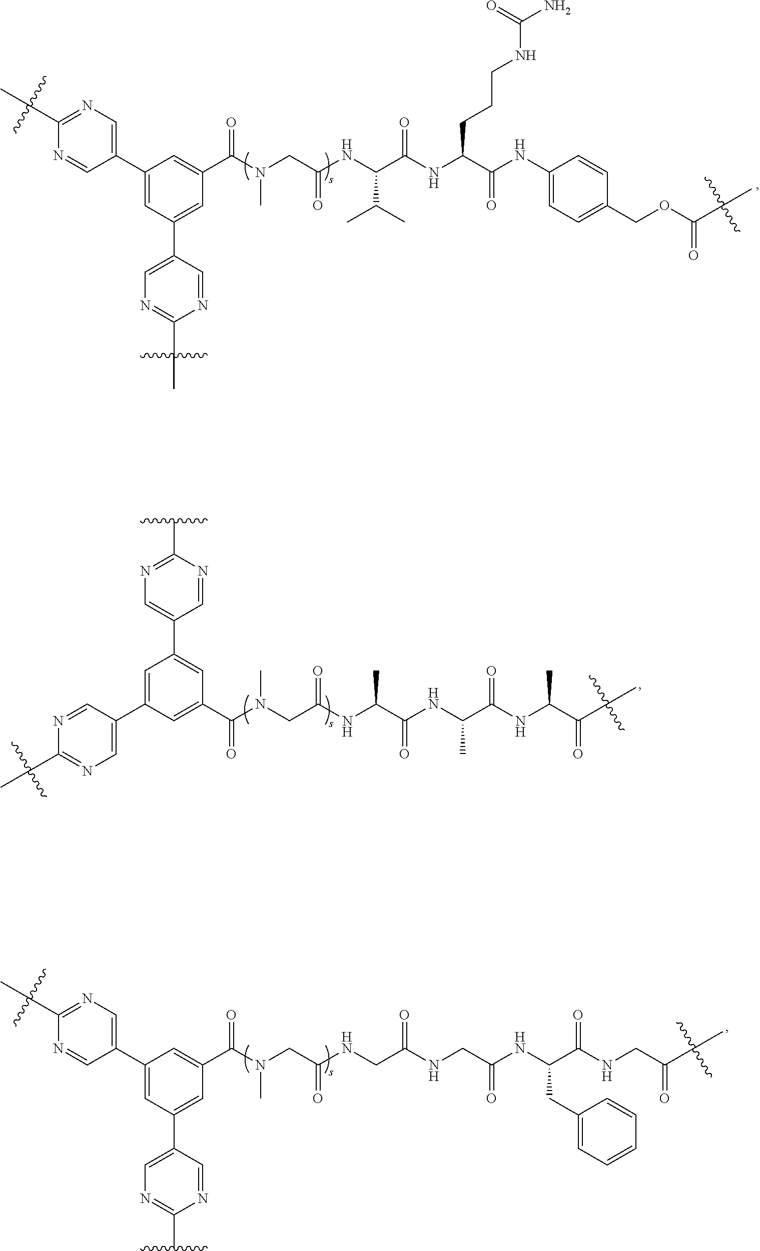

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments,

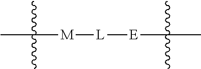

is selected from the group consisting of the following substituted or unsubstituted structures:

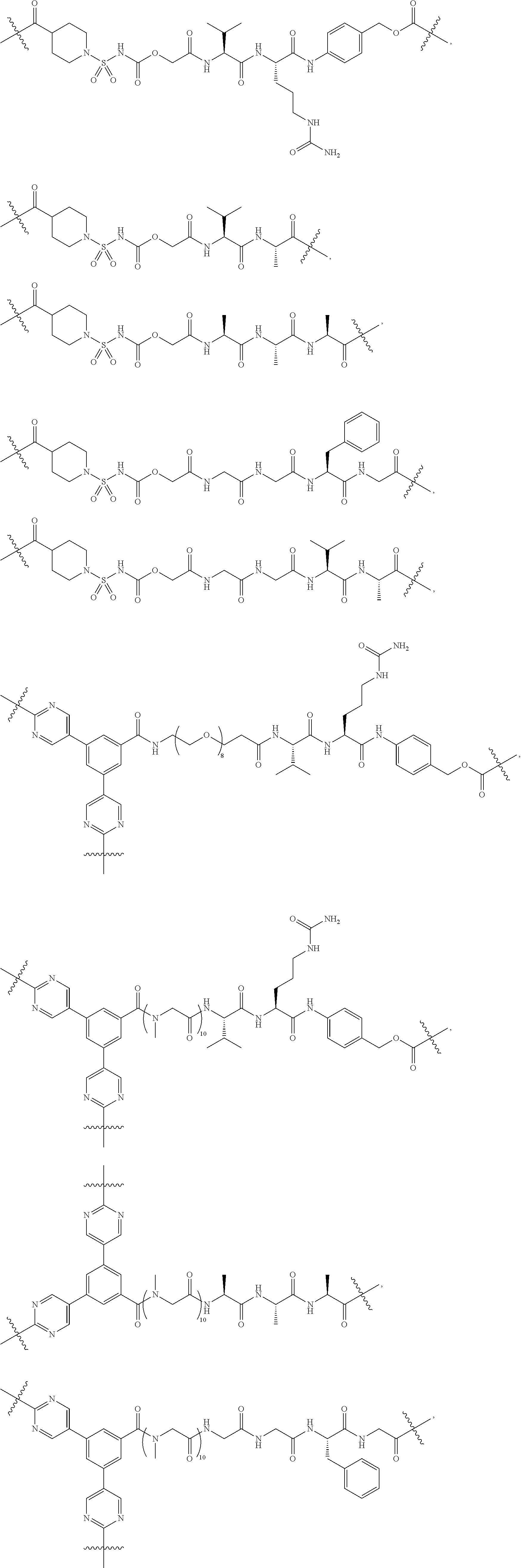

In some embodiments,

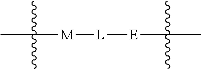

is selected from the group consisting of the following substituted or unsubstituted structures:

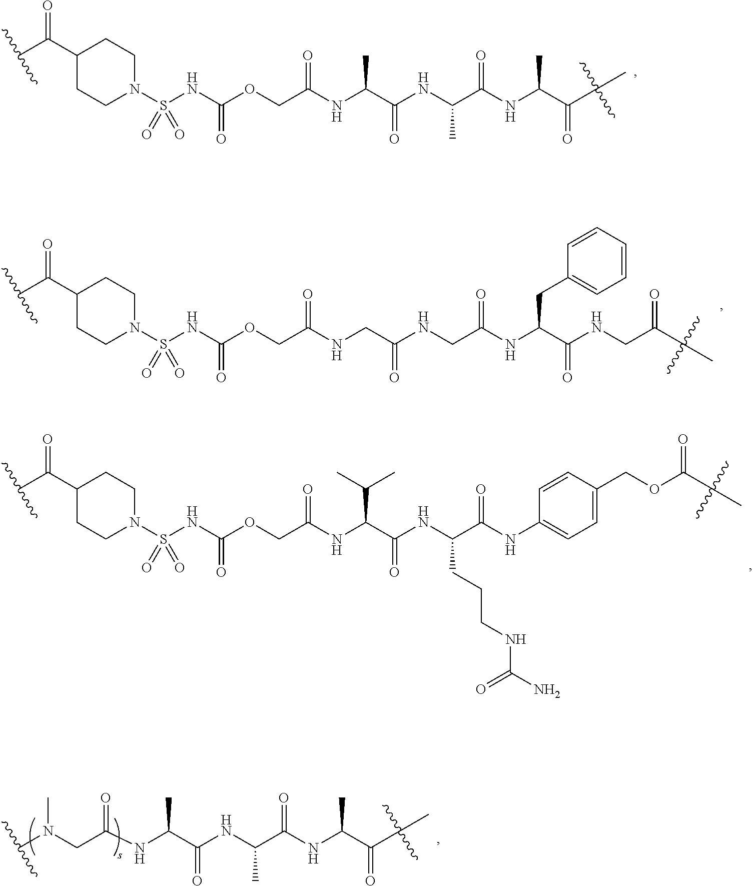

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

In some embodiments,

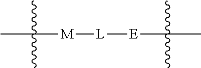

is selected from the group consisting of the following substituted or unsubstituted structures:

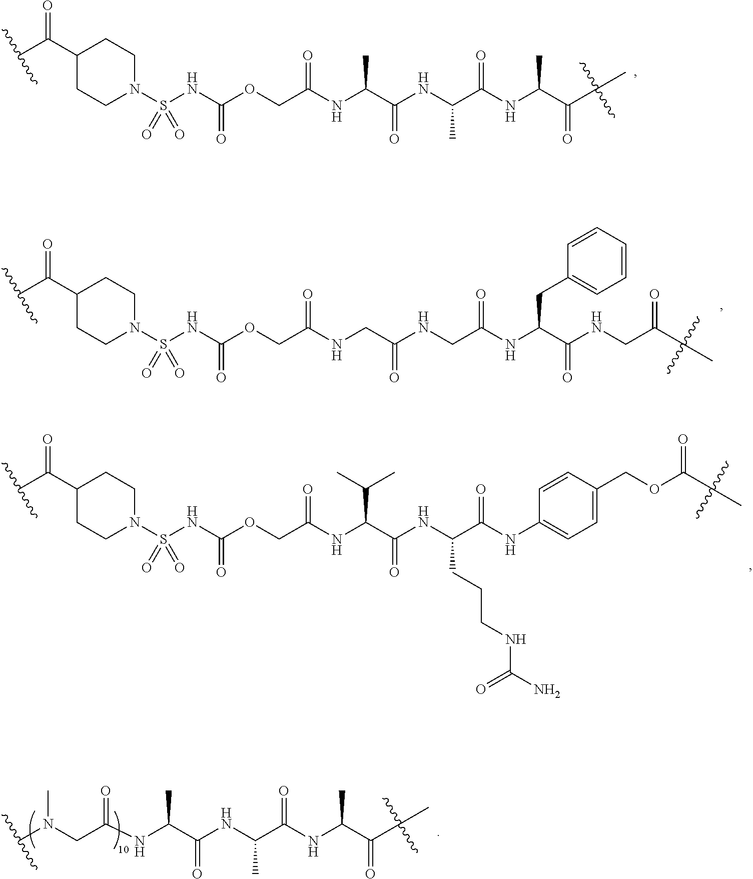

The cytotoxic drugs disclosed in the present application generally contain a variety of functional groups, such as hydroxyl (—OH), carboxyl (—COOH), primary amino (—NH₂), secondary amine (—NR₁H), tertiary amine (—NR₂R₃), wherein R₁, R₂, and R₃ here represent only non-hydrogen substituents on N, or sulfhydryl (—SH), and these functional groups can react with appropriate functional groups in the rest of the conjugate to achieve connection.

In some embodiments, the cytotoxic drug is connected through its —OH, primary amino, secondary amine or tertiary amine, or —SH to E in the antibody-drug conjugate. In some embodiments, D is a monovalent structure obtained by losing one H from —OH, —NH₂ or secondary amino on the cytotoxic drug.

In some embodiments, the cytotoxic drug is selected from the group consisting of eribulin compounds.

In some embodiments, the cytotoxic drug is selected from the group consisting of the following substituted or unsubstituted compounds or isotopically labeled compounds thereof:

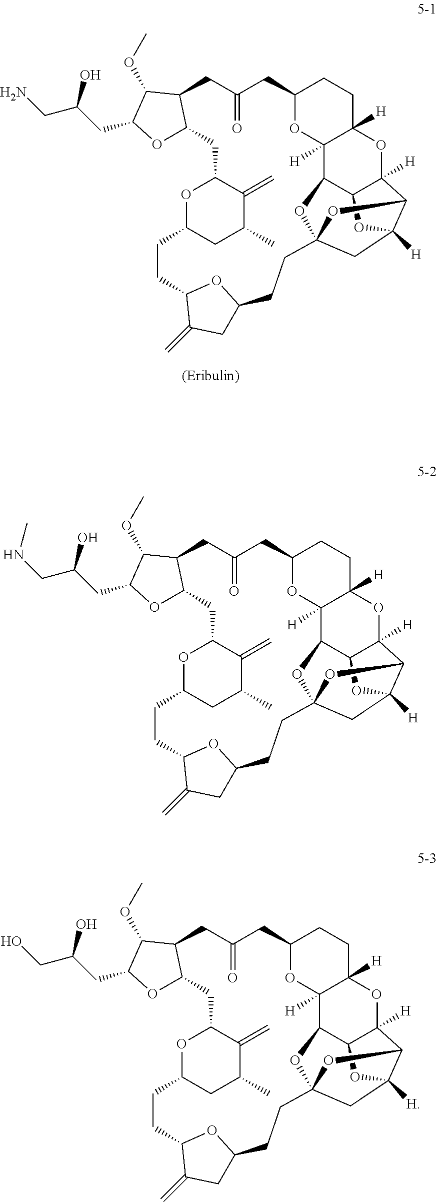

In some embodiments, D is selected from the group consisting of the following substituted or unsubstituted structures:

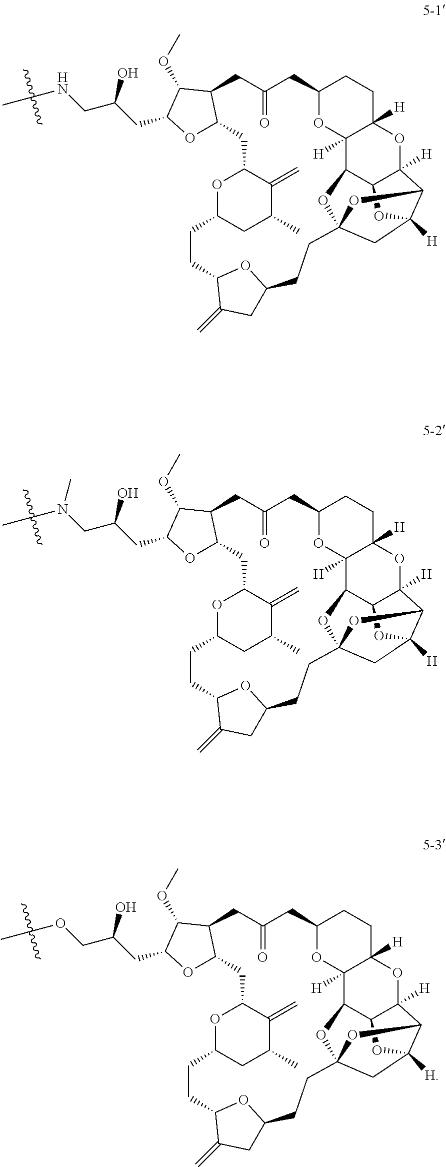

In some embodiments, D is the following structure:

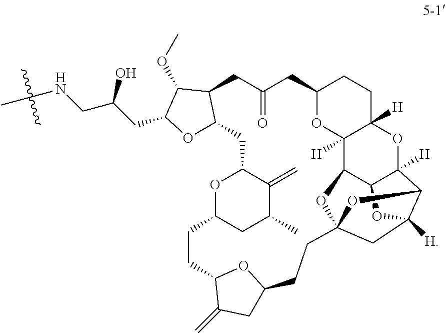

In some embodiments, in a compound having a structure represented by D-E-L-M′ or a pharmaceutically acceptable salt thereof,
M′ represents -M-Lg,
Lg is selected from the group consisting of halogen, substituted or unsubstituted C₁₋₆ alkylsulfonyl (C₁₋₆ alkyl-SO₂—), halogenated phenoxy, hydroxyl (—OH), mercapto (—SH) or amino (—NH₂), preferably Lg is selected from the group consisting of methylsulfonyl or pentafluorophenoxy;
M is selected from the group consisting of the following substituted or unsubstituted structural fragments:

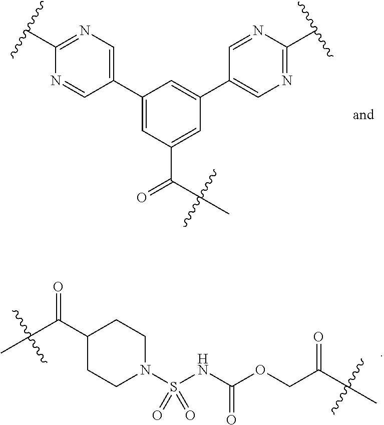 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

L is selected from the group consisting of the substituted or unsubstituted structural fragments composed of one or more of the following groups:

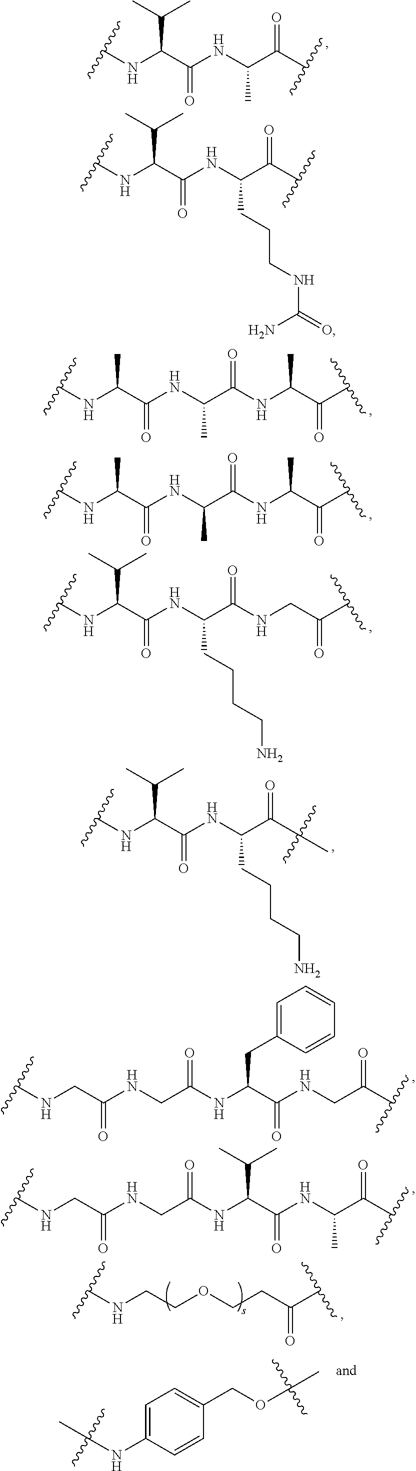

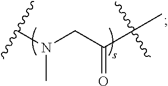

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
preferably, L is selected from the group consisting of the substituted or unsubstituted structural fragments composed of one or more of the following groups:

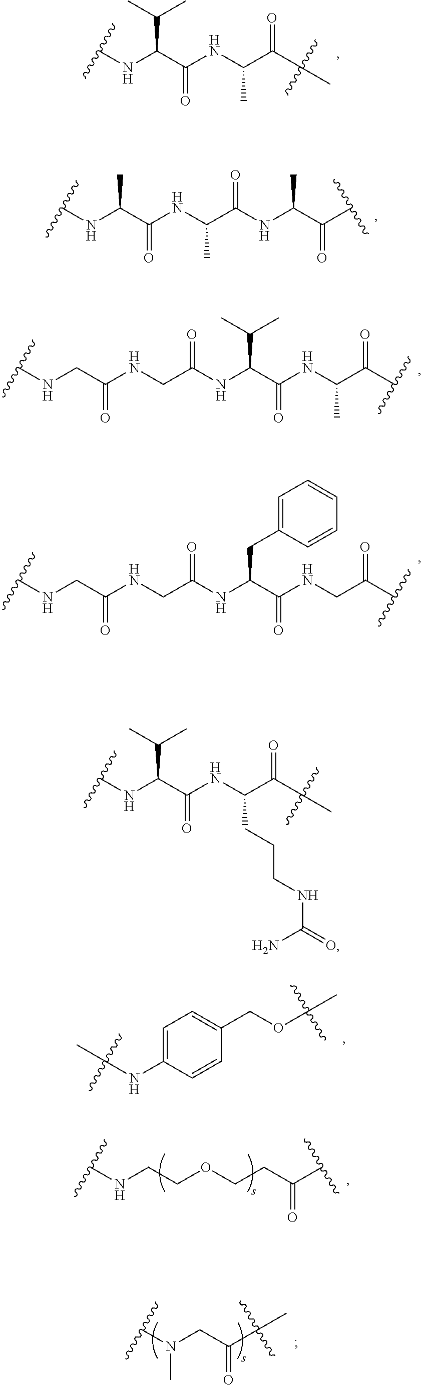

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
E is a single bond, —NH—CH₂— or

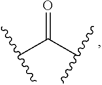 [SMILES: *C(*)=O]

preferably a single bond or

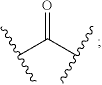 [SMILES: *C(*)=O]

D is selected from the group consisting of the following substituted or unsubstituted structures:

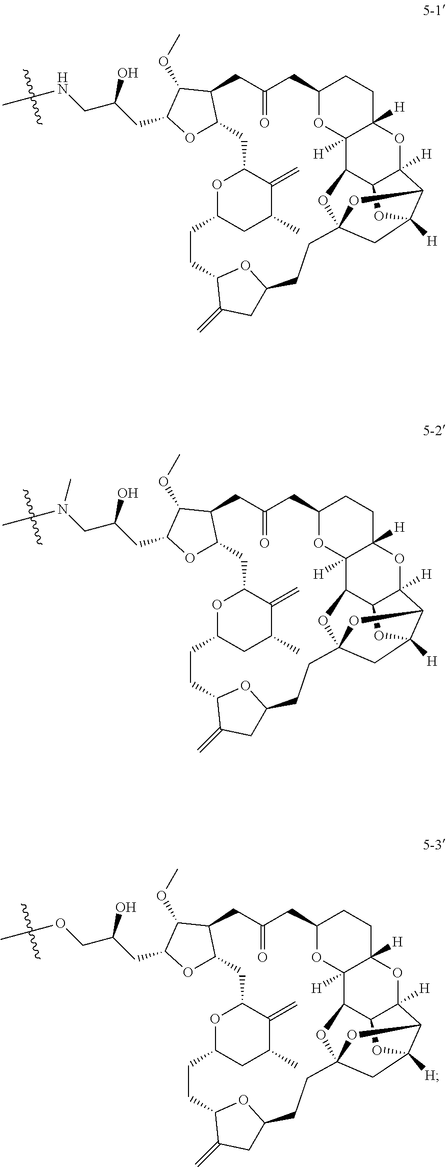

preferably, D is the following structure:

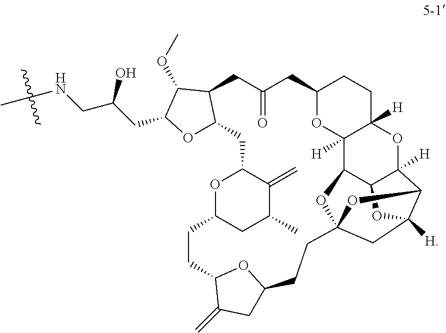

In some embodiments, in a compound having a structure represented by D-E-L-M′ or a pharmaceutically acceptable salt thereof,
M′ represented -M-Lg,
Lg is selected from the group consisting of C₁₋₆ alkylsulfonyl or halogenated phenoxy, preferably methylsulfonyl or pentafluorophenoxy;
M is selected from the group consisting of the following substituted or unsubstituted structural fragments:

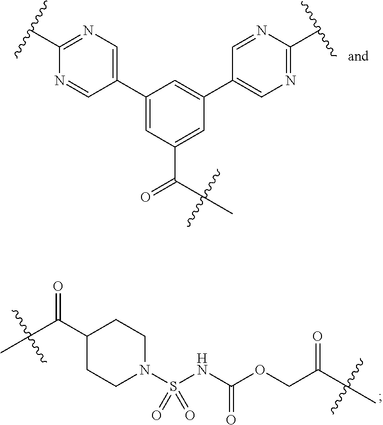 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

L is selected from the group consisting of the substituted or unsubstituted structural fragments composed of one or more of the following groups:

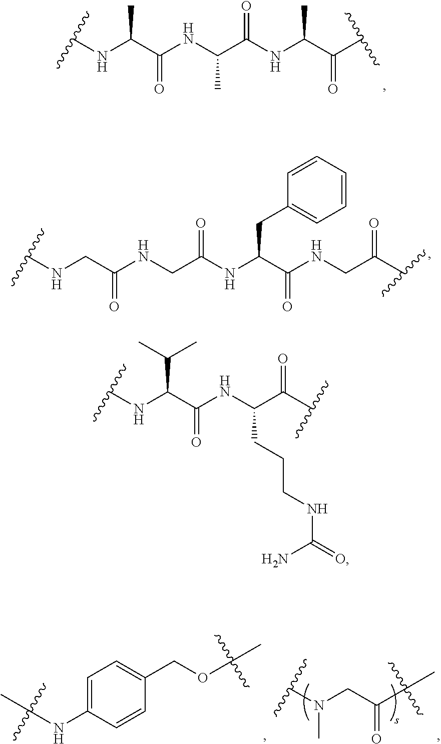

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
preferably, L is selected from the group consisting of the following substituted or unsubstituted structural fragments:

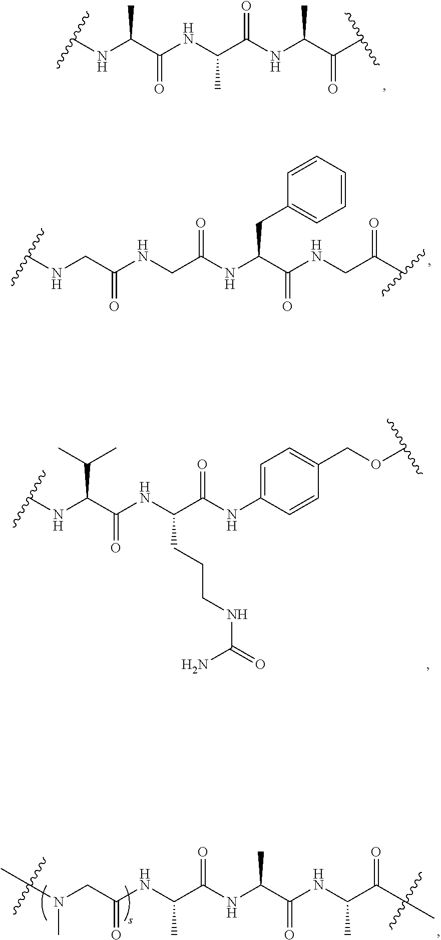

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
E is a single bond or

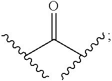 [SMILES: *C(*)=O ; ";"]

D is the following structure:

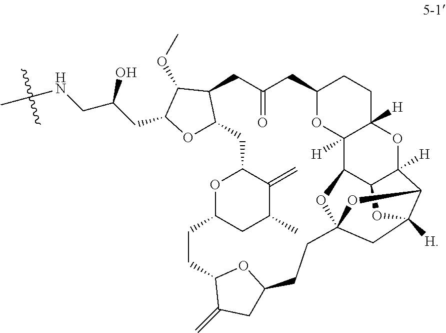

In some embodiments, the compound or a pharmaceutically acceptable salt thereof is selected from the group consisting of J-1 to J-9 and H-1 to H-15 shown below:

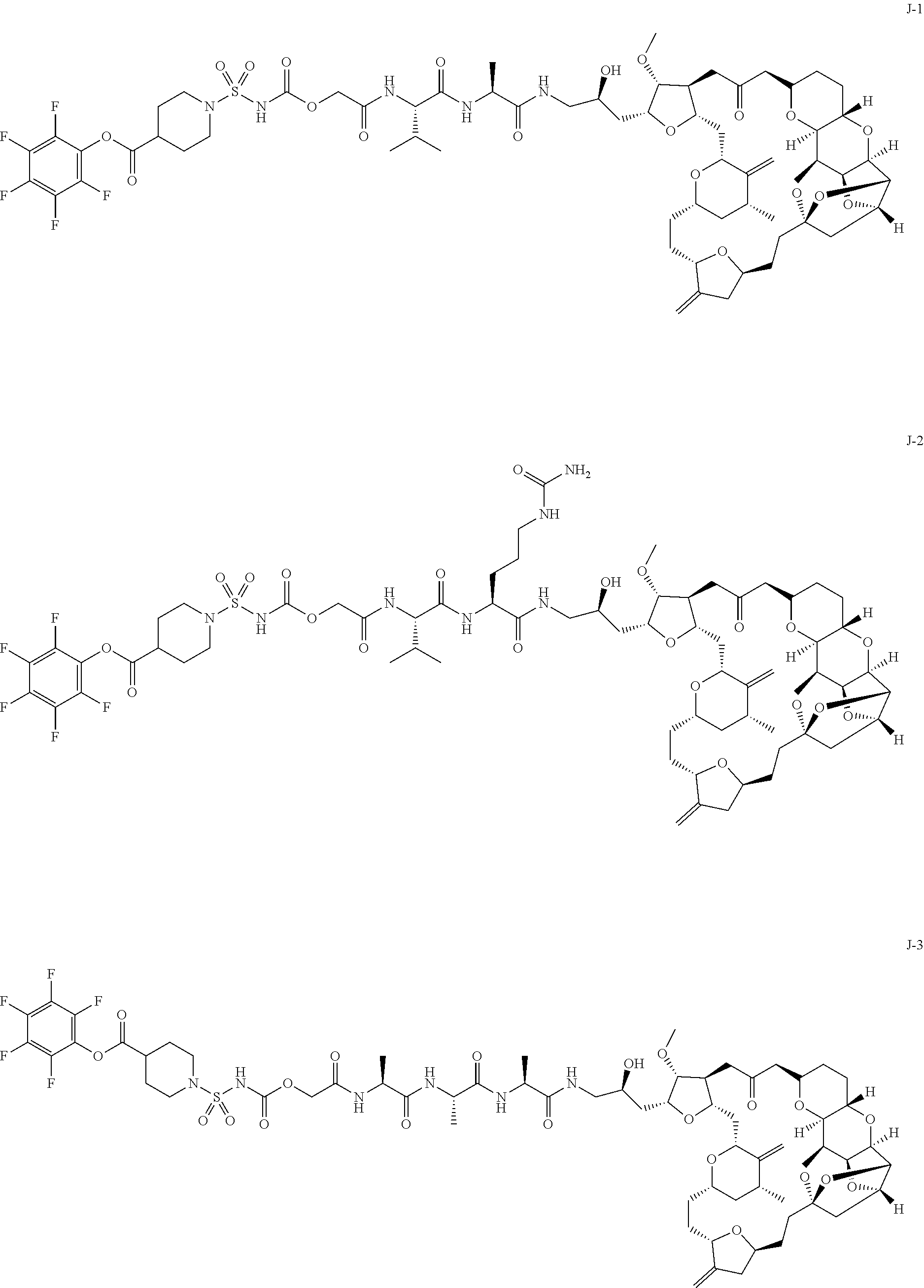

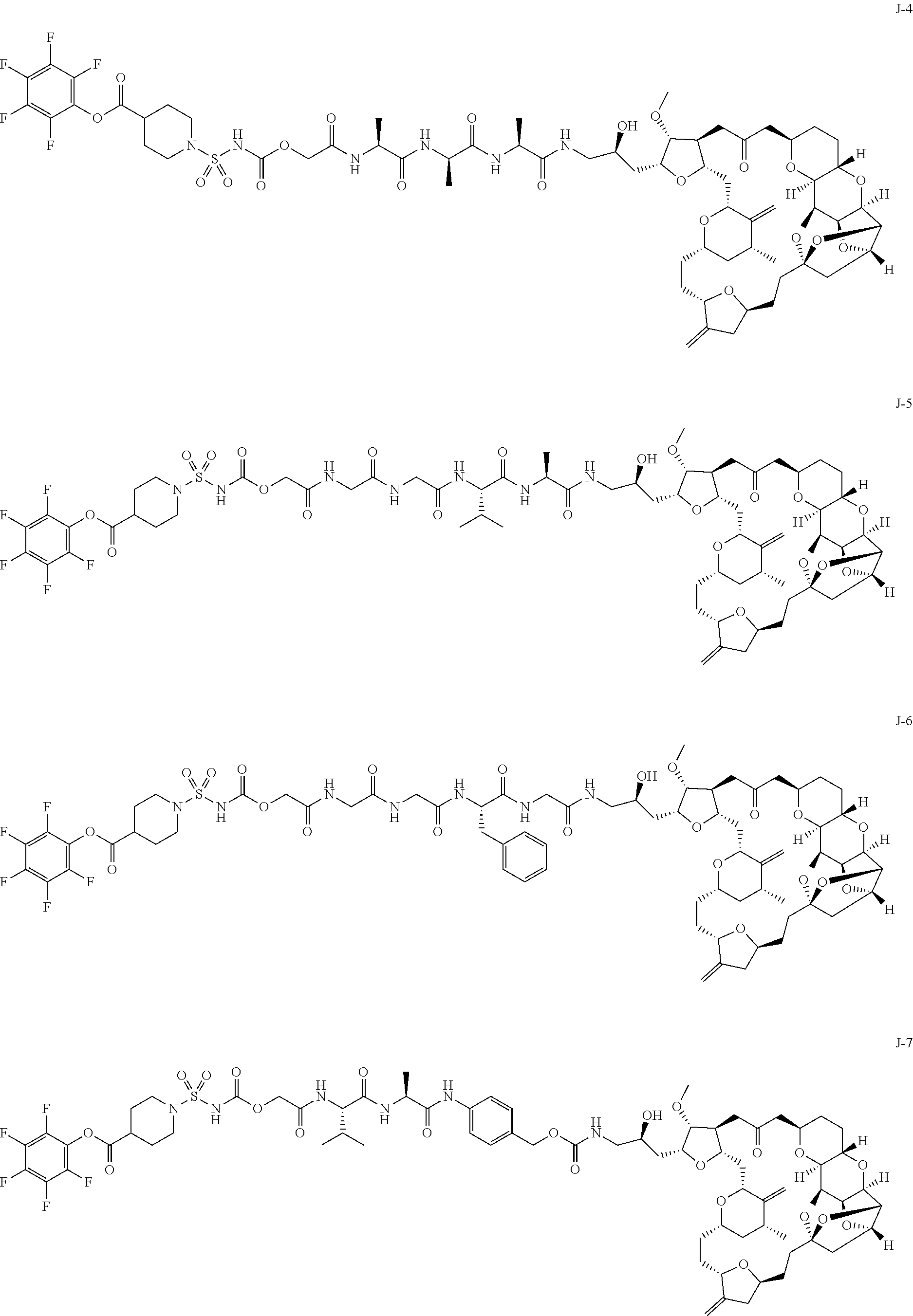

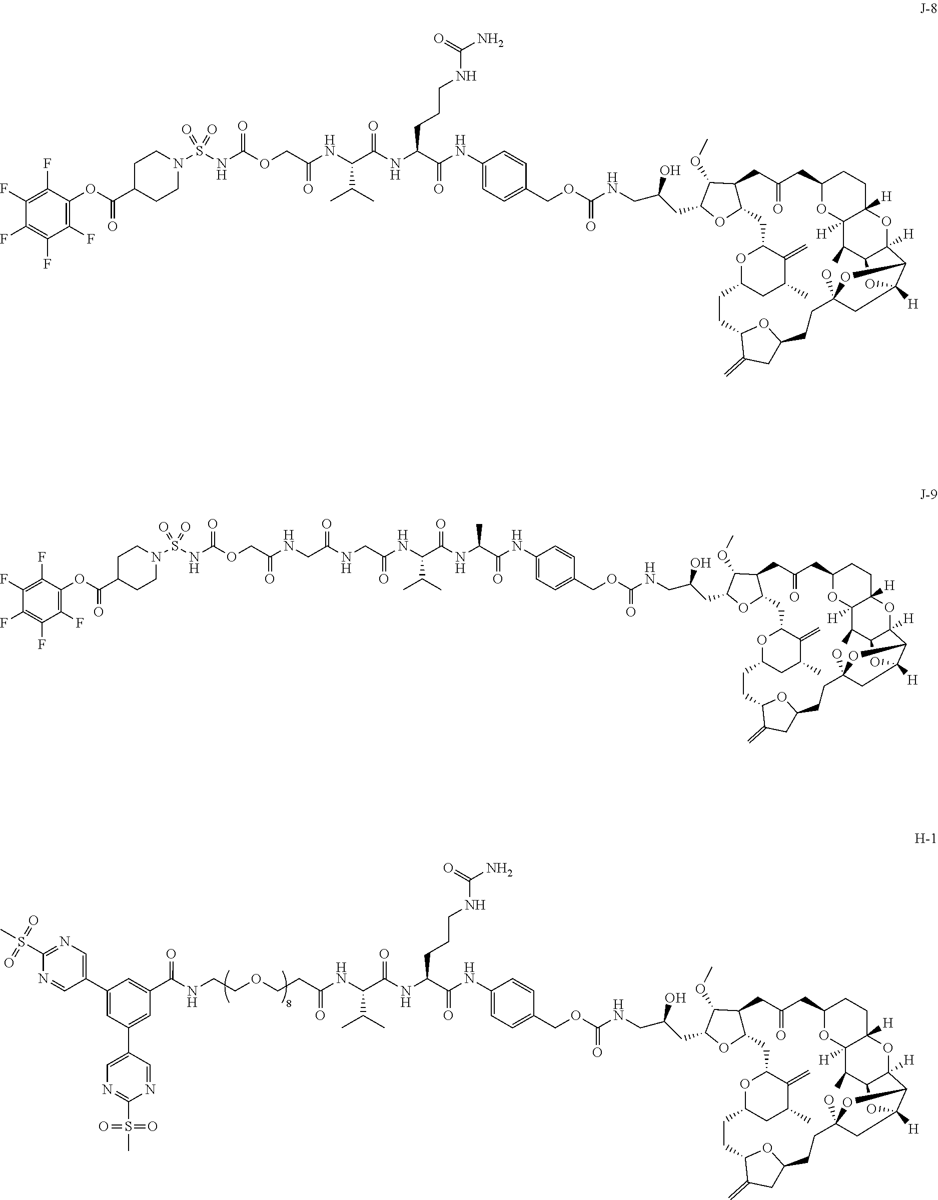

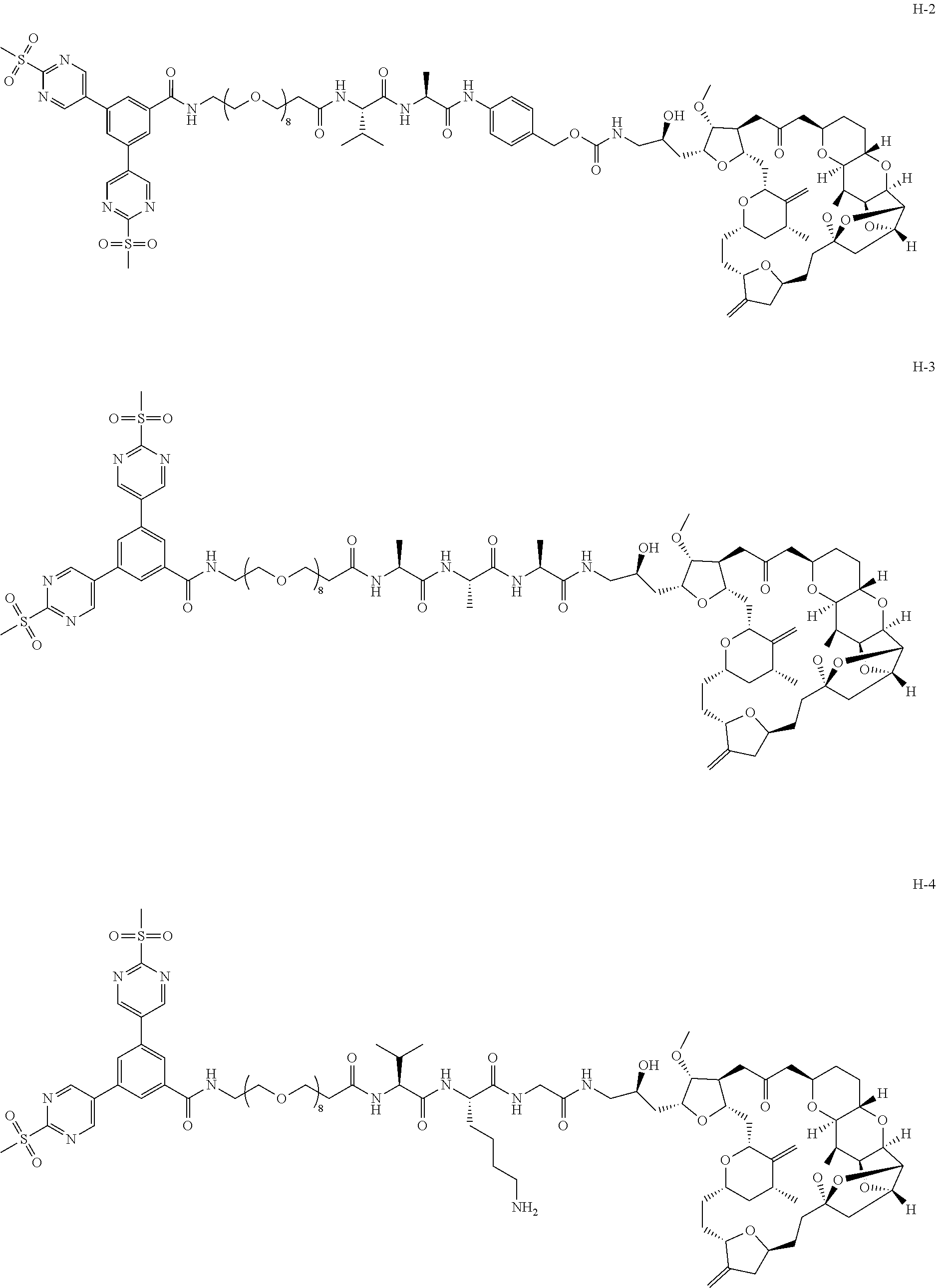

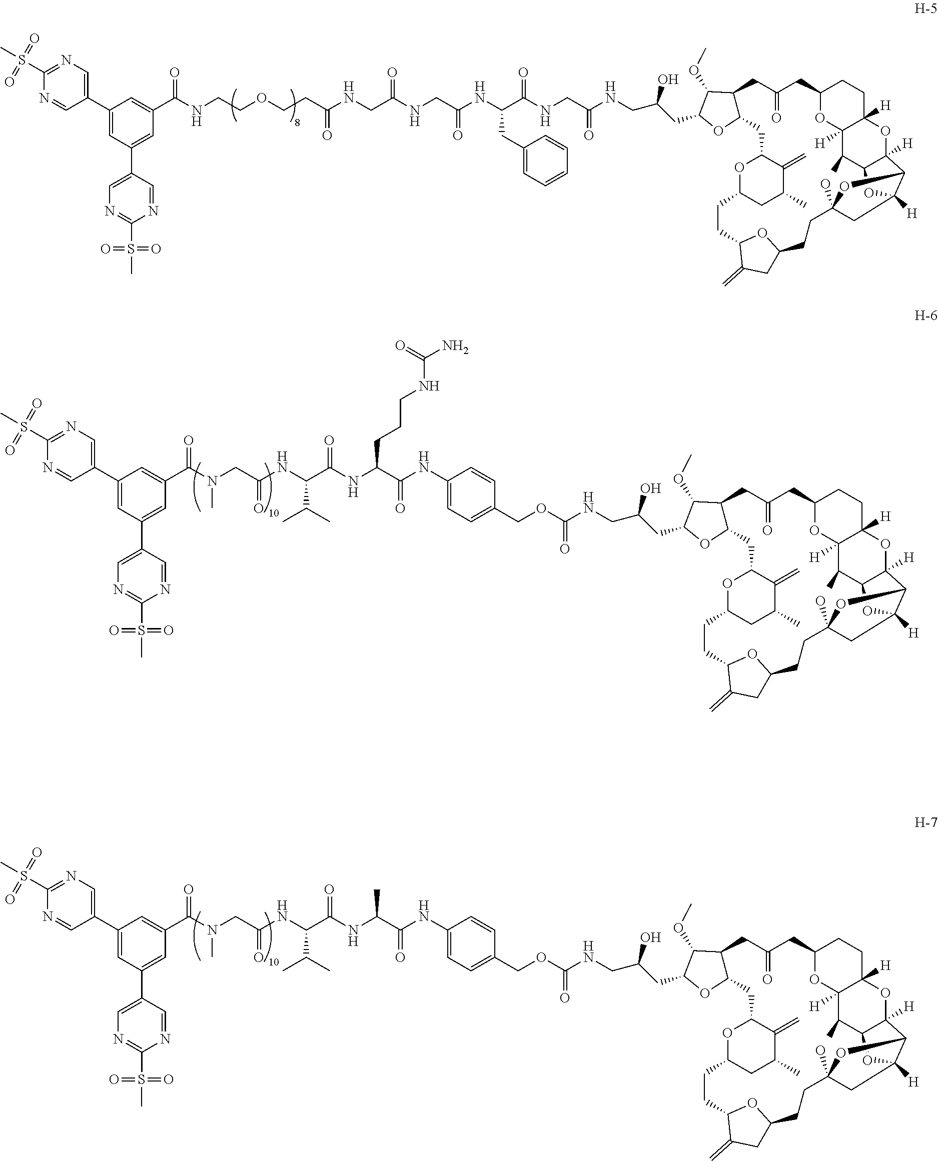

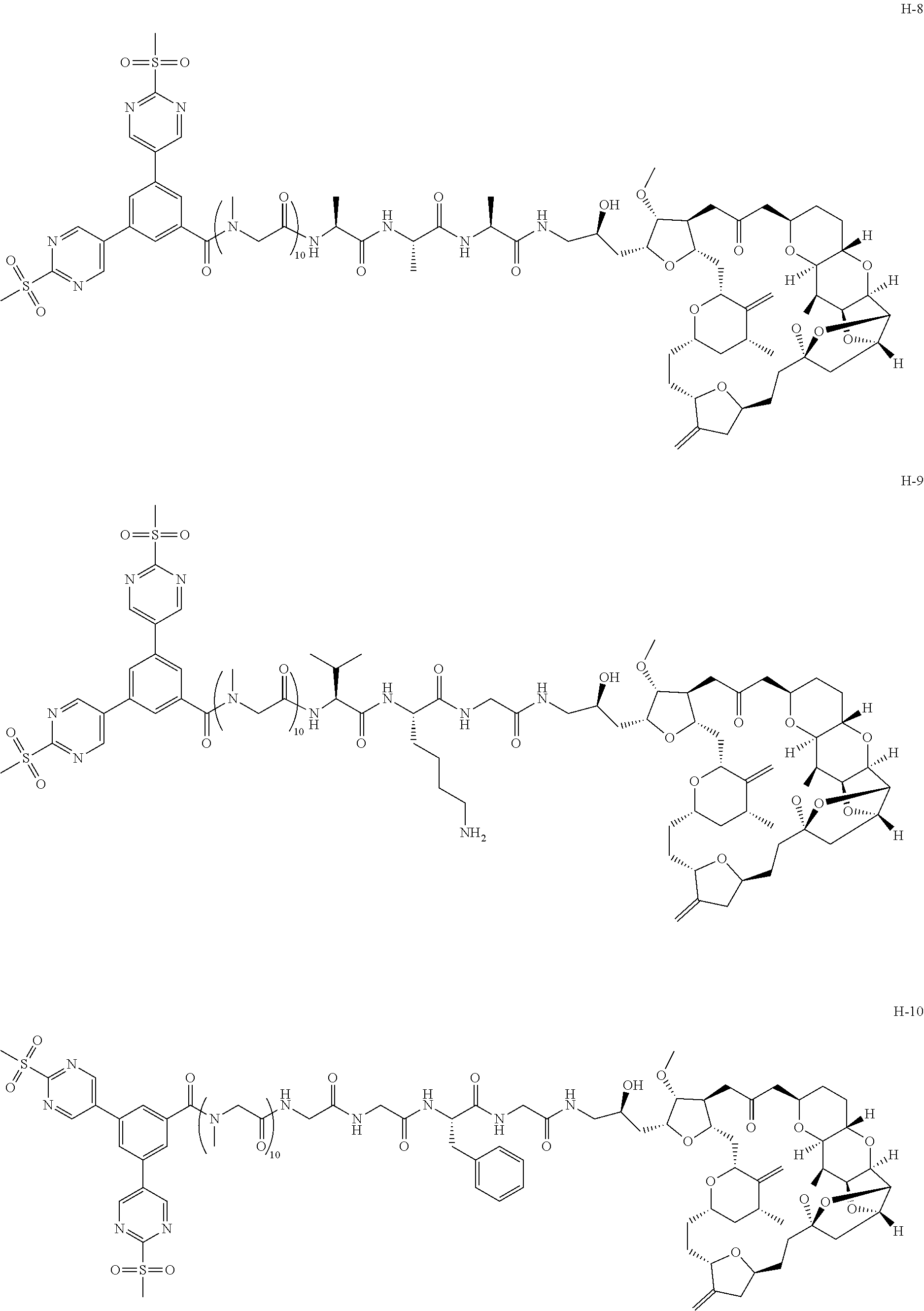

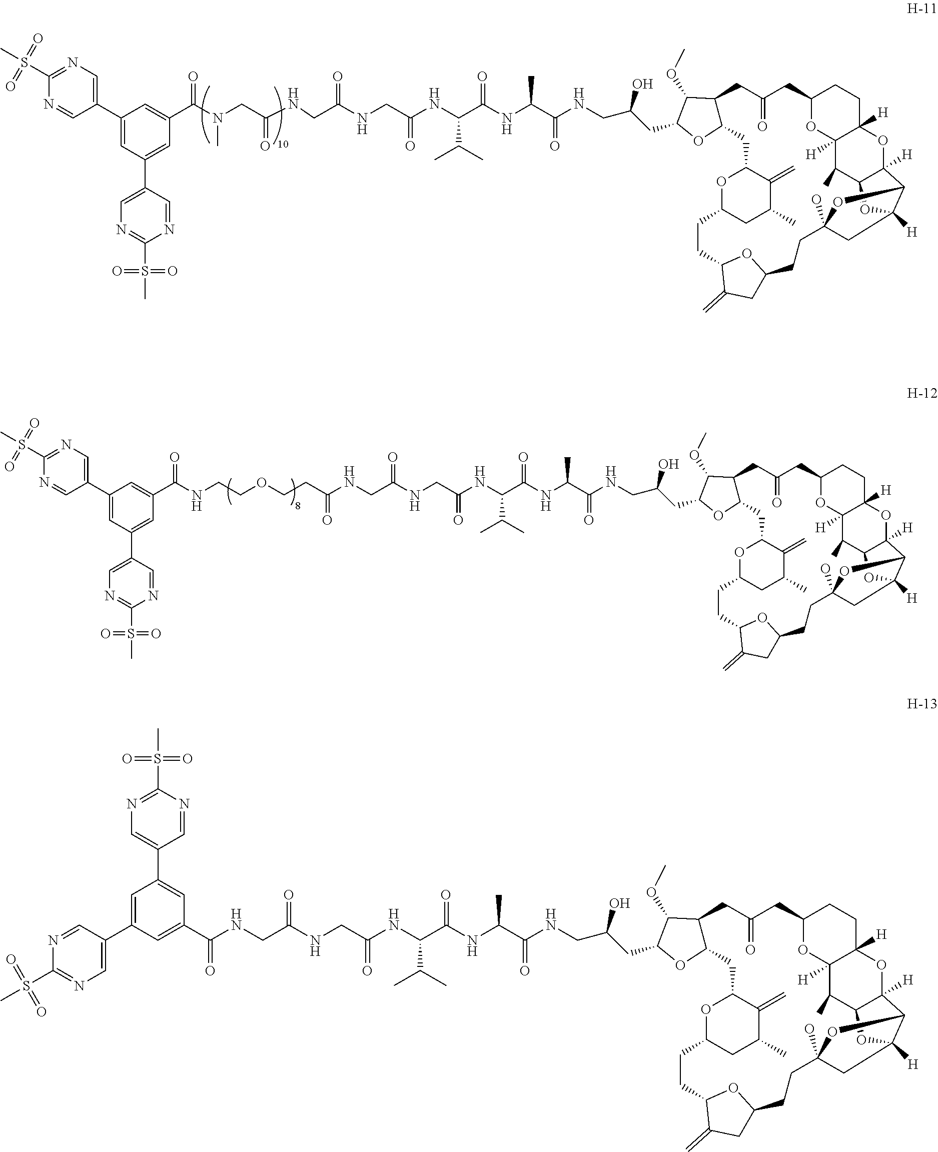

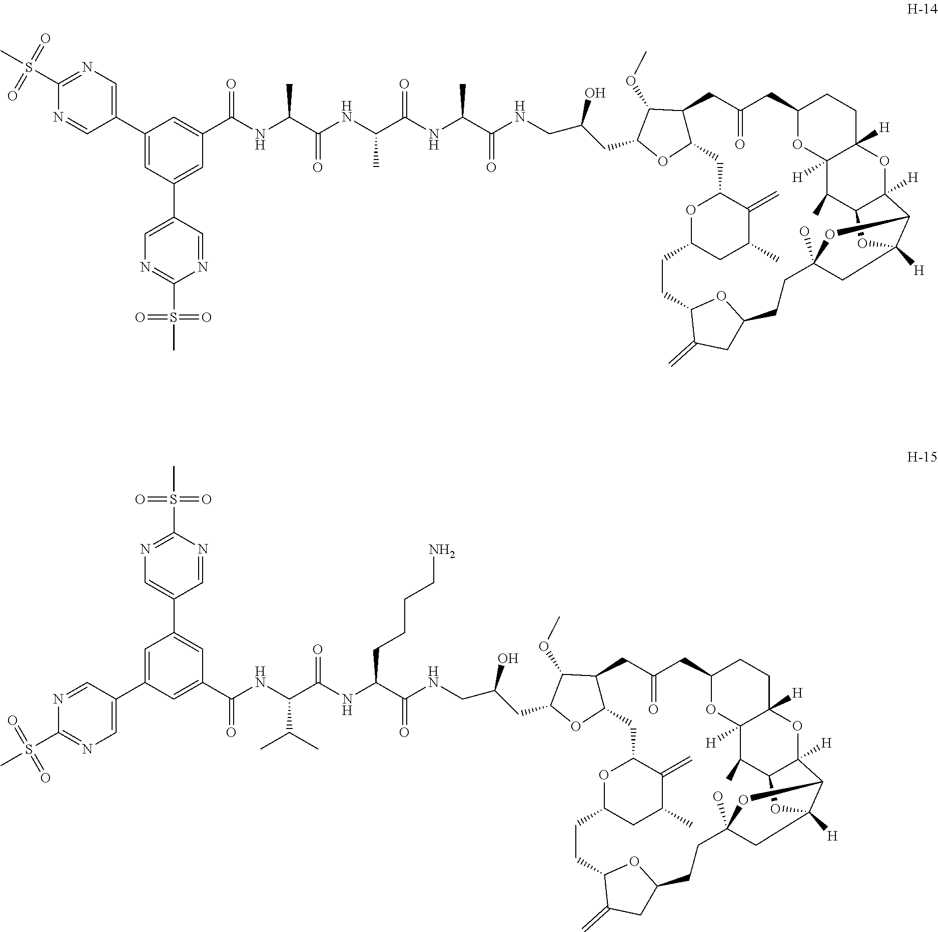

In some embodiments, the present application provides a compound or pharmaceutically acceptable salt thereof as shown below:

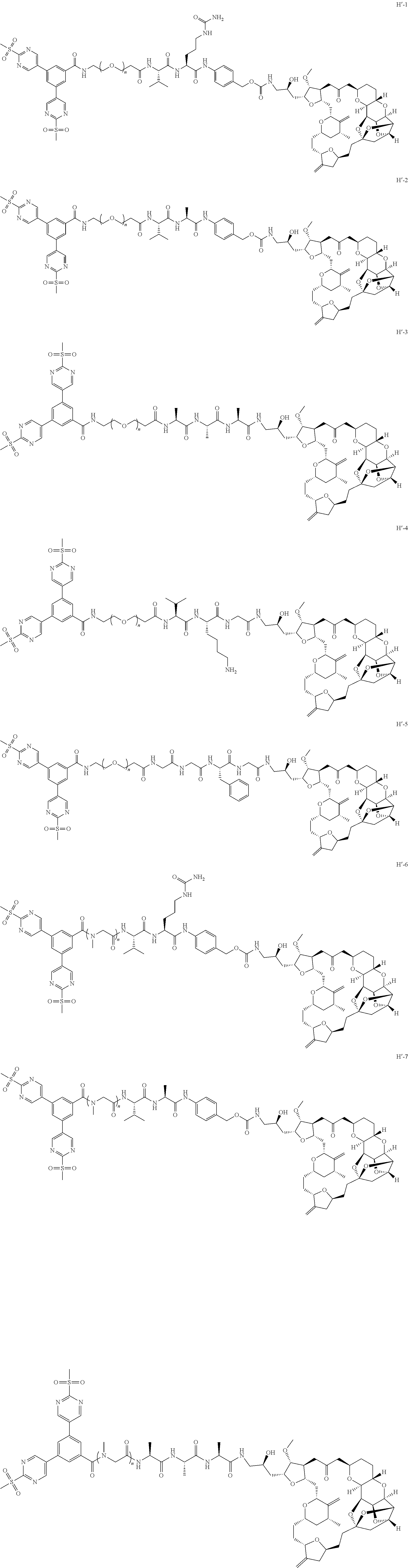

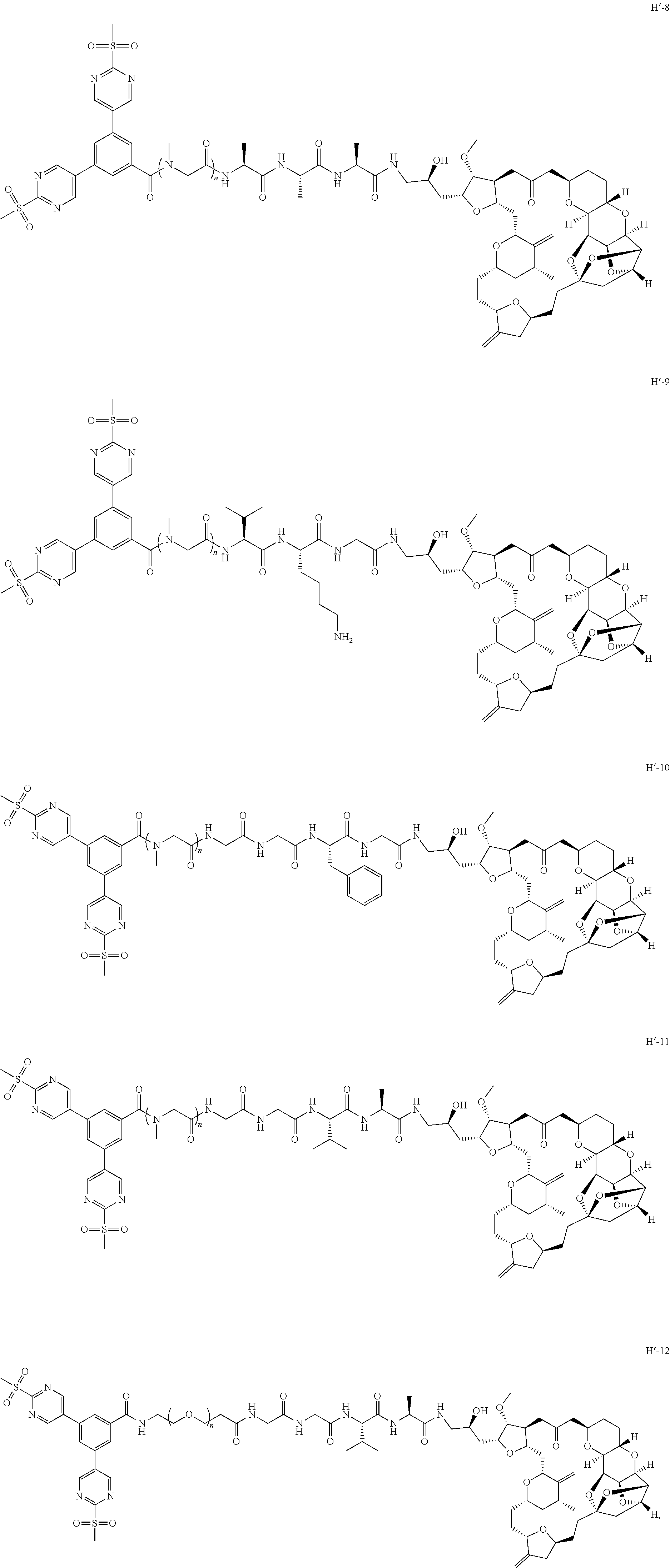

wherein n is an integer selected from 1 to 20, preferably an integer of 1 to 15, such as an integer of 1 to 12, 3 to 12, 5 to 10, 8 to 10, such as 1, 2, 3, 4, 5, 6, 7, 8, 9, 10, 11, 12, 13, 14 or 15, preferably 5, 8, 10.

In some embodiments, the present application provides the compounds as shown in J-1 to J-9, H-1 to H-15, H′-1 to H′-12 or pharmaceutically acceptable salts thereof, wherein n is an integer selected from 1 to 20, preferably an integer of 1 to 15, such as an integer of 1 to 12, 3 to 12, 5 to 10, 8 to 10, such as 1, 2, 3, 4, 5, 6, 7, 8, 9, 10, 11, 12, 13, 14 or 15, preferably 5, 8, 10.

In some embodiments, the present application provides the compounds as shown in J-1 to J-9, H′-1 to H′-12, H-13 to H-16 or pharmaceutically acceptable salts thereof, wherein n is an integer selected from 1 to 20, preferably an integer selected from 1 to 15, such as an integer of 1 to 12, 3 to 12, 5 to 10, 8 to 10, such as 1, 2, 3, 4, 5, 6, 7, 8, 9, 10, 11, 12, 13, 14 or 15, preferably 5, 8, 10.

In some embodiments, the present application provides the compounds as shown in J-1 to J-9, H-1 to H-15 or pharmaceutically acceptable salts thereof.

In some embodiments, the present application provides compounds as shown in J-1 to J-9 or pharmaceutically acceptable salts thereof.

In some embodiments, the compounds or pharmaceutically acceptable salts thereof described above may be optionally substituted with one or more suitable substituents.

### Conjugate

The second aspect of the present invention provides a conjugate represented by Formula (II), wherein:

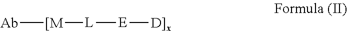

Ab represents a targeting moiety; M, L, E and D are as described above;
x is 1 to 10.

In some preferred embodiments, the target of Ab is selected from the group consisting of epidermal growth factor, Trop-2, CD37, HER2, CD70, EGFRvIII, Mesothelin, Folate eceoptor1, Mucin 1, CD138, CD20, CD19, CD30, SLTRK6, Nectin 4, Tissue factor, Mucin16, Endothelinreceoptor, STEAP1, SLC39A6, Guanylylcyclase C, PSMA, CCD79b, CD22, Sodium phosphate cotransporter 2B, GPNMB, Trophoblast glycoprotein, AGS-16, EGFR, CD33, CD66e, CD74, CD56, PD-L1, TACSTD2, DR5, E16, STEAP1, 0772P, MPF, Napi3b, Sema 5b, PSCA hlg, ETBR, MSG783, STEAP2, TrpM4, CRIPTO, CD21, CD79b, FcRH2, NCA, MDP, IL20Rα, Brevican, EphB2R, ASL G659, PSCA, GEDA, BAFF-R, CD22, CD79a, CXCR5, HLA-DOB, P2X5, CD72, LY64, FcRH1, IRTA2, TENB2, integrin α5β6, α4β7, FGF2, FGFR2, Her3, CD70, CA6, DLL3, DLL4, P-cadherin, EpCAM, pCAD, CD223, LYPD3, LY6E, EFNA4, R OR1, SLITRK6, 5T4, ENPP3, SLC39A6, Claudin18.2, BMPR1B, E16, STEAP1, Tyro7, 0772P, MPF, Napi3b, Sema 5b. PSCA hlg, ETBR, MSG783, STEAP2, TrpM4, CRIPTO, CD21, CD79b, FcRH2, NCA, MDP, IL20Rα, Brevican, EphB2R, ASLG659, PSCA, GEDA, CD22, CD79a, CXCR5, HLA-DOB, P2X5, CD72, LY64, FcRH1, IRTA2, c-Met, ApoE, CD11c, CD40, CD45 (PTPRC), CD49D (ITGA4), CD80, CSF1R, CTSD, GZMB, Ly86, MS4A7, PIK3AP1, PIK3CD, CCR5, IFNG, IL10RA1, IL-6, ACTA2, COL7A1, LOX, LRRC15, MCPT8, MMP10, NOG, SERPINE1, STAT1, TGFBR1, CTSS, PGF, VEGFA, C1QA, C1QB, ANGPTL4, EGLN, ANGPTL4, EGL N3, BNIP3, AIF1, CCL5, CXCL10, CXCL11, IFI6, PLOD2, KISS1R, STC2, DDIT4, PFKFB3, PGK1, PDK1, AKR1C1, AKR1C2, CA DM1, CDH11, COL6A3, CTGF, HMOX1, KRT33A, LUM, WNT5A, IGFBP3, MMP14, CDCP1, PDGFRA, TCF4, TGF, TGFB1, TGFB2, CD11b, ADGRE1, EMR2, TNFRSF21, UPK1B, TNFSF9, MMP16, MFI2, IGF-1R, RNF43, NaPi2b and TENB2.

In some preferred embodiments, Ab is a small molecule ligand, such as a folic acid derivative, a glutamate urea derivative, a somatostatin derivative, an aromatic sulfonamide derivative (e.g., a carbonic anhydrase IX inhibitor), a polyene connecting two aliphatic indoles, a cyanine dye, or IR-783 or derivative thereof.

In some preferred embodiments, Ab is an antibody, such as a monoclonal antibody or an antigen-binding fragment thereof, wherein the monoclonal antibody or antigen-binding fragment thereof comprises Fab, Fab′, F(ab′)₂, Fd, Fv, dAb, complementary determining region fragment, single-chain antibody (e.g., scFv), non-human antibody, humanized antibody, chimeric antibody, fully human antibody, probody, bispecific antibody, or multispecific antibody.

In some preferred embodiments, Ab is an anti-Her2 monoclonal antibody, such as trastuzumab or pertuzumab.

In some embodiments, Ab is an antibody or antigen-binding fragment thereof. In some embodiments, Ab is an antibody or antigen-binding fragment thereof capable of specifically binding to epidermal growth factor receptor 2 (Her2), a member of the ErbB family of receptor tyrosine kinases.

In some embodiments, the antibody or antigen-binding fragment thereof comprises:
(1) the following heavy chain variable region (VH) and/or light chain variable region (VL):
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some preferred embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable regions (VH) and/or light chain variable regions (VL), wherein the CDRs are defined according to the IMGT numbering system:
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, and a CDR-L2 having a sequence as set forth in SEQ ID NO: 17 or variant thereof. L2, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof,
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some embodiments, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, the antibody or antigen-binding fragment thereof comprises:
(a) a VH as set forth in SEQ ID NO: 1 or variant thereof, and/or, a VL as set forth in SEQ ID NO: 2 or variant thereof, or
(b) a VH as set forth in SEQ ID NO: 3 or variant thereof, and/or, a VL as set forth in SEQ ID NO: 4 or variant thereof, or
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

In some embodiments, the antibody or antigen-binding fragment thereof comprises:
(a) a VH as set forth in SEQ ID NO: 1, and a VL as set forth in SEQ ID NO: 2; or
(b) a VH as set forth in SEQ ID NO: 3, and a VL as set forth in SEQ ID NO: 4.

In some embodiments, the antibody or antigen-binding fragment thereof further comprises:
(a) a heavy chain constant region (CH) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids); and
(b) a light chain constant region (CL) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids).

In some embodiments, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region.

In some embodiments, the antibody or antigen-binding fragment thereof comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 35.

In some embodiments, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36.

In some embodiments, the antibody or antigen-binding fragment thereof comprises a heavy chain constant region (CH) as set forth in SEQ ID NO: 35 and a light chain constant region (CL) as set forth in SEQ ID NO: 36.

In some embodiments, the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

In some embodiments, the antibody or antigen-binding fragment thereof comprises:
(1) a heavy chain comprising the sequence as set forth in SEQ ID NO: 37, and, a light chain comprising the sequence as set forth in SEQ ID NO: 38; or
(2) a heavy chain comprising the sequence as set forth in SEQ ID NO: 39, and, a light chain comprising the sequence as set forth in SEQ ID NO: 40.

In some embodiments, the antibody or antigen-binding fragment thereof is selected from Trastuzumab or Pertuzumab. The query accession number (IMGT/mAb-DB ID) for the amino acid sequence of Trastuzumab in the IMGT database is: 97. The query accession number (IMGT/mAb-DB ID) for the amino acid sequence of Pertuzumab in the IMGT database is: 80.

In certain embodiments, the antibody or antigen-binding fragment disclosed herein has a heavy chain constant region that may comprise a C-terminal lysine lack a C-terminal lysine, or lack a C-terminal glycine-lysine dipeptide. In some embodiments, the N-terminal amino acid of the antibody or antigen-binding fragment thereof may be cyclized to pyroglutamic acid.

As known to those skilled in the art, pyroglutamic acid is the conjugate acid of pyroglutamate and is in equilibrium with pyroglutamate in solution.

In certain embodiments, provided herein is a composition comprising the antibody or antigen-binding fragments disclosed herein, wherein each antibody or antigen-binding fragment thereof may independently comprise a C-terminal lysine, lack a C-terminal lysine, lack a C-terminal glycine-lysine and/or comprise a N-terminal glutamine or glutamic acid, or a N-terminal amino acid cyclized to pyroglutamic acid or pyroglutamate.

In certain embodiments, the antibody or antigen-binding fragment disclosed herein comprises those capable of specifically binding to an antigen and may comprise post-translational modifications thereof (e.g., cleavage of the C-terminal lysine in the heavy chain, conversion of the N-terminal glutamine or glutamic acid in the heavy or light chain to pyroglutamic acid or pyroglutamate), which may occur during recombinant expression in a host cell (e.g., CHO cell) or arise during purification/storage.

In certain embodiments, the N-terminal glutamine of the VH having the sequence as set forth in SEQ ID NO: 1 or 3 or variant thereof or the heavy chain having the sequence as set forth in SEQ ID NO: 37 or 39 or variant thereof undergoes cyclization to form pyroglutamic acid or pyroglutamate.

In certain embodiments, the heavy chain constant region (CH) as set forth in SEQ ID NO: 35 or variant thereof or the heavy chain having the sequence as set forth in SEQ ID NO: 37 or 39 or variant thereof lacks C-terminal lysine.

In certain embodiments, M is connected to mercapto groups (—SH) or amino groups (—NH₂) on the Ab.

In certain embodiments, M is connected to mercapto groups (—SH) on the Ab.

In some embodiments, in the antibody drug conjugate having the structure represented by Ab-[M-L-E-D]ₓ, M-L-E-D is formed by a compound represented by D-E-L-M′, preferably by removing Lg from D-E-L-M′, wherein the compound represented by D-E-L-M′ is as defined above, Ab is as defined above, and x is 1 to 10.

In some embodiments, in the antibody drug conjugate having a structure represented by Ab-[M-L-E-D]ₓ, M-L-E-D is formed by a compound as shown in J-1 to J-9, H-1 to H-15, or H′-1 to H′-12 (e.g., J-1 to J-9, or H-1 to H-15), preferably by removing —SO₂Me or pentafluorophenoxy from the compound, Ab is as defined above, and x is 1 to 10.

In some embodiments, in the antibody-drug conjugate having the structure represented by Ab-[M-L-E-D]ₓ, M-L-E-D is formed by removing the —SO₂Me or pentafluorophenoxy of the compound as shown in J-3, J-6, J-8 or H-8, Ab is as defined above, and x is 1 to 10.

In some embodiments, in the antibody-drug conjugate having a structure represented by Ab-[M-L-E-D]ₓ,
M is selected from the group consisting of the following substituted or unsubstituted structural fragments:

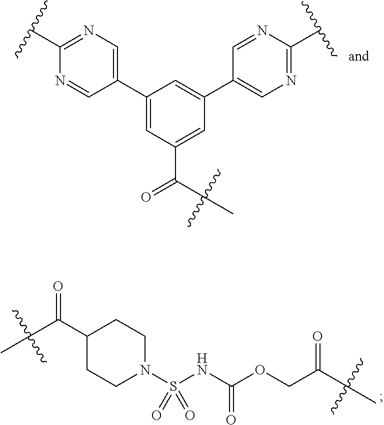 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

L is selected from the group consisting of the substituted or unsubstituted structural fragments composed of one or more of the following groups:

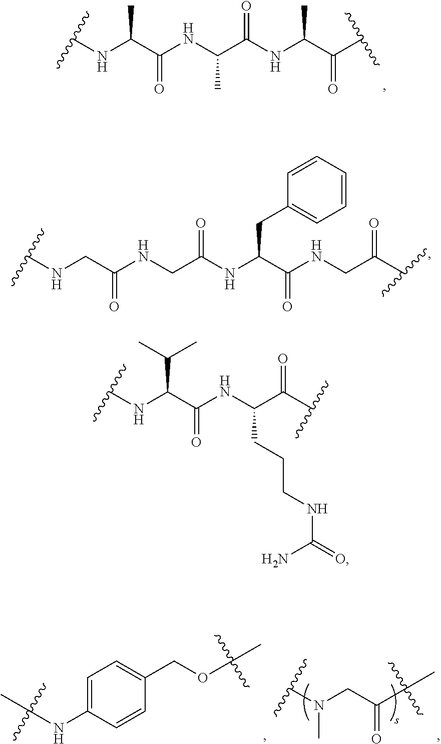

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
preferably, L is selected from the group consisting of the following substituted or unsubstituted structural fragments:

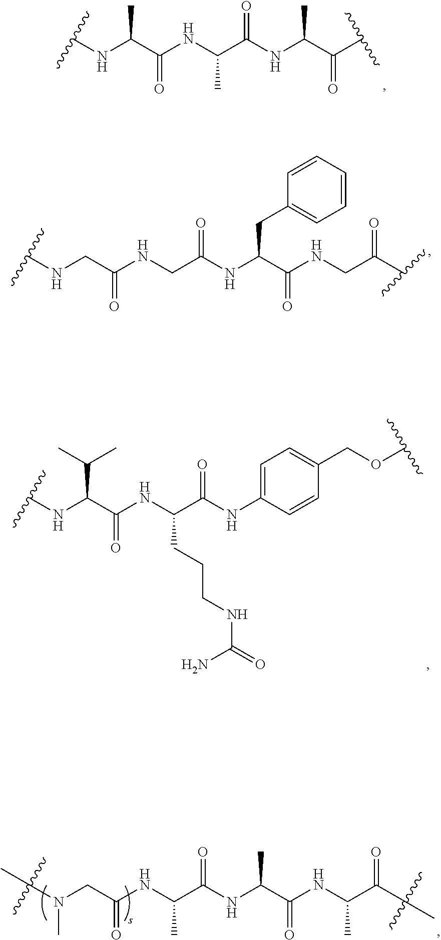

wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
E is a single bond or

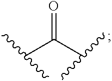 [SMILES: *C(*)=O ; ";"]

D is the following structure:

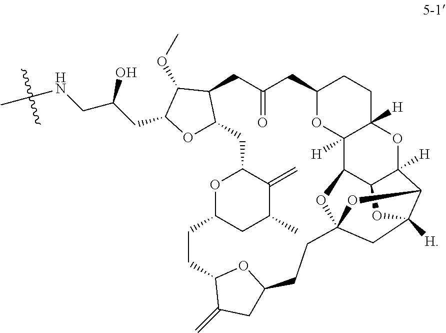

Ab is an antibody or antigen-binding fragment thereof, preferably an antibody or antigen-binding fragment thereof capable of specifically binding to epidermal growth factor receptor 2 (Her2), a member of the ErbB family of receptor tyrosine kinases,
the antibody or antigen-binding fragment thereof is as defined above,
preferably, the antibody or antigen-binding fragment thereof comprises:
(1) the following heavy chain variable region (VH) and/or light chain variable region (VL):
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
preferably, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof,
wherein, the variant described in any one of (1a) and (lb) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable regions (VH) and/or light chain variable regions (VL), wherein the CDRs are defined according to the IMGT numbering system:
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, and a CDR-L2 having a sequence as set forth in SEQ ID NO: 17 or variant thereof. L2, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
preferably, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25; or,
(4) the following heavy chain variable region (VH) and light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25;
preferably, the antibody or antigen-binding fragment thereof comprises:
(a) a VH as set forth in SEQ ID NO: 1 or variant thereof, and/or, a VL as set forth in SEQ ID NO: 2 or variant thereof, or
(b) a VH as set forth in SEQ ID NO: 3 or variant thereof, and/or, a VL as set forth in SEQ ID NO: 4 or variant thereof, or
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution;
preferably, the antibody or antigen-binding fragment thereof comprises:
(a) a VH as set forth in SEQ ID NO: 1, and a VL as set forth in SEQ ID NO: 2; or
(b) a VH as set forth in SEQ ID NO: 3, and a VL as set forth in SEQ ID NO: 4;
preferably, the antibody or antigen-binding fragment thereof is selected from Trastuzumab or Pertuzumab or antigen-binding fragment thereof.

In some embodiments, the antibody-drug conjugate is selected from the group consisting of ADC J-1 to ADC J-9 and ADC H-1 to ADC H-15 shown below:

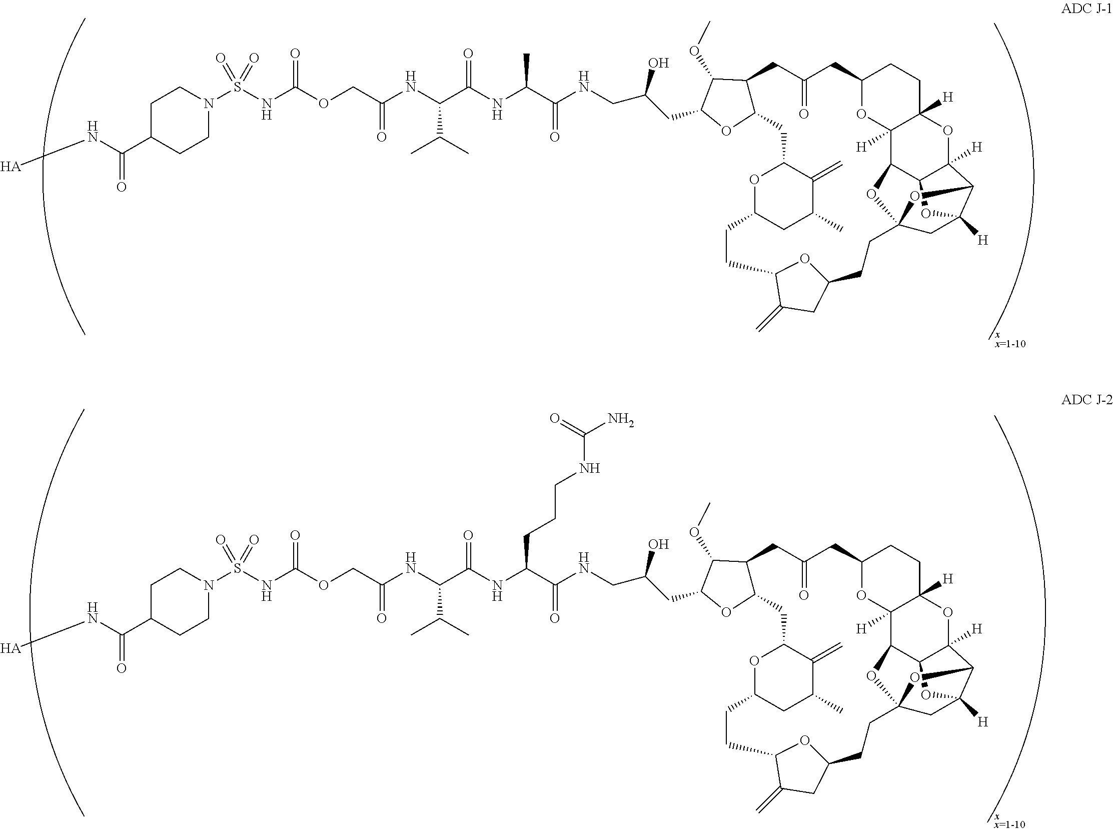

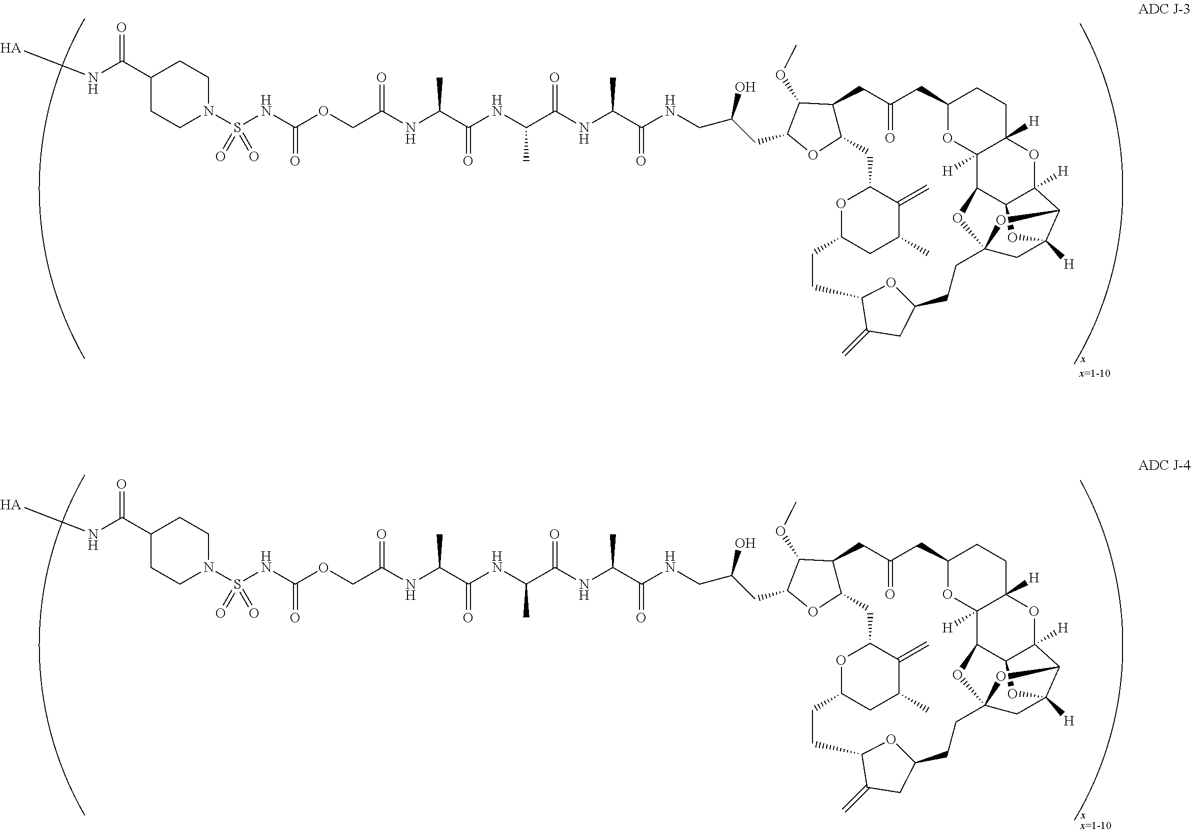

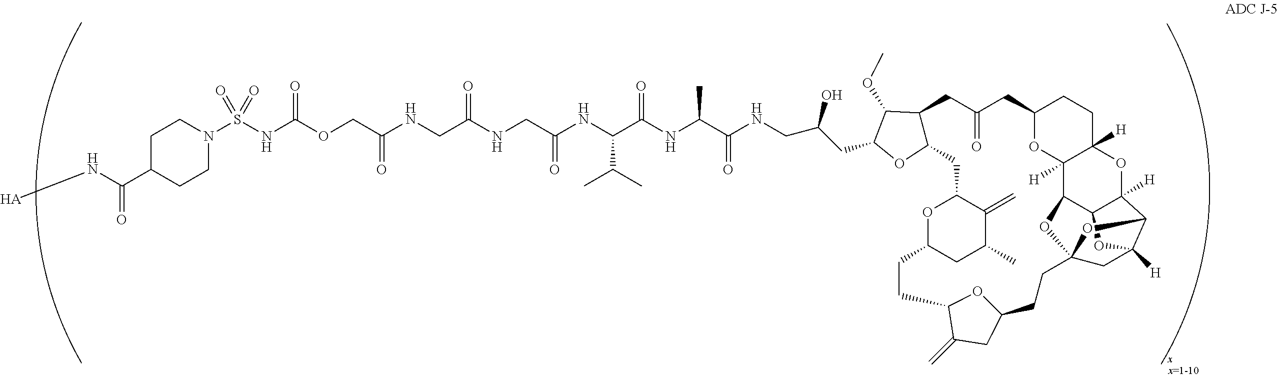

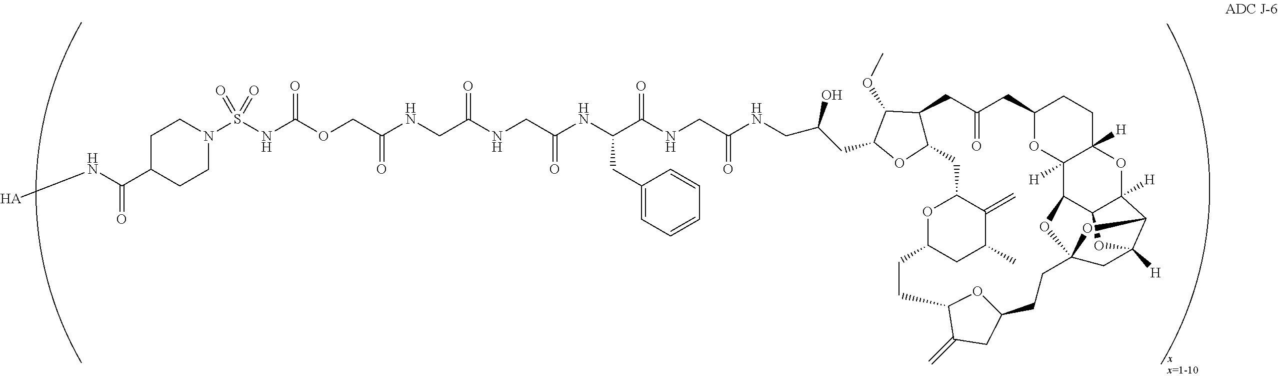

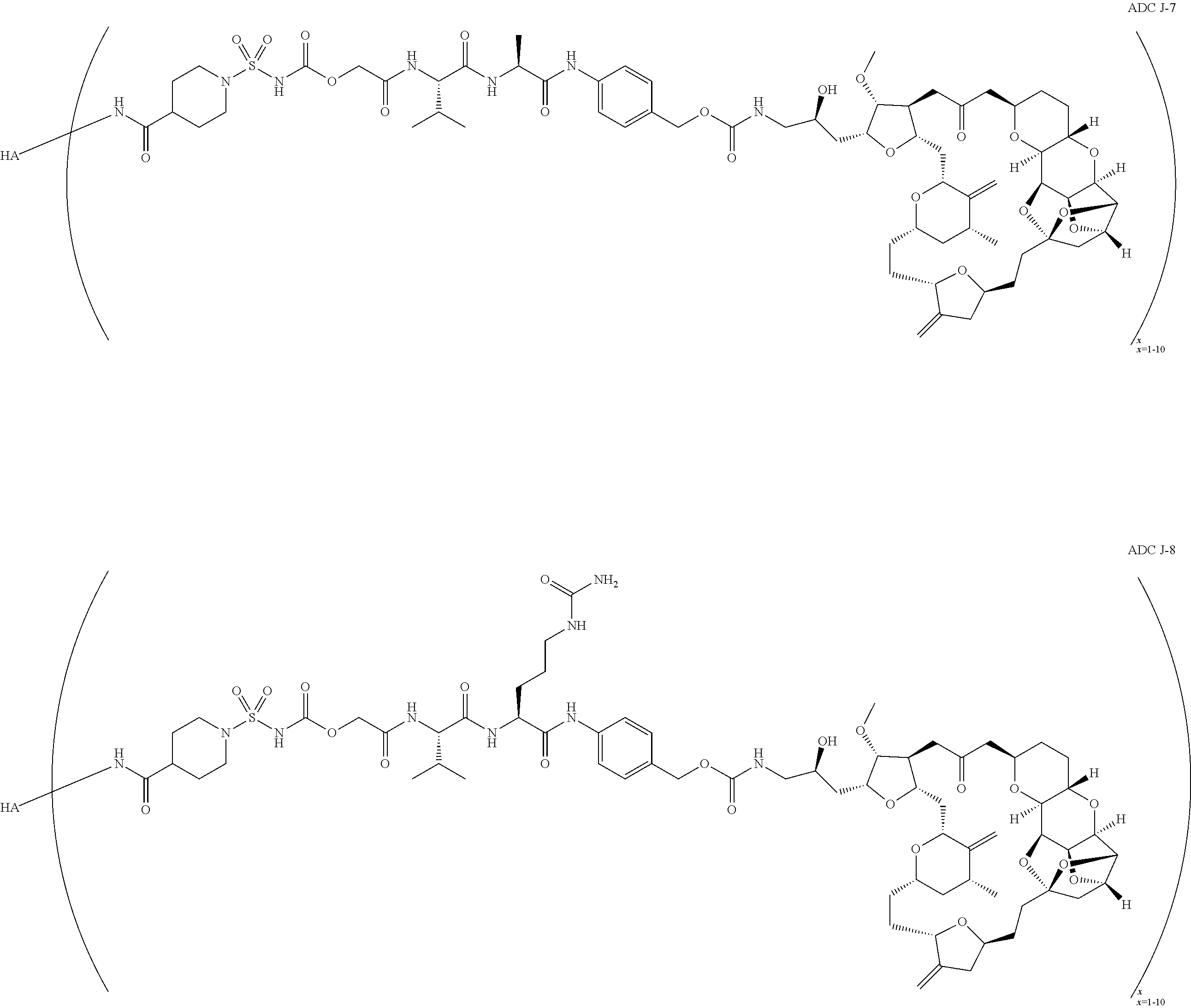

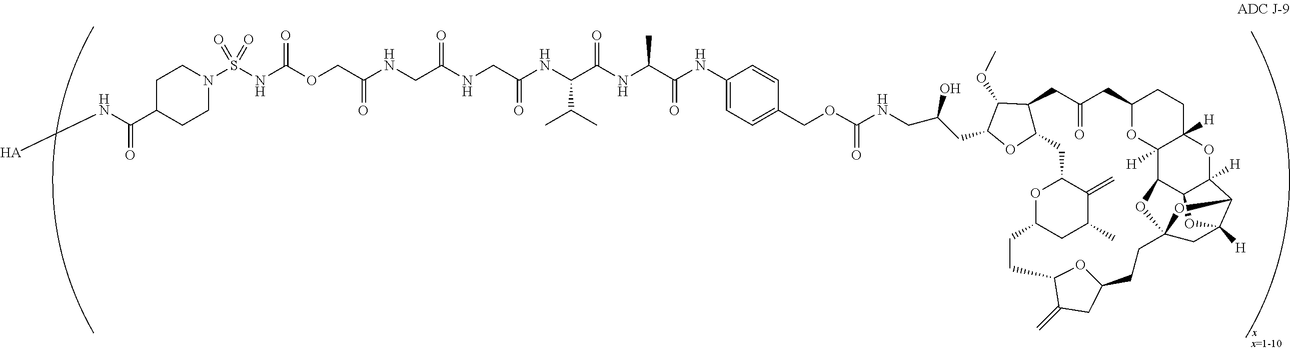

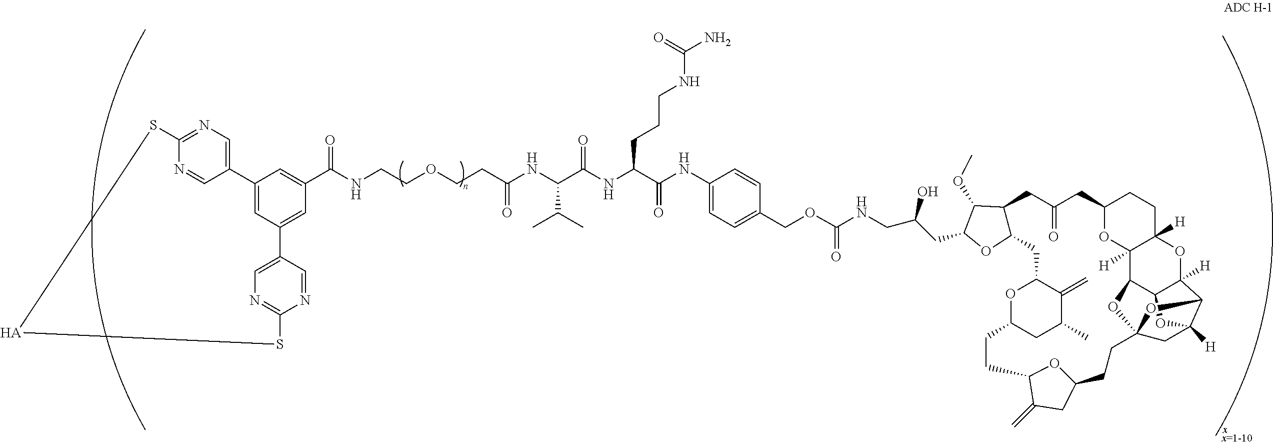

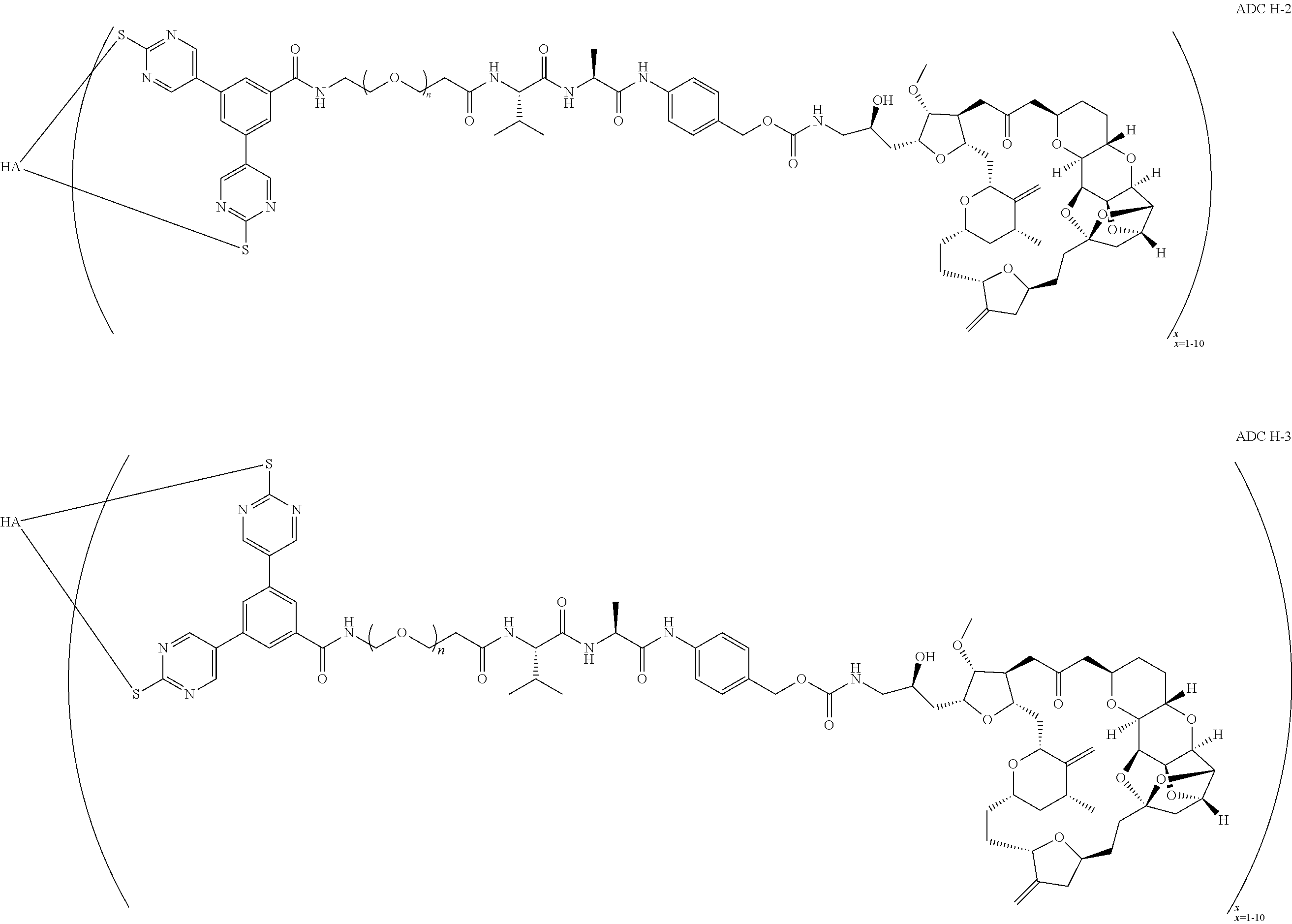

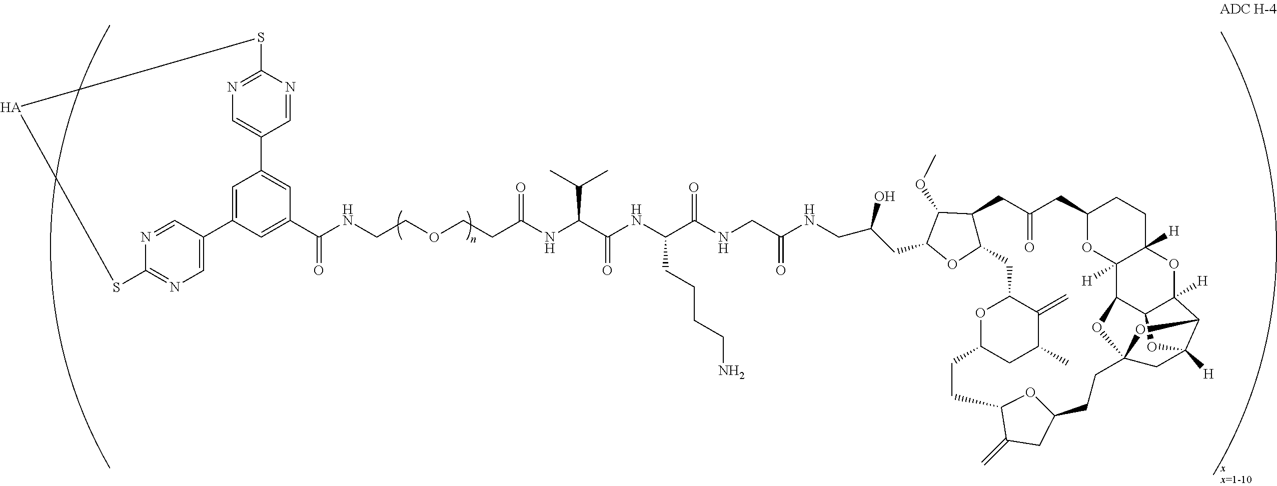

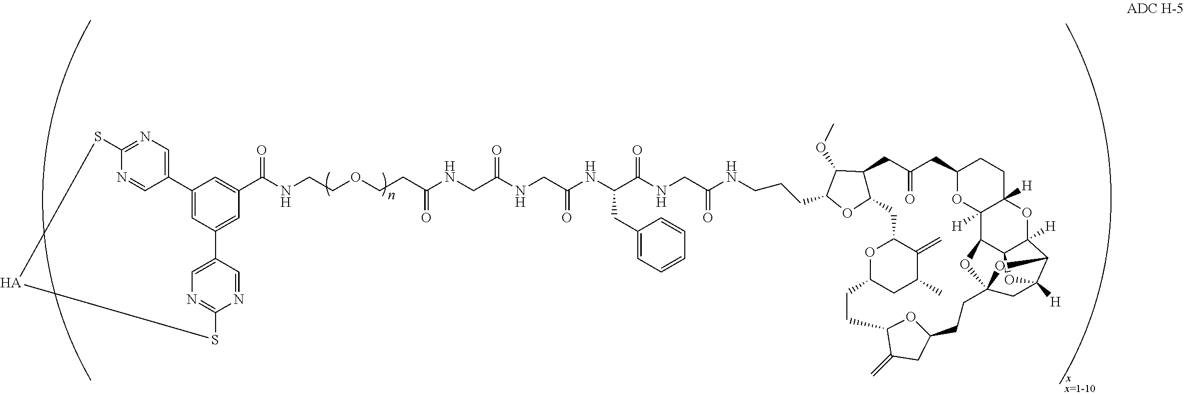

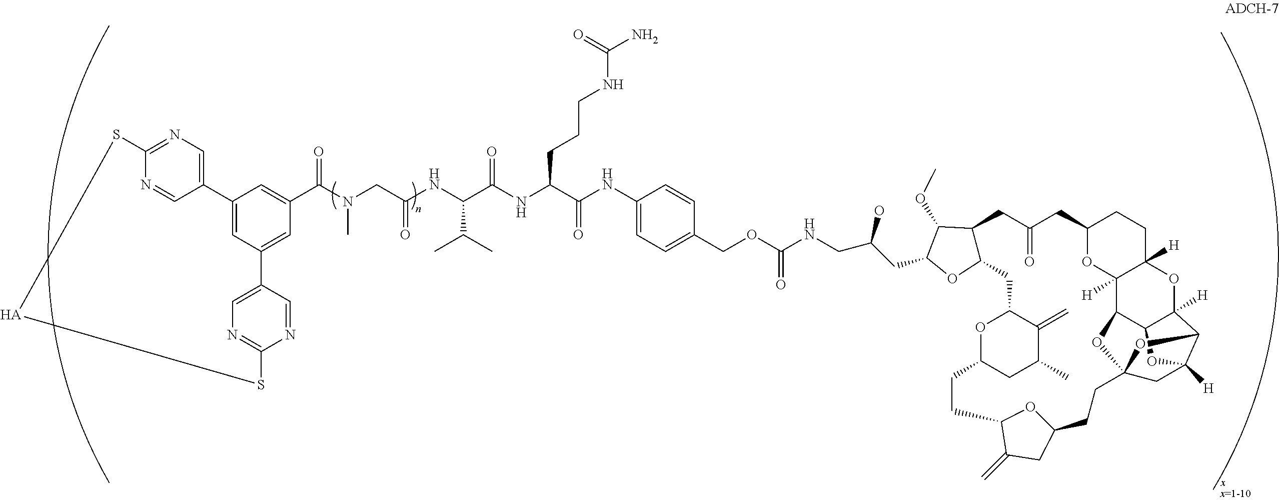

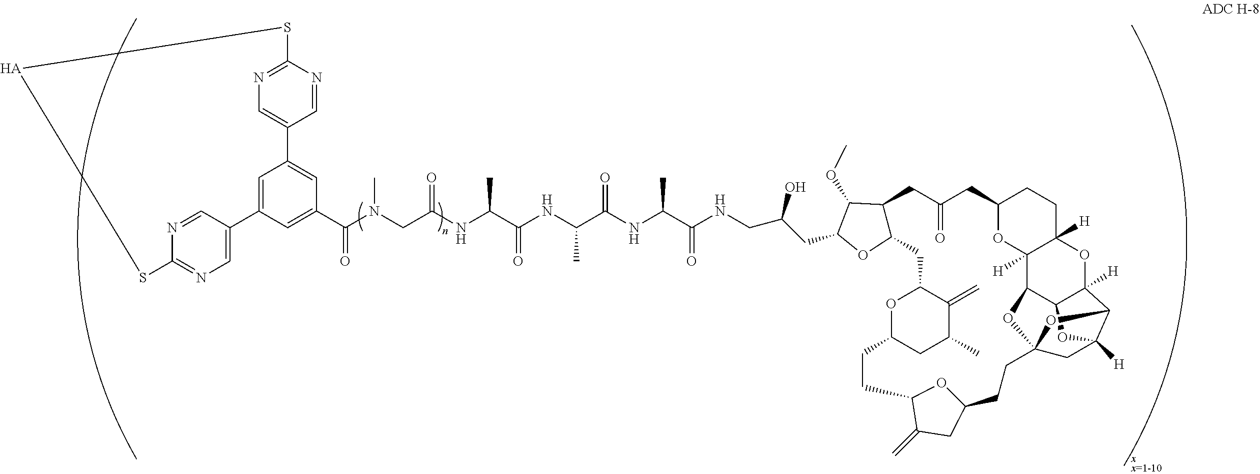

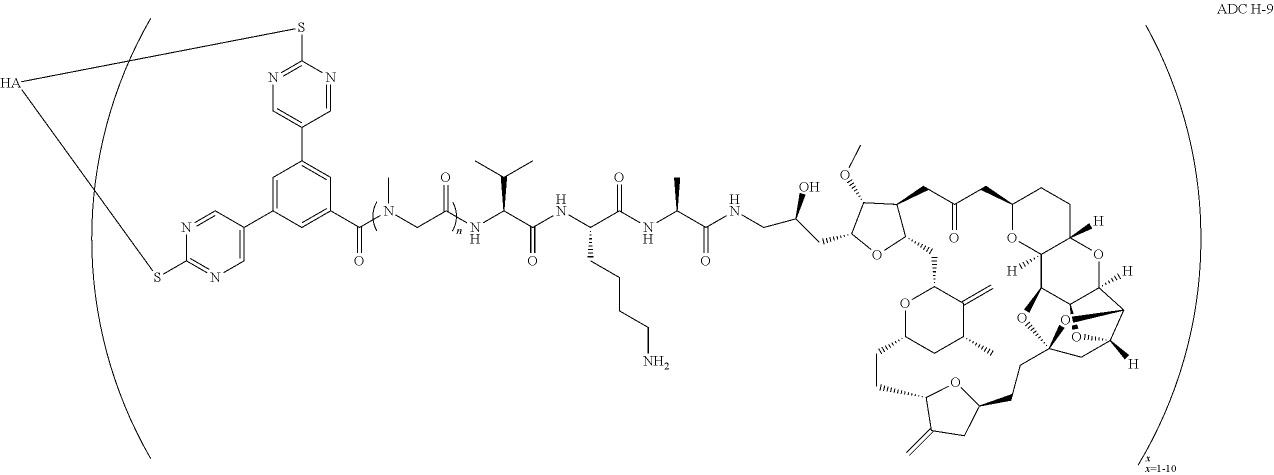

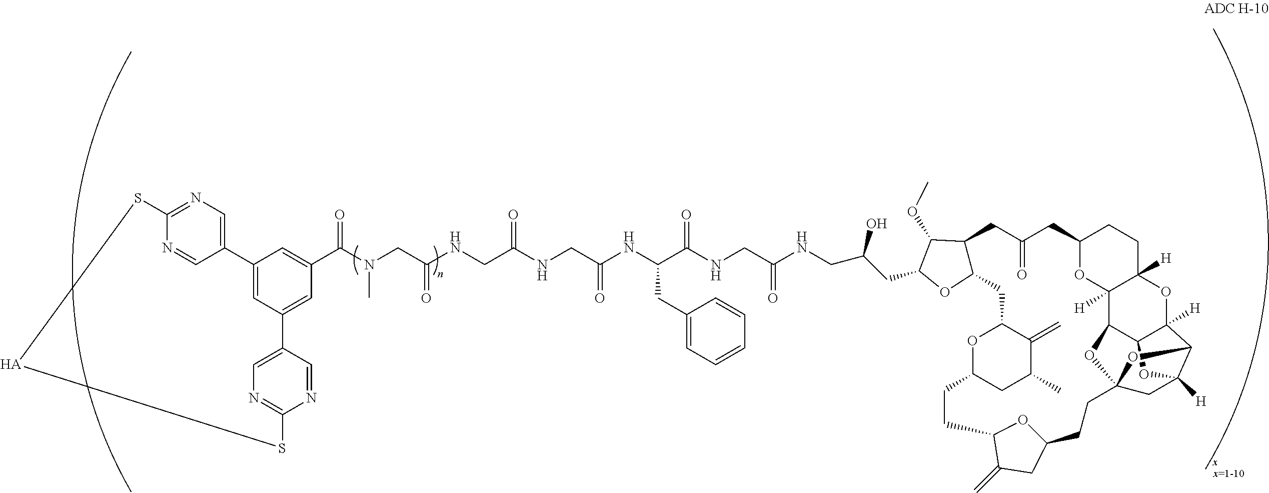

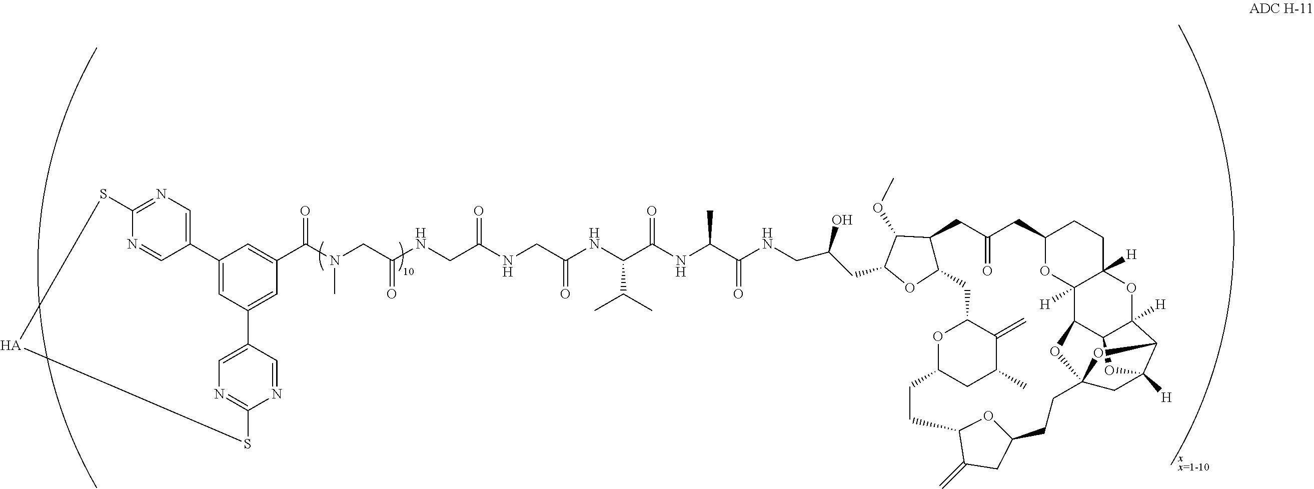

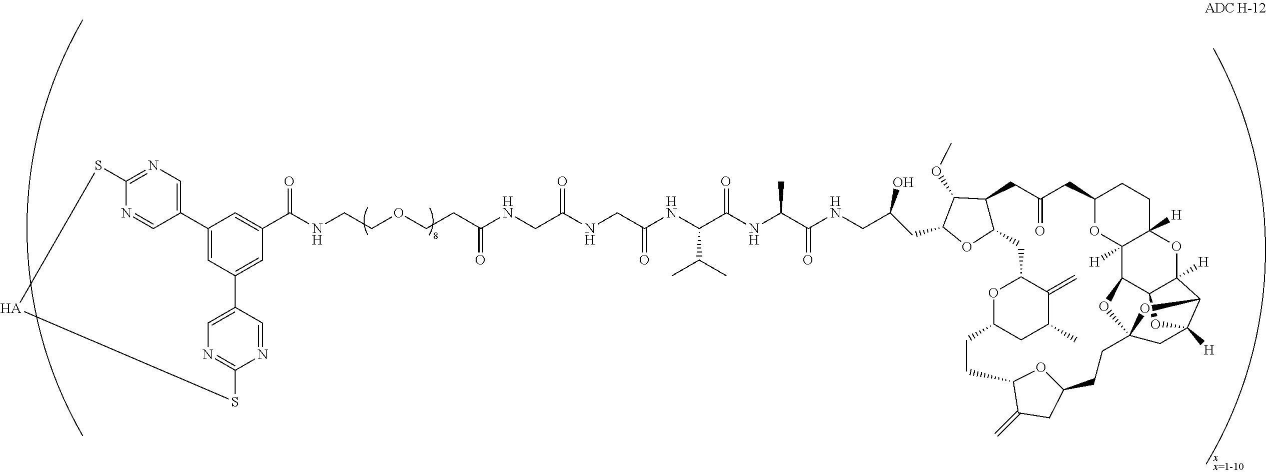

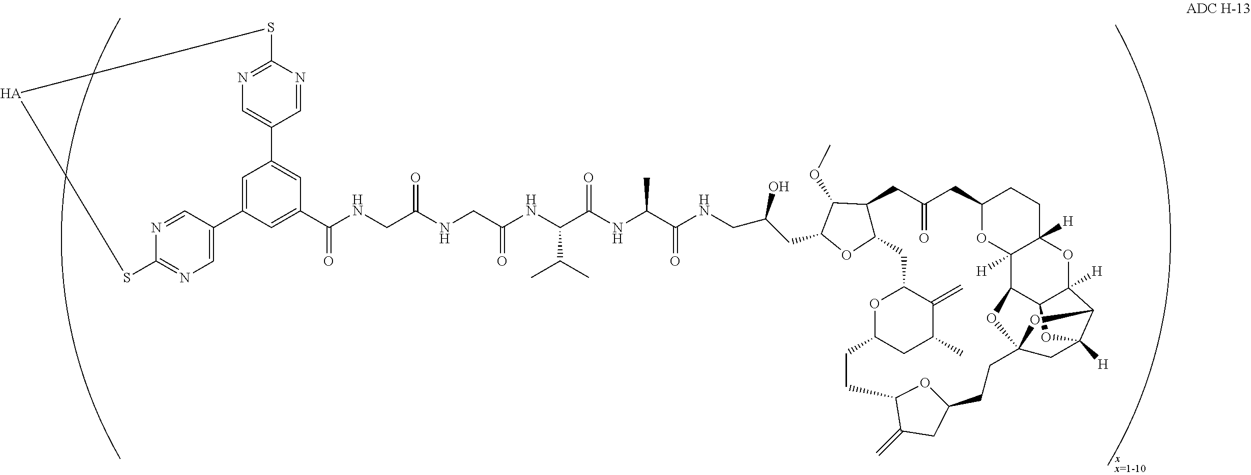

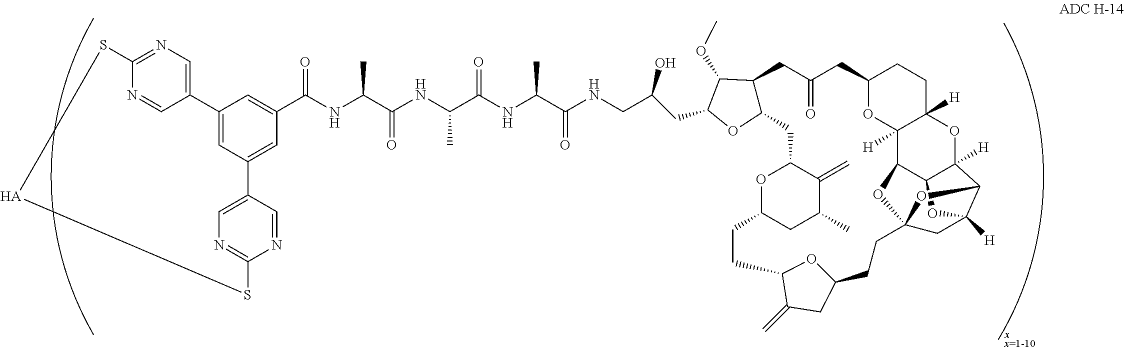

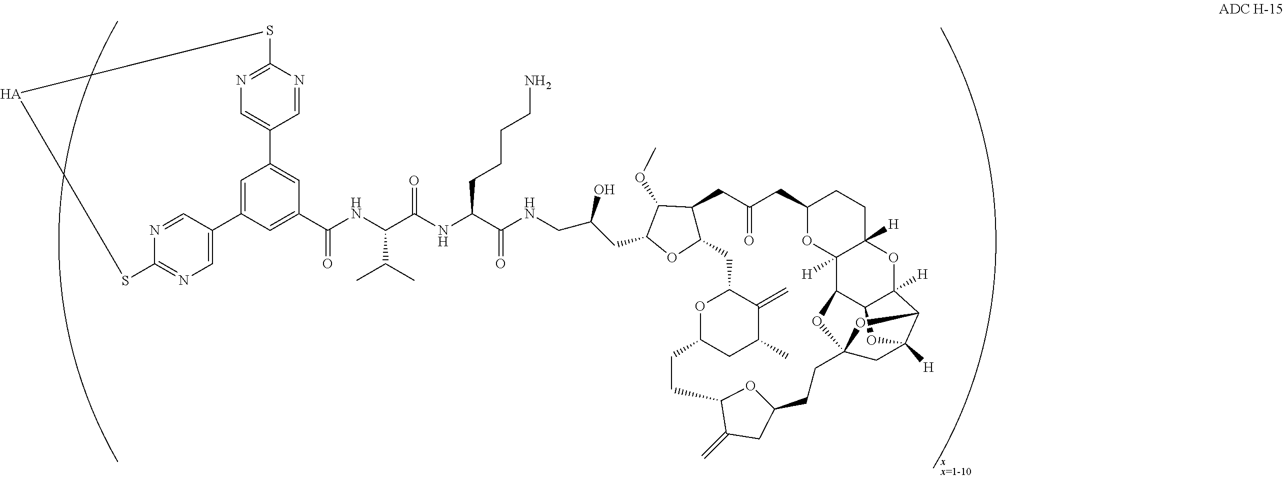

wherein, HA in each antibody-drug conjugate represents an antibody or antigen-binding fragment thereof, n is an integer selected from 1 to 20, preferably an integer of 1 to 15, such as an integer of 1 to 12, 3 to 12, 5 to 10, 8 to 10, such as 1, 2, 3, 4, 5, 6, 7, 8, 9, 10, 11, 12, 13, 14 or 15, preferably 5, 8, 10;
wherein,

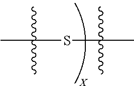 [SMILES: *S*]

represents the specific linkage between the mercapto group in the antibody or antigen-binding fragment thereof and the M fragment;

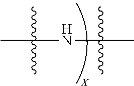 [SMILES: *N*]

represents the specific linkage between the amino group in the antibody or antigen-binding fragment thereof and the M fragment.

In some embodiments, the antibody-drug conjugate is selected from the group consisting of ADC H′-1 to ADC H′-12 shown below:

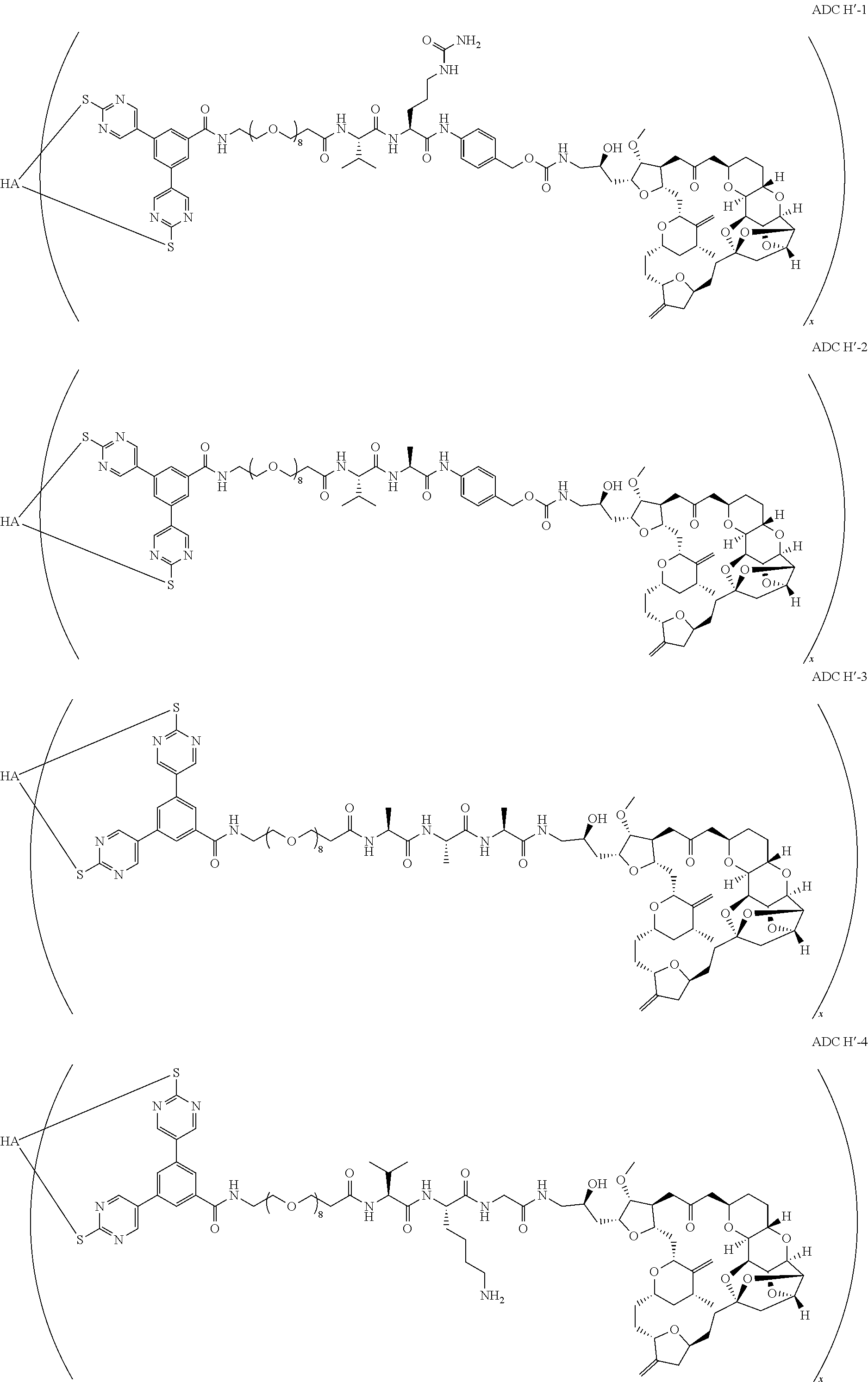

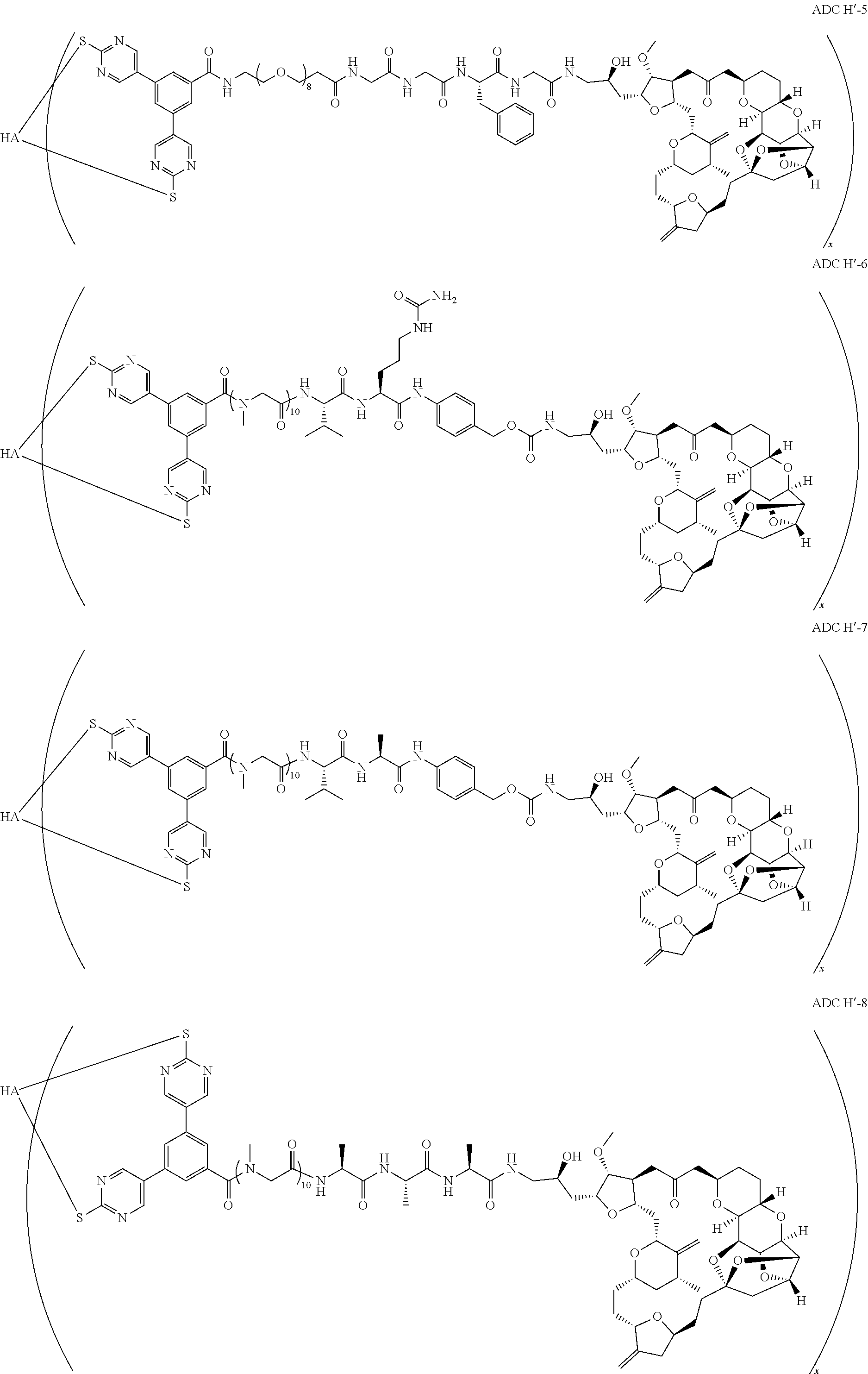

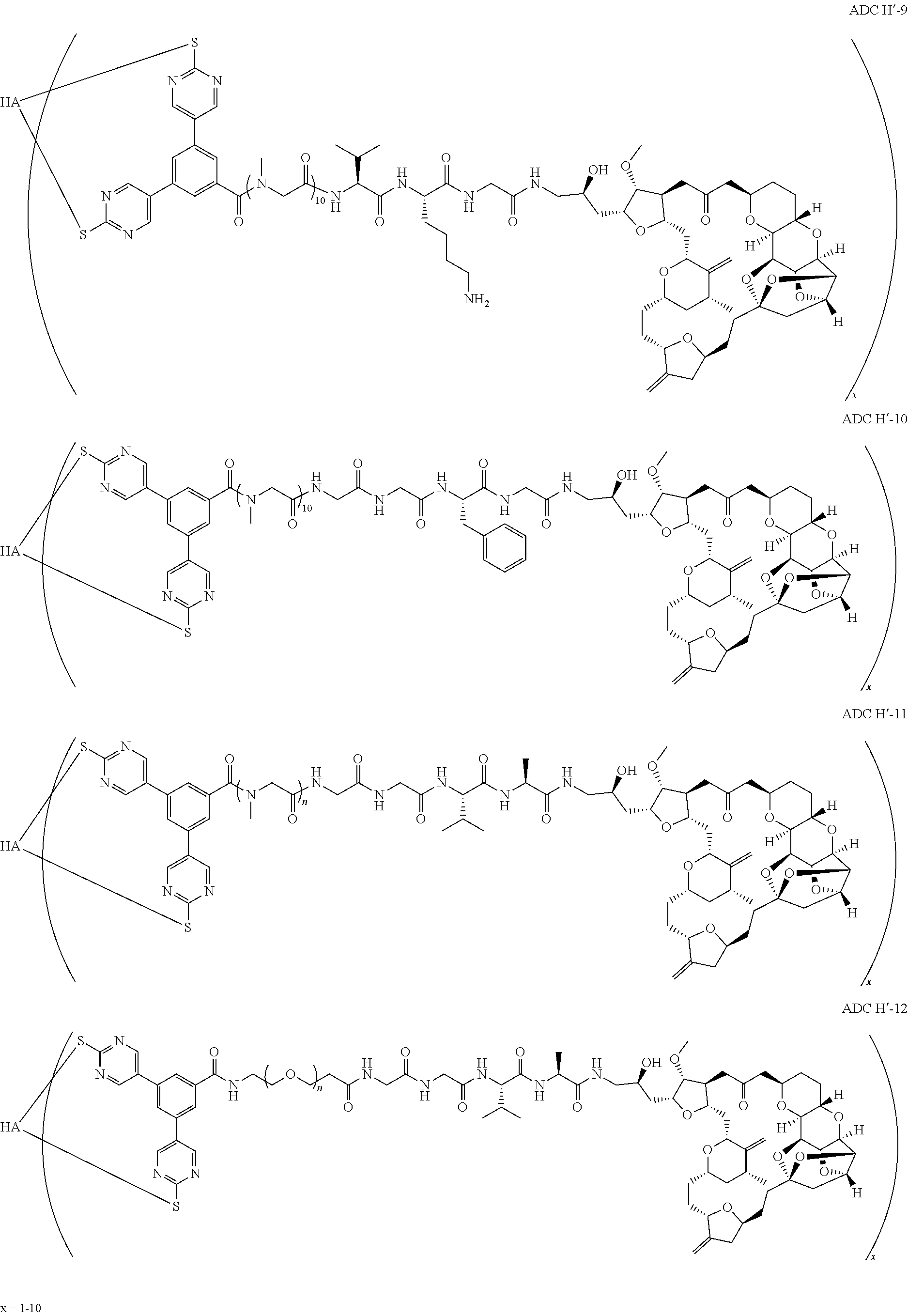

wherein, HA in each antibody-drug conjugate represents an antibody or antigen-binding fragment thereof; n is an integer selected from 1 to 20, preferably an integer of 1 to 15, such as an integer of 1 to 12, 3 to 12, 5 to 10, 8 to 10, such as 1, 2, 3, 4, 5, 6, 7, 8, 9, 10, 11, 12, 13, 14 or 15,preferably 5, 8, 10,

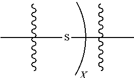

represents the specific linkage between the mercapto group in the antibody or antigen-binding fragment thereof and the M fragment,

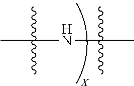 [SMILES: *N*]

represents the specific linkage between the amino group in the antibody or antigen-binding fragment thereof and the M fragment.

In some embodiments, the antibody or antigen-binding fragment thereof in each antibody- drug conjugate is defined as described above.

In some embodiments, X in the conjugate represented by Ab-[M-L-E-D]ₓ is 1 to 10, such as 1 to 2,1 to 3, 1 to 4, 1 to 5, 1 to 6, 1 to 7, 1 to 8, 1 to 9, 1 to 10, 2 to 3, 2 to 4, 2 to 5, 2 to 6, 2 to 7,2 to 8,2 to 9,2 to 10, 3 to 4, 3 to 5, 3 to 6, 3 to 7, 3 to 8, 3 to 9, 3 to 10, 4 to 5, 4 to 6, 4 to 7, 4 to 8, 4 to 9 or 4 to 10.

In some embodiments, X in the conjugate represented by Ab-[M-L-E-D]ₓ is 1, 2, 3, 4, 5, 6, 7, 8, 9 or 10.

In some embodiments, X in the conjugate represented by Ab-[M-L-E-D]ₓ is 1, 2, 3 or 4.

In some embodiments, X in the conjugate shown by Ab-[M-L-E-D]ₓ is 3, 4 or 5.

In some embodiments, X in the conjugate shown by Ab-[M-L-E-D]ₓ is 2 or 4.

In some embodiments, the conjugate described in the present invention is an antibody-drug conjugate (ADC).

In some embodiments, the conjugate described in the present invention is optionally substituted with one or more suitable substituents.

### Intermediates

In some embodiments, the present application provides the intermediate compounds having the following structures or salts, stereoisomers, tautomers, or isotopically labeled compounds thereof:

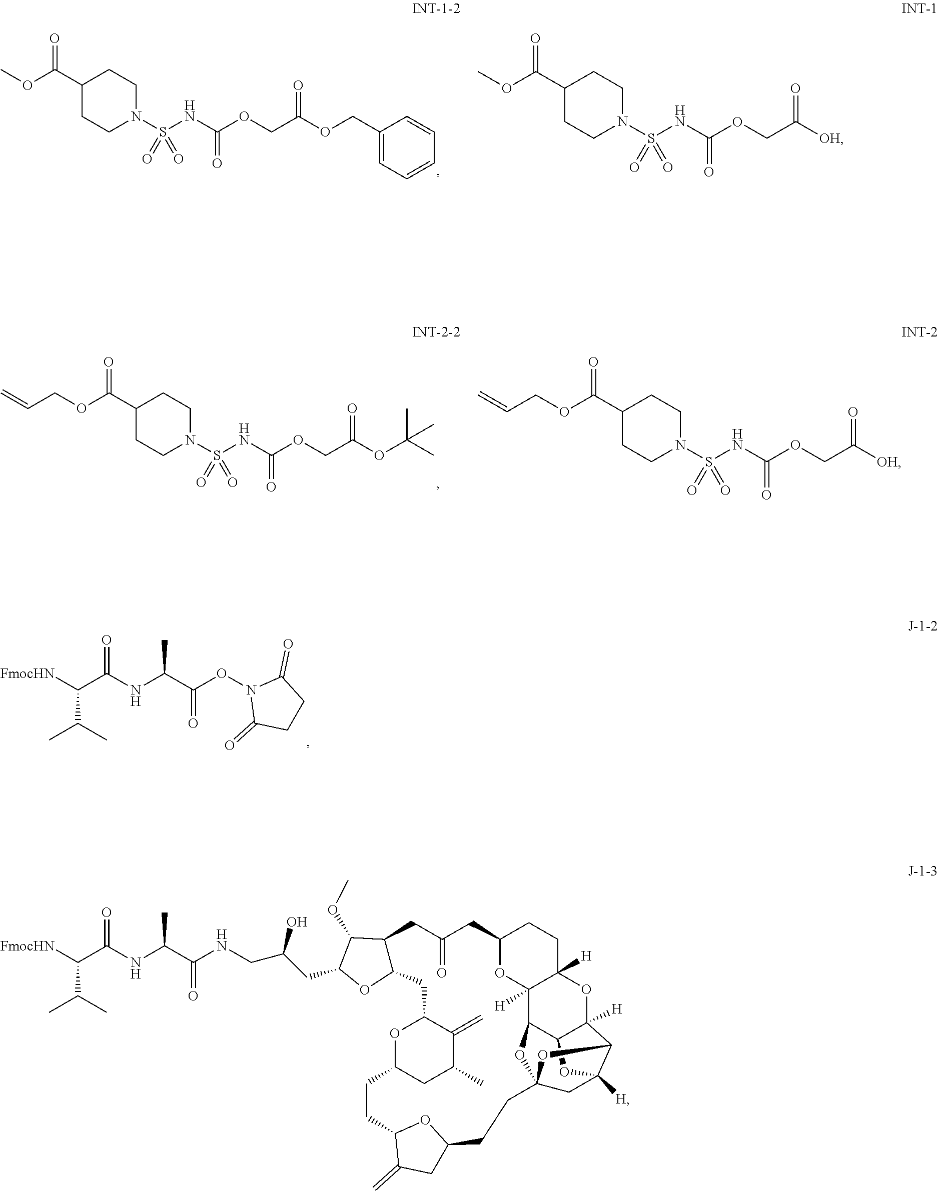

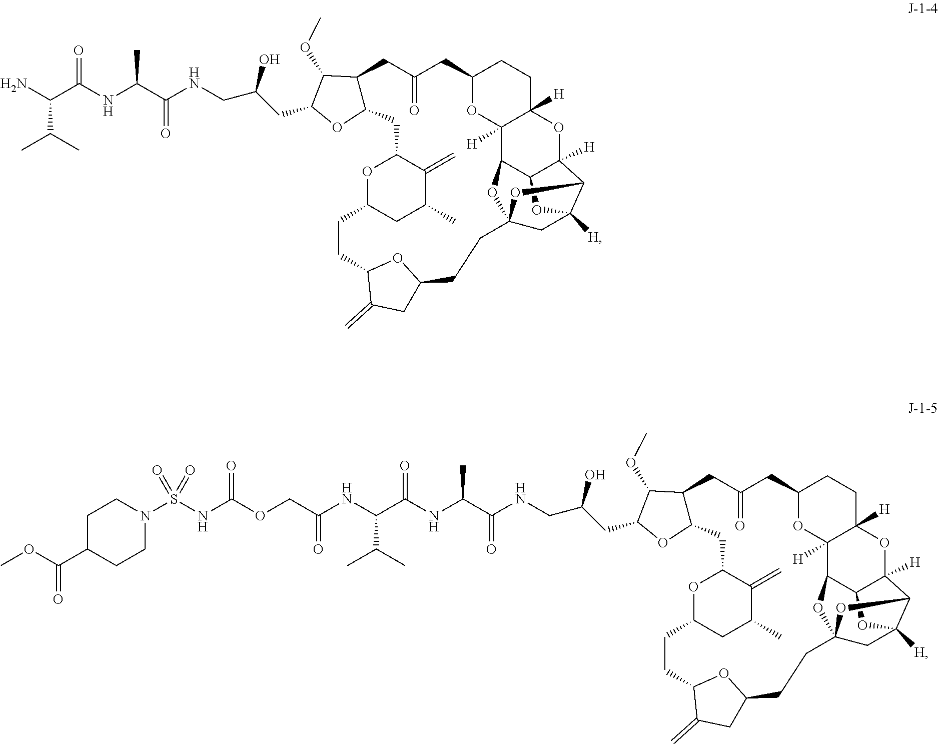

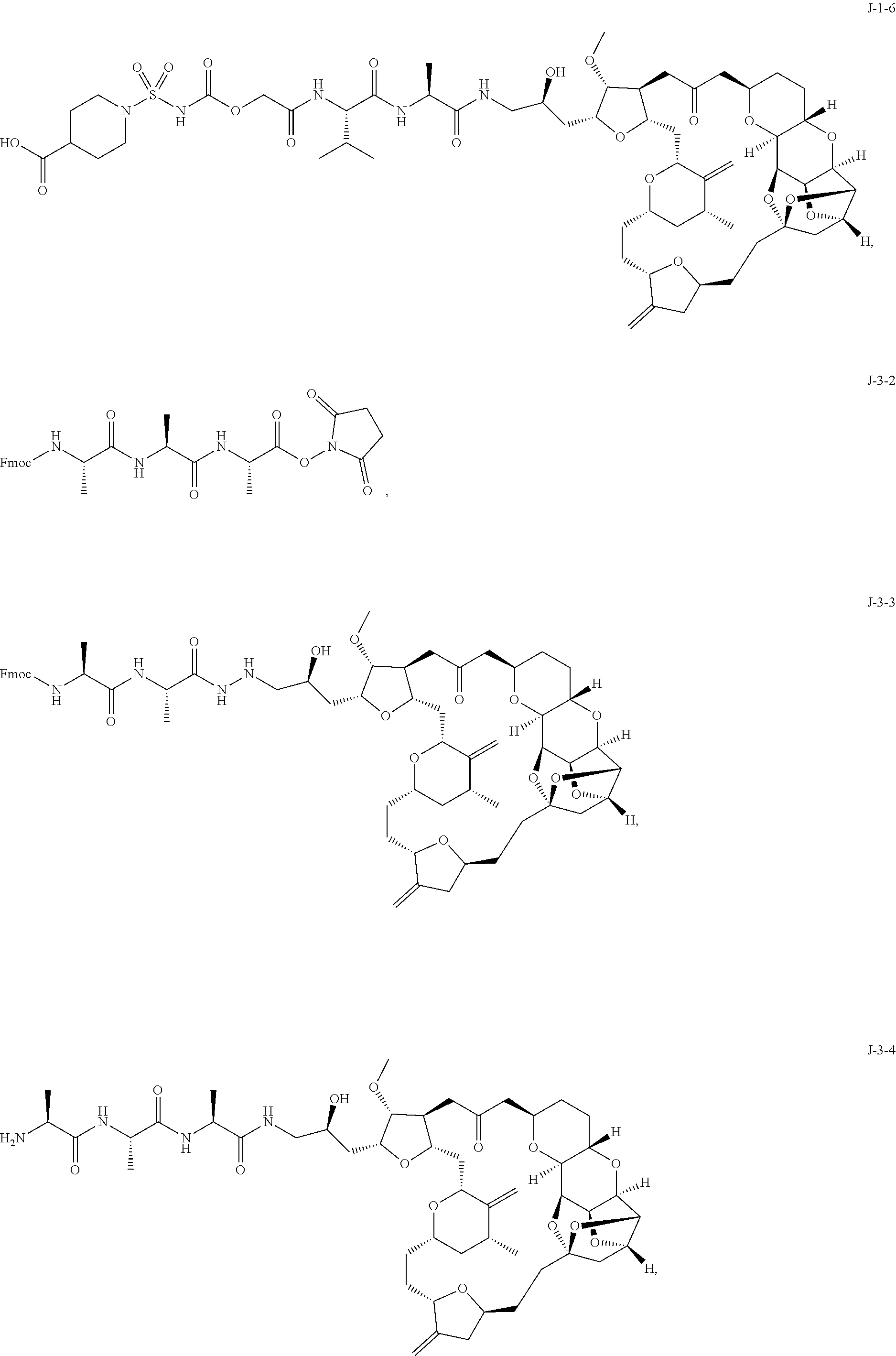

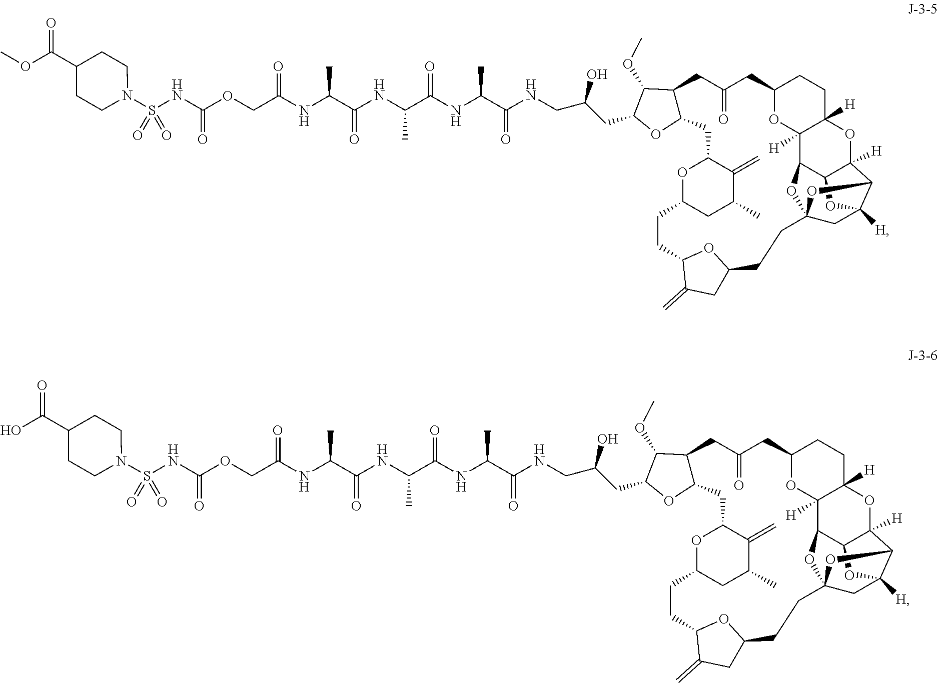

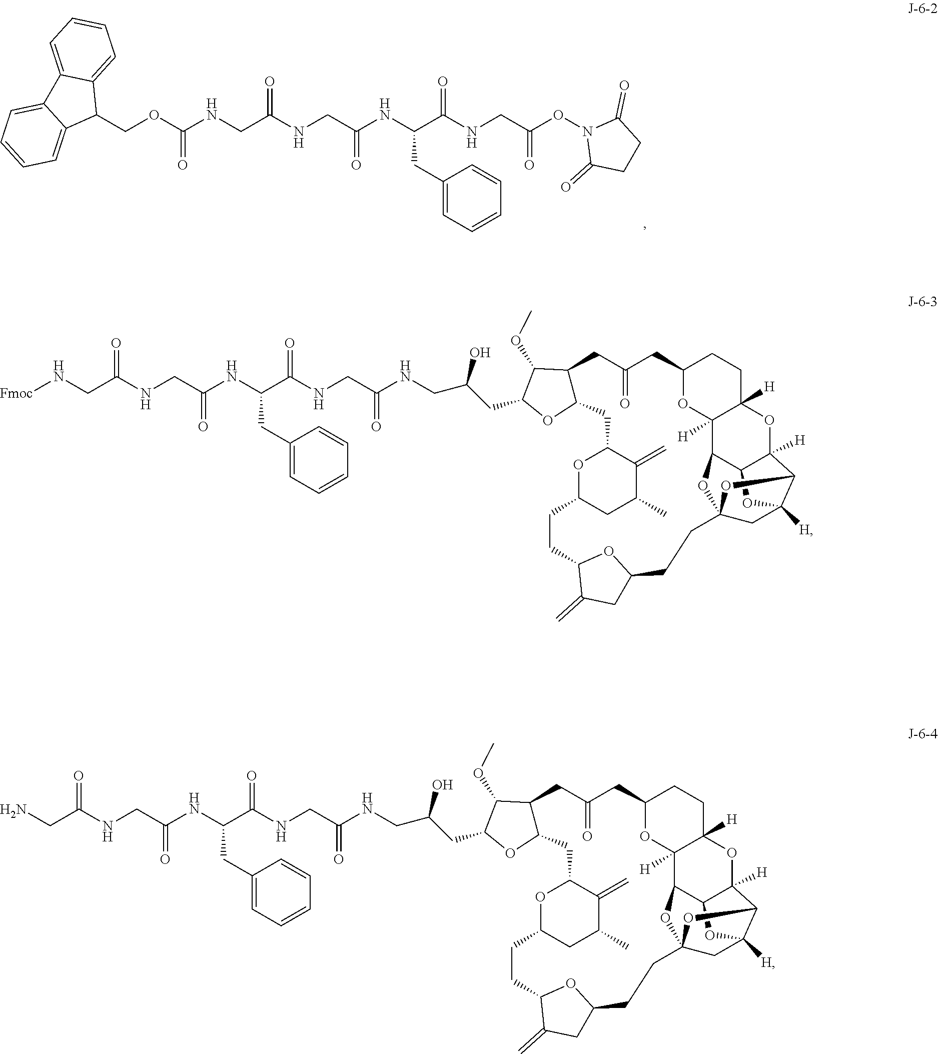

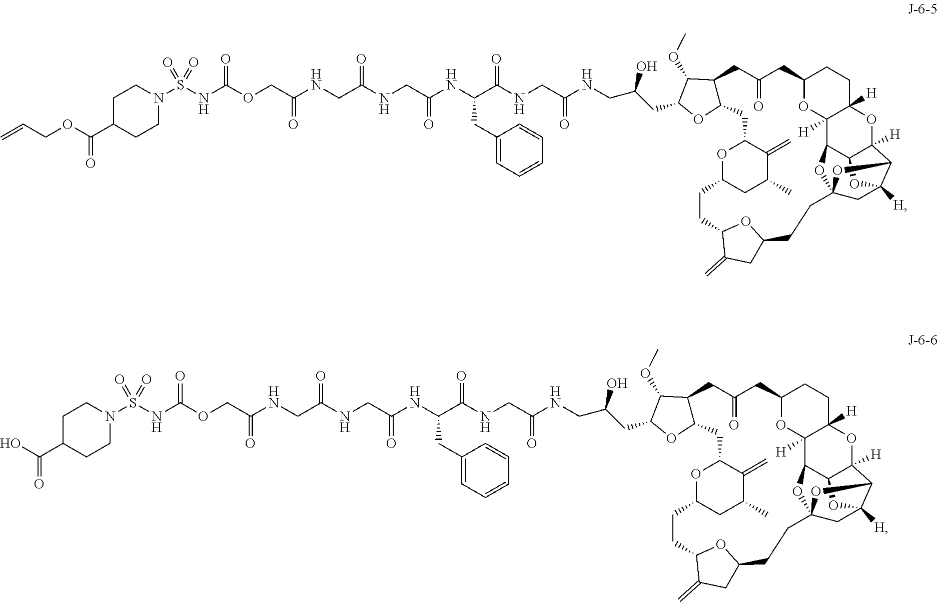

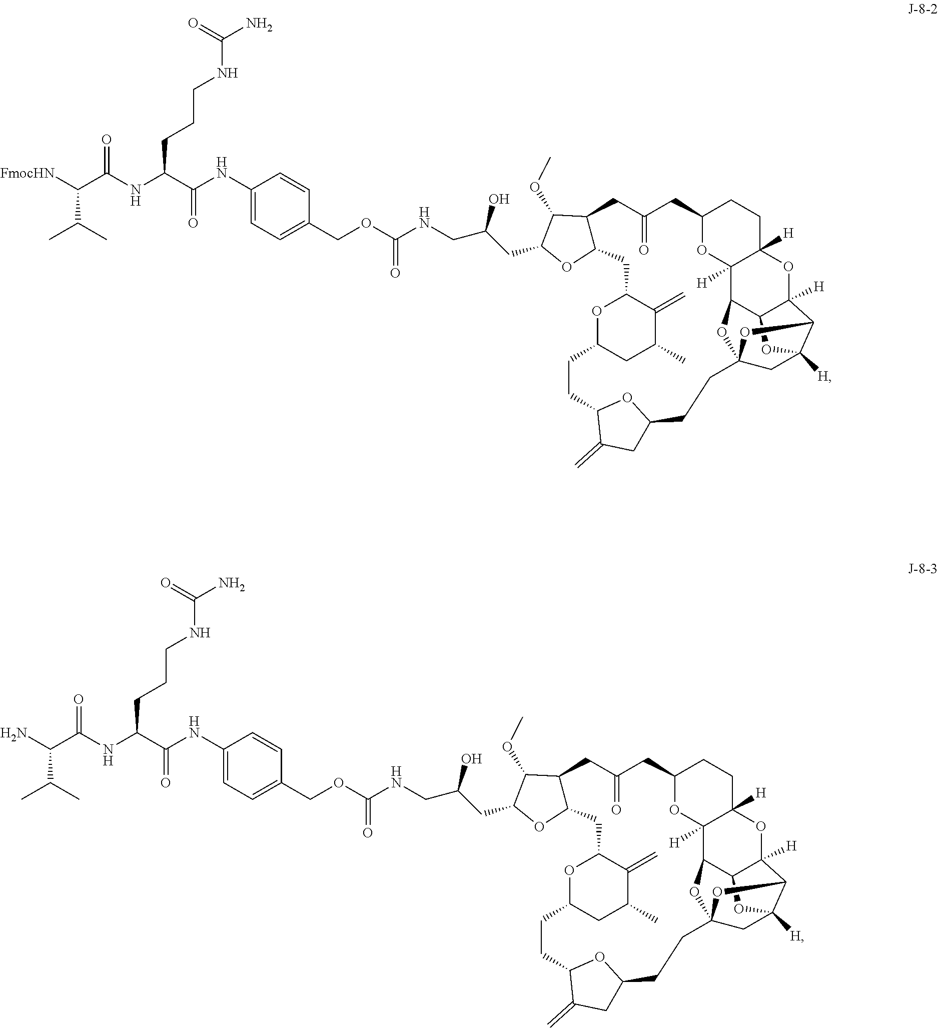

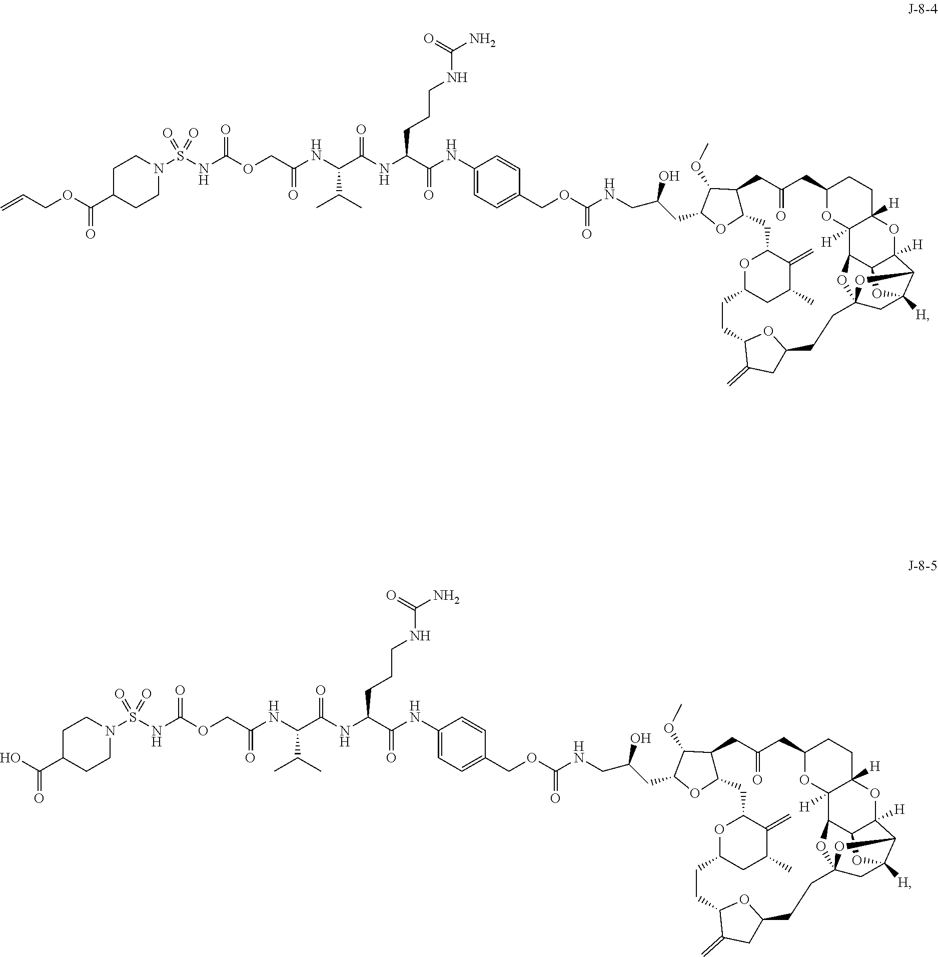

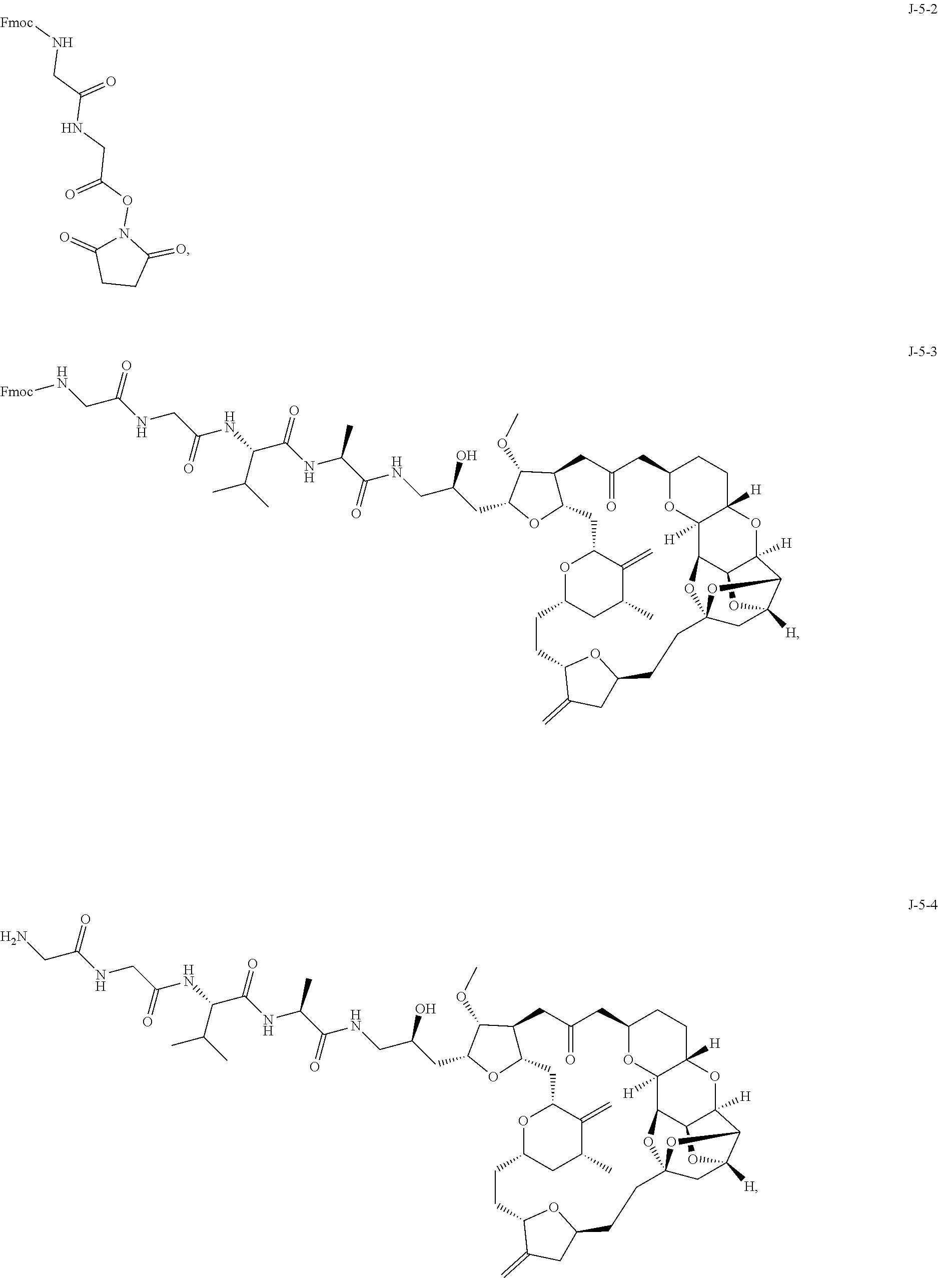

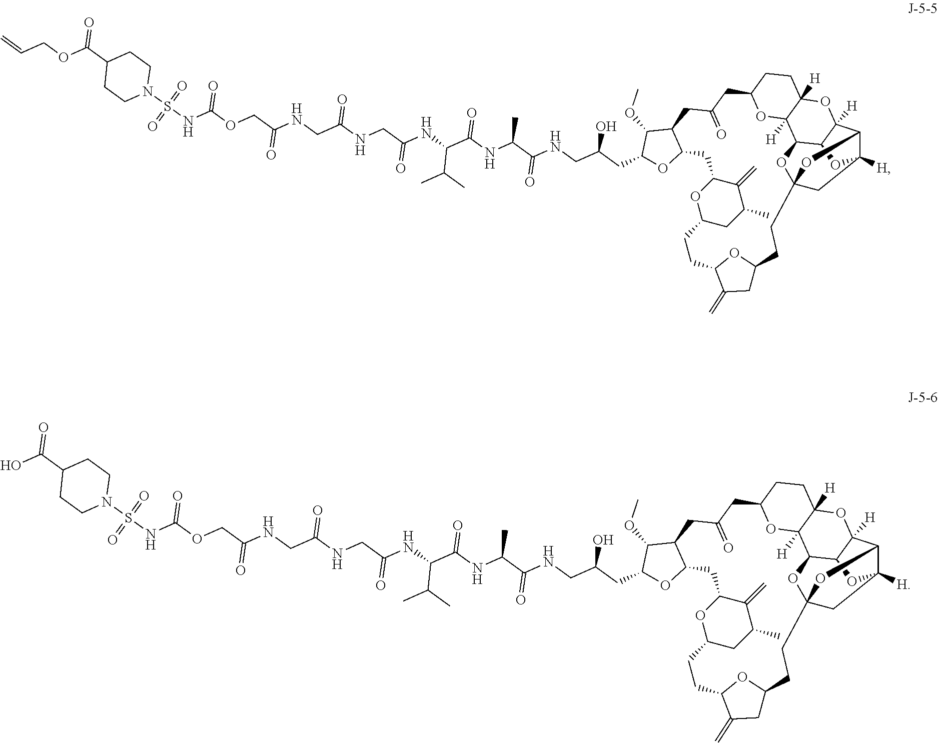

In some embodiments, the present application provides the intermediate compounds having the following structures or salts, stereoisomers, tautomers or isotopically labeled compounds thereof:

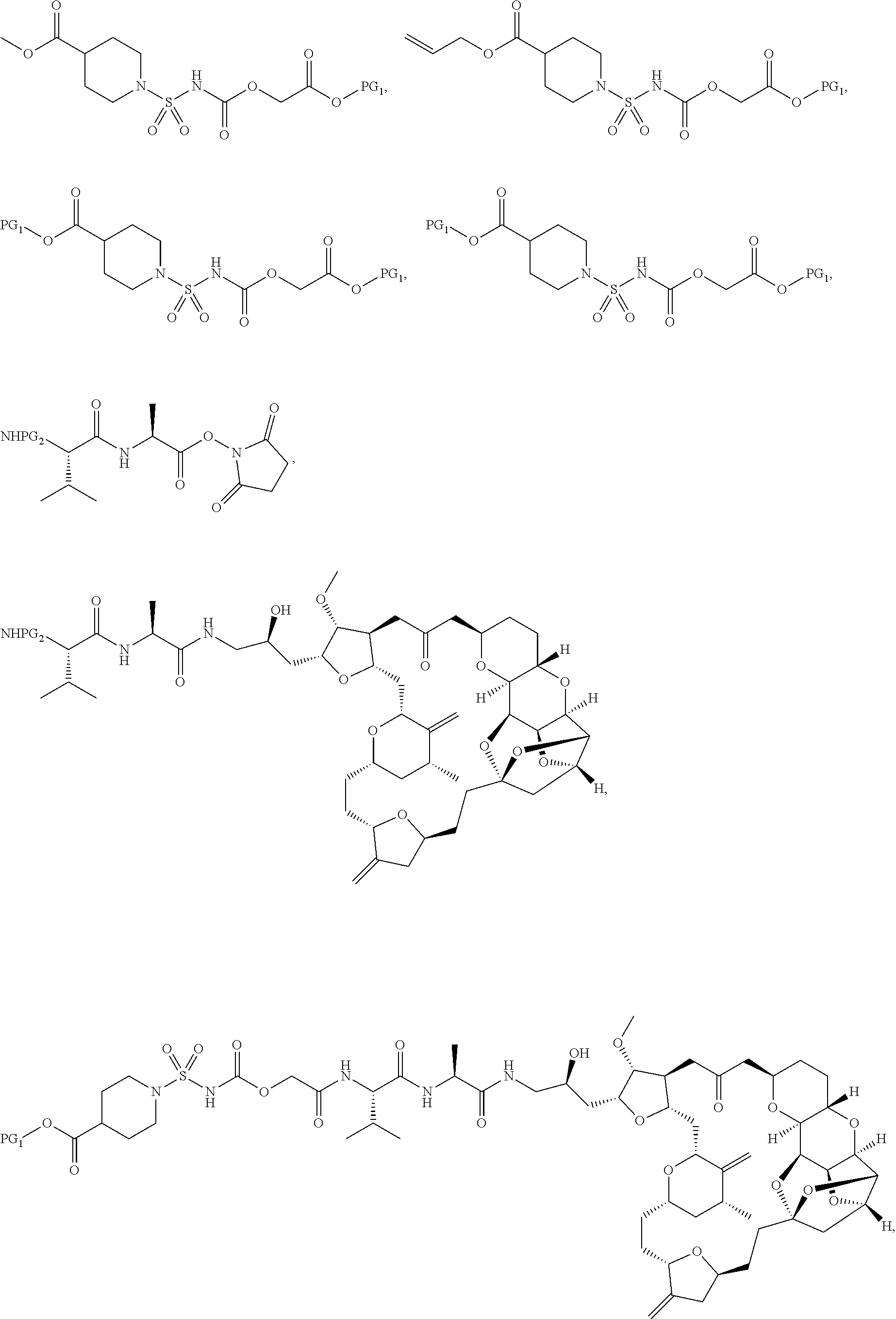

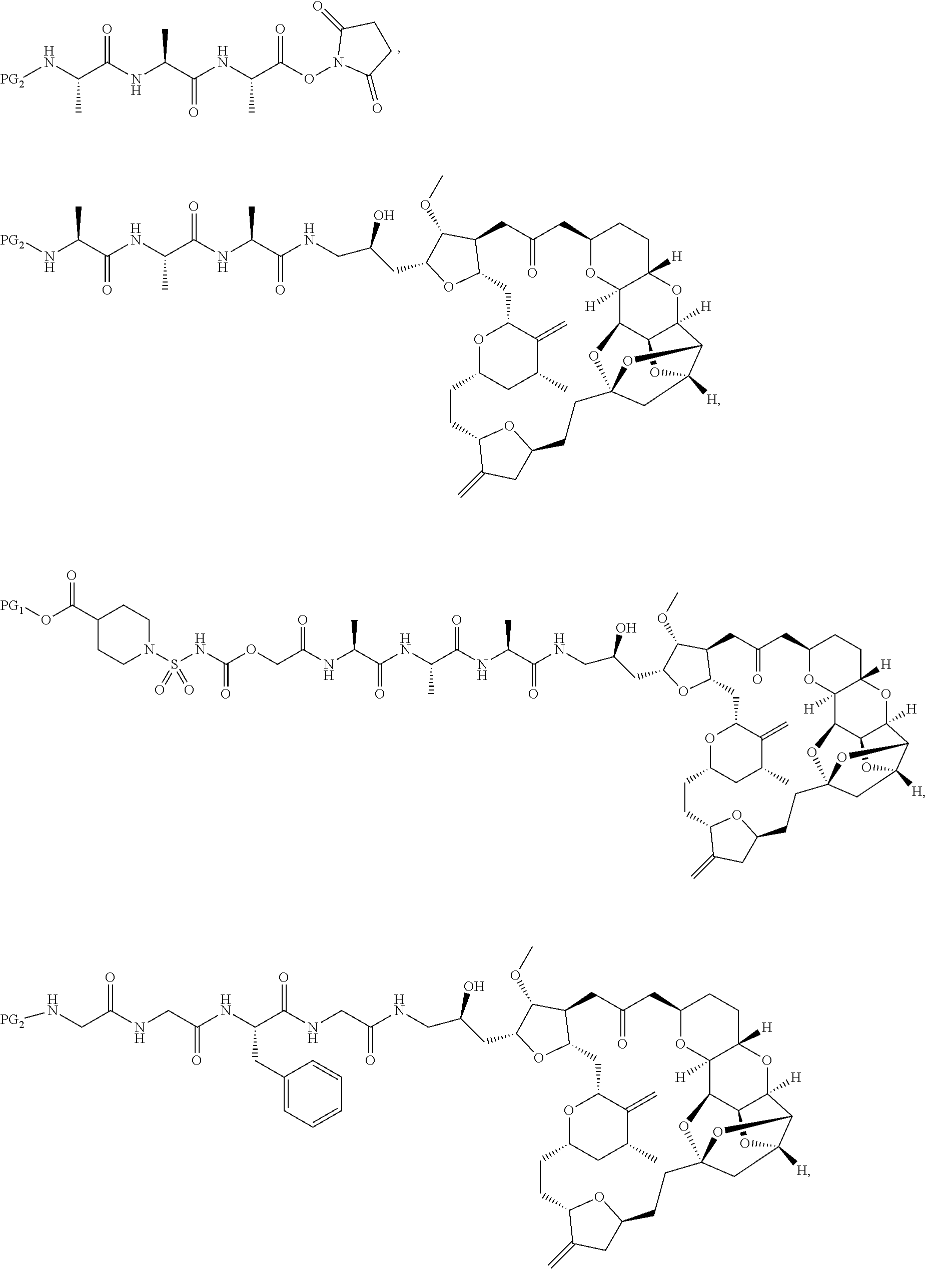

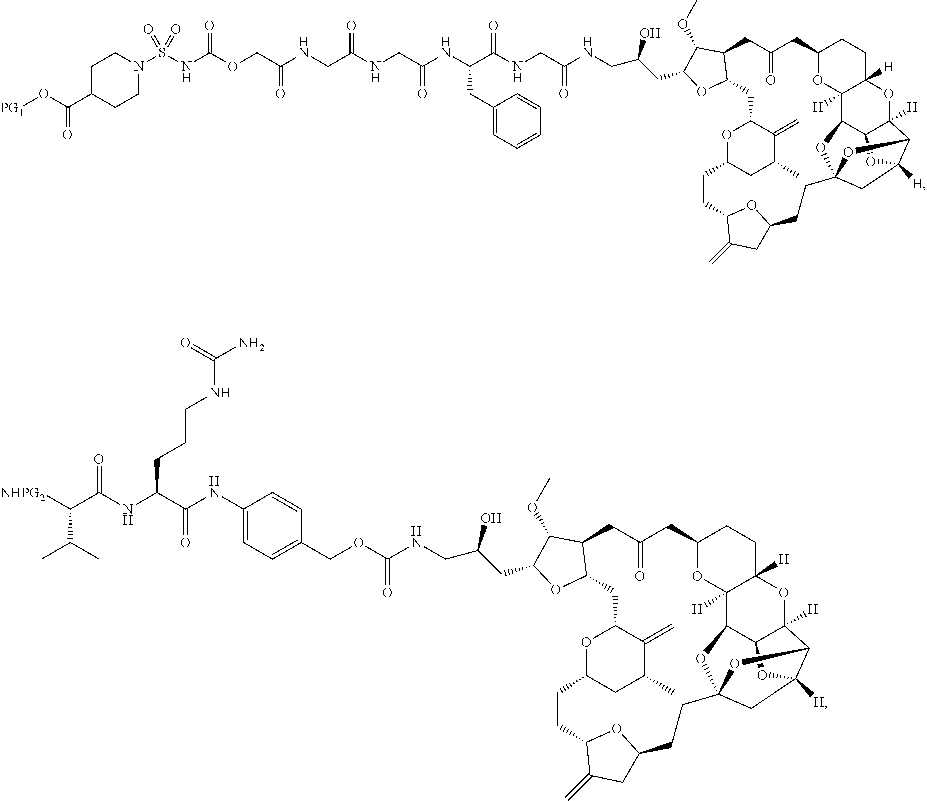

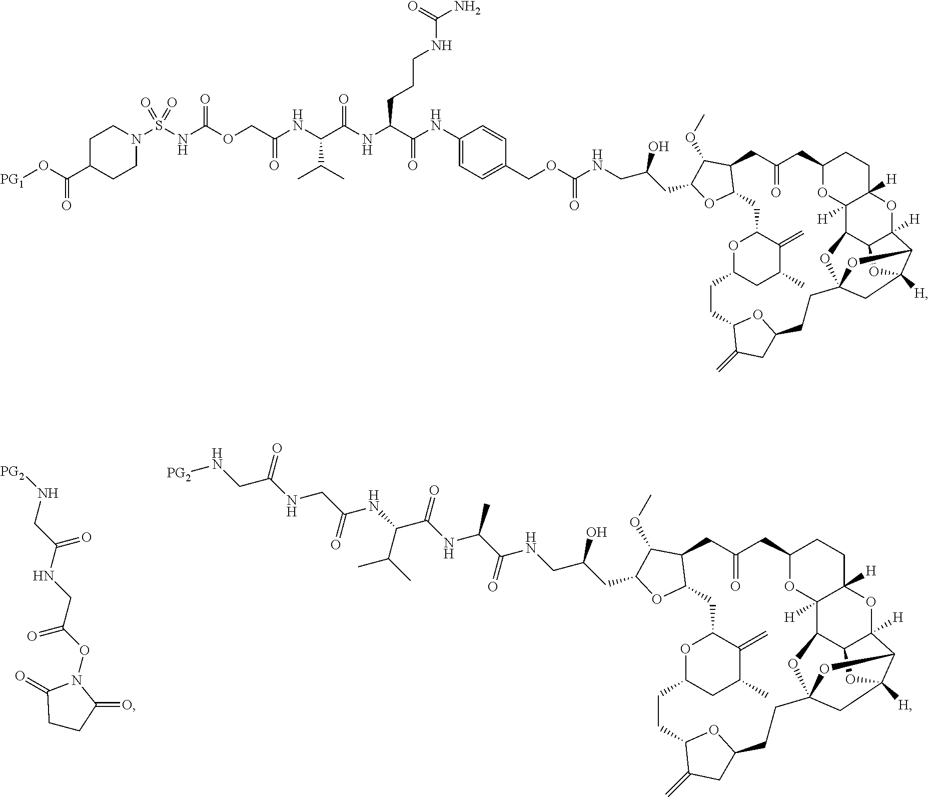

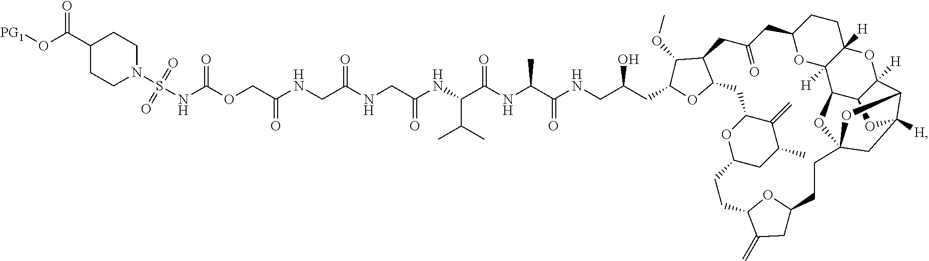

wherein
PG₁ is each independently H or a carboxyl protecting group, such as C₁₋₆ alkyl, allyl, benzyl, 2,4-dimethoxybenzyl, p-methoxybenzyl, methoxyethoxymethyl, pentafluorophenyl, 4-p-methylbenzyloxybenzyl;
PG₂ is each independently H or an amino protecting group, for example, an alkoxycarbonyl amino protecting group, such as benzyloxycarbonyl (Cbz), tert-butyloxycarbonyl (Boc), fluorenylmethoxycarbonyl (Fmoc), allyloxycarbonyl (Alloc), trimethylsilylethoxycarbonyl (Teoc), methyl(or ethyl)oxycarbonyl; an acyl amino protecting group, such as phthaloyl (Pht), p-toluenesulfonyl (Tos), trifluoroacetyl (Tfa), o-(p-)nitrobenzenesulfonyl (Ns), pivaloyl, benzoyl, tert-butyloxycarbonyl, 9-fluorenylmethoxycarbonyl, allyloxycarbonyl, trichloroethoxycarbonyl, trimethylsilylethoxycarbonyl, benzyloxycarbonyl, p-toluenesulfonyl, p-nitrobenzenesulfonyl, trifluoroacetyl, methoxycarbonyl, or ethoxycarbonyl; an alkyl amino protecting group, such as trityl (Trt), 2,4-dimethoxybenzyl (Dmb), 4-methoxybenzyl (PMB), benzyl (Bn).

In another aspect, the present application provides a use of the intermediate compounds or salts, stereoisomers, tautomers or isotopically labeled compounds thereof as described above in the manufacture of the compound or pharmaceutically acceptable salt thereof of the present invention.

### Composition

In another aspect, the present application provides a composition, which may comprise a plurality of the ADCs as described herein. Each antibody molecule in the composition can be conjugated to 1, 2, 3, 4, 5, 6, 7, 8, 9 or 10 compounds of the present invention. Therefore, the composition is characterized by a “drug-antibody ratio” (DAR) in the range of about 1 to about 10. Methods for determining DAR are well known to technicians, including methods using reverse phase chromatography or HPLC-MS.

For example, in any embodiment, the DAR of the ADC composition as described herein is about 1 to about 10 or any subrange therebetween, for example, about 1, about 2, about 3, about 4, about 5, about 6, about 7, about 8, about 9, about 10, about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, About 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, the DAR of the ADC composition as described herein is about 1 to 8, for example, about 1.0 to 1.5, about 1.5 to 2.0, about 2.0 to 2.5, about 2.5 to 3.0, about 3.0 to 3.5, about 3.5 to 4.0, about 4.0 to 4.5, about 4.5 to 5.0, about 5.0 to 5.5, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 8.0, about 6.5 to 8.5, about 7.0 to 7.5, about 7.0 to 8.0.

In certain embodiments, the DAR of the ADC compositions described herein is about 1 to 5, for example, about 1.0 to 1.5, about 1.5 to 2.0, about 2.0 to 2.5, about 2.5 to 3.0, about 3.0 to 3.5, about 3.5 to 4.0, about 4.0 to 4.5, about 4.5 to 5.0.

In certain embodiments, the DAR of the ADC compositions described herein is about 1 to 3, for example, about 1.0 to 1.5, about 1.0 to 2.0, about 1.0 to 2.5, about 1.0 to 3.0, about 1.5 to 2.0, about 1.5 to 2.5, about 1.5 to 3.0, about 2.0 to 2.5, about 2.0 to 3.0, about 2.5 to 3.0.

In certain embodiments, the DAR of the ADC compositions described herein is about 1.0 to 6.0, for example, about 1.0 to 5.5, about 1.0 to 5.0, about 1.5 to 6.0, about 1.5 to about 5.5, about 1.5 to 5.0, 2.0 to 5.5, about 2.0 to about 5.0, e.g., 1.0, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, about 1.2, about 1.21, about 1.22, about 1.23, about 1.24, about 1.25, about 1.26, about 1.27, about 1.28, about 1.29, about 1.3, about 1.31, about 1.32, about 1.33, about 1.34, about 1.35, about 1.36, about 1.37, about 1.38, about 1.39, about 1.4, about 1.41, about 1.42, about 1.43, about 1.44, about 1.45, about 1.46, about 1.47, about 1.48, about 1.49, about 1.5, about 1.51, about 1.52, about 1.53, about 1.54, about 1.55, about 1.56, about 1.57, about 1.58, about 1.59, about 1.6, about 1.61, about 1.62, about 1.63, about 1.64, about 1.65, about 1.66, about 1.67, about 1.68, about 1.69, about 1.7, about 1.71, about 1.72, about 1.73, about 1.74, about 1.75, about 1.76, about 1.77, about 1.78, about 1.79, about 1.8, about 1.81, about 1.82, about 1.83, about 1.84, about 1.85, about 1.86, about 1.87, about 1.88, about 1.89, about 1.9, about 1.91, about 1.92, about 1.93, about 1.94, about 1.95, about 1.96, about 1.97, about 1.98, about 1.99, about 2.0, about 2.01, about 2.02, about 2.03, about 2.04, about 2.05, about 2.06, about 2.07, about 2.08, about 2.09, about 2.1, about 2.11, about 2.12, about 2.13, about 2.14, about 2.15, about 2.16, about 2.17, about 2.18, about 2.19, about 2.2, about 2.21, about 2.22, about 2.23, about 2.24, about 2.25, about 2.26, about 2.27, about 2.28, about 2.29, about 2.3, about 2.31, about 2.32, about 2.33, about 2.34, about 2.35, about 2.36, about 2.37, about 2.38, about 2.39, about 2.4, about 2.41, about 2.42, about 2.43, about 2.44, about 2.45, about 2.46, about 2.47, about 2.48, about 2.49, about 2.5, about 2.51, about 2.52, about 2.53, about 2.54, about 2.55, about 2.56, about 2.57, about 2.58, about 2.59, about 2.6, about 2.61, about 2.62, about 2.63, about 2.64, about 2.65, about 2.66, about 2.67, about 2.68, about 2.69, about 2.7, about 2.71, about 2.72, about 2.73, about 2.74, about 2.75, about 2.76, about 2.77, about 2.78, about 2.79, about 2.8, about 2.81, about 2.82, about 2.83, about 2.84, about 2.85, about 2.86, about 2.87, about 2.88, about 2.89, about 2.9, about 2.91, about 2.92, about 2.93, about 2.94, about 2.95, about 2.96, about 2.97, about 2.98, about 2.99, about 3.0, about 3.01, about 3.02, about 3.03, about 3.04, about 3.05, about 3.06, about 3.07, about 3.08, about 3.09, about 3.1, about 3.11, about 3.12, about 3.13, about 3.14, about 3.15, about 3.16, about 3.17, about 3.18, about 3.19, about 3.2, about 3.21, about 3.22, about 3.23, about 3.24, about 3.25, about 3.26, about 3.27, about 3.28, about 3.29, about 3.3, about 3.31, about 3.32, about 3.33, about 3.34, about 3.35, about 3.36, about 3.37, about 3.38, about 3.39, about 3.4, about 3.41, about 3.42, about 3.43, about 3.44, about 3.45, about 3.46, about 3.47, about 3.48, about 3.49, about 3.5, about 3.51, about 3.52, about 3.53, about 3.54, about 3.55, about 3.56, about 3.57, about 3.58, about 3.59, about 3.6, about 3.61, about 3.62, about 3.63, about 3.64, about 3.65, about 3.66, about 3.67, about 3.68, about 3.69, about 3.7, about 3.71, about 3.72, about 3.73, about 3.74, about 3.75, about 3.76, about 3.77, about 3.78, about 3.79, about 3.8, about 3.81, about 3.82, about 3.83, about 3.84, about 3.85, about 3.86, about 3.87, about 3.88, about 3.89, about 3.9, about 3.91, about 3.92, about 3.93, about 3.94, about 3.95, about 3.96, about 3.97, about 3.98, about 3.99, about 4.0, about 4.01, about 4.02, about 4.03, about 4.04, about 4.05, about 4.06, about 4.07, about 4.08, about 4.09, about 4.1, about 4.11, about 4.12, about 4.13, about 4.14, about 4.15, about 4.16, about 4.17, about 4.18, about 4.19, about 4.2, about 4.21, about 4.22, about 4.23, about 4.24, about 4.25, about 4.26, about 4.27, about 4.28, about 4.29, about 4.3, about 4.31, about 4.32, about 4.33, about 4.34, about 4.35, about 4.36, about 4.37, about 4.38, about 4.39, about 4.4, about 4.41, about 4.42, about 4.43, about 4.44, about 4.45, about 4.46, about 4.47, about 4.48, about 4.49, about 4.5, about 4.51, about 4.52, about 4.53, about 4.54, about 4.55, about 4.56, about 4.57, about 4.58, about 4.59, about 4.6, about 4.61, about 4.62, about 4.63, about 4.64, about 4.65, about 4.66, about 4.67, about 4.68, about 4.69, about 4.7, about 4.71, about 4.72, about 4.73, about 4.74, about 4.75, about 4.76, about 4.77, about 4.78, about 4.79, about 4.8, about 4.81, about 4.82, about 4.83, about 4.84, about 4.85, about 4.86, about 4.87, about 4.88, about 4.89, about 4.9, about 4.91, about 4.92, about 4.93, about 4.94, about 4.95, about 4.96, about 4.97, about 4.98, about 4.99, about 5.0.

In certain embodiments, the DAR of the ADC compositions described herein is about 2.28, about 2.70, about 2.14, about 2.09, about 4.17, about 4.06, about 3.89, or about 4.02.

In certain embodiments, the DAR of the ADC composition described herein is 2.28, 2.70, 2.14, 2.09, 4.17, 4.06, 3.89 or 4.02.

### Pharmaceutical Composition

In another aspect, the present application provides a pharmaceutical composition, which comprises the compound or pharmaceutically acceptable salt, conjugate, composition thereof as described in any one of the above items, and one or more pharmaceutical excipients.

The compound or pharmaceutically acceptable salt, conjugate, composition thereof described herein can be formulated together with a pharmaceutically acceptable parenteral vehicle for parenteral use to form a unit injectable form, such as bolus injection, intravenous injection, intratumoral injection, etc. Optionally, the antibody-drug conjugate with the desired purity is mixed with a pharmaceutically acceptable diluent, carrier, excipient or stabilizer in the form of a lyophilized agent or solution (Remington's Pharmaceutical Sciences (1980) 16th edition, Osol, A. Ed.). The antibody-drug conjugate as described herein or the pharmaceutical composition containing the antibody-drug conjugate can be administered via any route suitable for the individual to be treated.

### Use

The present application provides a use of the compound or pharmaceutically acceptable salt, conjugate, composition, or pharmaceutical composition thereof as described above in the manufacture of a medicament for preventing or treating a cancer.

The present application provides the compound or pharmaceutically acceptable salt, conjugate, composition, or pharmaceutical composition thereof as described above, for use in preventing or treating a cancer.

The present application provides the compound or pharmaceutically acceptable salt, conjugate, composition, or pharmaceutical composition thereof as described above, for use in preventing or treating a solid tumor or hematological malignancy, such as gastric cancer, breast cancer, lung cancer (for example, non-small cell lung cancer, specifically lung adenocarcinoma) and urothelial carcinoma.

The present application provides a method for preventing or treating a cancer, which comprises administering to an individual in need thereof a prophylactically or therapeutically effective amount of the compound or pharmaceutically acceptable salt, conjugate, composition, or pharmaceutical composition thereof as described above.

In some embodiments, the cancer is selected from solid tumors or hematological malignancies, such as gastric cancer, breast cancer, lung cancer (for example, non-small cell lung cancer, specifically lung adenocarcinoma) and urothelial carcinoma.

In some embodiments, the compound or pharmaceutically acceptable salt, conjugate, composition, or pharmaceutical composition thereof is sufficient (e.g., in a subject) to:
(1) inhibit the proliferation of cells (e.g., tumor cells);
(2) inhibit tumor growth;
(3) induce and/or increase activity of antibody-dependent cellular cytotoxicity;
(4) inhibit signal transduction;
(5) prevent and/or treat a cancer; or
(6) any combination of (1) to (5) above.

In some embodiments, the cancer disease is selected from the group consisting of solid tumors or hematological malignancies; for example, selected from the group consisting of gastric cancer, breast cancer, lung cancer (e.g., non-small cell lung cancer, specifically lung adenocarcinoma) and urothelial carcinoma.

For all technical features disclosed in the description, such as the definitions of groups, etc., except for mutually exclusive technical features, all embodiments thereof can be combined in any way to obtain different general formula ranges or specific schemes. These ranges and schemes are within the scope of the present invention.

### Definitions

Unless otherwise defined below, the meanings of all technical and scientific terms used herein are intended to be the same as those generally understood by a skilled person in the art. References to the technology used herein shall mean the technology generally recognized in the art, including technological modifications or equivalent substitutions apparent to those skilled in the art. In addition, the laboratory procedures in genomics, nucleic acid chemistry, molecular biology, etc. used herein are all routine steps widely adopted in the respective fields. Although it is believed that the following terms are well understood by those skilled in the art, the following definitions are still provided herein to better explain the present invention.

The term “antibody” refers to an immunoglobulin molecule that is typically composed of two pairs of polypeptide chains, each pair having one light chain (LC) and one heavy chain (HC). Antibody light chains can be categorized into κ (kappa) and λ (lambda) types. Heavy chains may be categorized into μ, δ, γ, α or ε, with corresponding antibody isotypes defined as IgM, IgD, IgG, IgA and IgE, respectively. In the light chain and heavy chain, the variable region and the constant region are connected by a “J” region of about 12 or more amino acids, and the heavy chain also contains a “D” region of about 3 or more amino acids. Each heavy chain consists of a heavy chain variable region (V_{H}) and a heavy chain constant region (CH). The heavy chain constant region consists of 3 domains (CH1, CH2 and CH3). Each light chain consists of a light chain variable region (V_{L}) and a light chain constant region (CL). The light chain constant region consists of one domain CL. The constant domain does not directly participate in the binding of antibodies to antigens, but exhibits a variety of effector functions, such as mediating the binding of immunoglobulins to host tissues or factors, including various cells of the immune system (e.g., effector cells) and the first component (C1q) of the classical complement system. The V_{H} and V_{L} regions may also be subdivided into highly variable regions (called complementarity determining regions (CDRs)), interspersed with more conserved regions called framework regions (FRs). Each of VH and VL consists of three CDRs and four FRs arranged from the amino terminus to the carboxyl terminus in the following order: FR1, CDR1, FR2, CDR2, FR3, CDR3, FR4. The variable regions (V_{H} and V_{L}) of each heavy chain/light chain pair form antigen binding sites, respectively. Amino acid numbering in each region or domain may follow various numbering systems known in the art. The term “antibody” further includes variants where the heavy chain constant region has a C-terminal lysine, lacks a C-terminal lysine, or lacks a C-terminal glycine-lysine dipeptide. It also includes variants where the N-terminal amino acid of the variable region is cyclized to pyroglutamate. Thus, in a composition comprising the disclosed antibodies, each antibody may independently have a C-terminal lysine, lack a C-terminal lysine or C-terminal glycine-lysine, and/or have a N-terminal glutamine or glutamic acid, or pyroglutamate resulting from cyclization of the N-terminal amino acid.

The term “complementarity determining region” or “CDR” refers to the amino acid residues in the variable region of an antibody that are responsible for antigen binding. The variable regions of the heavy chain and light chain each contains three CDRs, designated CDR1, CDR2, and CDR3. The precise boundaries of these CDRs may be defined according to various numbering systems known in the art, for example according to the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883), the IMGT numbering system (Lefranc et al., Dev. Comparat. Immunol. 27:55-77, 2003), or the AbM numbering system (Martin ACR, Cheetham J C, Rees A R (1989) Modelling antibody hypervariable loops: A combined algorithm. Proc Natl Acad Sci USA 86:9268-9272). For a given antibody, a person skilled in the art will readily identify the CDRs defined by each numbering system. Furthermore, the correspondence between different numbering systems is well known to a person skilled in the art (e.g., see Lefranc et al., Dev. Comparat. Immunol. 27:55-77, 2003).

In the present invention, the CDRs comprised in an antibody or antigen-binding fragment thereof may be determined according to various numbering systems known in the art, such as by Kabat, Chothia, IMGT or AbM numbering systems. In certain embodiments, the CDRs comprised in an antibody or antigen-binding fragment thereof are determined by the Chothia numbering system.

The following general rules disclosed in www.bioinf.org.uk: Professor Andrew C. R. Martin's research group and produce below may be used to define CDRs in an antibody sequence that includes those amino acids that specifically interact with the amino acids comprising the epitope in the antigen to which the antibody binds. There are rare examples where these generally constant features do not occur, however, the Cy s residues are the most conserved features.

| Loop | Kabat | AbM | Chothia ¹ | Contact ² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat numbering | | | | |
| | system)³ | | | | |
| H1 | H31--H35 | H26--H35 | H26--H32 | H30--H35 | H26--H33 |
| | (Chothia numbering | | | | |
| | system) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹ Some of these numbering systems (particularly for Chothia numbering system) vary depending on the individual publication examined.
² Any of the numbering system can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering system places insertions at H35A and H35B).
If neither H35A nor H35B is present, the loop ends at H32;
If only H35A is present, the loop ends at H33;
If both H35A and H35B are present, the loop ends at H34.

The entire amino acid sequence of V_{H} is commonly numbered according to Kabat, whilte the three CDRs within the variable region may be defined according to any of the aforementioned numbering systems. In particular embodiments, the numbering of the amino acid positions in V_{H} may be sequential beginning with amino acid position 1 continuing sequentially to the end of the sequence or according to Kabat. Unless specified otherwise, the amino acid positions in V_{H} and V_{L} described herein are defined according to sequential numbering.

The numbering of amino acid positions in the heavy chain constant region may be sequential beginning with amino acid position 1 and continuing sequentially to the end of the sequence or according to Eu numbering. The IgG1 heavy chain constant region amino acid sequence has 330 amino acids sequentially numbered 1 to 330. The corresponding sequence numbered according to Eu begin with position number 118 and ends with position number 447. Unless specified otherwise, the amino acid positions in the heavy and light chains described herein are defined according to sequential numbering.

The term “framework region” or “FR” residues refers to those amino acid residues in the antibody variable region other than the CDR residues as defined above.

The term “antibody” is not limited to any particular method of producing the antibody. This includes, for example, recombinant antibodies, monoclonal antibodies, and polyclonal antibodies. The antibodies may be of different isotypes, for example, IgG (e.g., IgG1, IgG2, IgG3 or IgG4 subtypes), IgAQ1, IgA2, IgD, IgE or IgM antibodies.

The term “antigen-binding fragment” of an antibody refers to a polypeptide of a fragment of an antibody, such as a polypeptide of a fragment of a full-length antibody, which retains the ability to specifically bind to the same antigen bound by the full-length antibody and/or competes with the full-length antibody for specific binding to the antigen, which is also referred to as an “antigen-binding portion”. See generally, Fundamental Immunology, Ch. 7 (Paul, W., ed., 2nd ed., Raven Press, N.Y. (1989), which is incorporated herein by reference in its entirety for all purposes. An antigen-binding fragment of an antibody may be produced by a recombinant DNA technique or by an enzymatic or chemical cleavage of the intact antibody. Non-limiting examples of antigen-binding fragment include Fab fragment, Fab′ fragment, F(ab)′₂ fragment, F(ab)′₃ fragment, Fd, Fv, scFv, di-scFv, (scFv)₂, disulfide-stabilized Fv protein (“dsFv”), single domain antibody (sdAb, nanobody), and a polypeptide that comprises at least a portion of an antibody sufficient to confer specific antigen binding ability to the polypeptide. Engineered antibody variants are reviewed in Holliger et al., 2005; Nat Biotechnol, 23: 1126-1136.

The term “Fd” refers to an antibody fragment consisting of the V_{H} and CH1 domains; the term “dAb fragment” refers to an antibody fragment consisting of the V_{H} domain (Ward et al., Nature 341:544 546 (1989)); the term “Fab fragment” refers to an antibody fragment consisting of the VL, VH, CL and CH1 domains; the term “F(ab′)₂ fragment” refer to an antibody fragment comprising two Fab fragments connected by a disulfide bridge on the hinge region; the term “Fab′ fragment” refers to a fragment obtained after reducing the disulfide bonds connecting the two heavy chain fragments in the F(ab′)₂ fragment, which consists of a complete light chain and the Fd fragment of the heavy chain (consisting of the V_{H} and CH1 domains).

The term “Fv” refers to an antibody fragment consisting of the V_{L} and V_{H} domains of a single arm of an antibody. The Fv fragment is generally considered to be the smallest antibody fragment that may form a complete antigen binding site. It is generally believed, six CDRs confer antigen-binding specificity to an antibody. However, even a single variable region (e.g., an Fd fragment, which contains only three CDRs specific for an antigen) is able to recognize and bind to an antigen, although its affinity may be lower than that of the complete binding site.

The term “Fc” refers to an antibody fragment formed by disulfide bonding of the second and third constant regions of the first heavy chain of an antibody to the second and third constant regions of the second heavy chain. The Fc fragment of an antibody has a variety of different functions but is not involved in antigen binding.

The term “scFv” refers to a single polypeptide chain containing V_{L} and V_{H} domains connected by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore, eds., Springer-Verlag, New York, pp. 269-315 (1994)). Such scFv molecules may have a general structure: NH₂—VL-linker-VH-COOH or NH₂—VH-linker-VL-COOH. A suitable prior art peptide linker consists of the repeated GGGGS (SEQ ID NO: 46) amino acid sequence or its variants. For example, a linker having the amino acid sequence (GGGGS)₄ (SEQ ID NO: 47) may be used, but variants thereof may also be used (Holliger et al. (1993), Proc. Natl. Acad. Sci. USA 90: 6444-6448). Other linkers that may be used in the present invention are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, a disulfide bond may also exist between the V_{H} and V_{L} of the scFv. In certain embodiments, the V_{H} and V_{L} domains may be positioned relative to each other in any suitable arrangement. For example, a scFv may comprise NH₂—VH-VH-COOH, NH₂-VL-VL-COOH.

The term “single-domain antibody (sdAb)” has the meaning commonly understood by those skilled in the art, which refers to an antibody fragment composed of a single monomeric variable antibody domain (e.g., a single heavy chain variable region) that retains the ability to specifically bind to the same antigen bound by a full-length antibody (Holt, L. et al., Trends in Biotechnology, 21(11):484-490, 2003). Single-domain antibody is also called nanobody.

The above-mentioned antibody fragments all retain the ability to specifically bind to the same antigen bound by a full-length antibody, and/or compete with the full-length antibody for specific binding to the antigen.

As used therein, unless the context clearly indicates otherwise, when referring to the term “antibody”, it includes not only an intact antibody but also an antigen-binding fragment of the antibody.

An antigen-binding fragment of antibody (e.g., the above-mentioned antibody fragment) may be obtained from a given antibody (e.g., the antibody provided by the present invention) using a conventional technique known to a skilled person in the art (e.g., recombinant DNA technology or enzymatic or chemical cleavage method), and the antigen-binding fragment of antibody may be screened for specificity in the same manner as for the intact antibody.

The term “murine antibody” refers to an antibody obtained by the following method: fusing B cells of immunized mice with myeloma cells, screening for murine hybrid fusion cells that may both proliferate indefinitely and secrete antibodies, followed by screening, antibody preparation and antibody purification; or refers to an antibody secreted by plasma cells formed by differentiation and proliferation of B cells after an antigen invades mice.

The term “humanized antibody” refers to a non-human antibody modified by genetic engineering, of which amino acid sequence is modified to increase the homology with the sequence of a human antibody. Generally speaking, all or part of the CDR region of a humanized antibody comes from a non-human antibody (donor antibody), and all or part of the non-CDR region (e.g., variable region FR and/or constant region) comes from a human immunoglobulin (recipient antibody). the humanized antibody generally retains the expected properties of the donor antibody, including, but not limited to, antigen specificity, affinity, reactivity, ability to increase immune cell activity, ability to enhance immune response, etc. The donor antibody may be a mouse, rat, rabbit or non-human primate (e.g., cynomolgus monkey) antibody with the expected properties (e.g., antigen specificity, affinity, reactivity, ability to increase immune cell activity and/or ability to enhance immune response).

The term “identity” is used to refer to a match of sequences between two polypeptides or between two nucleic acids. When a position in both sequences being compared is occupied by the same base or amino acid monomer subunit (e.g., a position in each of two DNA molecules is occupied by adenine, or a position in each of two polypeptides is occupied by lysine), then the molecules are identical at that position. “Percent identity” between two sequences is a function of the number of matching positions common to the two sequences divided by the number of positions compared×100. For example, if 6 out of 10 positions of two sequences match, then the two sequences are 60% identical. For example, the DNA sequences CTGACT and CAGGTT share 50% identity (3 positions match out of 6 total positions). Typically, comparisons are made when two sequences are aligned to yield maximum identity. Such alignments can be achieved using, for example, the method of Needleman et al. (1970) J. Mol. Biol. 48: 443-453, which can be conveniently performed by computer programs such as the Align program (DNAstar, Inc.). The algorithm of E. Meyers and W. Miller (Comput. Appl Biosci., 4:11-17 (1988)) which has been incorporated into the ALIGN program (version 2.0), can be used to determine the percent identity between two amino acid sequences by using the PAM120 weight residue table, a gap length penalty of 12, and a gap penalty of 4. Alternatively, the algorithm of Needleman and Wunsch (J Mol Biol. 48:444-453 (1970)) which has been integrated into the GAP program of the GCG software package (available at www.gcg.com) and can be used to determine the percent identity between two amino acid sequences by using the Blossum 62 matrix or PAM250 matrix with a gap weight of 16, 14, 12, 10, 8, 6, or 4, and a length weight of 1, 2, 3, 4, 5, or 6.

The term “conservative substitution” refers to an amino acid substitution that does not adversely affect or change the expected properties of the protein/polypeptide comprising the amino acid sequence. For example, a conservative substitution may be introduced by a standard technique known in the art such as site-directed mutagenesis and PCR-mediated mutagenesis. Conservative amino acid substitutions include substitutions in which amino acid residues are substituted with amino acid residues having similar side chains, such as substitutions with residues that are physically or functionally similar to the corresponding amino acid residues (e.g., having similar size, shape, charge, chemical properties, including the ability to form covalent bonds or hydrogen bonds, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Thus, it is preferred to replace a corresponding amino acid residue with another amino acid residue from the same side chain family. Methods for identifying conservative substitutions of amino acids are well known in the art (see, for example, Brummell et al., Biochem. 32:1180-1187 (1993); Kobayashi et al. Protein Eng. 12(10):879-884 (1999); and Burks et al. Proc. Natl Acad. Set USA 94:412-417 (1997), which are incorporated herein by reference).

The twenty conventional amino acids used herein are written in accordance with conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, amino acids are generally represented by single-letter and three-letter abbreviations known in the art. For example, alanine may be represented by A or Ala.

The term “linker” refers to a structural fragment that connects a cytotoxic drug to an antibody or antigen-binding fragment thereof. For example, it refers to the -M-L-E- structural fragment in the formula Ab-[M-L-E-D]ₓ.

The term “drug-linker” refers to a structure of the cytotoxic drug and linker described in the present invention before being connected to an antibody or antigen-binding fragment thereof. For example, “drug-linker” refers to M′-L-E-D, wherein M′ is the structural form of M before covalently connecting to an antibody or antigen-binding fragment thereof. “Drug-linker” is covalently connected to an antibody or antigen-binding fragment thereof to obtain the antibody-drug conjugate described in the present application.

The “drug-linker” also includes all pharmaceutically acceptable isotopically labeled compounds thereof, which are the same as the “drug-linker” compound of the present invention, except that one or more atoms are replaced by atoms having the same atomic number but an atomic mass or mass number different from the atomic mass or mass number that predominates in nature.

Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

The terms “include,” “comprises,” “have,” “contain,” or “involve,” and other variations thereof as used herein, are inclusive or open-ended and do not exclude other unlisted elements or method steps.

As used herein,

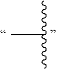

indicates the position where the structural fragment is connected to other parts of the molecule.

The term “alkyl” refers to a group, such as “C₁₋₂₀ alkyl”, “C₁₋₁₀ alkyl”, “C₁₋₆ alkyl”, “C₁₋₄ alkyl”, “C₁₋₃ alkyl”, etc., which is obtained by removing one hydrogen atom from a straight or branched hydrocarbonyl. Its specific examples include, but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkenyl” refers to a straight or branched hydrocarbonyl containing at least one carbon-carbon double bond, including, for example, “C₂₋₆ alkenyl”, “C₂₋₄ alkenyl”, etc. Its examples include, but are not limited to, vinyl, 1-propenyl, 2-propenyl, 1-butenyl, 2-butenyl, 1,3-butadienyl, 1-pentenyl, 2-pentenyl, 3-pentenyl, 1,3-pentadienyl, 1,4-pentadienyl, 1-hexenyl, 2-hexenyl, 3-hexenyl, 1,4-hexadienyl, etc.

The term “alkynyl” refers to a straight or branched hydrocarbonyl containing at least one carbon-carbon triple bond, including, for example, “C₂₋₆ alkynyl”, “C₄₋₆ alkynyl”, etc. Its examples include, but are not limited to, ethynyl, 1-propynyl, 2-propynyl, 1-butynyl, 2-butynyl, 1,3-butadiynyl, 1-pentynyl, 2-pentynyl, 3-pentynyl, 1,3-pentadiynyl, 1,4-pentadiynyl, 1-hexynyl, 2-hexynyl, 3-hexynyl, 1,4-hexadiynyl, etc.

The term “cycloalkyl” refers to a saturated cyclic hydrocarbonyl, including but not limited to monocyclic alkyl and bicyclic alkyl (e.g., spiro cycloalkyl, fused cycloalkyl, and bridged cycloalkyl). The term “C₃₋₆ cycloalkyl” refers to a cycloalkyl having 3 to 6 ring carbon atoms, such as cyclopropyl, cyclobutyl, cyclopentyl, cyclohexyl, etc., which may be optionally substituted with 1 or more (e.g., 1, 2 or 3) suitable substituents, for example, methyl-substituted cyclopropyl.

The term “heterocyclyl” or “heterocycle” refers to a saturated or partially saturated, monocyclic or polycyclic (e.g., bicyclic) non-aromatic cyclic structure, whose ring atoms consist of carbon atoms and at least one (e.g., 1, 2, or 3) heteroatom selected from the group consisting of nitrogen, oxygen, and sulfur. If the valence bond requirements are met, the heterocyclyl can be connected to the rest of the molecule via any one of the ring atoms. The heterocyclyl in the present invention is preferably a 3- to 6-membered heterocyclyl. The term “3- to 6-membered heterocyclyl” used in the present invention refers to a heterocyclyl having 3 to 6 ring atoms, including 3-membered heterocyclyl, 4-membered heterocyclyl, 5-membered heterocyclyl, and 6-membered heterocyclyl, including nitrogen-containing heterocyclyl, oxygen-containing heterocyclyl, such as 4- to 6-membered heterocyclyl, such as 4- to 6-membered nitrogen-containing heterocyclyl, 4- to 6-membered oxygen-containing heterocyclyl. Common heterocyclic groups include (but are not limited to) azetidinyl, oxetanyl, tetrahydrofuryl, pyrrolidinyl, pyrrolidinonyl, imidazolidinyl, pyrazolidinyl, tetrahydropyranyl, piperidinyl, piperazinyl, morpholinyl. The heterocyclyl in the present invention may be optionally substituted with one or more substituents described in the present invention. The heterocyclyl in the present invention is optionally fused with one or more aromatic or non-aromatic rings.

The term “oxygen-containing heterocycle” refers to a heterocycle as described above in which one or more (e.g., 1, 2, or 3) ring atoms are oxygen atoms, such as 5- to 6-membered oxygen-containing heterocycle, and specific examples include but are not limited to oxirane ring, tetrahydrofuran ring, furan ring, tetrahydropyran ring, pyran ring, and the like. The “nitrogen-containing heterocycle” described in the present invention refers to a heterocycle as described above in which one or more (e.g., 1, 2, or 3) ring atoms are nitrogen atoms.

The term “alkoxy” refers to a group having an “alkyl-O—” structure, wherein the alkyl is defined as described above. Its examples include C₁₋₆ alkoxy, C₁₋₄ alkoxy, C₁₋₃ alkoxy, or C₁₋₂ alkoxy, etc. Common alkoxy groups include (but are not limited to) methoxy, ethoxy, n-propoxy, isopropoxy, n-butoxy, isobutoxy, tert-butoxy, pentyloxy, hexyloxy, etc. The alkoxy in the present invention is optionally substituted by one or more substituents described in the present invention.

The term “halogenated” or “halogen” group is defined to include F, Cl, Br, or I.

The term “isotopically labeled compound” means that the compound is identical in structure to the compound of the present invention except that one or more atoms are replaced by atoms having the same atomic number but an atomic mass or mass number different from the atomic mass or mass number that prevails in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

As used herein, the term “suitable substituent” refers to a modification that a person skilled in the art can make to a compound according to the need of the compound substituent. “Suitable substituent” includes oxo (═O), halogen, cyano, NR⁸R⁹, carboxyl, mercapto, hydroxyl, ester (e.g., —C₁₋₆ alkyl-C(═O)—OC₁₋₆ alkyl), C₁₋₆ alkyl, C₂₋₆ alkenyl, C₂₋₆ alkynyl, C₁₋₆ alkyl-O—C₁₋₆ alkyl, C₁₋₆ haloalkyl, C₁₋₆ alkoxy, C₃₋₆ cycloalkyl, 3- to 10-membered heterocyclyl, 5- to 10-membered heteroaryl, C₆₋₁₀ aryl, benzyl, hydroxy-substituted benzyl, indolylmethylene and C₁₋₆ haloalkoxy, wherein R⁸, R⁹ are each independently selected from the group consisting of H, C₁₋₆ alkyl, C₃₋₆ cycloalkyl, 3- to 10-membered heterocyclyl, 5- to 10-membered heteroaryl, C₆₋₁₀ aryl, C₁₋₆ alkoxy, C₁₋₆ haloalkyl, C₁₋₆ haloalkoxy, halogen, hydroxyl, carboxyl and ester (e.g. —C₁₋₆ alkyl-C(═O)—OC₁₋₆ alkyl).

The term “substituted” means that one or more (e.g. 1, 2, 3, 4 or 5) hydrogen atoms on the specified compound or structural fragment are replaced by a substituent, provided that the normal valence of the specified atom in the current situation is not exceeded and the substitution forms a stable compound. Combinations of substituents and/or variables are permissible only if they yield stable compounds. In some embodiments, the substituent is each independently composed of one or more of the following structures: —O—, —S—, —NR′—, halogen, —CN, —OH, —NH₂, —NO₂, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 8-membered heterocyclyl(ene), C₆-C₁₀ aryl(ene) and 5- to 10-membered heteroaryl(ene), etc. In some embodiments, the substituent is each independently composed of one or more of the following structures: NR⁸R⁹, —O—, —S—, —NR′—, halogen, —CN, —OH, —SH, —NH₂, —NO₂, —C(O)—, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 10-membered heterocyclyl(ene), C₆-C₁₀ aryl(ene), and 5- to 10-membered heteroaryl(ene), etc., wherein R⁸, R⁹ and R′ are as defined above. For example, the substituents may be suitable substituents as described above.

If a functional group or structural fragment is described as “substituted or unsubstituted”, the functional group or structural fragment may be (1) unsubstituted or (2) substituted.

As used herein, the term “one or more” means 1 or more than 1, such as 2, 3, 4, 5 or 10, under reasonable conditions.

Unless otherwise specified, as used herein, the site for attachment of a substituent may be from any suitable position of the substituent.

The pharmaceutically acceptable salts of the compounds of the present invention include acid addition salts and base addition salts thereof. Suitable acid addition salts are formed from acids capable of forming pharmaceutically acceptable salts, including aspartate, fumarate, glucoheptonate, gluconate, glucuronate, hexafluorophosphate, etc. Suitable base addition salts are formed from bases capable of forming pharmaceutically acceptable salts, including aluminum salt, arginine salt, choline salt, diethylamine salt, etc. For a review of suitable salts, see Stahl and Wermuth's “Handbook of Pharmaceutical Salts: Properties, Selection, and Use” (Wiley-VCH, 2002). Methods for preparing pharmaceutically acceptable salts of the compounds of the present invention are known to those skilled in the art.

Whether or not explicitly indicated, all numerical values in the present application are modified by the term “about”. The term “about” refers to a range of ±20% of the stated value, ±10% of the stated value, preferably ±5% of the stated value, and more preferably ±2% of the stated value.

### BRIEF DESCRIPTION OF THE DRAWINGS

FIG. 1 shows the efficacy results of anti-human HER2 antibody-drug conjugates on the NCI-N87 cell subcutaneous tumor-bearing mouse model.

FIG. 2 shows the changes in body weight of each group of mice in the human gastric cancer cell NCI-N87 CDX model.

FIG. 3 shows the efficacy results of anti-human HER2 antibody-drug conjugates on JIMT-1 breast cancer transplant model.

FIG. 4 shows the changes in body weight of each group of mice in the JIMT-1 breast cancer transplant model.

### SPECIFIC MODELS FOR CARRYING OUT THE PRESENT INVENTION

The present invention is further described below through the description of specific examples, but this is not a limitation on the present invention. Based on the teachings of the present invention, those skilled in the art can make various modifications or improvements without departing from the basic idea and scope of the present invention.

The information of the sequences involved in the present invention is described in the following table:

| SEQ | | |
|---|---|---|
| ID | | |
| NO: | Sequence name | Sequence information |
| 1 | Trastuzumab | EVQLVESGGGLVQPGGSLRLSCAASGFNIKDTYIHW |
| | VH amino acid | VRQAPGKGLEWVARIYPTNGYTRYADSVKGRFTISADTS |
| | sequence | KNTAYLQMNSLRAEDTAVYYCSRWGGDGFYAMDYWG |
| | | QGTLVTVSS |
| 2 | Trastuzumab | DIQMTQSPSSLSASVGDRVTITCRASQDVNTAVAWY |
| | VL amino acid | QQKPGKAPKLLIYSASFLYSGVPSRFSGSRSGTDFTLTISS |
| | sequence | LQPEDFATYYCQQHYTTPPTFGQGTKVEIK |
| 3 | Pertuzumab | EVQLVESGGGLVQPGGSLRLSCAASGFTFTDYTMD |
| | VH amino acid | WVRQAPGKGLEWVADVNPNSGGSIYNQRFKGRFTLSVD |
| | sequence | RSKNTLYLQMNSLRAEDTAVYYCARNLGPSFYFDYWGQ |
| | | GTLVTVSS |
| 4 | Pertuzumab | DIQMTQSPSSLSASVGDRVTITCKASQDVSIGVAWY |
| | VL amino acid | QQKPGKAPKLLIYSASYRYTGVPSRFSGSGSGTDFTLTISS |
| | sequence | LQPEDFATYYCQQYYIYPYTFGQGTKVEIK |
| 5 | Trastuzumab | GFNIKDT |
| | Chothia CDR H1 | |
| 6 | Trastuzumab | YPTNGY |
| | Chothia CDR H2 | |
| 7 | Trastuzumab | WGGDGFYAMDY |
| | Chothia/kabat/AbM | |
| | CDR H3 | |
| 8 | Trastuzumab | RASQDVNTAVA |
| | Chothia/Kabat/AbM | |
| | CDR L1 | |
| 9 | Trastuzumab | SASFLYS |
| | Chothia/Kabat/AbM | |
| | CDR L2 | |
| 10 | Trastuzumab | QQHYTTPPT |
| | Chothia/Kabat/IMGT/ | |
| | AbM CDR L3 | |
| 11 | Trastuzumab | DTYIH |
| | Kabat CDR H1 | |
| 12 | Trastuzumab | RIYPTNGYTRYADSVKG |
| | Kabat CDR H2 | |
| 13 | Trastuzumab | GFNIKDTY |
| | IMGT CDR H1 | |
| 14 | Trastuzumab | IYPTNGYT |
| | IMGT CDR H2 | |
| 15 | Trastuzumab | SRWGGDGFYAMDY |
| | IMGT CDR H3 | |
| 16 | Trastuzumab | QDVNTA |
| | IMGT CDR LI | |
| 17 | Trastuzumab | SAS |
| | IMGT CDR L2 | |
| 18 | Trastuzumab | GFNIKDTYIH |
| | AbM CDR H1 | |
| 19 | Trastuzumab | RIYPTNGYTR |
| | AbM CDR H2 | |
| 20 | Pertuzumab | GFTFTDY |
| | Chothia CDR H1 | |
| 21 | Pertuzumab | NPNSGG |
| | Chothia CDR H2 | |
| 22 | Pertuzumab | NLGPSFYFDY |
| | Chothia/Kabat/AbM | |
| | CDR H3 | |
| 23 | Pertuzumab | KASQDVSIGVA |
| | Chothia/Kabat/AbM | |
| | CDR L1 | |
| 24 | Pertuzumab | SASYRYT |
| | Chothia/Kabat/AbM | |
| | CDR L2 | |
| 25 | Pertuzumab | QQYYIYPYT |
| | Chothia/Kabat/IMGT/ | |
| | AbM CDR L3 | |
| 26 | Pertuzumab | DYTMD |
| | Kabat CDR H1 | |
| 27 | Pertuzumab | DVNPNSGGSIYNQRFKG |
| | Kabat CDR H2 | |
| 28 | Pertuzumab | GFTFTDYT |
| | IMGT CDR H1 | |
| 29 | Pertuzumab | VNPNSGGS |
| | IMGT CDR H2 | |
| 30 | Pertuzumab | ARNLGPSFYFDY |
| | IMGT CDR H3 | |
| 31 | Pertuzumab | QDVSIG |
| | IMGT CDR L1 | |
| 32 | Pertuzumab | SAS |
| | IMGT CDR L2 | |
| 33 | Pertuzumab | GFTFTDYTMD |
| | AbM CDR H1 | |
| 34 | Pertuzumab | DVNPNSGGSI |
| | AbM CDR H2 | |
| 35 | Heavy chain | ASTKGPSVFPLAPSSKSTSGGTAALGCLVKDYFPEPV |
| | constant region | TVSWNSGALTSGVHTFPAVLQSSGLYSLSSVVTVPSSSLG |
| | amino acid | TQTYICNVNHKPSNTKVDKKVEPKSCDKTHTCPPCPAPE |
| | sequence of | LLGGPSVFLFPPKPKDTLMISRTPEVTCVVVDVSHEDPEV |
| | Trastuzumab or | KFNWYVDGVEVHNAKTKPREEQYNSTYRVVSVLTVLH |
| | Pertuzumab | QDWLNGKEYKCKVSNKALPAPIEKTISKAKGQPREPQVY |
| | | TLPPSREEMTKNQVSLTCLVKGFYPSDIAVEWESNGQPE |
| | | NNYKTTPPVLDSDGSFFLYSKLTVDKSRWQQGNVFSCSV |
| | | MHEALHNHYTQKSLSLSPGK |
| 36 | Light chain | RTVAAPSVFIFPPSDEQLKSGTASVVCLLNNFYPREA |
| | constant region | KVQWKVDNALQSGNSQESVTEQDSKDSTYSLSSTLTLSK |
| | amino acid | ADYEKHKVYACEVTHQGLSSPVTKSFNRGEC |
| | sequence of | |
| | Trastuzumab or | |
| | Pertuzumab | |
| 37 | Heavy chain | EVQLVESGGGLVQPGGSLRLSCAASGFNIKDTYIHW |
| | amino acid | VRQAPGKGLEWVARIYPTNGYTRYADSVKGRFTISADTS |
| | sequence of | KNTAYLQMNSLRAEDTAVYYCSRWGGDGFYAMDYWG |
| | Trastuzumab | QGTLVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKKVEPKSCDKTHTC |
| | | PPCPAPELLGGPSVFLFPPKPKDTLMISRTPEVTCVVVDV |
| | | SHEDPEVKFNWYVDGVEVHNAKTKPREEQYNSTYRVVS |
| | | VLTVLHQDWLNGKEYKCKVSNKALPAPIEKTISKAKGQP |
| | | REPQVYTLPPSREEMTKNQVSLTCLVKGFYPSDIAVEWE |
| | | SNGQPENNYKTTPPVLDSDGSFFLYSKLTVDKSRWQQG |
| | | NVFSCSVMHEALHNHYTQKSLSLSPGK |
| 38 | Light chain | DIQMTQSPSSLSASVGDRVTITCRASQDVNTAVAWY |
| | amino acid | QQKPGKAPKLLIYSASFLYSGVPSRFSGSRSGTDFTLTISS |
| | sequence of | LQPEDFATYYCQQHYTTPPTFGQGTKVEIKRTVAAPSVFI |
| | Trastuzumab | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 39 | Heavy chain | EVQLVESGGGLVQPGGSLRLSCAASGFTFTDYTMD |
| | amino acid | WVRQAPGKGLEWVADVNPNSGGSIYNQRFKGRFTLSVD |
| | sequence of | RSKNTLYLQMNSLRAEDTAVYYCARNLGPSFYFDYWGQ |
| | Pertuzumab | GTLVTVSSASTKGPSVFPLAPSSKSTSGGTAALGCLVKDY |
| | | FPEPVTVSWNSGALTSGVHTFPAVLQSSGLYSLSSVVTVP |
| | | SSSLGTQTYICNVNHKPSNTKVDKKVEPKSCDKTHTCPP |
| | | CPAPELLGGPSVFLFPPKPKDTLMISRTPEVTCVVVDVSH |
| | | EDPEVKFNWYVDGVEVHNAKTKPREEQYNSTYRVVSVL |
| | | TVLHQDWLNGKEYKCKVSNKALPAPIEKTISKAKGQPRE |
| | | PQVYTLPPSREEMTKNQVSLTCLVKGFYPSDIAVEWESN |
| | | GQPENNYKTTPPVLDSDGSFFLYSKLTVDKSRWQQGNV |
| | | FSCSVMHEALHNHYTQKSLSLSPGK |
| 40 | Light chain | DIQMTQSPSSLSASVGDRVTITCKASQDVSIGVAWY |
| | amino acid | QQKPGKAPKLLIYSASYRYTGVPSRFSGSGSGTDFTLTISS |
| | sequence of | LQPEDFATYYCQQYYIYPYTFGQGTKVEIKRTVAAPSVFI |
| | Pertuzumab | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 41 | Short peptide | GGFG |
| 42 | Short peptide | GGVA |
| 43 | Short peptide | GFLG |
| 44 | Short peptide | EAAA |
| 45 | Short peptide | GGGGG |
| 46 | Linker subunit | GGGGS |
| 47 | (GGGGS)₄ | GGGGSGGGGSGGGGSGGGGS |

The abbreviations used herein have the following meanings:

| Abbreviation | Meaning | Abbreviation | Meaning |
|---|---|---|---|
| HATU | N,N,N′,N′-Tetramethyl-O- | EDCI | 1-(3- |
| | (7-azabenzotriazol-1-yl)uronium | | Dimethylaminopropyl)-3- |
| | hexafluorophosphate | | ethylcarbodiimide hydrochloride |
| EEDQ | 2-Ethoxy-1- | HOBt | 1-hydroxybenzotriazole |
| | ethoxycarbonyl-1,2- | | |
| | dihydroquinoline | | |
| PyBOP | 1H-Benzotriazol-1- | DMTMM | 4-(4,6-Dimethoxytriazin-2- |
| | yloxytripyrrolidinyl | | yl)-4-methylmorpholine |
| | hexafluorophosphate | | hydrochloride |
| DIPEA | Diisopropylethylamine | DBU | 1,8- |
| | | | Diazobispiro[5.4.0]undec-7-ene |
| DMAP | 4-Dimethylaminopyridine | DCC | N,N′- |
| | | | dicyclohexylcarbodiimide |
| HOSu | N-hydroxysuccinimide | PPTS | Pyridinium p-toluenesulfonate |
| TsOH | p-Toluenesulfonic acid | TFA | Trifluoroacetic acid |
| AcOH | Acetic acid | LiOH•H₂O | Lithium hydroxide monohydrate |
| NaHCO₃ | Sodium bicarbonate | Pd(PPh₃)₄ | Tetratriphenylphosphine |
| | | | palladium |
| Pb(OAc)₄ | Lead tetraacetate | MMAE | Monomethyl auristatin E |
| AE | Auristatin E | Cit | Citrulline |
| Val | Valine | Boc | tert-Butyloxycarbonyl |
| PABC | p-Aminobenzyl alcohol | Pt/C | Platinum on carbon |
| Pd/C | Palladium on carbon | HPLC | High performance liquid |
| | | | chromatography |
| LCMS | Liquid chromatography- | Rf | Retention factor |
| | mass spectrometry | | |
| Pre- | Preparative thin layer | DMSO | Dimethyl sulfoxide |
| TLC | chromatography | | |
| PE | Petroleum ether | EA | Ethyl acetate |
| THF | Tetrahydrofuran | DMF | N,N-Dimethylformamide |
| MeOH | Methanol | DCM | Dichloromethane |
| IPA | Isopropanol | MeCN | Acetonitrile |
| MTBE | Methyl tert-butyl ether | H₂O | Water |

The structures of the compounds described in the following examples were determined by nuclear magnetic resonance (¹H NMR) or mass spectrometry (MS).

Nuclear magnetic resonance (¹H NMR) was measured using a Bruker 400 MHz nuclear magnetic resonance instrument; the deuterated reagent was hexadeuterated dimethyl sulfoxide (DMSO-d6); the internal standard substance was tetramethylsilane (TMS).

The abbreviations in the nuclear magnetic resonance (NMR) spectra used in the examples are shown below.

s: singlet, d: doublet, t: triplet, q: quartet, m: multiplet, br: broad, J: coupling constant, Hz: Hertz, DMSO-d6: deuterated dimethyl sulfoxide. 6 values are expressed in ppm.

The mass spectrometer (MS) was determined using an Agilent (ESI) mass spectrometer, model: Agilent 6120B.

### Intermediate Preparation Example 1: 2-((((4-(Methoxycarbonyl)piperidin-1-yl)sulfonyl) carbamoyl)oxy)acetic acid (INT-1)

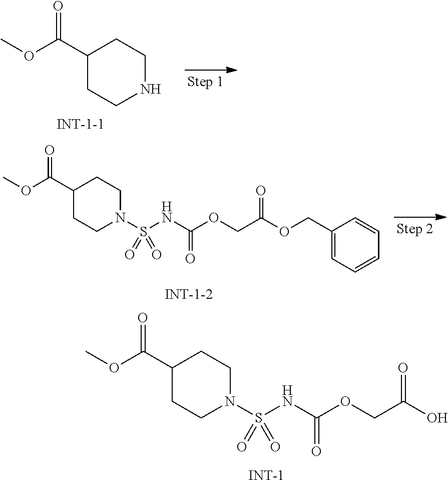

### Step 1: Preparation of methyl 1-(N-((2-(benzyloxy)-2-oxoethoxy)carbonyl)sulfamoyl) piperidine-4-carboxylate (INT-1-2)

Chlorosulfonyl isocyanate (500 mg, 3.53 mmol, 307 μL) was dissolved in dichloromethane (10.0 mL) at 0° C., added with benzyl glycolate (533 mg, 3.21 mmol, 455 μL), and continuously stirred for 1 hour. Then, a solution of methyl 4-piperidinylcarboxylate (459 mg, 3.21 mmol) and triethylamine (974 mg, 9.63 mmol, 1.34 mL) in dichloromethane (5.00 mL) was added to the above reaction solution, heated to 25° C. and stirred for 1 hour. The reaction solution was added with water (100 mL), and extracted with dichloromethane 3 times (50.0 mL×3). The organic phases were combined, washed with cooled 1N dilute hydrochloric acid (20.0 mL), dried over anhydrous sodium sulfate, filtered and concentrated to obtain the title compound as a crude product (1.30 g, 3.14 mmol), which was used directly in the next step without purification.

The structural characterization data are as follows:

ESI-MS (m/z): 414.9 [M+H]⁺

### Step 2: Preparation of 2-((((4-(methoxycarbonyl)piperidin-1-yl)sulfonyl)carbamoyl) oxy)acetic acid (INT-1)

Under nitrogen atmosphere, 10% Pd/C (0.70 g) was added to a solution of compound INT-1-2 (700 mg, 1.69 mmol) in methanol (20.0 mL), subjected to replacement with hydrogen three times, and reacted at 25° C. for 3 hours (15 PSI). The reaction solution was filtered, the filter cake was rinsed with methanol three times (100 mL×3), and the filtrate was concentrated to obtain the title compound as a crude product (590 mg).

The structural characterization data are as follows:

ESI-MS (m/z): 325.1 [M+H]⁺

### Intermediate Preparation Example 2: 2-((((4-((Allyloxy)carbonyl)piperidin-1-yl)sulfonyl) carbamoyl)oxy)acetic acid (INT-2)

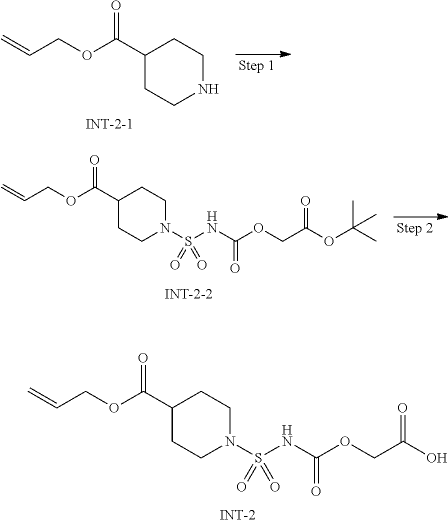

### Step 1: Preparation of allyl 1-(N-((2-(tert-butyloxy)-2-oxoethoxy)carbonyl)sulfamoyl) piperidine-4-carboxylate (INT-2-2)

Chlorosulfonyl isocyanate (1.76 g, 12.41 mmol) was dissolved in acetonitrile (80.0 mL) at 0° C., added with tert-butyl glycolate (1.56 g, 11.82 mmol, 455 μL), and stirred continuously for 1 hour. Then, allyl 4-piperidinylcarboxylate (2 g, 11.82 mmol) and pyridine (1.12 g, 14.18 mmol) were added to the above reaction solution, heated to 25° C. and stirred for 1 hour. The reaction solution was added with water (100 mL), and extracted with ethyl acetate 3 times (25.0 mL×3). The organic phases were combined, washed with cooled 1N dilute hydrochloric acid (20.0 mL) and saturated sodium chloride aqueous solution (30.0 mL), dried over anhydrous sodium sulfate, filtered and concentrated to obtain the title compound as a crude product (2.3 g, 5.66 mmol), which was used directly in the next step without purification.

Its structural characterization data are as follows:

ESI-MS (m/z): 407.1 [M+H]⁺

### Step 2: Preparation of 2-((((4-((allyloxy)carbonyl)piperidin-1-yl)sulfonyl)carbamoyl) oxy)acetic acid (INT-2)

Compound INT-2-2 (2.3 g, 5.66 mmol) was dissolved in dichloromethane (10 mL), added with TFA (2 mL), and reacted at 25° C. for 2 hours. The reaction solution was concentrated, added with 1N sodium bicarbonate aqueous solution to adjust the pH to 8, and extracted with ethyl acetate twice (25.0 mL×2). The aqueous phases were combined and adjusted to pH 3 with 1N diluted hydrochloric acid, and then extracted with ethyl acetate three times (25.0 mL×3). The organic phases were combined, dried over anhydrous sodium sulfate, filtered and concentrated to obtain the title compound as a crude product (1.6 g, 4.57 mmol).

Its structural characterization data are as follows:

ESI-MS (m/z): 351.1 [M+H]⁺

### Intermediate Preparation Example 3: 1-(3,5-Bis(2-(methylsulfonyl)pyrimidin-5-yl)phenyl)-1-oxo-5,8,11,14,17,20,23,26-octaoxa-2-azanonacosan-29-oic acid (INT-3)

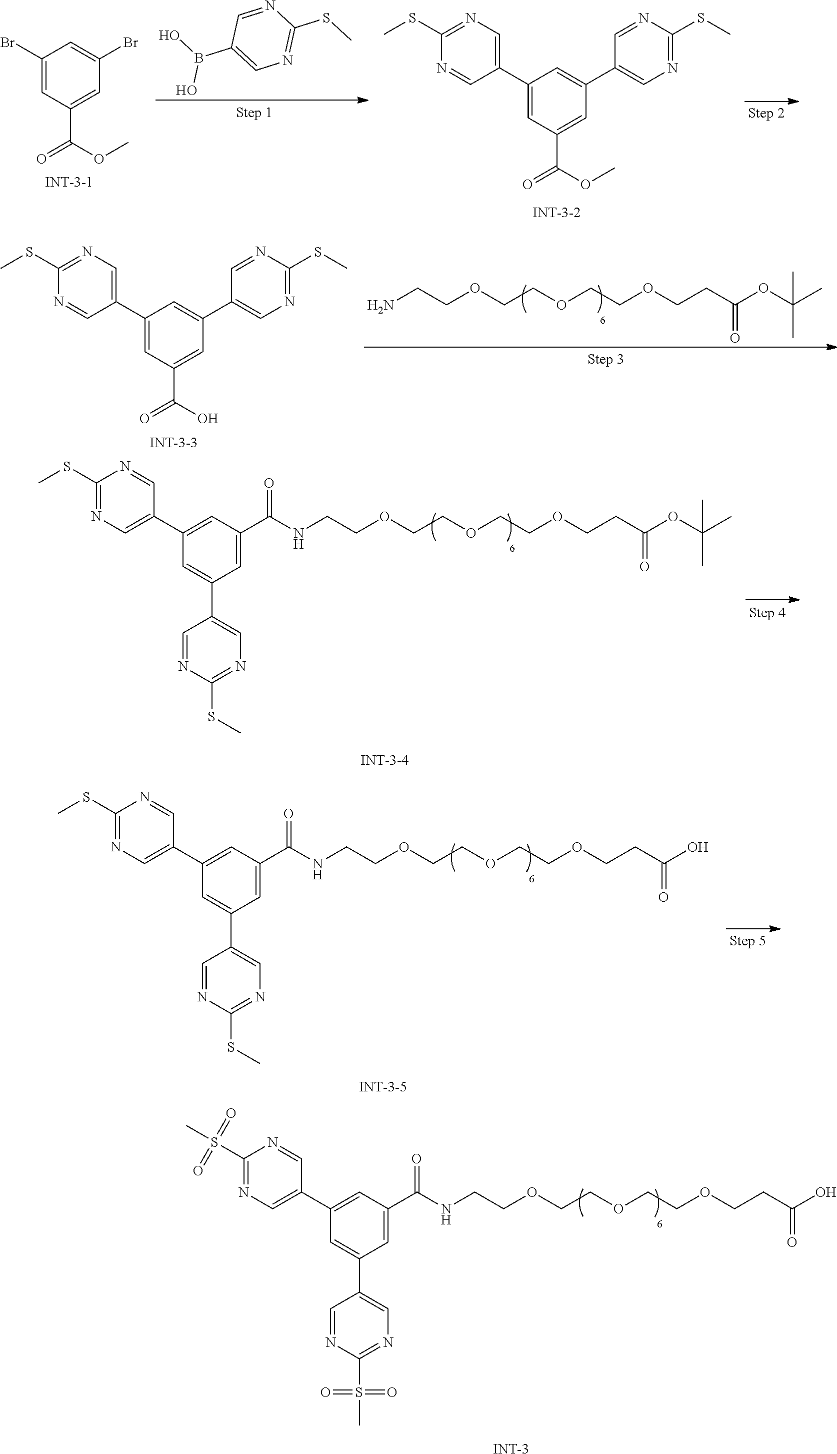

### Step 1: Preparation of methyl 3,5-bis(2-(methylthio)pyrimidin-5-yl)benzoate (INT-3-2)

Methyl 3,5-dibromobenzoate (720 mg, 2.45 mmol), 2-methylthiopyrimidine-5-boric acid (874 mg, 5.14 mmol), XPhosPd G3 (207 mg, 245 μmol), K₃PO₄ (1.56 g, 7.35 mmol) were added to dioxane (12 mL) and water (4 mL), and the reaction system was stirred at 90° C. for 3 hours under nitrogen atmosphere. The reaction was monitored by LC-MS, the reaction mixture was filtered with Celite pad, and the filtrate was added with water and ethyl acetate, extracted and concentrated to obtain a crude product, which was purified by column chromatography (EA/PE=0-25%) to obtain 710 mg of methyl 3,5-di(2-(methylthio)pyrimidin-5-yl)benzoate.

Its structural characterization data are as follows:

ESI-MS (m/z): 385.1 [M+H]⁺.

### Step 2: Preparation of 3,5-di(2-(methylthio)pyrimidin-5-yl)benzoic acid (INT-3-3)

Methyl 3,5-di(2-(methylthio)pyrimidin-5-yl)benzoate (650 mg, 1.69 mol) and lithium hydroxide (121 mg, 5.07 mmol) were dissolved in THF (2 mL), MeOH (2 mL) and H₂O (2 mL). The reaction mixture was stirred at 25° C. for 2 hours. The reaction was monitored by LC-MS. The pH of the reaction system was adjusted to about 2 with 1N HCl, and a large amount of solid was precipitated. After filtration, the filter cake was collected and dried to obtain 560 mg of 3,5-di(2-(methylthio)pyrimidin-5-yl)benzoic acid.

The structural characterization data are as follows:

ESI-MS (m/z): 371.1 [M+H]⁺.

### Step 3: Preparation of tert-butyl 1-(3,5-bis(2-(methylthio)pyrimidin-5-yl)phenyl)-1-oxo-5,8,11,14,17,20,23,26-octaoxa-2-azanonacosan-29-oate (INT-3-4)

3,5-Bis(2-(methylthio)pyrimidin-5-yl)benzoic acid (3.00 g, 8.10 mmol) and tert-butyl 1-amino-3,6,9,12,15,18,21,24-octaoxaheptan-27-oate (4.03 g, 8.10 mmol) were added to DMF (40 mL), and then added in sequence with HOBt (3.28 g, 24.3 mmol), EDCI (4.66 g, 24.3 mmol) and DIPEA (4.19 g, 32.4 mmol, 5.64 mL). The reaction system was stirred at 60° C. for 2 hours. The reaction solution was added with water (100 mL) and ethyl acetate (60 mL×3) for extraction. The organic phases were combined and dried over anhydrous sodium sulfate, filtered, and concentrated to obtain tert-butyl 1-(3,5-bis(2-(methylthio)pyrimidin-5-yl)phenyl)-1-oxo-5,8,11,14,17,20,23,26-octaoxa-2-azanonacosan-29-oate (4.20 g, 4.14 mmol), which was used directly in the next step without purification.

### Step 4: Preparation of 1-(3,5-bis(2-(methylthio)pyrimidin-5-yl)phenyl)-1-oxo-5,8,11,14,17,20,23,26-octaoxa-2-azanonacosan-29-oic acid (INT-3-5)

Tert-butyl 1-(3,5-bis(2-(methylthio)pyrimidin-5-yl)phenyl)-1-oxo-5,8,11,14,17,20,23,26-octaoxa-2-azanonacosan-29-oate (3.60 g, 4.24 mmol) was dissolved in dichloromethane (30 mL), and added with TFA (15.3 g, 134 mmol, 10 mL). The reaction system was stirred at 25° C. for 6 hours. The reaction solution was added with water (60 mL) and ethyl acetate (40 mL×3) for extraction. The organic phases were combined and dried over anhydrous sodium sulfate, filtered, and concentrated to obtain a crude product, which was purified by preparative high performance liquid chromatography and freeze-dried to obtain 1-(3,5-bis(2-(methylthio)pyrimidin-5-yl)phenyl)-1-oxo-5,8,11,14,17,20,23,26-octaoxa-2-azanonacosan-29-oic acid (2.93 g, 3.63 mmol).

Its structural characterization data are as follows:

ESI-MS (m/z): 794.3 [M+H]⁺.

Its purification method was as follows:

Chromatographic column: Phenomenex luna C18 (250 mm*70 mm*10 μm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 30 |
| 15.00 | 60 | 40 | 30 |

### Step 5: Preparation of 1-(3,5-bis(2-(methylsulfonyl)pyrimidin-5-yl)phenyl)-1-oxo-5,8,11,14,17,20,23,26-octaoxa-2-azanonacosan-29-oic acid (INT-3)

1-(3,5-Bis(2-(methylthio)pyrimidin-5-yl)phenyl)-1-oxo-5,8,11,14,17,20,23,26-octaoxa-2-azanonacosan-29-oic acid (148 mg, 0.186 mmol) was added to acetonitrile (15 mL) and water (7.5 mL), then the reaction system was added with sodium periodate (398.71 mg, 1.86 mmol) and ruthenium trichloride hydrate (15.47 mg, 74.56 μmol), and the reaction was carried out under stirring at 25° C. for 30 minutes. The reaction system was extracted with water and ethyl acetate, and concentrated to obtain the title compound (155 mg).

Its structural characterization data are as follows:

ESI-MS (m/z): 858.3 [M+H]⁺.

### Intermediate Preparation Example 4: 1-(3,5-Bis(2-(methylsulfonyl)pyrimidin-5-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (INT-4)

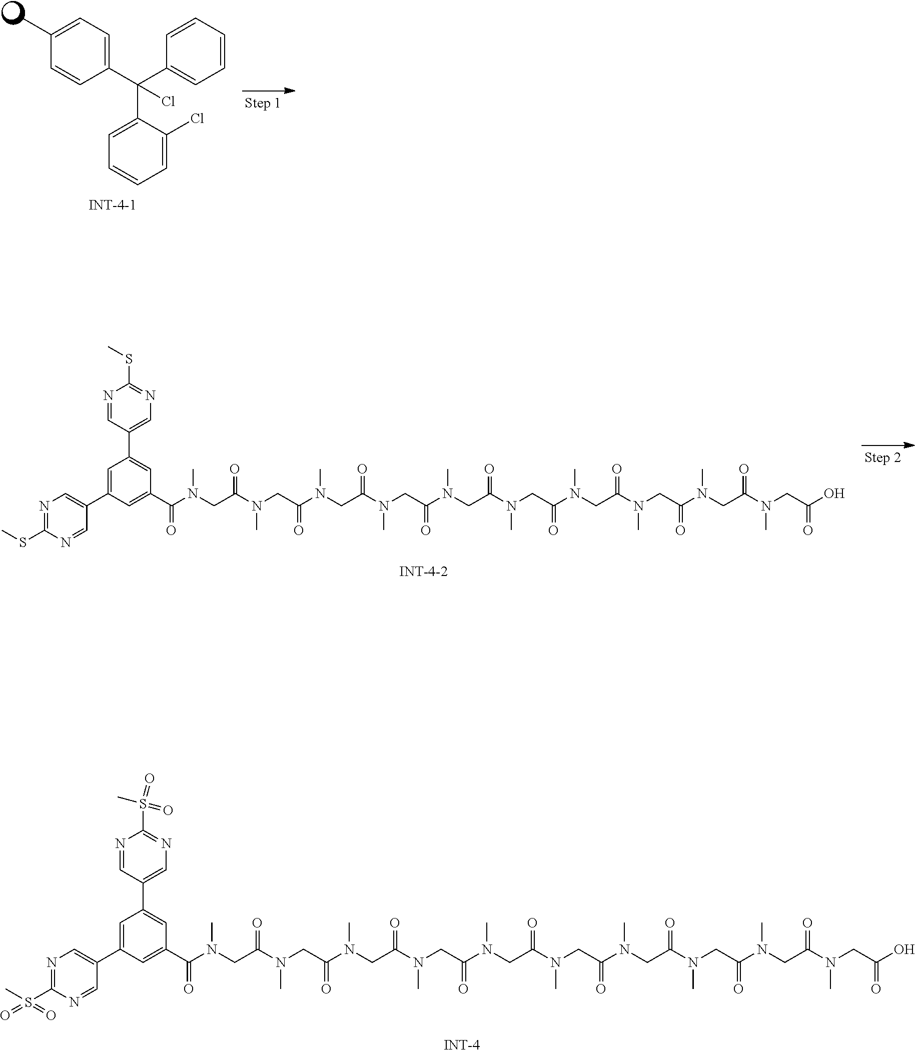 [SMILES: Cc1ccc(C(Cl)(c2ccccc2)c2ccccc2Cl)cc1 ; CSc1ncc(-c2cc(C(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc(-c3cnc(SC)nc3)c2)cn1 ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)c1cc(-c2cnc(S(C)(=O)=O)nc2)cc(-c2cnc(S(C)(=O)=O)nc2)c1 ; "INT-4"]

### Step 1: Preparation of 1-(3,5-bis(2-(methylthio)pyrimidin-5-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (INT-4-2)

A standard solid phase synthesis method was used:

1) Resin preparation: 2-CTC resin (3.00 mmol, 3.70 g, 0.81 mmol/g) and N-(((9H-fluoren-9-yl)methoxy)carbonyl)-N-methylglycine (3.00 mol, 933 mg, 3.00 equivalents) and DIPEA (4.00 equivalents) were added to dichloromethane (10.0 mL), and reacted under nitrogen atmosphere for 2 hours. Then, MeOH (1.0 mL) was added to the resin under nitrogen bubbling atmosphere within 30 minutes, and the resin was obtained by filtration.

2) Coupling: Under nitrogen bubbling, a DMF (10.0 mL) solution of N-(((9H-fluoren-9-yl)methoxy)carbonyl)-N-methylglycine (5.60 g, 6.00 equiv.) and HATU (6.58 g, 5.70 equiv.) was added to the resin. After DIPEA (6.00 equiv.) was added dropwise, the reaction system was subjected to nitrogen bubbling at 20° C. for 30 minutes. The resin was washed with DMF (30.0 m×5) before proceeding to the next step.

3) Deprotection: The resin was added with a 20% piperidine DMF solution (30.0 mL), and subjected to nitrogen bubbling at 20° C. for 30 minutes. The resin was then washed with DMF (30.0 mL)×5.

4) Steps 2 and 3 were repeated using the amino acids in Table 1: numbers 2 to 10 in Table 1.

5) The resin was then washed with DMF (30.0 mL×5) and MeOH (30.0 mL×5) and then dried under vacuum.

TABLE 1

| Material | Coupling reagent |
|---|---|
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| N-(((9H-fluoren-9- | HATU (5.70 equiv.) and DIPEA (6.00 equiv.) |
| yl)methoxy)carbonyl)-N-methylglycine | |
| (6.00 equiv.) | |
| 3,5-Bis(2-(methylthio)pyrimidin-5- | HATU (1.23 equiv.) and DIPEA (2.60 equiv.) |
| yl)benzoic acid (1.30 equiv.) | |

### Cleavage and Purification of Peptides:

1) A cleavage solution (TFA/DCM, 1/100, v/v, 200.0 mL) was added to a flask containing the peptides with protected side chains at room temperature, and stirred for 3 minutes twice.

2) After filtration, the filtrates were combined and concentrated.

3) The crude product was purified by high performance liquid chromatography and freeze-dried to obtain the title compound (1117.1 mg, TFA salt).

Its structural characterization data are as follows:

ESI-MS (m/z): 1010.4 (M+H)⁺.

Its purification method was as follows:

Separation conditions

| | |
|---|---|
| Dissolution conditions | DMF |
| Instrument | Auno DAC-100 |
| Mobile phase | A: water (0.1% TFA in H2O) |
| | B: acetonitrile |
| Gradient | 20-50%--42 min. Retention time: 21 min |
| Preparative column | Phenomenex Luna C18 250*100 mm, 10 um, 100Å |
| Flow rate | 250 mL/min |
| Detection wavelength | 220/254 nm |
| Temperature | 25° C. |

### Step 2: Preparation of 1-(3,5-bis(2-(methylsulfonyl)pyrimidin-5-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (INT-4)

1-(3,5-Bis(2-(methylthio)pyrimidin-5-yl)phenyl)-2,5,8,11,14,17,20,23,26-nonamethyl-1,4,7,10,13,16,19,22,25-nonaoxo-2,5,81,14,17,0,23,26-nonaazaoctacosan-28-ynoic acid (665 mg, 615.04 mol) was added to water (7.5 mL) and acetonitrile (15 mL), added with sodium periodate (1.32 g, 6.15 mmol) and ruthenium trichloride hydrate (51.03 mg, 246.02 μmol), and then reacted at 25° C. for 1 hour. The reaction solution was directly purified by reverse phase chromatography (acetonitrile/water (0.05% formic acid)=0-25%), and freeze-dried to obtain the title compound (515 mg, 449.69 μmol).

Its structural characterization data are as follows:

ESI-MS (m/z): 1074.4 (M+H)⁺.

### Intermediate Preparation Example 5: Preparation of 1-(3,5-bis(2-(methylsulfonyl) pyrimidin-5-yl)phenyl)-1-oxo-5,8,11,14,17-pentaoxa-2-azaeicosan-20-oic acid (INT-5)

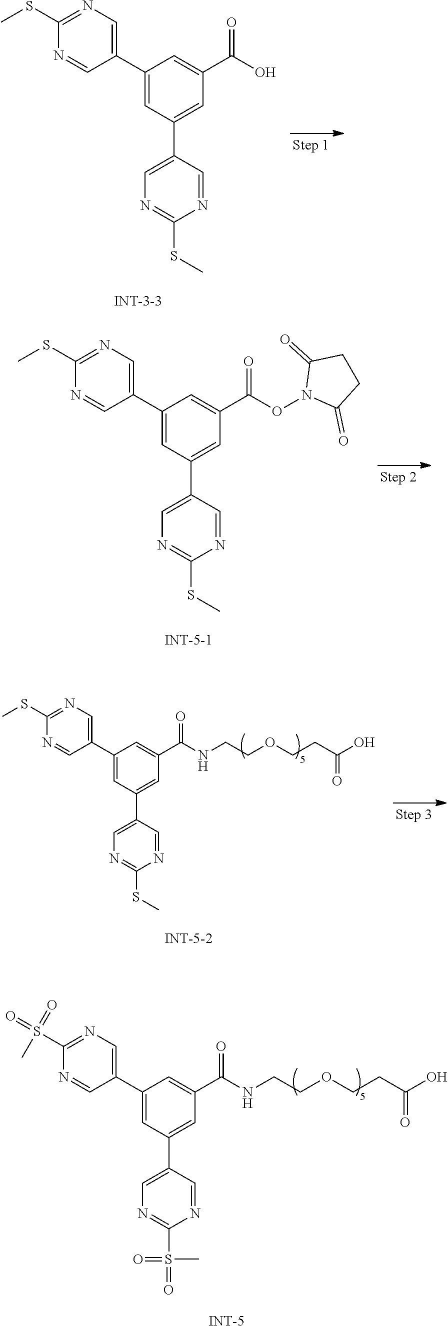

### Step 1: Preparation of 2,5-dioxopyrrolidin-1-yl 3,5-bis(2-(methylthio)pyrimidin-5-yl)benzoate (INT-5-1)

3,5-Bis(2-(methylthio)pyrimidin-5-yl)benzoic acid (0.6 g, 1.62 mmol) was dissolved in THF (15 mL), added with N-hydroxysuccinimide (278.89 mg, 2.43 mmol), then added with dicyclohexylcarbodiimide (0.4 g, 1.94 mmol), and stirred at room temperature for 2 hours. After the reaction was completed, the filtrate was collected after filtration, and concentrated under reduced pressure to obtain the title compound as a crude product (1.5 g, 3.42 mmol), which was directly used in the next reaction without purification.

### Step 2: Preparation of 1-(3,5-Bis(2-(methylthio)pyrimidin-5-yl)phenyl)-1-oxo-5,8,11,14,17-pentaoxa-2-azaeicosan-20-oic acid (INT-5-2)

2,5-Dioxopyrrolidin-1-yl 3,5-bis(2-(methylthio)pyrimidin-5-yl)benzoate (331.5 mg, 0.99 mmol) was dissolved in DMF (4 mL), added with 1-amino-3,6,9,12,15-pentaoxaoctadecan-18-oic acid (0.3 g, 0.67 mmol) and DIPEA (588.24 mg, 4.56 mol), and stirred at room temperature for 2 hours. After the reaction was completed, the title compound (315.50 mg, 0.44 mmol) was obtained by flash column chromatography (C18, water/acetonitrile=0.5).

Its structural characterization data are as follows:

MS m/z (ESI): 662.2 [M+H]⁺

### Step 3: Preparation of 1-(3,5-Bis(2-(methylsulfonyl)pyrimidin-5-yl)phenyl)-1-oxo-5,8,11,14,17-pentaoxa-2-azaeicosan-20-oic acid (INT-5)

1-(3,5-Bis(2-(methylthio)pyrimidin-5-yl)phenyl)-1-oxo-5,8,11,14,17-pentaoxa-2-azaeicosan-20-oic acid (315.50 mg, 0.44 mmol) was dissolved in acetonitrile (3 mL) and water (1.5 mL), added with sodium periodate (470.50 mg, 2.20 mmol) and ruthenium trichloride monohydrate (9.11 mg, 0.04 mmol), and stirred at room temperature for 0.5 hours. After the reaction was completed, the title compound (115.50 mg, 0.16 mmol) was obtained by flash column chromatography (C18, water/acetonitrile=2/1) and freeze-drying.

Its structural characterization data are as follows:

MS m/z (ESI): 726.1 [M+H]⁺

### Intermediate Preparation Example 6: Preparation of allyl (S)-(5-(2-((((9H-fluoren-9-yl) methoxy)carbonyl)amino)propionamido)-2-((((4-nitrophenoxy)carbonyl)oxy)methyl)benzyl)(methyl)carbamate (INT-6)

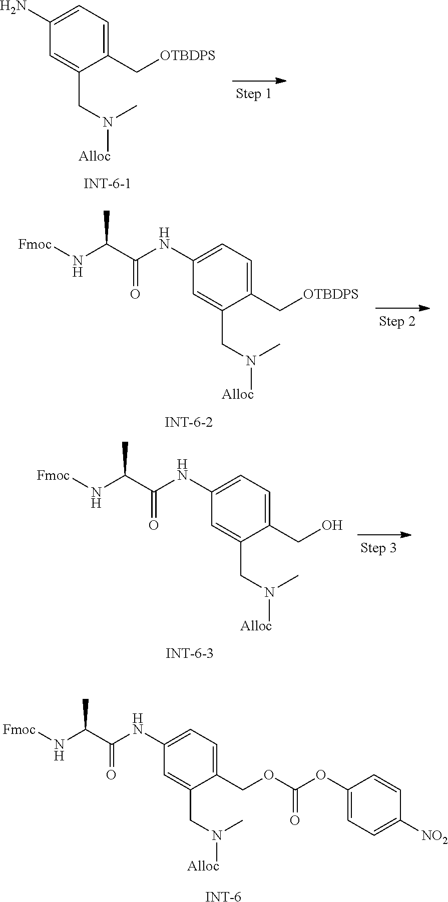

### Step 1: Preparation of allyl (S)-(5-(2-((((9H-fluoren-9-yl)methoxy)carbonyl)amino) propionamido)-2-(((tert-butyldiphenylsilyl)oxy)methyl)benzyl)(methyl)carbamate (INT-6-2)

Allyl (5-amino-2-(((tert-butyldiphenylsilyl)oxy)methyl)benzyl)(methyl)carbamate (150.0 mg, 0.31 mmol) and (((9H-fluoren-9-yl)methoxy)carbonyl)-L-alanine (115.1 mg, 0.37 mmol) were dissolved in DCM (8 mL) and MeOH (2 mL), added with 2-ethoxy-1-ethoxycarbonyl-1,2-dihydroquinoline (229.7 mg, 0.93 mmol), and stirred at room temperature for 15 hours. The reaction mixture was concentrated under reduced pressure to obtain a crude product, which was purified by silica gel column chromatography (ethyl acetate/petroleum ether=0 to 90%) and concentrated under reduced pressure again to obtain the title compound (194.2 mg, 0.25 mmol)

The structural characterization data are as follows:

MS m/z (ESI): 782.2 [M+H]⁺

### Step 2: Preparation of (S)-(5-(2-((((9H-fluoren-9-yl)methoxy)carbonyl)amino) propionamido)-2-(hydroxymethyl)benzyl)(methyl)carbamate (INT-6-3)

Allyl (S)-(5-(2-((((9H-fluoren-9-yl)methoxy)carbonyl)amino)propionamido)-2-(((tert-butyldiphenylsilyl)oxy)methyl)benzyl)(methyl)carbamate (194.2 mg, 0.25 mmol) was dissolved in DMF (5 mL), added with pyridine hydrofluoride (390.2 mg, 3.93 mmol), and stirred at room temperature for 15 hours. After the reaction was completed, the reaction solution was added with 20 mL of water, extracted with ethyl acetate 3 times (10 mL×3), washed with 10 mL of brine, dried over anhydrous sodium sulfate and concentrated under reduced pressure to obtain the crude product of the title compound, which was purified by flash column chromatography (C18, water/acetonitrile=2/1) and freeze-dried to obtain the title compound (109.3 mg, 0.21 mmol).

Its structural characterization data are as follows:

MS m/z (ESI): 566.1 [M+Na]⁺

### Step 3: Preparation of allyl (S)-(5-(2-((((9H-fluoren-9-yl)methoxy)carbonyl)amino) propionamido)-2-((((4-nitrophenoxy)carbonyl)oxy)methyl)benzyl)(methyl)carbamate (INT-6)

(S)-(5-(2-((((9H-fluoren-9-yl)methoxy)carbonyl)amino)propionamido)-2-(hydroxymethyl) benzyl)(methyl)carbamate (109.3 mg, 0.21 mmol) was dissolved in DMF (5 mL), added with DIPEA (81.3 mg, 0.63 mmol), then added with p-nitrophenyl chloroformate (50.8 mg, 0.25 mmol), and stirred at room temperature for 2 hours. After the reaction was completed, the title compound (134.50 mg, 0.19 mmol) was obtained by purification by flash column chromatography (C18, water/acetonitrile=2/1) and freeze-drying.

Its structural characterization data are as follows:

MS m/z (ESI): 731.2 [M+Na]⁺

### Example 1: Pentafluorophenyl 1-(N-((9S,13S)-13-hydroxy-6-isopropyl-14-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-9-methyl-4,7,10-trioxo-2-oxa-5,8,11-triazatetradecanoyl)sulfamoyl)piperidin-4-carboxylate (J-1)

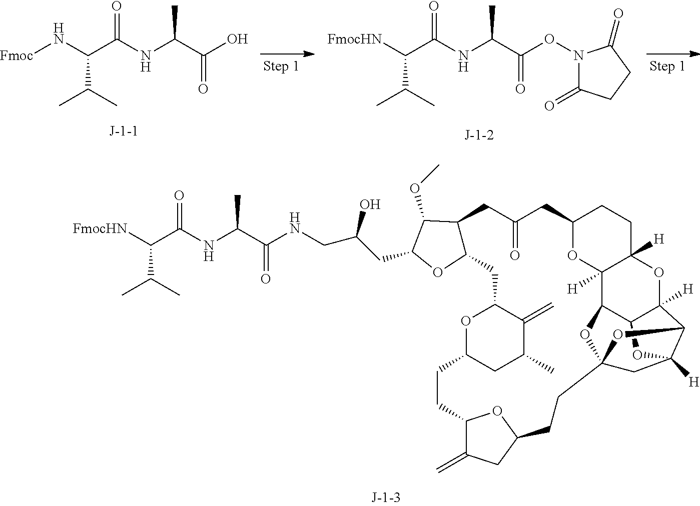

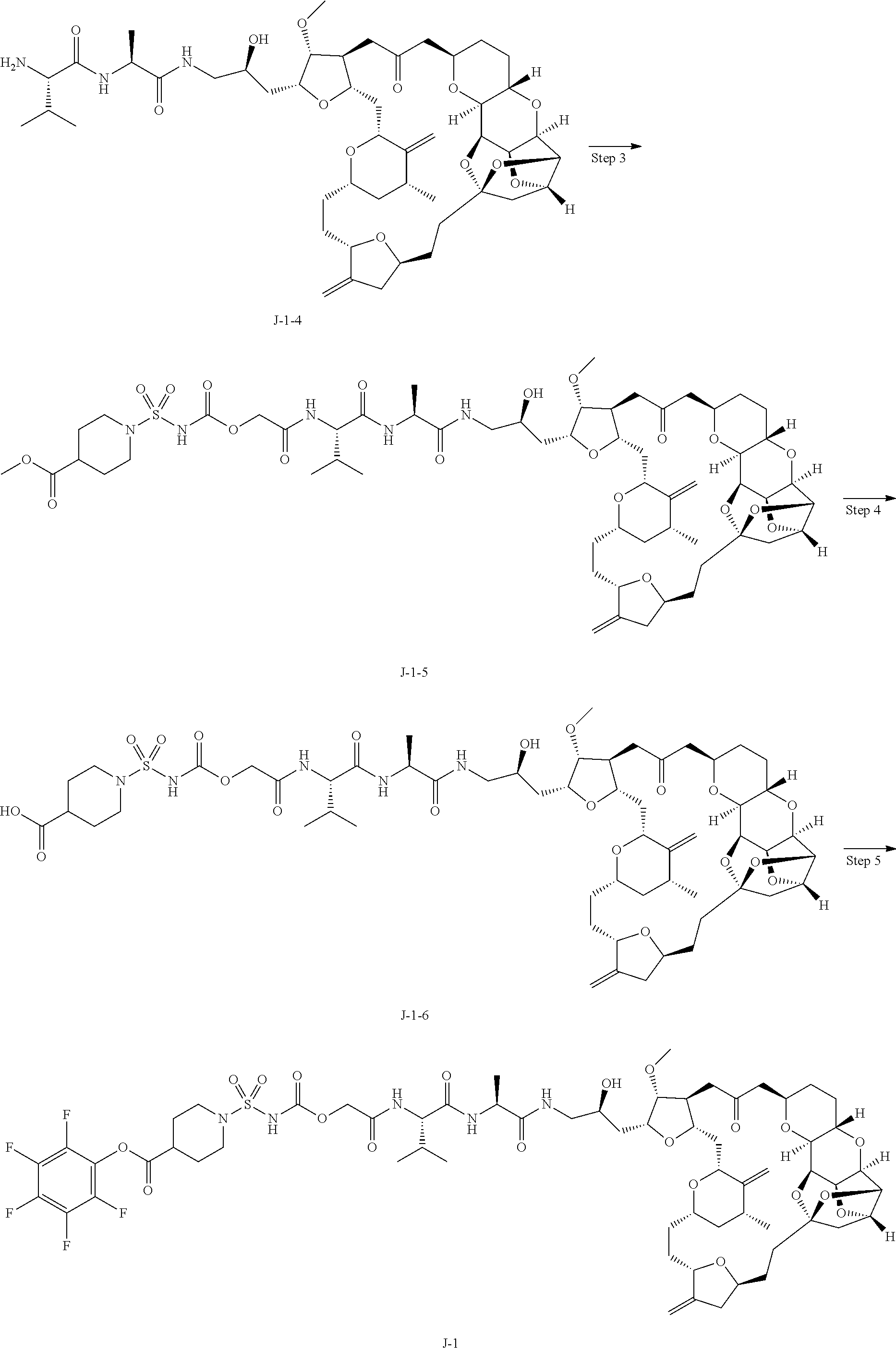

### Step 1: Preparation of 2,5-dioxopyrrolidin-1-yl (((9H-fluoren-9-yl)methoxy)carbonyl)-D-valyl-L-alaninate (J-1-2)

(((9H-Fluoren-9-yl)methoxy)carbonyl)-L-valyl-L-alanine (300 mg, 730.88 μmol), N-hydroxysuccinimide (84.12 mg, 730.88 μmol) and DCC (165.88 mg, 803.97 μmol) were added to THF (20 mL), and reacted at 25° C. for 18 hours. A large amount of white solid precipitated during the reaction. The solid was removed by filtration, and the filtrate was collected and concentrated to dryness to obtain 515 mg of the title compound with a purity of 70%. It was used directly in the next step without purification.

### Step 2: Preparation of (9H-fluoren-9-yl)methyl ((S)-1-(((S)-1-(((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]-dioxolan-18-yl)propyl)amino)-1-oxopropan-2-yl)amino)-3-methyl-1-oxobutan-2-yl)carbamate (J-1-3)

J-1-2 (198.67 mg, 274.01 μmol, 70%), eribulin (50 mg, 68.50 μmol), and DIPEA (35.41 mg, 274.01 μmol) were added to DMF (10 mL), and stirred at 25° C. for 3 hours. The reaction was monitored by LC-MS, and the reaction solution was concentrated to obtain a crude product, which was purified by preparative high performance liquid chromatography and freeze-dried to obtain 47 mg of the title compound.

Its structural characterization data are as follows:

ESI-MS (m/z): 1122.7 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 60 | 40 | 28 |
| 2.00 | 60 | 40 | 28 |
| 22.00 | 90 | 10 | 28 |

### Step 3: Preparation of (S)-2-amino-N—((S)-1-(((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxolan-18-yl)propyl)amino)-1-oxopropan-2-yl)-3-methylbutanamide (J-1-4)

J-1-3 (47 mg, 41.88 μmol) and DBU (12.75 mg, 83.75 μmol) were added to DMF (5 mL), and reacted at 25° C. for 2h. Formic acid (7.71 mg, 167.51 μmol) was added to the reaction system, and reacted under stirring for 20 minutes. Water and MTBE were added to the reaction system for extraction to remove some impurities. The aqueous phase was freeze-dried to obtain 55 mg (crude product) of the title compound, which was used directly in the next step without further purification.

Its structural characterization data are as follows:

ESI-MS (m/z): 900.6[M+H]⁺.

### Step 4: Preparation of methyl 1-(N-((9S,13S)-13-hydroxy-6-isopropyl-14-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-9-methyl-4,7,10-trioxo-2-oxa-5,8,11-triazatetradecanoyl)sulfamoyl)piperidin-4-carboxylate (J-1-5)

J-1-4 (53.85 mg, 38.88 μmol, crude product) and DIPEA (25.13 mg, 194.42 μmol) were added to DMF (10 mL), and reacted under stirring for 5 minutes. Then, 2-[(4-methoxycarbonyl-1-piperidinyl)sulfonylaminoformyloxy]acetic acid (25.22 mg, 77.77 μmol) and HATU (29.57 mg, 77.77 μmol) were added to the reaction system, and reacted at 25° C. for 18 hours. The reaction was monitored by LC-MS. The reaction solution was concentrated to obtain a crude product, which was purified by column chromatography (MeOH/EA=0-15%) to obtain 28 mg of the title compound.

Its structural characterization data are as follows:

ESI-MS (m/z): 1206.6[M+H]⁺.

### Step 5: Preparation of 1-(N-((9S,13S)-13-hydroxy-6-isopropyl-14-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-9-methyl-4,7,10-trioxo-2-oxa-5,8,11-triazatetradecanoyl)sulfamoyl)piperidin-4-carboxylic acid (J-1-6)

J-1-5 (30 mg, 24.87 μmol) and lithium hydroxide monohydrate (40.74 mg, 994.70 μmol) were added to MeOH (12 mL) and water (6 mL), and reacted at 35° C. for 4 hours. The reaction was monitored by LC-MS. The reaction system was adjusted to about pH 3 with 0.5 N HCl, extracted with ethyl acetate, and concentrated to dryness to obtain 28 mg of the title compound, which was used directly in the next step without further purification.

Its structural characterization data are as follows:

ESI-MS (m/z): 1193.5[M+H]⁺.

### Step 6: Preparation of pentafluorophenyl 1-(N-((9S,13S)-13-hydroxy-6-isopropyl-14-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-9-methyl-4,7,10-trioxo-2-oxa-5,8,11-triazatetradecanoyl)sulfamoyl)piperidin-4-carboxylate (J-1)

Compound J-1-6 (28 mg, 23.48 μmol) and EDCI (45.02 mg, 234.83 μmol) were added to DMF (5 mL), and reacted at 25° C. for 16 hours. The reaction was monitored by LC-MS. The reaction solution was concentrated to obtain a crude product, which was purified by preparative high performance liquid chromatography and freeze-dried to obtain 0.50 mg of the title compound.

Its structural characterization data are as follows:

ESI-MS (m/z): 1358.6 [M+H]⁺.

Its preparation method was as follows:

Chromatographic column: Waters XBridge Prep C180BD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 50 | 50 | 30 |
| 2.00 | 50 | 50 | 30 |
| 16.00 | 95 | 5 | 30 |

### Example 2: pentafluorophenyl 1-(N-((6S,9S,12S,16S)-16-hydroxy-17-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-6,9,12-trimethyl-4,7,10,13-tetraoxo-2-oxa-5,8,11,14-tetraazaheptadecanoyl)sulfamoyl)piperidin-4-carboxylate (J-3)

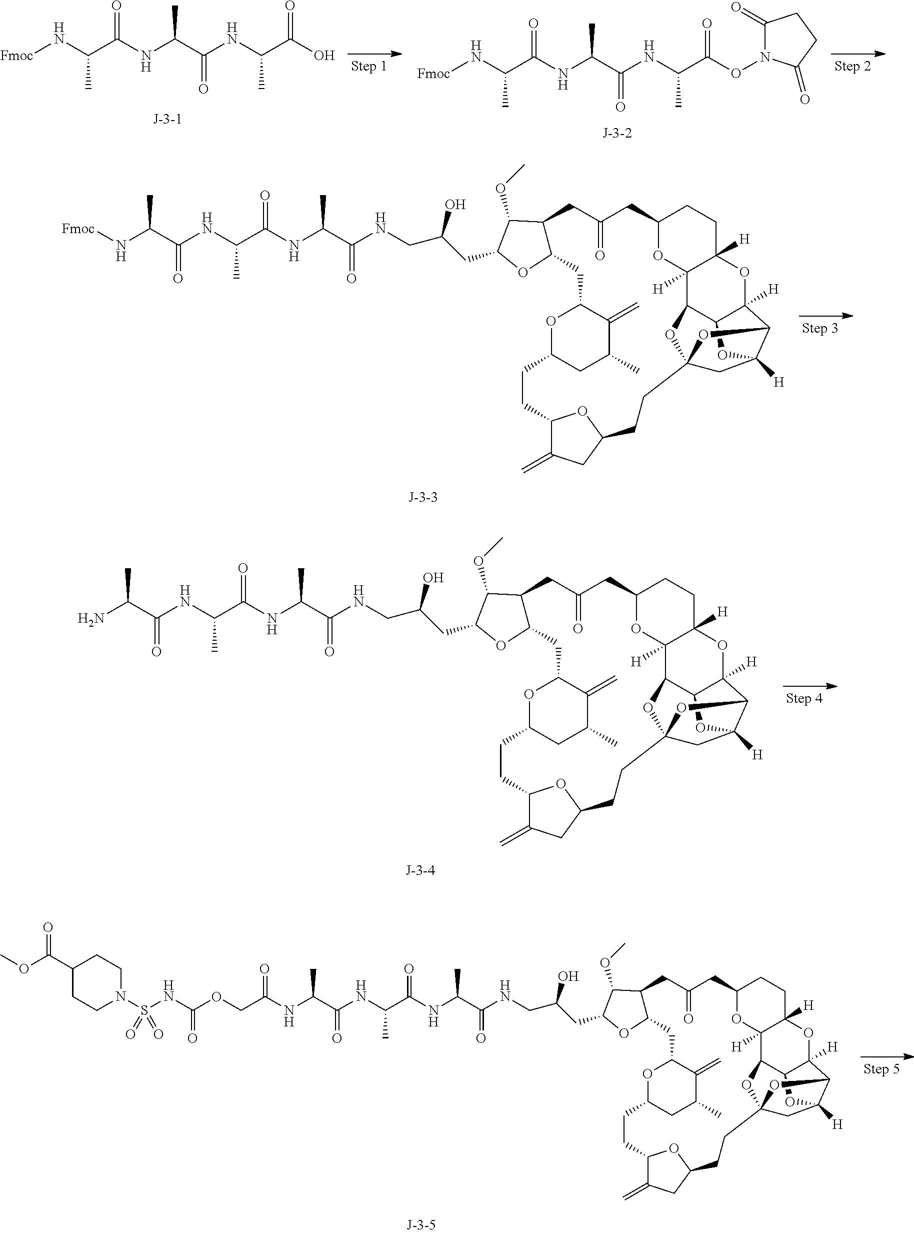

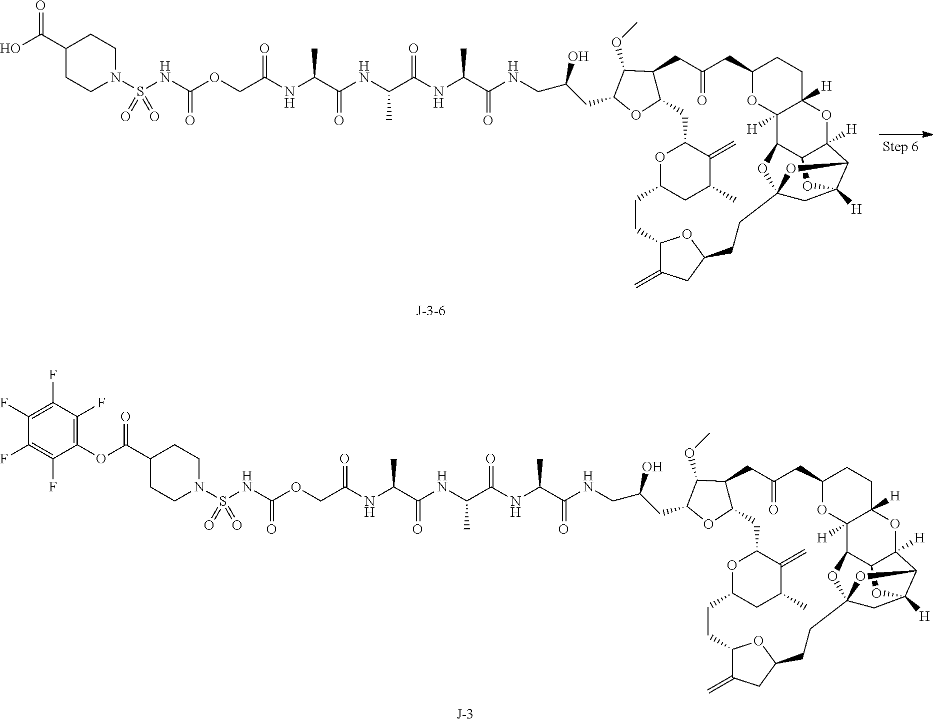

### Step 1: Preparation of 2,5-dioxopyrrolidin-1-yl (((9H-fluoren-9-yl)methoxy)carbonyl)-L-alanyl-L-alanyl-L-alaninate (J-3-2)

(((9H-Fluoren-9-yl)methoxy)carbonyl)-L-alanyl-L-alanyl-L-alanine (200.00 mg, 441.03 μmol), N-hydroxysuccinimide (101.51 mg, 882.05 μmol, FR) and EDCI (169.09 mg, 882.05 μmol) were added to DMF (10 mL), and reacted at 25° C. for 1 hour. The reaction solution was added with water and ethyl acetate, extracted, dried over sodium sulfate, and concentrated to obtain 206 mg of the title compound with a purity of 90%. It was used directly in the next step without purification.

Its structural characterization data are as follows:

ESI-MS (m/z): 551.3 [M+H]⁺.

### Step 2: Preparation of (9H-fluoren-9-yl)methyl ((S)-1-(((S)-1-(((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)amino)-1-oxopropan-2-yl)amino)-1-oxopropan-2-yl)amino)-1-oxopropan-2-yl)carbamate (J-3-3)

Compound J-3-2 (178.65 mg, 308.26 μmol, purity 90%), eribulin (90 mg, 123.31 μmol) and DIPEA (47.81 mg, 369.92 μmol) were added to DMF (10 mL), and reacted under stirring at 25° C. for 18 hours. The reaction solution was directly concentrated to obtain a crude product, which was purified by preparative high performance liquid chromatography and freeze-dried to obtain 67 mg of the title compound.

Its structural characterization data are as follows:

ESI-MS (m/z): 1165.7 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 30 |
| 3.00 | 40 | 60 | 30 |
| 18.00 | 90 | 10 | 30 |

### Step 3: Preparation of (S)-2-amino-N—((S)-1-(((S)-1-(((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)amino)-1-oxopropan-2-yl)amino)-1-oxopropan-2-yl)propanamide (J-3-4)

Compound J-3-3 (66 mg, 56.63 μmol) and DBU (17.24 mg, 113.27 μmol) was added to DMF (10 mL) and reacted at 25° C. for 2 hours. The reaction system was added with formic acid (10.43 mg, 226.54 μmol) and stirred for 20 minutes. The reaction system was added with water and MTBE for extraction to remove some impurities. The aqueous phase was freeze-dried to obtain 73 mg (crude product) of the title compound, which was used directly in the next step without further purification.

Its structural characterization data are as follows:

ESI-MS (m/z): 943.6 [M+H]⁺.

### Step 4: Preparation of methyl 1-(N-((6S,9S,12S,16S)-16-hydroxy-17-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-6,9,12-trimethyl-4,7,10,13-tetraoxo-2-oxa-5,8,11,14-tetraazaheptadecanoyl)sulfamoyl)piperidin-4-carboxylate (J-3-5)

Compound J-3-4 (65 mg, 48.24 μmol, crude product) and DIPEA (31.18 mg, 241.22 μmol) were added to DMF (10 mL), reacted under stirring for 10 minutes, then the reaction system was added with 2-[(4-methoxycarbonyl-1-piperidinyl)sulfonylaminoformyloxy]acetic acid (31.29 mg, 96.49 μmol) and HATU (36.69 mg, 96.49 μmol), and reacted at 25° C. for 16 hours. The reaction solution was directly concentrated to obtain a crude product, which was purified by column chromatography (MeOH/DCM=0-10%) to obtain 32 mg of the title compound.

Its structural characterization data are as follows:

ESI-MS (m/z): 1250.6 [M+H]⁺.

### Step 5: Preparation of 1-(N-((6S,9S,12S,16S)-16-hydroxy-17-((2S,5S,8S,11S,13R,15R,16aS, 18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-6,9,12-trimethyl-4,7,10,13-tetraoxo-2-oxa-5,8,11,14-tetraazaheptadecanoyl)sulfamoyl)piperidin-4-carboxylic acid (J-3-6)

Compound J-3-5 (39 mg, 31.21 μmol, FR) and lithium hydroxide monohydrate (38.85 mg, 936.44 μmol) were added to MeOH (15 mL) and water (7.5 mL), reacted at 35° C. for 4h, adjusted with 0.5 N HCl so that the system pH was about 3, extracted with ethyl acetate, and concentrated to dryness to obtain 34 mg of the title compound, which was used directly in the next step without further purification.

### Step 6: Preparation of pentafluorophenyl 1-(N-((6S,9S,12S,16S)-16-hydroxy-17-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-6,9,12-trimethyl-4,7,10,13-tetraoxo-2-oxa-5,8,11,14-tetraazaheptadecanoyl)sulfamoyl)piperidin-4-carboxylate (J-3)

Compound J-3-6 (34 mg, 27.52 μmol), 2,3,4,5,6-pentafluorophenol (75.99 mg, 412.82 μmol) and EDCI (94.97 mg, 495.39 μmol) were added to DMF (10 mL), and reacted at 25° C. for 5 hours. The reaction solution was directly concentrated to obtain a crude product, which was purified by preparative high performance liquid chromatography and freeze-dried to obtain 3 mg of the title compound.

Its structural characterization data are as follows:

ESI-MS (m/z): 1401.6 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 50 | 50 | 30 |
| 2.00 | 50 | 50 | 30 |
| 16.00 | 95 | 5 | 30 |

### Example 3: Pentafluorophenyl 1-(N-((12S,19S)-12-benzyl-19-hydroxy-20-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosyl-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-4,7,10,13,16-pentaoxo-2-oxa-5,8,11,14,17-pentaazaeicosanoyl)sulfamoyl)piperidin-4-carboxylate (J-6)

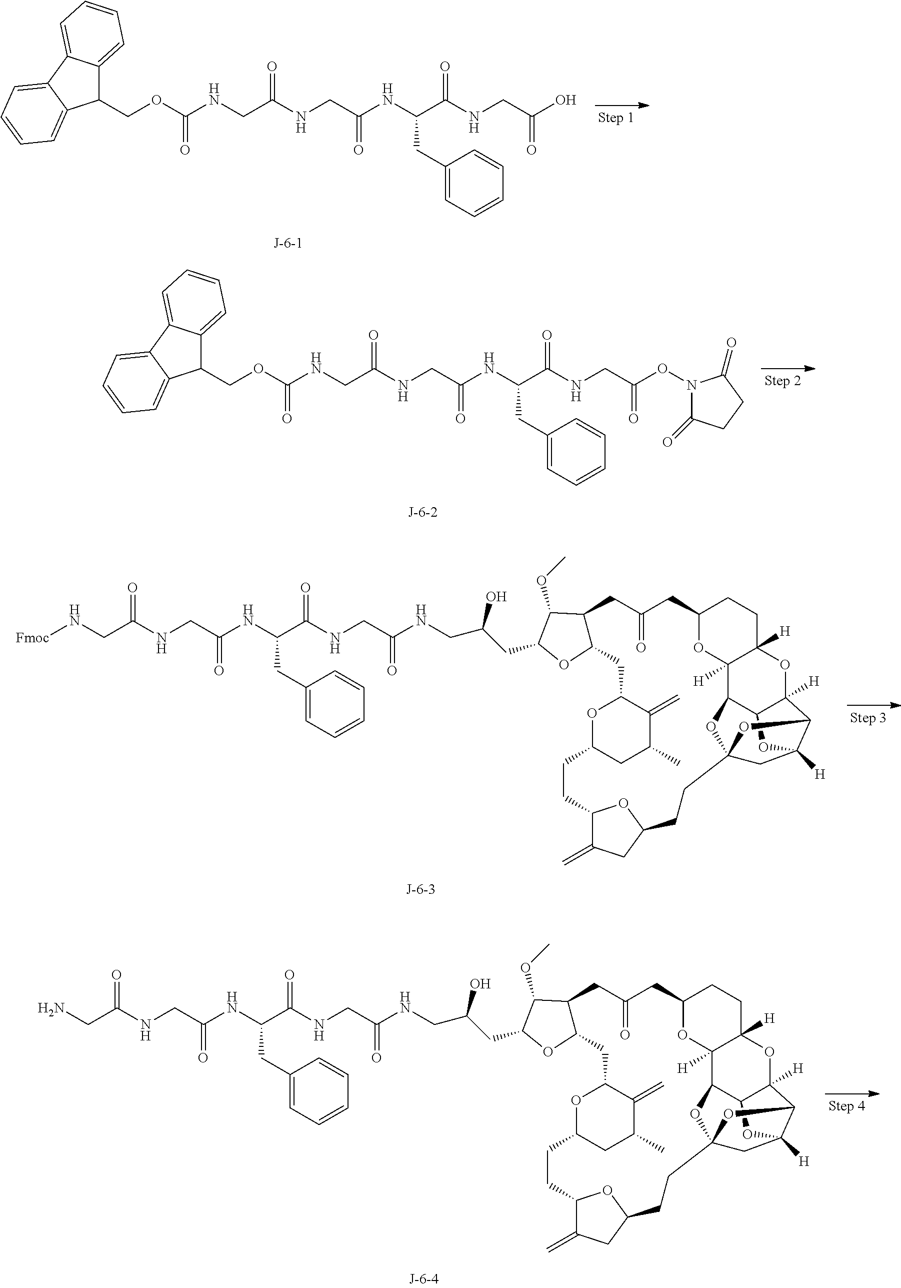

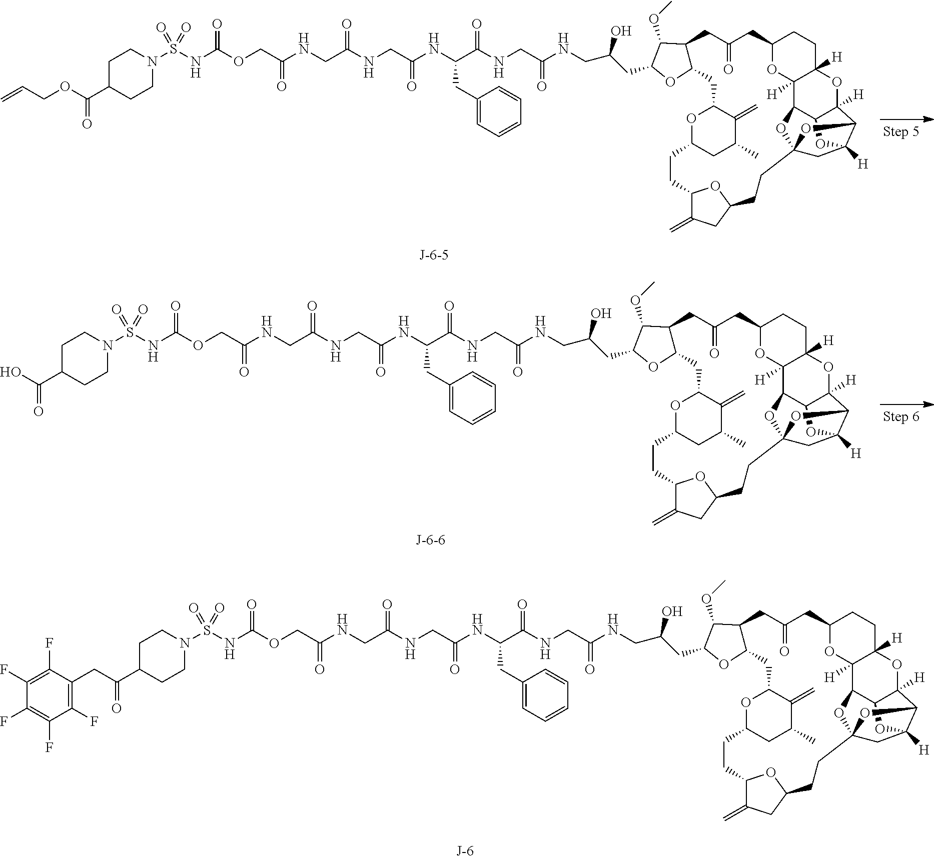

### Step 1: Preparation of 2,5-dioxopyrrolidin-1-yl (((9H-fluoren-9-yl)methoxy)carbonyl)glycylglycyl-L-phenylalanylglycinate (J-6-2)

(((9H-Fluoren-9-yl)methoxy)carbonyl)glycylglycyl-L-phenylalanylglycine (500.00 mg, 895.13 μmol), N-hydroxysuccinimide (206.04 mg, 1.79 mmol) and EDCI (343.19 mg, 1.79 mmol) were added to DMF (10 mL), reacted at 25° C. for 2 hours, added with water and ethyl acetate for extraction, dried over sodium sulfate, and concentrated to obtain 640 mg of the title compound with a purity of 90%. It was directly used in the next reaction without purification.

Its structural characterization data are as follows:

ESI-MS (m/z): 673.3 [M+18]⁺.

### Step 2: Preparation of (9H-fluoren-9-yl)methyl ((7S,14S)-7-benzyl-14-hydroxy-15-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-2,5,8,11-tetrahydro-3,6,9,12-tetraazapentadecyl)carbamate (J-6-3)

Compound J-6-2 (349.33 mg, 479.52 μmol, the purity was 90%), eribulin (100 mg, 137.01 μmol) and DIPEA (70.83 mg, 548.02 μmol) were added to DMF (10 mL), and stirred at 25° C. for 18 hours. The reaction solution was directly concentrated to obtain a crude product, which was purified by preparative high performance liquid chromatography and freeze-dried to obtain 125 mg of the title compound.

Its structural characterization data are as follows:

ESI-MS (m/z): 1270.7 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 45 | 55 | 28 |
| 2.00 | 45 | 55 | 28 |
| 22.00 | 90 | 10 | 28 |

### Step 3: Preparation of (S)-2-(2-(2-aminoacetamido)acetamido)-N-(2-(((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)amino)-2-oxoethyl)-3-phenylpropanamide (J-6-4)

Compound J-6-3 (125 mg, 98.39 μmol) and DBU (44.94 mg, 295.17 μmol) were added to DMF (8 mL), and reacted at 25° C. for 2 hours. The reaction solution was directly concentrated to obtain a crude product, which was purified by preparative high performance liquid chromatography and freeze-dried to obtain 64 mg of the title compound.

Its structural characterization data are as follows:

ESI-MS (m/z): 1048.6 [M+H]⁺.

The preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 15 | 85 | 28 |
| 2.00 | 15 | 85 | 28 |
| 22.00 | 90 | 10 | 28 |

### Step 4: Preparation of allyl 1-(N-((12S,19S)-12-benzyl-19-hydroxy-20-((2S,5S,8S,11S, 13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-4,7,10,13,16-pentaoxo-2-oxa-5,8,11,14,17-pentaazaeicosanoyl)sulfamoyl)piperidin-4-carboxylate (J-6-5)

Compound J-6-4 (53 mg, 48.44 μmol) and DIPEA (31.30 mg, 242.18 μmol) were added to DMF (10 mL), and reacted under stirring for 5 min. Then 2-[(4-allyloxycarbonyl-1-piperidinyl)sulfonylcarbamoyloxy]acetic acid (37.71 mg, 96.87 μmol, purity 90%) and HATU (36.83 mg, 96.87 μmol) were added to the reaction system, and reacted at 25° C. for 3 hours. The reaction system was directly concentrated to obtain a crude product, which was purified by preparative high performance liquid chromatography and freeze-dried to obtain 33 mg of the title compound.

Its structural characterization data are as follows:

ESI-MS (m/z): 1381.6 [M+H]⁺.

The preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 30 |
| 3.00 | 40 | 60 | 30 |
| 18.00 | 95 | 5 | 30 |

### Step 5: Preparation of 11-(N-((12S,19S)-12-benzyl-19-hydroxy-20-((2S,5S,8S,11S,13R,15R, 16aS,18R,19R,19aS,23R,24aS,25S,26aR,27S,28R, 29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-4,7,10,13,16-pentaoxo-2-oxa-5,8,11,14,17-pentaazaeicosanoyl)sulfamoyl)piperidin-4-carboxylic acid (J-6-5)

Compound J-6-5 (30 mg, 21.73 μmol), morpholine (18.93 mg, 217.30 μmol) and tetrakistriphenylphosphine palladium (15.07 mg, 13.04 μmol) were dissolved in THF (20 mL), vacuumed, subjected to replacement with nitrogen three times, and reacted at 25° C. for 16 hours. The reaction solution was directly concentrated to obtain a crude product, which was purified by preparative high performance liquid chromatography and freeze-dried to obtain 11 mg of the title compound.

Its structural characterization data are as follows:

ESI-MS (m/z): 1341.5 [M+H]⁺.

Its preparation method was as follows:

Chromatographic column: Waters XBridge Prep C18OBD (5 μm*19 mm*150 mm)

Mobile phase A: acetonitrile; Mobile phase B: water(0.05% formic acid)

| Time | Mobile phase A | Mobile phase B | Flow rate |
|---|---|---|---|
| [min] | [%] | [%] | [mL/min] |
| 0.00 | 30 | 70 | 32 |
| 4.00 | 30 | 70 | 32 |
| 24.00 | 90 | 10 | 32 |

### Step 6: Preparation of pentafluorophenyl 1-(N-((12S,19S)-12-benzyl-19-hydroxy-20-((2S,5S, 8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-4,7,10,13,16-pentaoxo-2-oxa-5,8,11,14,17-pentaazaeicosanoyl)sulfamoyl)piperidine-4-oate (J-6)

Compound J-6-6 (16 mg, 11.94 μmol), 2,3,4,5,6-pentafluorophenol (32.95 mg, 179.04 μmol), and EDCI (41.19 mg, 214.85 μmol) were added to DMF (5 mL), and reacted at 25° C. for 4 hours.

The reaction solution was directly concentrated to obtain a crude product, which was purified by preparative high performance liquid chromatography and freeze-dried to obtain 2.60 mg of the title compound.

Its structural characterization data are as follows: ESI-MS (m/z): 1506.6 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 50 | 50 | 30 |
| 2.00 | 50 | 50 | 30 |
| 18.00 | 95 | 5 | 30 |

### Example 4: Pentafluorophenyl 1-(N-((6S,9S)-14-amino-9-((4-(((((S)-2-hydroxy-3-((2S,5S, 8S,11S,13R,15R,16aS,18R,19R,19aS,23R, 24aS, 25S, 26aR, 27S,28R, 29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamoyl)oxy)methyl)phenyl)carbamoyl)-6-isopropyl-4,7,14-trioxo-2-oxa-5,8,13-triazatetradecanoyl)sulfamoyl)piperidin-4-carboxylate (J-8)

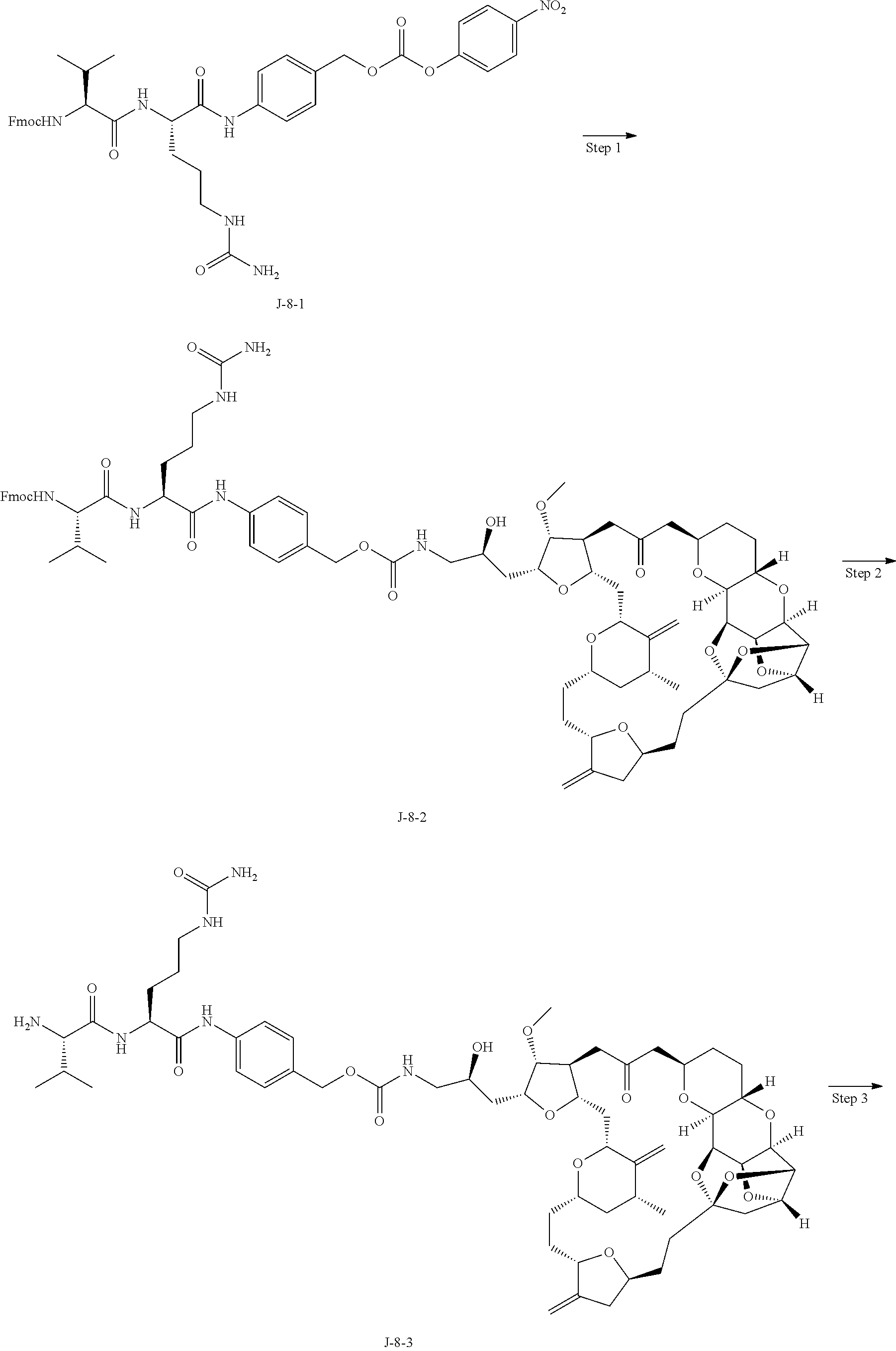

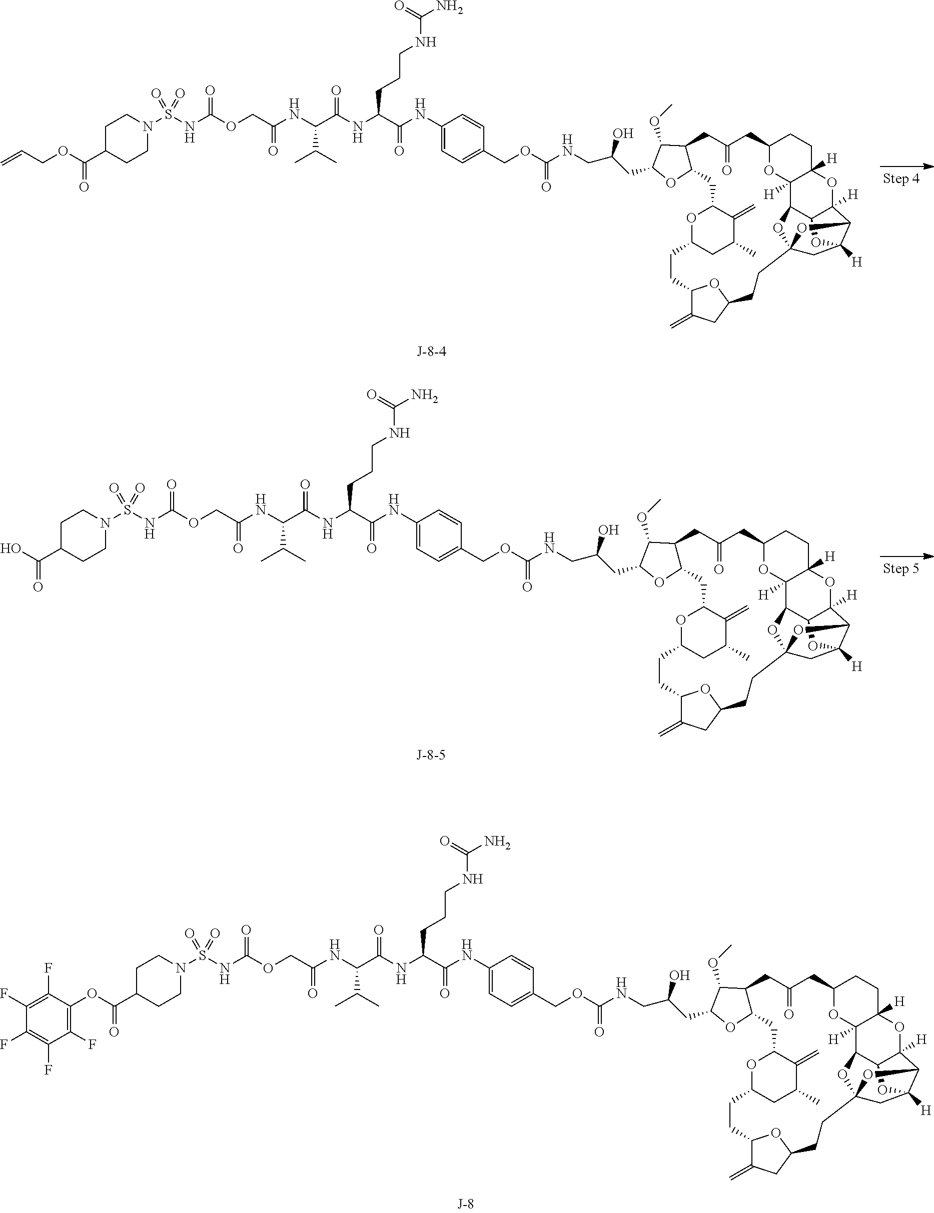

### Step 1: Preparation of 4-((S)-2-((S)-2-((((9H-fluoren-9-yl)methoxy)carbonyl)amino)-3-methylbutyrylamido)-5-ureidopentanamido)benzyl ((S)-2-hydroxy-3-((2S, 5S, 8S,11 S,13R,15R, 16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamate (J-8-2)

Eribulin (90 mg, 123.31 μmol), (9H-fluoren-9-yl)methyl ((S)-3-methyl-1-(((S)-1-((4-((((4-nitrophenoxy)carbonyl)oxy)methyl)phenyl)amino))-1-oxo-5-ureidopentan-2-yl)amino)-1-oxobutan-2-yl)carbamate (199.05 mg, 246.61 mol, purity 95%), HOBT (33.32 mg, 246.61 μmol) and pyridine (48.77 mg, 616.53 μmol) were added to DMF (10 mL), stirred at 25° C. for 18 hours. The reaction solution was directly concentrated to obtain a crude product, which is purified by preparative high performance liquid chromatography and freeze-dried to obtain 100 mg of the title compound.

Its structural characterization data are as follows:

ESI-MS (m/z): 1357.8 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 30 |
| 2.00 | 40 | 60 | 30 |
| 18.00 | 90 | 10 | 30 |

### Step 2: Preparation of 4-((S)-2-((S)-2-amino-3-methylbutyramido)-5-ureidopentanamido)benzyl ((S)-2-hydroxy-3-((2S,5S,8S,11S,13R,15R,16aS,18R,19R,19aS,23R, 24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamate (J-8-3)

Compound J-8-2 (100 mg, 73.66 μmol) was added to DMF (10 mL) and diethylamine (2 mL), stirred at 25° C. for 50 minutes. The reaction solution was directly concentrated to obtain a crude product, which was purified by preparative high performance liquid chromatography and freeze-dried to obtain 64 mg of the title compound.

Its structural characterization data are as follows:

ESI-MS (m/z): 1135.6 [M+H]⁺.

The preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 20 | 80 | 32 |
| 4.00 | 20 | 80 | 32 |
| 24.00 | 90 | 10 | 32 |

### Step 3: Preparation of allyl 1-(N-((6S,9S)-14-amino-9-((4-(((((S)-2-hydroxy-3-((2S,5S,8S, 11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamoyl)oxy)methyl)phenyl)carbamoyl)-6-isopropyl-4,7,14-trioxo-2-oxa-5,8,13-triazatetradecanoyl)sulfamoyl)piperidin-4-carboxylate (J-8-4)

Compound J-8-3 (27 mg, 22.85 μmol, crude product) and DIPEA (14.77 mg, 114.77 μmol) were added to DMF (5 mL), and stirred for 5 minutes. Then 2-[(4-allyloxycarbonyl-1-piperidinyl)sulfonylcarbamoyloxy]acetic acid (17.79 mg, 45.71 μmol, purity 90%) and HATU (17.38 mg, 45.71 μmol) were added to the reaction system, and reacted at 25° C. for 16 hours. The reaction solution was directly concentrated to obtain a crude product, which was purified by preparative high performance liquid chromatography and freeze-dried to obtain 19 mg of the title compound.

Its structural characterization data are as follows:

ESI-MS (m/z): 1467.7 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 32 |
| 4.00 | 40 | 60 | 32 |
| 24.00 | 90 | 10 | 32 |

### Step 4: Preparation of 1-(N-((6S,9S)-14-amino-9-((4-(((((S)-2-hydroxy-3-((2S,5S,8S,11S, 13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamoyl)oxy)methyl)phenyl)carbamoyl)-6-isopropyl-4,7,14-trioxo-2-oxa-5,8,13-triazatetradecanoyl)sulfamoyl)piperidin-4-carboxylic acid (J-8-5)

Compound J-8-4 (35 mg, 23.85 μmol), morpholine (20.78 mg, 238.47 μmol) and tetrakistriphenylphosphine palladium (16.53 mg, 14.31 μmol) were dissolved in THF (25 mL), vacuumed, subjected to replacement with nitrogen 3 times, and reacted at 25° C. for 2 hours. The reaction solution was directly concentrated to obtain a crude product, which was purified by preparative high performance liquid chromatography and freeze-dried to obtain 18 mg of the title compound.

Its structural characterization data are as follows:

ESI-MS (m/z): 1427.7 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 35 | 65 | 28 |
| 2.00 | 35 | 65 | 28 |
| 22.00 | 90 | 10 | 28 |

### Step 5: Preparation of pentafluorophenyl 1-(N-((6S,9S)-14-amino-9-((4-(((((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamoyl)oxy)methyl)phenyl)carbamoyl)-6-isopropyl-4,7,14-trioxo-2-oxa-5,8,13-triazatetradecanoyl)sulfamoyl)piperidin-4-carboxylate (J-8)

Compound 5 (18 mg, 12.61 μmol), 2,3,4,5,6-pentafluorophenol (34.81 mg, 189.13 μmol), and EDCI (43.51 mg, 226.95 μmol) were added to DMF (5 mL), and reacted at 25° C. for 3 hours. The reaction solution was directly concentrated to obtain a crude product, which was purified by preparative high performance liquid chromatography and freeze-dried to obtain 6 mg of the title compound.

The structural characterization data are as follows:

ESI-MS (m/z): 1594.7 [M+H]⁺.

The preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 28 |
| 2.00 | 40 | 60 | 28 |
| 22.00 | 90 | 10 | 28 |

### Example 5: 4-((31S,34S)-1-(3,5-Bis(2-(methylsulfonyl)pyrimidin-5-yl)phenyl)-31-isopropyl-1,29,32-trioxo-34-(3-ureidopropyl))-5,8,11,14,17,20,23,26-octaoxa-2,30,33-triazapentatriacontan-35-amido)benzyl((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamate (H-1)

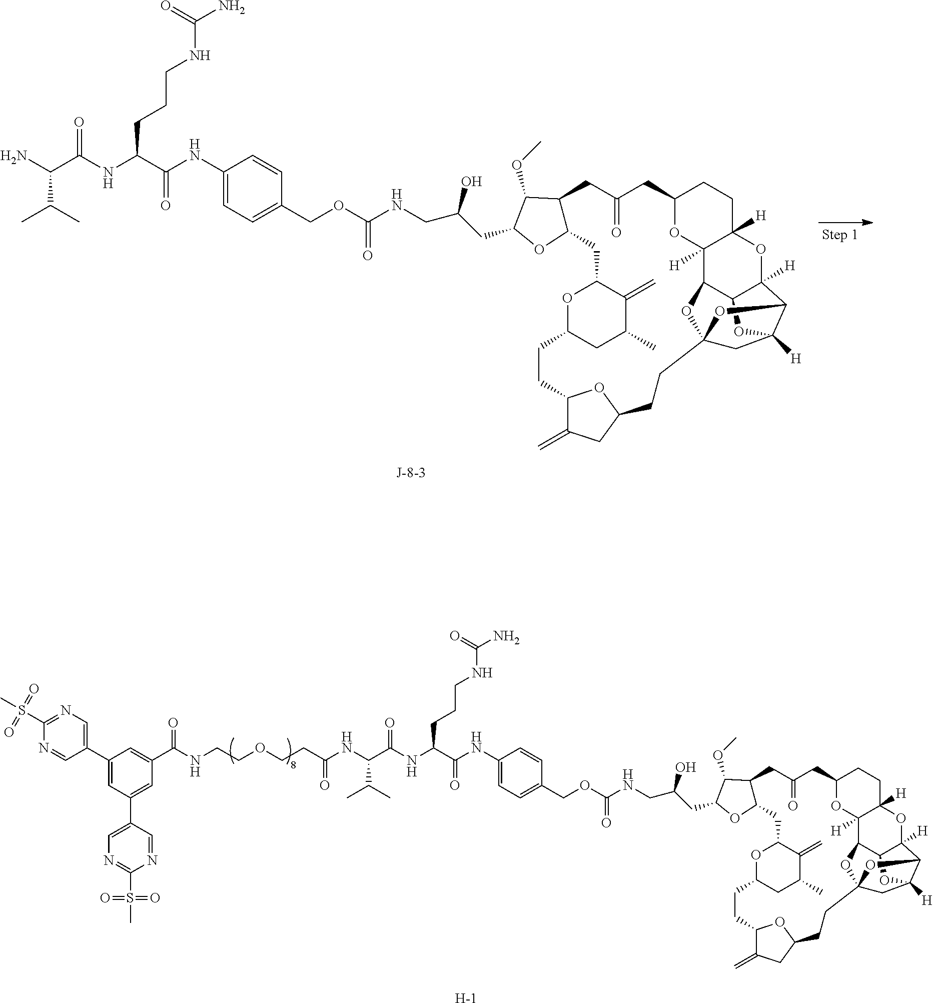

Compound J-8-3 (10 mg, 8.46 μmol) and DIPEA (5.47 mg, 42.32 μmol) were added to DMF (3 mL), and stirred for 10 min. Then 1-(3,5-bis(2-(methylsulfonyl)pyrimidin-5-yl)phenyl)-1-oxo-5,8,11,14,17,20,23,26-octaoxa-2-azanonacosan-29-oic acid (14.52 mg, 16.93 μmol) and HATU (6.44 mg, 16.93 μmol) were added to the reaction system, and reacted at 25° C. for 3.5 hours. The reaction solution was directly purified by preparative high performance liquid chromatography and freeze-dried to obtain 10.70 mg of the title compound.

Its structural characterization data are as follows:

ESI-MS (m/z): 1997.1 [M+Na]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 32 |
| 2.00 | 30 | 70 | 32 |
| 22.00 | 90 | 10 | 32 |

### Example 6: 4-((33S,36S)-1-(3,5-Bis(2-(methylsulfonyl)pyrimidin-5-yl)phenyl)-33-isopropyl-2,5,8,11,14,17,20,23,26,29-decamethyl-1,4,7,10,13,16,19,22,25,28,31,34-dodecaoxo-36-(3-ureidopropyl)-2,5,8,11,14,17,20,23,26,29,32,35-dodecaazaheptatriacontan-37-amido)benzyl((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)propyl)carbamate (H-6)

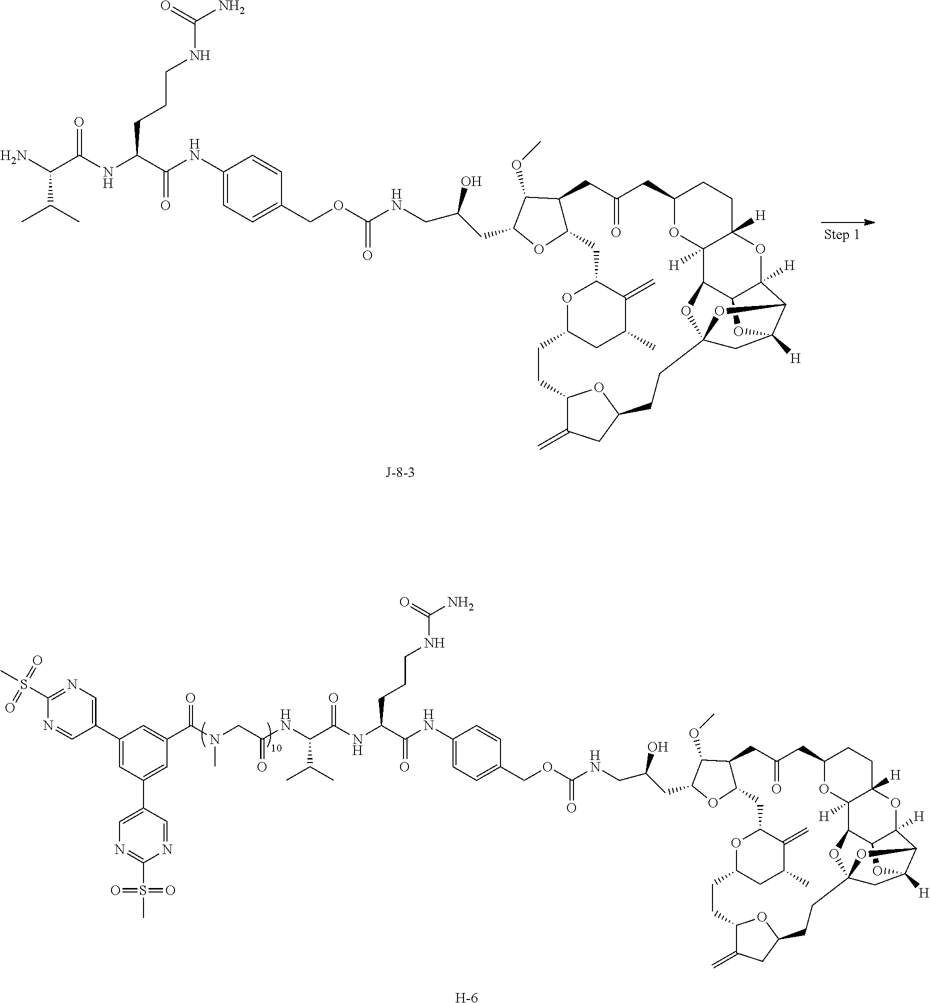

Compound J-8-3 (10 mg, 8.46 μmol), 1-(3,5-bis(2-(methylsulfonyl)pyrimidin-5-yl)phenyl)-2,5,8,11,14,17,20,23,26,29-decamethyl-1,4,7,10,13,19,22,25,28-decaoxo-2,5,8,11,14,17,20,23,26,29-decaazahentriacontan-31-oic acid (19.39 mg, 16.93 μmol), HATU (6.44 mg, 16.93 μmol), DIPEA (5.47 mg, 42.32 μmol) were added to DMF (3 mL), and reacted at 25° C. for 3 hours. The reaction solution was directly purified by high performance liquid chromatography and freeze-dried to obtain 16 mg of the title compound.

Its structural characterization data are as follows:

ESI-MS (m/z): 1132.3 [1/2M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 32 |
| 4.00 | 30 | 70 | 32 |
| 24.00 | 90 | 10 | 32 |

### Example 7: N-((31S,34S,37S,41S)-41-hydroxy-42-((2S,5S,8S,11S,13R,15R,16aS,18R, 19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-3,6,9,12,15,18,21,24,27,31,34,37-dodecamethyl-2,5,8,11,14,17,20,23,26,29,32,35,38-tridecaoxo-3,6,9,12,15,18,21,24,27,30,33,36,39-tridecaazadotetracontanyl)-N-methyl-3,5-bis(2-(methylsulfonyl)pyrimidin-5-yl)benzamide (H-8)

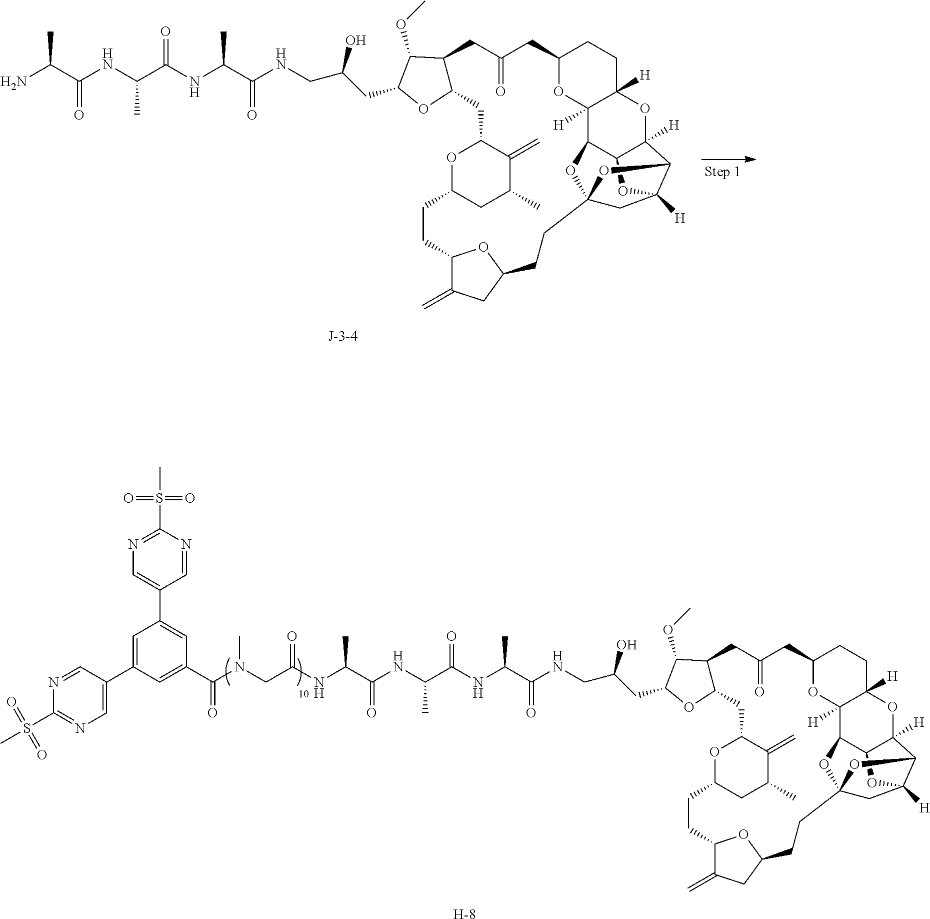

Compound J-3-4 (12 mg, 12.13 μmol), 1-(3,5-bis(2-(methylsulfonyl)pyrimidin-5-yl) phenyl)-2,5,8,11,14,17,20,23,26,29-decamethyl-1,4,7,10,13,19,22,25,28-decaoxo-2,5,8,11,14,17,20,23,26,29-decaazahentriacontan-31-oic acid (27.79 mg, 24.26 μmol), HATU (9.23 mg, 24.26 μmol) and DIPEA (7.84 mg, 60.66 μmol) were added to DMF (3 mL). After reacting at 25° C. for one hour, some of the raw materials remained. HATU (9.23 mg, 24.26 μmol) and DIPEA (7.84 mg, 60.66 μmol) were added, and the reaction continued for 1 hour. The reaction solution was directly concentrated to obtain a crude product, which was purified by preparative high performance liquid chromatography and freeze-dried to obtain 14 mg of the title compound.

Its structural characterization data are as follows:

ESI-MS (m/z): 1044.6[1/2(M+18)]⁺.

Its preparation method was as follows:

Chromatographic column: Waters XBridge Prep C180BD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 32 |
| 4.00 | 30 | 70 | 32 |
| 24.00 | 90 | 10 | 32 |

### Example 8: N-((37S, 44S)-37-benzyl-44-hydroxy-45-((2S,5S,8S,1IS,13R,15R,16aS,18R,19R, 19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosin-18-yl)-3,6,9,12,15,18,21,24,27-nonamethyl-2,5,8,11,14,17,20,23,26,29,32,35,38,41-tetradecaoxo-3,6,9,12,15,18,21,24,27,30,33,36,39,42-tetradecaazapentatetracontanyl)-N-methyl-3,5-bis(2-(methylsulfonyl)pyrimidin-5-yl)benzamide (H-10)

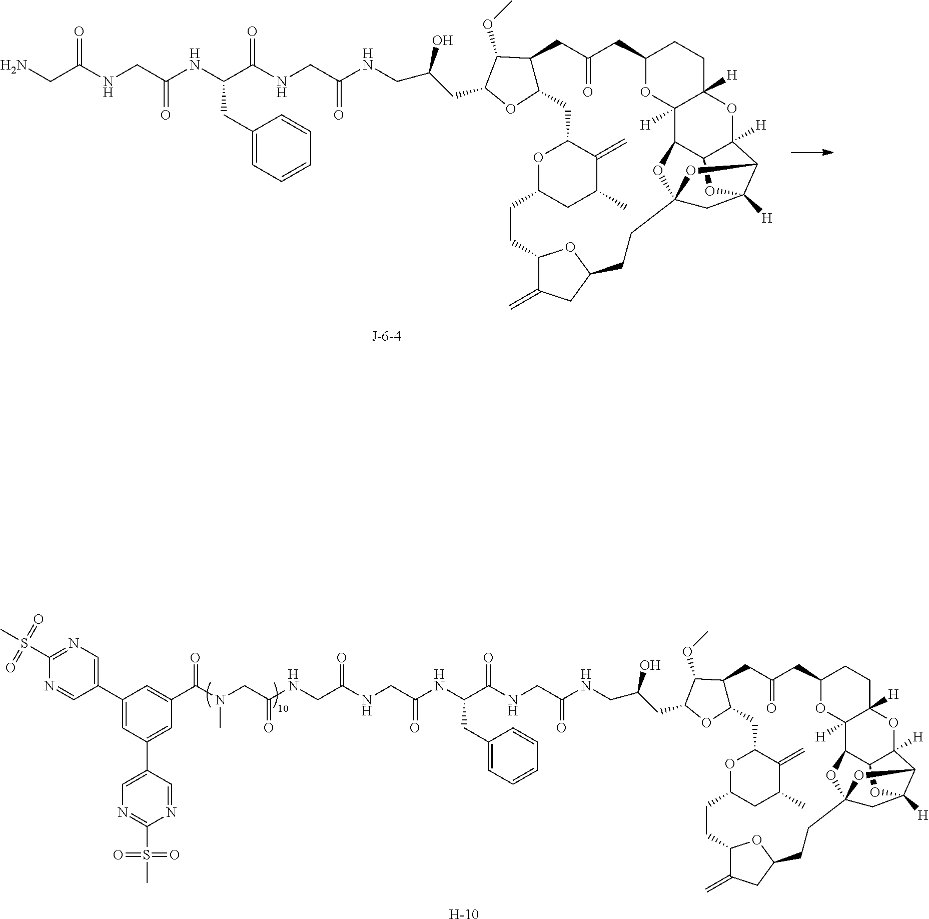

1-(3,5-Bis(2-(methylsulfonyl)pyrimidin-5-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (52.33 mg, 45.69 μmol) was dissolved in DMF (5 mL), added with HATU (17.37 mg, 45.69 μmol), DIPEA (14.76 mg, 114.23 μmol) and compound J-6-4 (25.0 mg, 22.85 μmol, formate), and reacted at 25° C. for 2 hours. The reaction solution was directly purified by high performance liquid chromatography and freeze-dried to obtain the title compound (18.0 mg, 8.19 μmol).

Its structural characterization data are as follows:

ESI-MS (m/z): 1096.6 (1/2M+H)+.

Its purification method was as follows:

Chromatographic column: Waters SunFire Prep C18 OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 30 |
| 2.00 | 30 | 70 | 30 |
| 18.00 | 90 | 10 | 30 |

### Example 9: Preparation of pentafluorophenyl 1-(N-((12S,15S,19S)-19-hydroxy-12-isopropyl-20-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxa cyclopentacosan-18-yl)-15-methyl-4,7,10,13,16-pentaoxo-2-oxa-5,8,11,14,17-pentaazaeicosanoyl)sulfamoyl)piperidin-4-carboxylic acid (J-5)

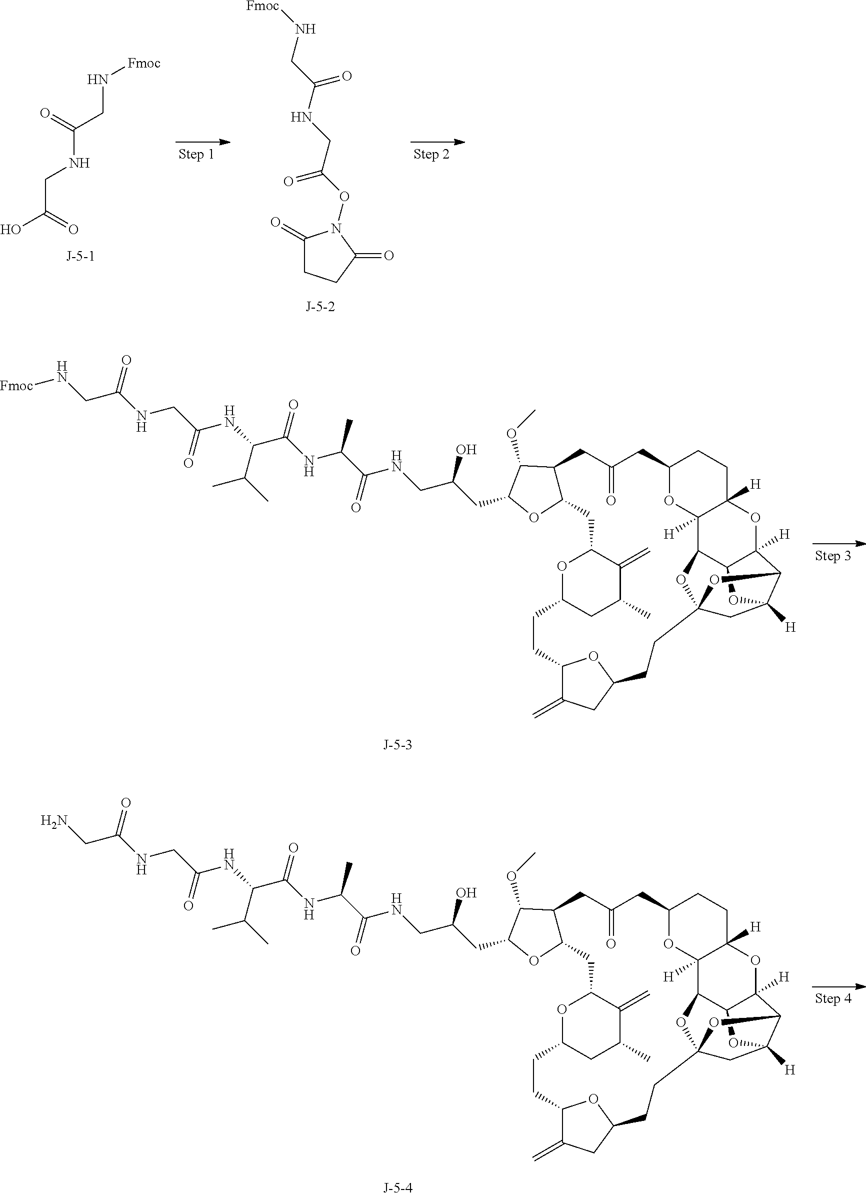

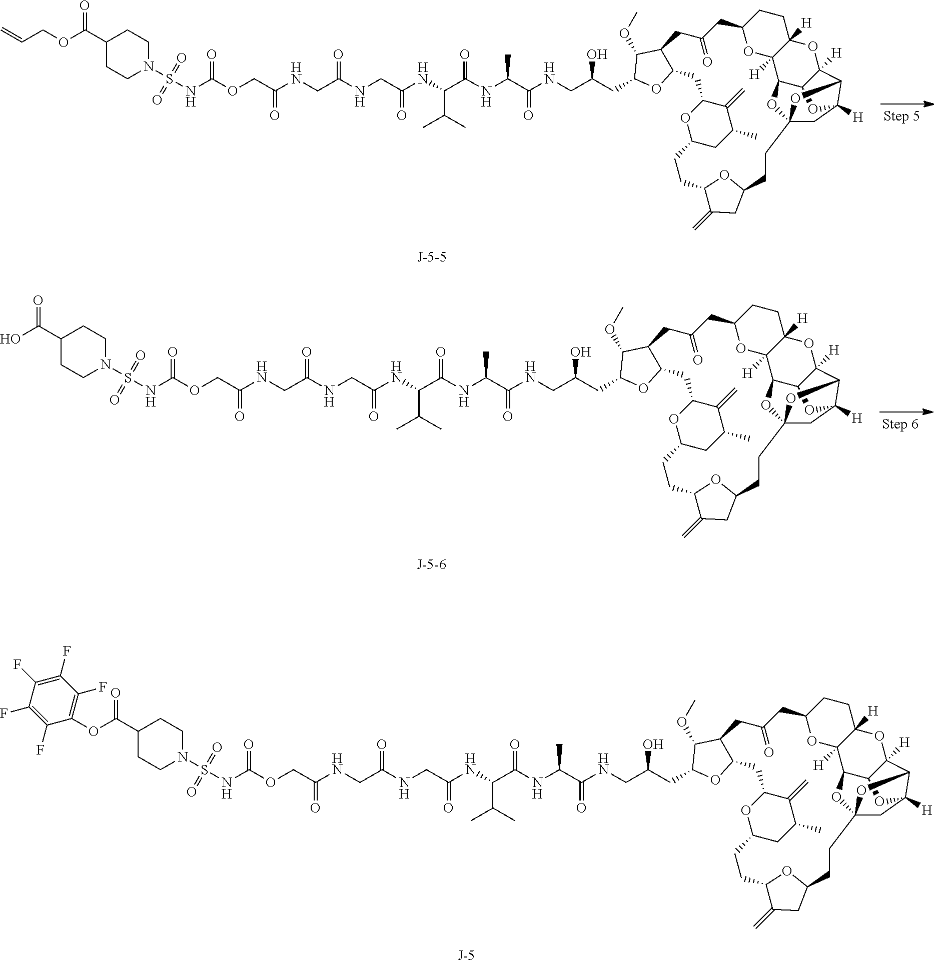

### Step 1: Preparation of 2,5-dioxopyrrolidin-1-yl (((9H-fluoren-9-yl)methoxy)carbonyl)glycylglycinate (J-5-2)

(((9H-Fluoren-9-yl)methoxy)carbonyl)glycylglycine (1.50 g, 4.23 mmol), N-hydroxysuccinimide (974.34 mg, 8.47 mmol) and EDCI (1.62 g, 8.47 mmol) were added to DMF (20 mL), reacted at 25° C. for 2 hours, added with water and ethyl acetate for extraction, and concentrated to dryness to obtain 1.9 g of the title compound, which was directly used in the next step without purification.

Its structural characterization data are as follows:

MS m/z (ESI): 474.1 [M+Na]⁺

### Step 2: Preparation of (9H-fluoren-9-yl)methyl ((7S,10S,14S)-14-hydroxy-7-isopropyl-15-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosan-18-yl)-10-methyl-2,5,8,11-tetraoxo-3,6,9,12-tetraazapentadecanyl)carbamate (J-5-3)

J-5-2 (127.87 mg, 283.26 μmol), J-1-4 (67 mg, 70.81 μmol, formate) and DIPEA (27.46 mg, 212.44 μmol) were added to DMF (5 mL), and reacted under stirring at 25° C. for 6 hours. The reaction solution was concentrated to obtain a crude product, which was purified by preparative high performance liquid chromatography and freeze-dried to obtain 49 mg of the title compound.

Its structural characterization data are as follows:

ESI-MS (m/z): 1237.8 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 40 | 60 | 30 |
| 4.00 | 40 | 60 | 30 |
| 24.00 | 90 | 10 | 30 |

### Step 3: Preparation of (S)-2-(2-(2-aminoacetamido)acetamido)-N—((S)-1-(((S)-2-hydroxy-3-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosan-18-yl)propyl)amino)-1-oxoprop-2-yl)-3-methylbutanamide (J-5-4)

J-5-3 (49 mg, 39.63 μmol) and DBU (18.10 mg, 118.89 μmol) were added to DMF (3 mL), and reacted at 25° C. for 1 hours. The reaction solution was concentrated to obtain a crude product, which was purified by reverse phase liquid chromatograph (ACN/0.05% ammonium bicarbonate aqueous solution=0 to 60%) and freeze-dried to obtain 40 mg of the title compound.

Its structural characterization data are as follows:

ESI-MS (m/z): 1014.6 [M+H]⁺.

### Step 4: Preparation of allyl 1-(N-((12S,15S,19S)-19-hydroxy-12-isopropyl-20-((2S,5S,8S, 11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosan-18-yl)-15-methyl-4,7,10,13,16-pentaoxo-2-oxa-5,8,11,14,17-pentaazaeicosanoyl)sulfamoyl)piperidin-4-carboxylate (J-5-5)

J-5-4 (40 mg, 39.44 μmol), INT2 (41.45 mg, 118.32 μmol), HATU (15.00 mg, 78.88 μmol) and DIPEA (25.49 mg, 197.20 μmol) were added to DMF (8 mL), and reacted at 25° C. for 5 hours. The reaction solution was concentrated to obtain a crude product, which was purified by reverse phase liquid chromatography (ACN/H₂O=0-50%, 0.05% formic acid) and freeze-dried to obtain 48 mg of the title compound.

Its structural characterization data are as follows:

MS m/z (ESI): 1346.6 [M+H]⁺

### Step 5: Preparation of 1-(N-((12S,15S,19S)-19-hydroxy-12-isopropyl-20-((2S,5S,8S,1IS, 13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosan-18-yl)-15-methyl-4,7,10,13,16-pentaoxo-2-oxa-5,8,11,14,17-pentaazaeicosanoyl)sulfamoyl)piperidin-4-carboxylic acid (J-5-6)

J-5-5 (48 mg, 35.65 μmol), morpholine (31.06 mg, 356.47 μmol) and tetrakistriphenylphosphine palladium (24.72 mg, 21.39 μmol) were dissolved in THF (10 mL), vacuumed, subjected to replacement with nitrogen 3 times, and reacted at 25° C. for 2 hours. The reaction solution was directly concentrated to obtain a crude product, which was purified by reverse phase liquid chromatography (ACN/H₂O=0-50%, 0.05% formic acid) and freeze-dried to obtain 16 mg of the title compound.

Its structural characterization data are as follows:

MS m/z (ESI): 1306.5 [M+H]⁺

### Step 6: Preparation of pentafluorophenyl 1-(N-((12S,15S,19S)-19-hydroxy-12-isopropyl-20-((2S,5S,8S,11S,13R,15R,16aS,18R,19R,19aS,23R,24aS,25S,26aR,27S,28R,29aR,29bS)-19-methoxy-13-methyl-7,14-dimethylene-21-oxooctacosahydro-25H-2,27:5,8:11,15-triepoxy-23,25-ethano-2,28-methanofuro[3,2-i]furo[2′,3′:5,6]pyrano[4,3-b][1,4]dioxacyclopentacosan-18-yl)-15-methyl-4,7,10,13,16-pentaoxo-2-oxa-5,8,11,14,17-pentaazaeicosanoyl)sulfamoyl)piperidin-4-carboxylate (J-5)

Compound J-5-5 (16 mg, 12.25 μmol), 2,3,4,5,6-pentafluorophenol (33.81 mg, 183.70 μmol) and EDCI (42.26 mg, 220.44 μmol) were added to DMF (5 mL), and reacted at 25° C. for 4 hours.

The reaction solution was directly concentrated to obtain a crude product, which was purified by preparative high performance liquid chromatography and freeze-dried to obtain 6.00 mg of the title compound.

The structural characterization data are as follows:

ESI-MS (m/z): 1472.6 [M+H]⁺.

Its preparation method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile phase A | Mobile phase B | Flow rate |
|---|---|---|---|
| [min] | [%] | [%] | [mL/min] |
| 0.00 | 30 | 70 | 30 |
| 2.00 | 30 | 70 | 30 |
| 18.00 | 90 | 10 | 30 |

### II. Preparation of Antibody and Determination of Binding Activity

### 1. Acquisition and Purification of Antibodies

According to the amino acid sequence of Trastuzumab (IMGT/mAb-DB ID: 97) and the amino acid sequence of Pertuzumab (IMGT/mAb-DB ID: 80) in the IMGT database, codon optimization and synthesis of coding genes were performed, which were constructed into expression vectors, transfected into CHO cells and screened under pressure to construct stable expression cell lines. After expression, the supernatants were collected, and the corresponding antibodies were purified by Protein A affinity filler. The amino acid sequence information of Trastuzumab and Pertuzumab is shown above.

III. Conjugation of Compound Containing Cell Bioactive Molecule and Linker with Antibody

The antibodies Trastuzumab and Pertuzumab involved in the antibody-drug conjugates prepared in the following examples were Trastuzumab and Pertuzumab described in the second part above.

The conjugation preparation of the antibody-drug conjugate sample was as follows:

### 1. Example: Preparation of Trastuzumab-J-3

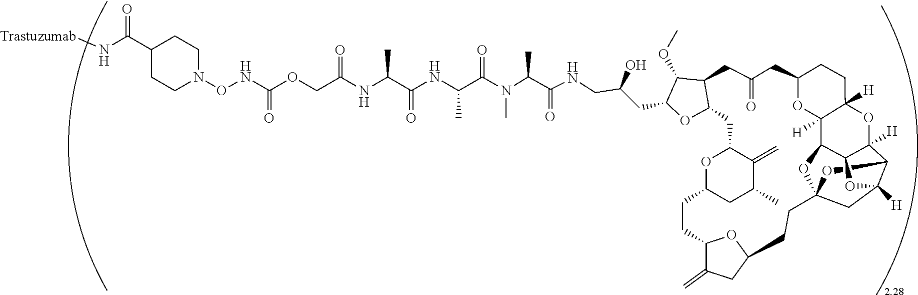

0.926 mL of Trastuzumab antibody (16.2 mg/mL) was taken, then adjusted with 1M Na₂HPO₄ solution to pH 7.40, then added with J-3 (54.39 μL, 10 mM, equivalent to 5 times the amount of antibody) dissolved in dimethyl sulfoxide, mixed well, and allowed to stand at room temperature for 18 hours. After the completion, NAP-5 gel column (Cytiva) was used to replace the buffer with 20 mM histidine buffer solution at pH 6.0 to obtain an antibody-drug conjugate (i.e. ADC Trastuzumab-J-3). The DAR value determined by mass spectrometry was 2.28.

TABLE 1
Measured molecular weight and DAR
calculation of Trastuzumab-J-3

| | | Maximum signal | | |
|---|---|---|---|---|
| Payload | MW_{Exp.} | intensity | Ratio | DAR |
| Naked antibody | 148056.00 | 0.00 | 0.00% | 2.28 |
| DAR1 | 149278.09 | 18.97 | 5.03% | |
| DAR2 | 150492.44 | 254.12 | 67.43% | |
| DAR3 | 151715.19 | 82.61 | 21.92% | |
| DAR4 | 152943.43 | 21.17 | 5.62% | |
| DAR5 | 154161.43 | 0.00 | 0.00% | |
| DAR6 | 155379.43 | 0.00 | 0.00% | |

### 2. Example: Preparation of Trastuzumab-J-5

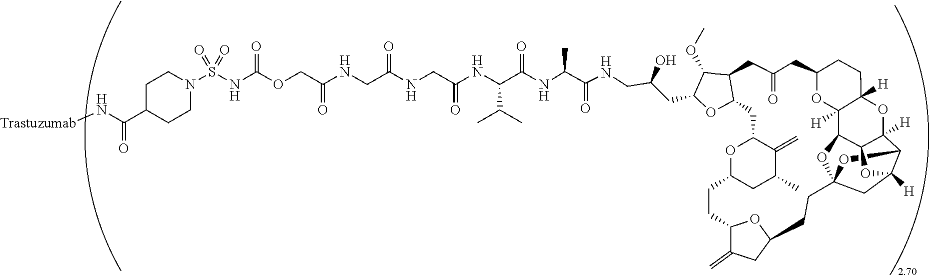

0.27 mL of Trastuzumab antibody (14.8 mg/mL) was taken, then adjusted with 1M Na₂HPO₄ solution to pH 7.40, then added with J-5 (14.5 μL, 10 mM, equivalent to 5 times the amount of antibody) dissolved in dimethyl sulfoxide, mixed well, and allowed to stand at room temperature for 4 hours. After completion, NAP-5 gel column (Cytiva) was used to replace the buffer with 20 mM histidine buffer solution at pH 6.0 to obtain an antibody-drug conjugate (i.e. ADC Trastuzumab-J-5). The DAR value determined by mass spectrometry was 2.70.

TABLE 2
Measured molecular weight and DAR
calculation of KB106-Her2-1089

| | | Maximum | | |
|---|---|---|---|---|
| Payload | MW_{Exp.} | signal intensity | Ratio | DAR |
| Naked antibody | 148,222.00 | 0.00 | 0.00% | 2.70 |
| DAR1 | 149,510.00 | 0.00 | 0.00% | |
| DAR2 | 150,801.31 | 386.87 | 47.23% | |
| DAR3 | 152,090.54 | 308.88 | 37.71% | |
| DAR4 | 153,379.68 | 103.82 | 12.68% | |
| DAR5 | 154,679.52 | 19.49 | 2.38% | |
| DAR6 | 155,967.52 | 0.00 | 0.00% | |

### 3. Example: Preparation of Trastuzumab-J-6

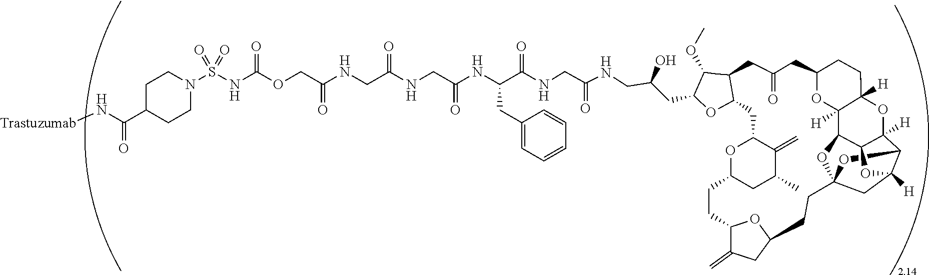

0.926 mL of Trastuzumab antibody (16.2 mg/mL) was taken, then adjusted with 1M Na₂HPO₄ solution to pH 7.40, then add with J-6 (50.11 μL, 10 mM, equivalent to 4.8 times the amount of antibody) dissolved in dimethyl sulfoxide, mixed well, and allowed to stand at room temperature for 18 hours. After completion, NAP-5 gel column (Cytiva) was used to replace the buffer with 20 mM histidine buffer solution at pH 6.0 to obtain an antibody-drug conjugate (i.e. ADC Trastuzumab-J-6). The DAR value determined by mass spectrometry was 2.14.

TABLE 3
Measured molecular weight and DAR
calculation of Trastuzumab-J-6

| | | Maximum | | |
|---|---|---|---|---|
| Payload | MW_{Exp.} | signal intensity | Ratio | DAR |
| Naked antibody | 148220.75 | 13.76 | 1.64% | 2.14 |
| DAR1 | 149541.47 | 85.84 | 10.24% | |
| DAR2 | 150865.00 | 507.33 | 60.52% | |
| DAR3 | 152187.36 | 231.30 | 27.59% | |
| DAR4 | 153509.36 | 0.00 | 0.00% | |
| DAR5 | 154831.36 | 0.00 | 0.00% | |
| DAR6 | 156153.36 | 0.00 | 0.00% | |

### 4. Example: Preparation of Trastuzumab-J-8

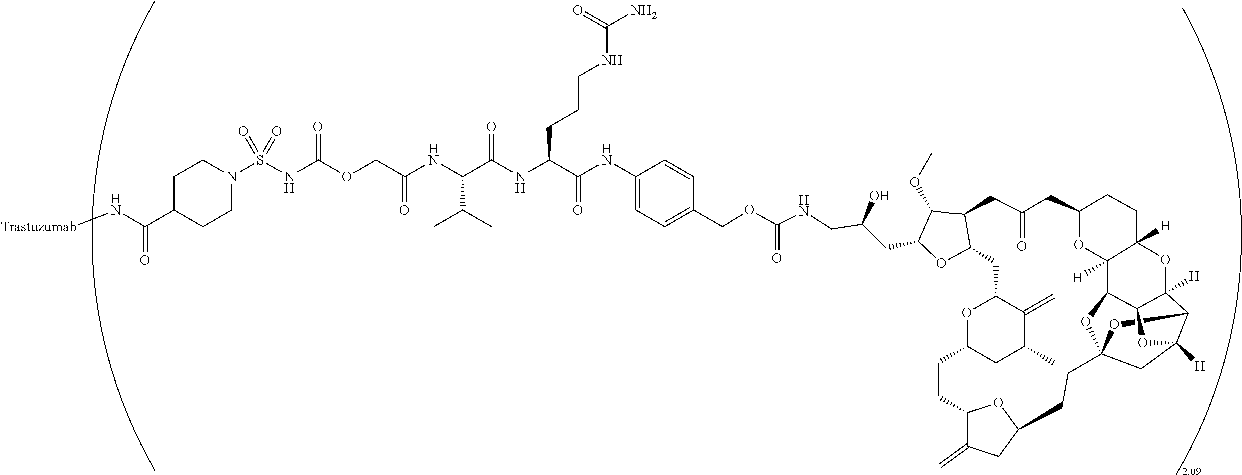

0.926 mL of Trastuzumab antibody (16.2 mg/mL) was taken, then adjusted with 1M Na₂HPO₄ solution to pH 7.40, then added with J-8 (53.27 μL, 10 mM, equivalent to 5 times the amount of antibody) dissolved in dimethyl sulfoxide, mixed well, and allowed to stand at room temperature for 18 hours. NAP-5 gel column (Cytiva) was used to replace the buffer with 20 mM histidine buffer solution at pH 6.0 to obtain an antibody-drug conjugate (i.e. ADC Trastuzumab-J-8). The DAR value determined by mass spectrometry was 2.09.

TABLE 4
Measured molecular weight and DAR
calculation of Trastuzumab-J-8

| | | Maximum signal | | |
|---|---|---|---|---|
| Payload | MW_{Exp.} | intensity | Ratio | DAR |
| Naked antibody | 148064.07 | 0.00 | 0.00% | 2.09 |
| DAR1 | 149473.07 | 54.24 | 14.31% | |
| DAR2 | 150878.51 | 237.73 | 62.70% | |
| DAR3 | 152282.49 | 87.20 | 23.00% | |
| DAR4 | 153691.49 | 0.00 | 0.00% | |
| DAR5 | 155100.49 | 0.00 | 0.00% | |
| DAR6 | 156509.49 | 0.00 | 0.00% | |

### 5. Example: Preparation of Trastuzumab-H-1

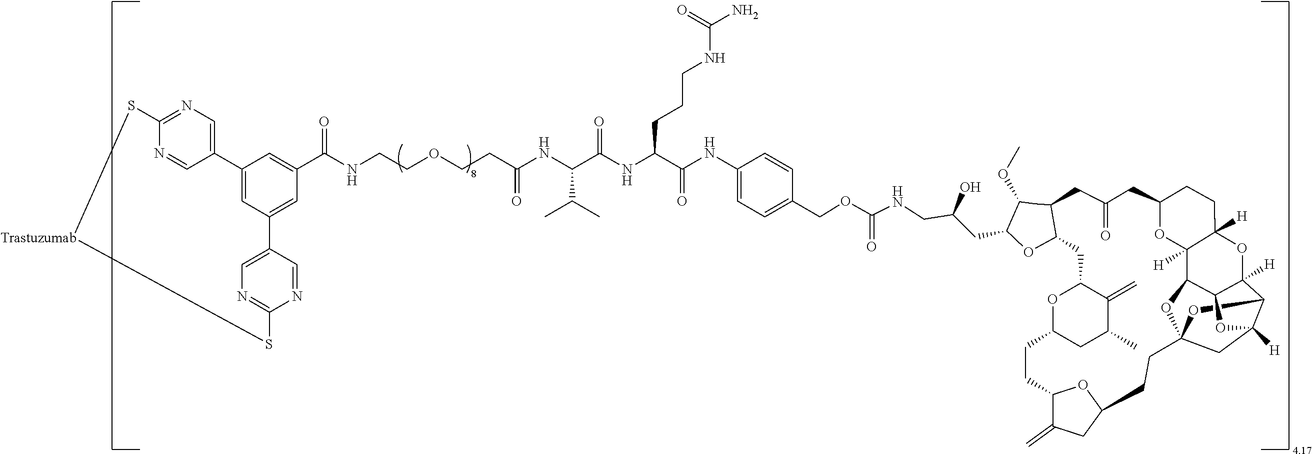

0.75 mL of Trastuzumab antibody (16.2 mg/mL) was taken, diluted with 37.5 μL of 20 mM PB+0.1M EDTA (pH 7.60), then adjusted with 1M Na₂HPO₄ solution to pH 7.60, added with 10 mM TCEP (tri(2-carboxyethyl)phosphine, 46 μL, pH 7.60) solution, mixed well, and allowed to stand at room temperature for 1.5 hours. Then H-1 (50.73 μL, 10 mM, equivalent to 6 times the amount of antibody) dissolved in dimethyl sulfoxide solution was added sequentially, mixed well, and allowed to stand at room temperature for 18 hours. After completion, NAP-5 gel column (Cytiva) was used to replace the buffer with 20 mM histidine buffer solution at pH 6.0 to obtain an antibody-drug conjugate (i.e. ADC Trastuzumab-H-1). The DAR value determined by mass spectrometry was 4.17.

TABLE 5
Measured molecular weight and DAR
calculation of Trastuzumab-H-1

| | | Maximum signal | | |
|---|---|---|---|---|
| Payload | MW_{Exp.} | intensity | Ratio | DAR |
| Naked antibody | 148,212.00 | 0.00 | 0.00% | 4.17 |
| DAR1 | 150,027.00 | 0.00 | 0.00% | |
| DAR2 | 151,842.00 | 0.00 | 0.00% | |
| DAR3 | 153,657.00 | 0.00 | 0.00% | |
| DAR4 | 155,526.84 | 0.58 | 82.53% | |
| DAR5 | 157,460.64 | 0.12 | 17.47% | |
| DAR6 | 159,156.84 | 0.00 | 0.00% | |
| DAR7 | 160,971.84 | 0.00 | 0.00% | |
| DAR8 | 162,786.84 | 0.00 | 0.00% | |

### 6. Example: Preparation of Trastuzumab-H-6

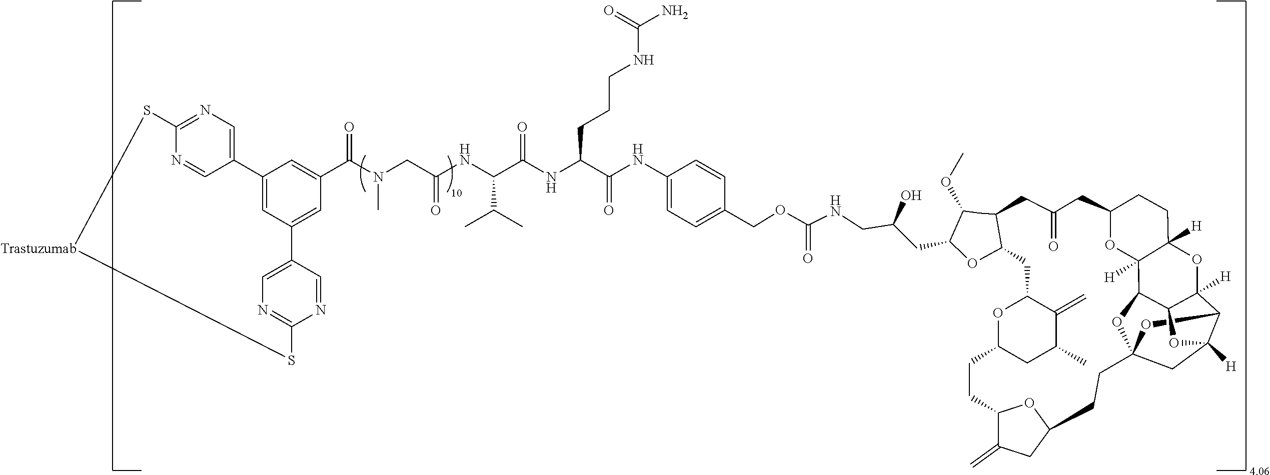

0.185 mL of Trastuzumab antibody (16.2 mg/mL) was taken, diluted with 19.3 μL of 20 mM PB+0.1M EDTA (pH 7.60), then adjusted with 1M Na₂HPO₄ solution to pH 7.60, added with 30 mM TCEP (tris(2-carboxyethyl)phosphine, 3.79 μL, pH 7.60) solution, mixed well, and allowed to stand at room temperature for 1.5 hours. Then, H-6 (12.54 μL, 10 mM, equivalent to 6 times the amount of antibody) dissolved in dimethyl sulfoxide was added sequentially, mixed well, and allowed to stand at room temperature for 18 hours. After completion, NAP-5 gel column (Cytiva) was used to replace the buffer with a 20 mM histidine buffer solution at pH 6.0 to obtain an antibody-drug conjugate (i.e., ADC Trastuzumab-H-6). The DAR value determined by mass spectrometry was 4.06.

TABLE 6
Measured molecular weight and DAR
calculation of Trastuzumab-H-6

| | | Maximum signal | | |
|---|---|---|---|---|
| Payload | MW_{Exp.} | intensity | Ratio | DAR |
| Naked antibody | 148,056.00 | 0.00 | 0.00% | 4.06 |
| DAR1 | 150,158.00 | 0.00 | 0.00% | |
| DAR2 | 152,260.00 | 0.00 | 0.00% | |
| DAR3 | 154,362.00 | 0.00 | 0.00% | |
| DAR4 | 156,474.14 | 15.51 | 94.46% | |
| DAR5 | 158,736.95 | 0.91 | 5.54% | |
| DAR6 | 160,678.14 | 0.00 | 0.00% | |
| DAR7 | 162,780.14 | 0.00 | 0.00% | |
| DAR8 | 164,882.14 | 0.00 | 0.00% | |

### 7. Example: Preparation of Trastuzumab-H-8

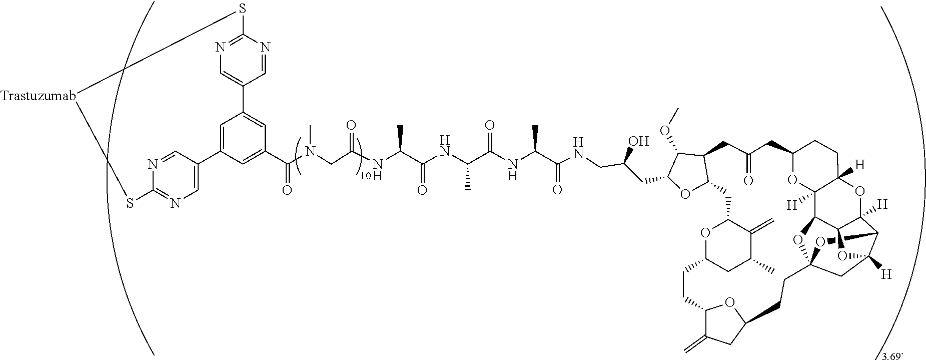

0.926 mL of Trastuzumab antibody (16.2 mg/mL) was taken, diluted with 46.3 μL of 20 mM PB+0.1M EDTA (pH 7.60), then adjusted with 1M Na₂HPO₄ solution to pH 7.60, added with 10 mM TCEP (tris(2-carboxyethyl)phosphine, 56.84 μL, pH 7.60) solution, mixed well, and allowed to stand at room temperature for 1.5 hours. Then, H-8 (62.63 μL, 10 mM, equivalent to 6 times the amount of the antibody) dissolved in dimethyl sulfoxide was added sequentially, mixed well, and allowed to stand at room temperature for 18 hours. After completion, the buffer was replaced with a 20 mM histidine buffer solution at pH 6.0 using a NAP-5 gel column (Cytiva) to obtain an antibody-drug conjugate (i.e., ADC Trastuzumab-H-8). The DAR value determined by mass spectrometry was 3.89.

TABLE 7
Measured molecular weight and DAR
calculation of Trastuzumab-H-8

| | | Maximum signal | | |
|---|---|---|---|---|
| Payload | MW_{Exp.} | intensity | Ratio | DAR |
| Naked antibody | 148223.00 | 0.00 | 0.00% | 3.89 |
| DAR1 | 150133.00 | 0.00 | 0.00% | |
| DAR2 | 152043.00 | 0.00 | 0.00% | |
| DAR3 | 153955.42 | 15.09 | 10.77% | |
| DAR4 | 155871.08 | 125.08 | 89.23% | |
| DAR5 | 157781.08 | 0.00 | 0.00% | |
| DAR6 | 159691.08 | 0.00 | 0.00% | |
| DAR7 | 161601.08 | 0.00 | 0.00% | |
| DAR8 | 163511.08 | 0.00 | 0.00% | |

### 8. Example: Preparation of Trastuzumab-H-10

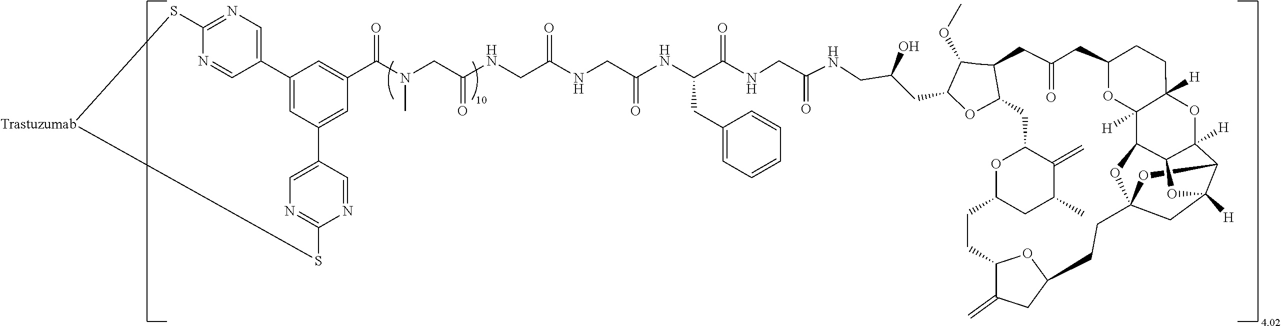

0.926 mL of Trastuzumab antibody (16.2 mg/mL) was taken, diluted with 46.3 μL of 20 mM PB+0.1M EDTA (pH 7.60), then adjusted with 1M Na₂HPO₄ solution to pH 7.60, added with 10 mM TCEP (tris(2-carboxyethyl)phosphine, 56.84 μL, pH 7.60) solution, mixed well, and allowed to stand at room temperature for 1.5 hours. Then, H-10 (62.63 μL, 10 mM, equivalent to 6 times the amount of the antibody) dissolved in dimethyl sulfoxide was added sequentially, mixed well, and allowed to stand at room temperature for 18 hours. After completion, the buffer was replaced with a 20 mM histidine buffer solution at pH 6.0 using a NAP-5 gel column (Cytiva) to obtain an antibody-drug conjugate (i.e., ADC Trastuzumab-H-10). The DAR value determined by mass spectrometry was 4.02.

TABLE 8
Measured molecular weight and DAR
calculation of Trastuzumab-H-10

| | | Maximum signal | | |
|---|---|---|---|---|
| Payload | MW_{Exp.} | intensity | Ratio | DAR |
| Naked antibody | 148223.00 | 0.00 | 0.00% | 4.02 |
| DAR1 | 150238.00 | 0.00 | 0.00% | |
| DAR2 | 152253.00 | 0.00 | 0.00% | |
| DAR3 | 154268.00 | 0.00 | 0.00% | |
| DAR4 | 156283.36 | 148.68 | 97.62% | |
| DAR5 | 158370.75 | 3.63 | 2.38% | |
| DAR6 | 160385.75 | 0.00 | 0.00% | |
| DAR7 | 162400.75 | 0.00 | 0.00% | |
| DAR8 | 164415.75 | 0.00 | 0.00% | |

### IV. Evaluation of Tumor Growth Inhibition Effect of Antibody-Drug Conjugate on Subcutaneous Transplanted Tumor Model in Mice

The preparations containing the ADC of the present invention were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADC of the present invention on tumor-bearing mice was calculated.

### Test Drug

Drug name, source, preparation method: An appropriate amount of the ADC of the present invention was taken; based on the administration volume of 1 mg/kg, 0.9% NaCl solution for injection was used to dilute the mother solution to obtain the administration solution. 0.9% NaCl solution for injection was used as the solvent control (Vehicle).

### Experimental Animal and Cell Line

Balb/c Nude mice (Chengdu Yaokang Biotechnology Co., Ltd.)

Human gastric cancer cells NCI-N87 (ATCC)

### Experimental Grouping and Evaluation Method

The tumor-bearing mice with tumors having an average volume of about 150 mm³ were selected, and randomly divided into groups (the number of groups was determined according to the number of samples). According to the groups, 0.9% NaCl injection (hereinafter referred to as solvent control, Vehicle) and the ADC of the present invention were respectively administered.

The frequencies of administration were shown in the specific examples. The administration model was tail vein injection, and the administration volume was 10 ml/kg. After administration, the tumor diameter was measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADC of the present invention:

$${V}_{T end} > {V}_{T 0} , TGI ( \% ) = [ 1 - ( {V}_{T end} - {V}_{T 0} ) / ( {V}_{C end} - {V}_{C 0} ) ] * 100 \% or$$

$${V}_{T end} \leq {V}_{T 0} , TGI ( \% ) = [ 1 - ( {V}_{T end} - {V}_{T 0} ) / {V}_{T 0} ] * 100 \% .$$

wherein,
V_{T end}: mean of tumor volume at the end of the treatment group experiment
V_{T0}: mean of tumor volume at the beginning of the treatment group administration
V_{C end}: mean of tumor volume at the end of the negative control group experiment
V_{C0}: mean of tumor volume at the beginning of the negative control group administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) .$$

### (1) Detection of Efficacy of Anti-Human Her2 Antibody-Drug Conjugate in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c Nude mice to establish a gastric cancer model. When the average tumor volume was about 150 mm³, the mice were randomly divided into groups according to the tumor size, including in order: a vehicle control group (i.e., negative control, Vehicle group), and a Trastuzumab-J-8 at 1 mg/kg group, a Trastuzumab-J-6 at 1 mg/kg group, a Trastuzumab-J-3 at 1 mg/kg group, and a Trastuzumab-H-8 at 0.5 mg/kg group of the present invention, in which administration was carried out by tail vein injection (i.v.) on Day 0 and Day 7, twice in total.

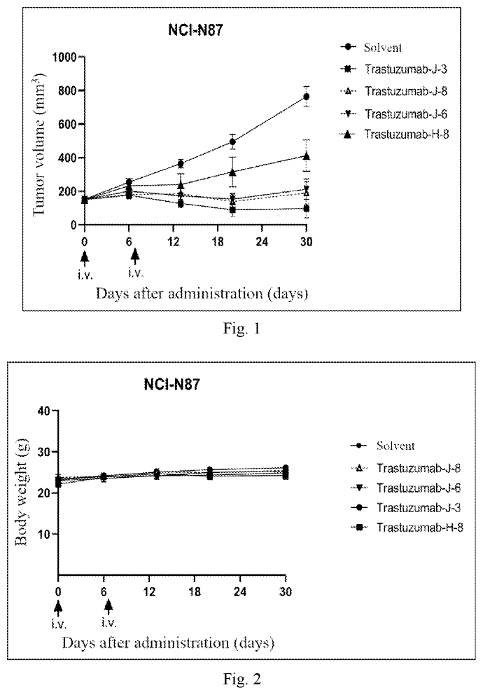

The ADC of the present invention exhibited a significant inhibitory effect on the tumor growth in the NCI-N87 gastric cancer xenograft model. Compared with the Vehicle group, the tumor growth inhibition rate (TGI) values of the Trastuzumab-J-8 at 1 mg/kg group, Trastuzumab-J-6 at 1 mg/kg group, Trastuzumab-J-3 at 1 mg/kg group, and Trastuzumab-H-8 at 0.5 mg/kg group of the present invention were 93.82%, 90.25%, 135.66% and 57.29%, respectively. Until Day 30, there was no animal death and significant animal weight loss in the treatment groups, and no obvious drug toxicity reaction was observed. The mice had good tolerance to the ADCs of the present invention during the treatment period. The specific results are shown in Table 9, FIG. 1 and FIG. 2.

TABLE 9
Human gastric cancer cell NCI-N87 CDX model

| | | Day 30 | | | |
|---|---|---|---|---|---|
| | | Tumor | | | |
| | Dose | volume (mm³) | TGI | T/C | P value |
| Group | (mg/kg) | (x̅ ± SEM) | (%) | (%) | (vs. Vehicle) |
| Vehicle | — | 763.99 ± 59.56 | — | — | — |
| Trastuzumab-J-8 | 1 | 189.91 ± 67.34 | 93.82 | 24.88 | <0.01 |
| Trastuzumab-J-6 | 1 | 212.03 ± 60.74 | 90.25 | 30.98 | <0.01 |
| Trastuzumab-J-3 | 1 | 97.0 ± 56.18 | 135.66 | 12.82 | <0.001 |
| Trastuzumab-H-8 | 0.5 | 411.87 ± 93.73 | 57.29 | 54.50 | <0.05 |

Note:
TGI is the tumor growth inhibition rate, T/C is the relative tumor proliferation rate, the same below.

### V. Evaluation of Tumor Growth Inhibition Effect of Antibody-Drug Conjugate on Subcutaneous Transplanted Tumor Model in Mice

The preparations containing the ADC of the present invention were administered via tail vein injection to the mouse CDX model subcutaneously transplanted with human breast cancer cell JIMT-1. The changes in tumor volume and animal body weight were measured twice weekly, and the tumor inhibition efficacy of the ADC of the present invention on tumor-bearing mice was calculated.

### Test Drug

Drug name, source, preparation method: An appropriate amount of the ADC of the present invention was taken; based on the administration volume of 3 mg/kg, 0.9% NaCl solution for injection was used to dilute the mother solution to form the administration solution. 0.9% NaCl solution for injection was used as the solvent control (Vehicle).

### Experimental Animal and Cell Line

NOD SCID mice (Chengdu Yaokang Biotechnology Co., Ltd., production license number: SCXK (Chuan) 2020-0034, animal certificate number: 511214900024561)

### Human Breast Cancer Cells JIMT-1 (Nanjing Kebai)

### Experimental Grouping and Evaluation Method

Tumor-bearing mice with an average tumor volume of about 150 mm³ were selected and randomly divided into groups (the number of groups was determined according to the number of samples). According to the groups, 0.9% NaCl solution for injection (hereinafter referred to as solvent control, Vehicle) and the ADCs of the present invention were administered, respectively.

The administration frequencies were shown in the specific examples. The administration model was tail vein injection, and the administration volume was 10 ml/kg. After administration, the tumor diameter was measured with a vernier caliper twice a week, and the tumor volume was calculated according to the following calculation formula: V=0.5 a×b², where a and b represent the long diameter and short diameter of the tumor, respectively. Animal deaths were observed and recorded every day.

The following formula was used to calculate the tumor growth inhibition rate TGI (%), which was used to evaluate the tumor inhibition efficacy of the ADCs of the present invention:

$${V}_{T end} > {V}_{T 0} , TGI ( \% ) = [ 1 - ( {V}_{T end} - {V}_{T 0} ) / ( {V}_{C end} - {V}_{C 0} ) ] * 100 \% or$$

$${V}_{T end} \leq {V}_{T 0} , TGI ( \% ) = [ 1 - ( {V}_{T end} - {V}_{T 0} ) / {V}_{T 0} ] * 100 \% .$$

wherein,
V_{T end}: mean of tumor volume at the end of the treatment group experiment
V_{T0}: mean of tumor volume at the beginning of the treatment group administration
V_{C end}: mean of tumor volume at the end of the negative control group experiment
V_{C0}: mean of tumor volume at the beginning of the negative control group administration

The following formula was used to calculate the tumor relative proliferation rate T/C (%), which was used to evaluate the tumor inhibition efficacy of the ADCs of the present invention:

$$T / C = ( {V}_{T end} / {V}_{T 0} ) / ( {V}_{C end} / {V}_{C 0} ) .$$

### (1) Detection of Efficacy of Anti-Human Her2 Antibody-Drug Conjugate in JIMT-1 Model

JIMT-1 cells were cultured in DMEM medium containing 10% fetal bovine serum at 37° C. and 5% CO₂. JIMT-1 cells in the exponential growth phase were collected, resuspended in PBS containing 50% matrigel to a suitable concentration, and inoculated subcutaneously in female NOD SCID mice to establish a breast cancer model. When the average tumor volume was about 150 mm³, the mice were randomly divided into groups according to the tumor size, including in order: a vehicle control group (i.e., negative control, Vehicle group), and a Trastuzumab-J-8 at 3 mg/kg group, a Trastuzumab-J-6 at 3 mg/kg group, and a Trastuzumab-J-3 at 3 mg/kg group of the present invention, in which administration was carried out by tail vein injection (i.v.) on Day 0, once in total.

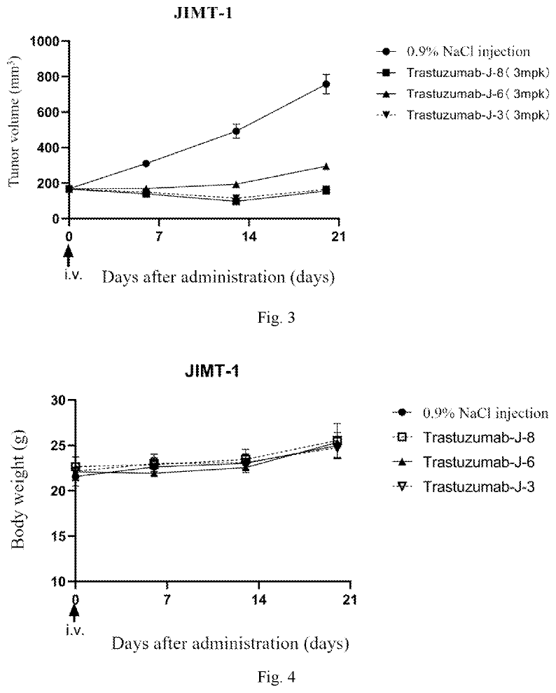

The ADCs of the present invention exhibited significant inhibitory effects on the tumor growth in the JIMT-1 breast cancer xenograft model. Compared with the Vehicle group, the tumor growth inhibition rate (TGI) values of the Trastuzumab-J-8 at 3 mg/kg group, the Trastuzumab-J-6 at 3 mg/kg group, and the Trastuzumab-J-3 at 3 mg/kg group of the present invention were 106.33%, 78.33%, 102.14%, 45.30%, respectively. Until Day 20, there was no animal death and significant animal weight loss in the treatment groups, and no obvious drug toxicity reaction was observed. The mice had good tolerance to the ADCs of the present invention during the treatment period. The specific results are shown in Table 10, FIGS. 3 and 4.

TABLE 10
Human breast cancer cell JIMT-1 CDX model

| | | Day 20 | | | |
|---|---|---|---|---|---|
| | | Tumor | | | |
| | Dose | volume (mm³) | TGI | T/C | P value |
| Group | (mg/kg) | (x̅ ± SEM) | (%) | (%) | (vs. Vehicle) |
| Vehicle | — | 757.30 ± 55.17 | — | — | — |
| Trastuzumab-J-8 | 3 | 156.11 ± 8.37 | 106.33 | 20.84 | <0.001 |
| Trastuzumab-J-6 | 3 | 295.28 ± 15.58 | 78.33 | 39.18 | <0.001 |
| Trastuzumab-J-3 | 3 | 164.67 ± 13.06 | 102.14 | 21.77 | <0.001 |

Note:
TGI is the tumor growth inhibition rate, T/C is the relative tumor proliferation rate, the same below.

Although the specific embodiments of the present invention have been described in detail, those skilled in the art will understand that various modifications and substitutions can be made to those details based on all the teachings disclosed, and these changes are within the scope of protection of the present invention. The full scope of the present invention is given by the appended claims and any equivalents thereof.

1. A compound represented by Formula (I) or a pharmaceutically acceptable salt thereof, 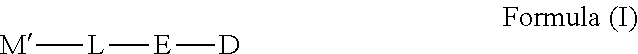
wherein:
M′ represents -M-Lg, wherein Lg is a leaving group for a nucleophilic substitution reaction, and M represents a structural fragment that connects to a targeting moiety;
L represents a structural fragment connecting M and E;
E represents a structural fragment connecting L and D;
D represents a cytotoxic drug fragment.

2. The compound or pharmaceutically acceptable salt thereof according to claim 1, wherein the M structure is substituted or unsubstituted

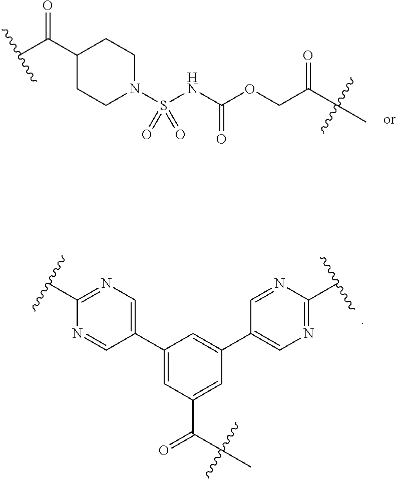 [SMILES: *C(=O)COC(=O)NS(=O)(=O)N1CCC(C(*)=O)CC1 ; *C(=O)c1cc(-c2cnc(*)nc2)cc(-c2cnc(*)nc2)c1]

3. The compound or pharmaceutically acceptable salt thereof according to claim 1, wherein Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, hydroxyl (—OH), mercapto (—SH), amino (—NH₂), nitro, azido, cyano, alkenyl, alkynyl and alkynyl-containing structural fragment, and the halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, alkenyl, alkynyl and alkynyl-containing structural fragment are optionally substituted by one or more suitable substituents;
preferably, Lg is selected from the group consisting of halogen, substituted or unsubstituted C₁₋₆ alkylsulfonyl (C₁₋₆ alkyl-SO₂—), halogenated phenoxy, hydroxyl (—OH), mercapto (—SH) or amino (—NH₂);
preferably, Lg is selected from the group consisting of halogen, substituted or unsubstituted methylsulfonyl, halogenated phenoxy, hydroxyl (—OH), mercapto (—SH) or amino (—NH₂);
preferably, Lg is selected from methylsulfonyl or pentafluorophenoxy.

4. The compound or pharmaceutically acceptable salt thereof according to claim 1, wherein L is selected from the group consisting of substituted or unsubstituted divalent structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids and analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly, Ala-Ala-Glu), 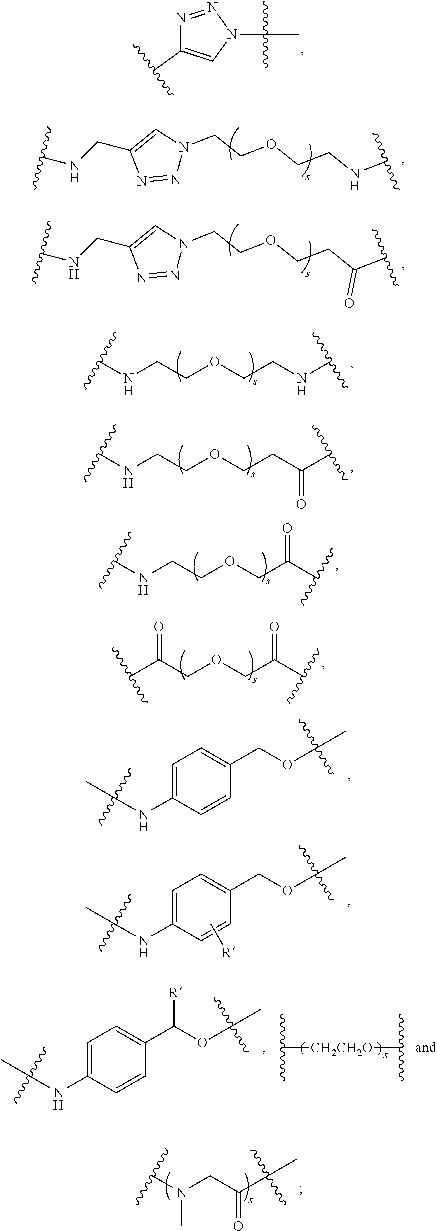
wherein R′ is composed of one or more of the following groups, including but not limited to hydrogen, C₁₋₆ alkyl, C₁₋₆ alkylene, amino, hydroxyl, carboxyl, acyl, —O—, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, polyethylene glycol fragment containing 1 to 10 EO units (i.e. —(CH₂CH₂O)₁₋₁₀—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′N″-triacetic acid residue), wherein r is an integer selected from 1 to 20, e.g., an integer of 1 to 15, such as 1 to 12, 3 to 12, 1 to 10, 1 to 8, 3 to 8, 1 to 6, 1 to 4, 1 to 2, such as 1, 2, 3, 4, 5, 6, 7, 8, 9, 10, 11, 12, 13, 14, 15, 16, 17, 18, 19 or 20; s is an integer selected from 1 to 20, e.g., an integer of 1 to 15, such as an integer of 1 to 12, 3 to 12, 5 to 10, an integer of 8 to 10, such as 1, 2, 3, 4, 5, 6, 7, 8, 9, 10, 11, 12, 13, 14, 15, 16, 17, 18, 19 or 20.

5. The compound or pharmaceutically acceptable salt thereof according to claim 1, wherein L is selected from the group consisting of the substituted or unsubstituted structural fragments composed of one or more of the following groups: 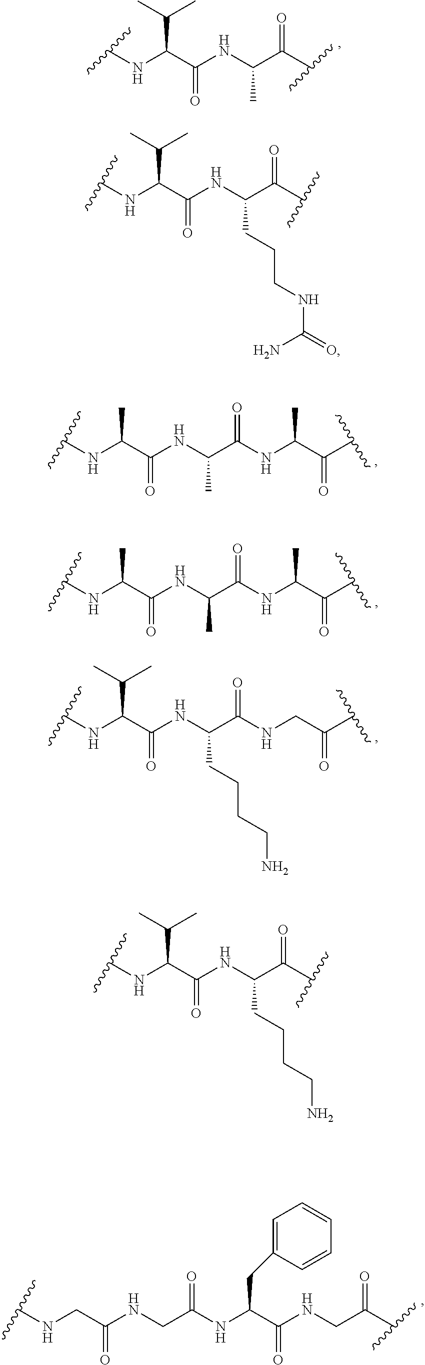 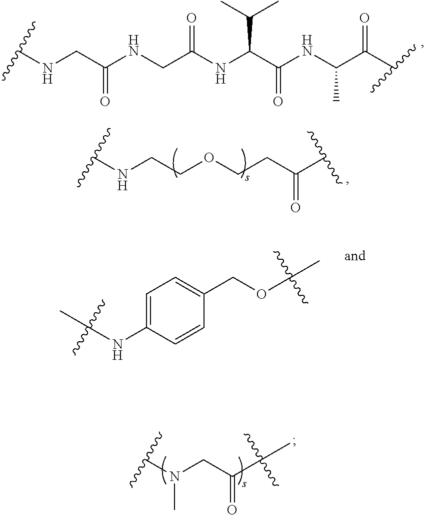
wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
preferably, L is selected from the group consisting of the substituted or unsubstituted structural fragments composed of one or more of the following groups: 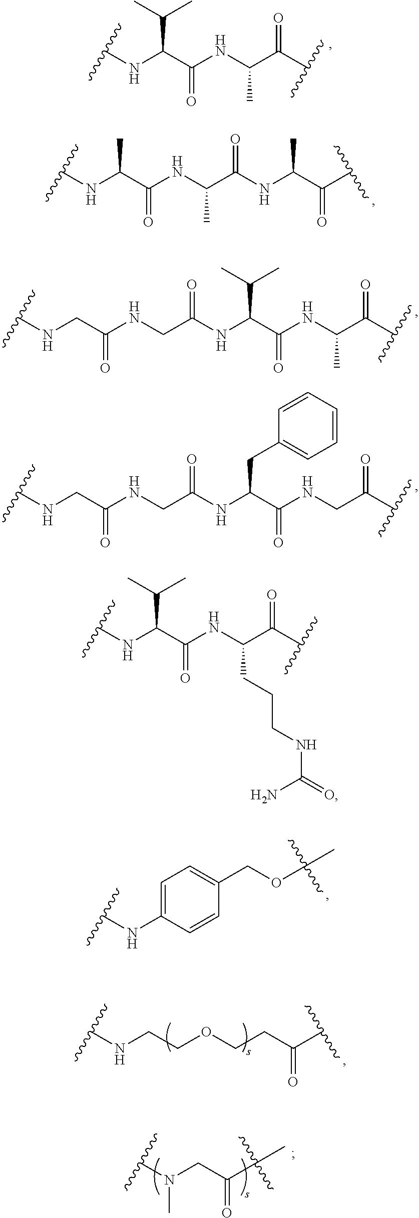
wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15;
preferably, L is selected from the group consisting of the substituted or unsubstituted structural fragments composed of one or more of the following groups: 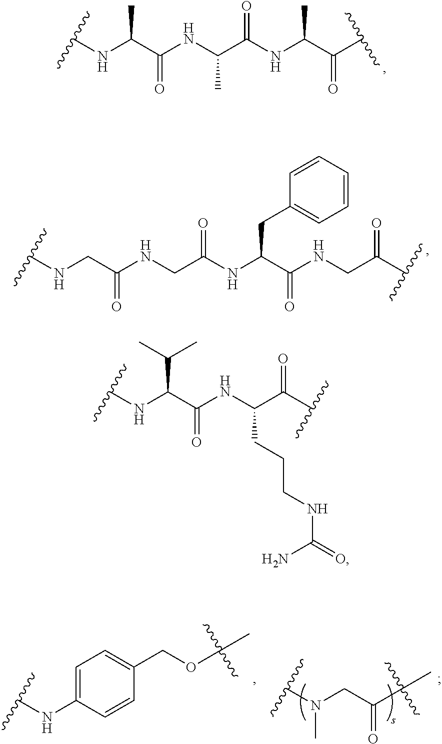
wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

6. The compound or pharmaceutically acceptable salt thereof according to claim 1, wherein L is selected from the group consisting of the following substituted or unsubstituted structural fragments: 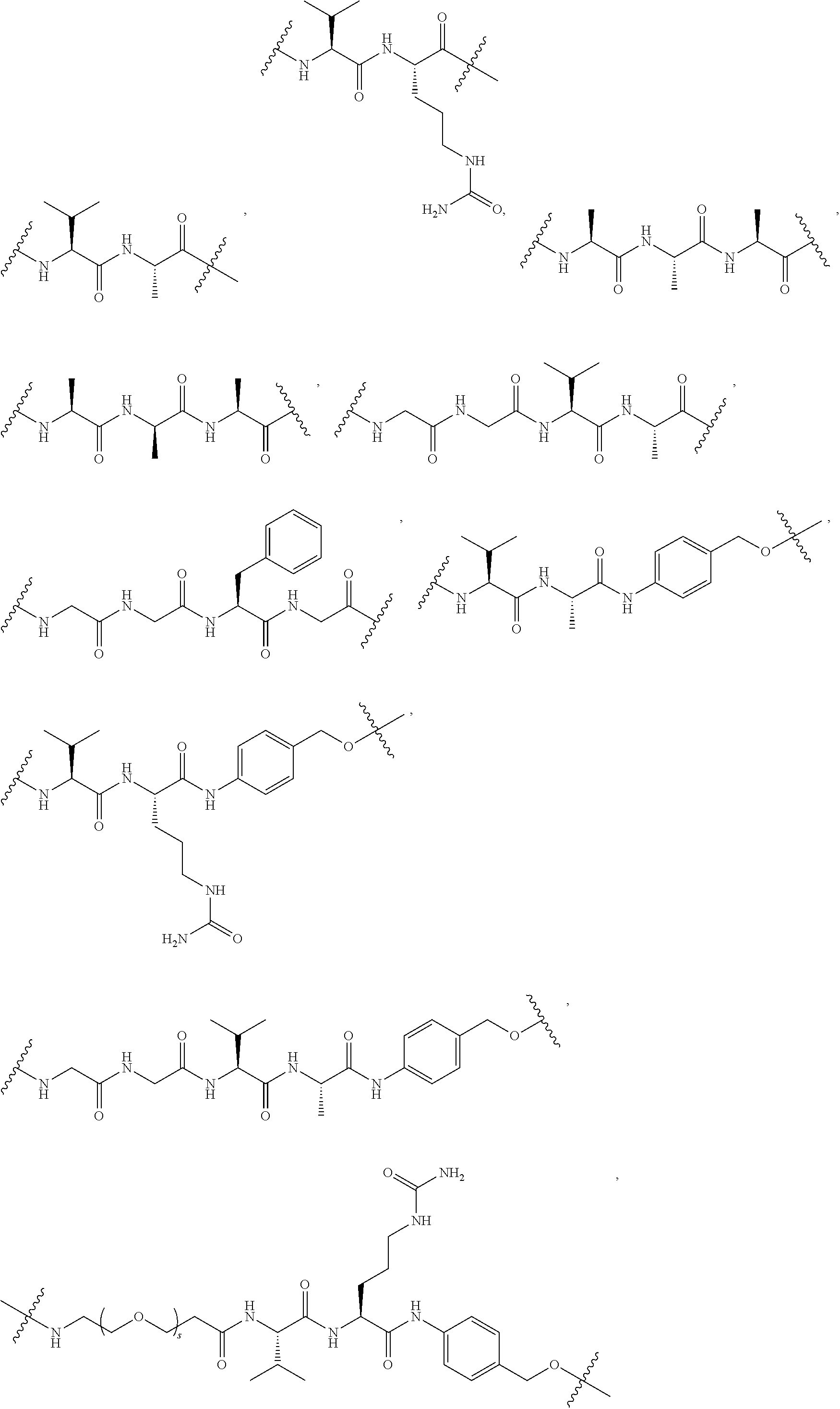 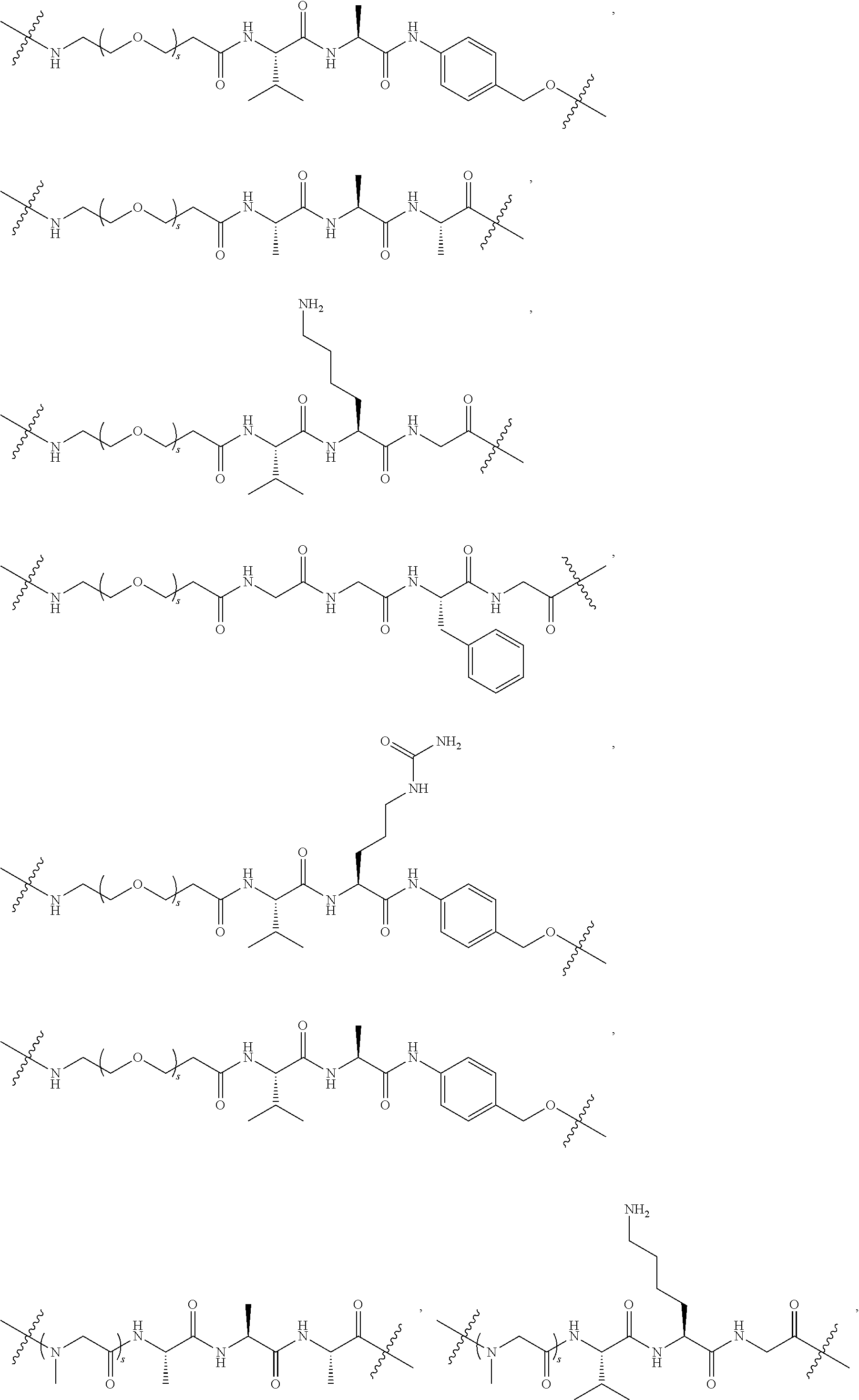 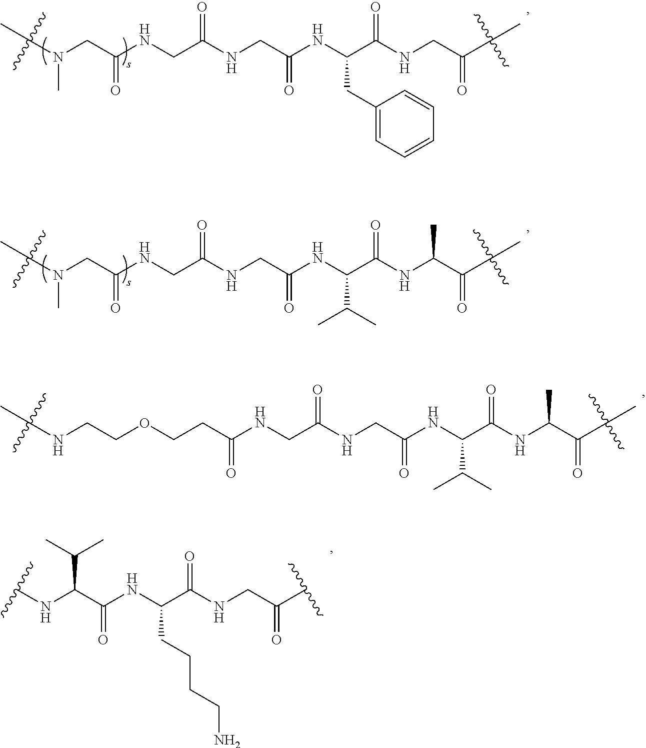 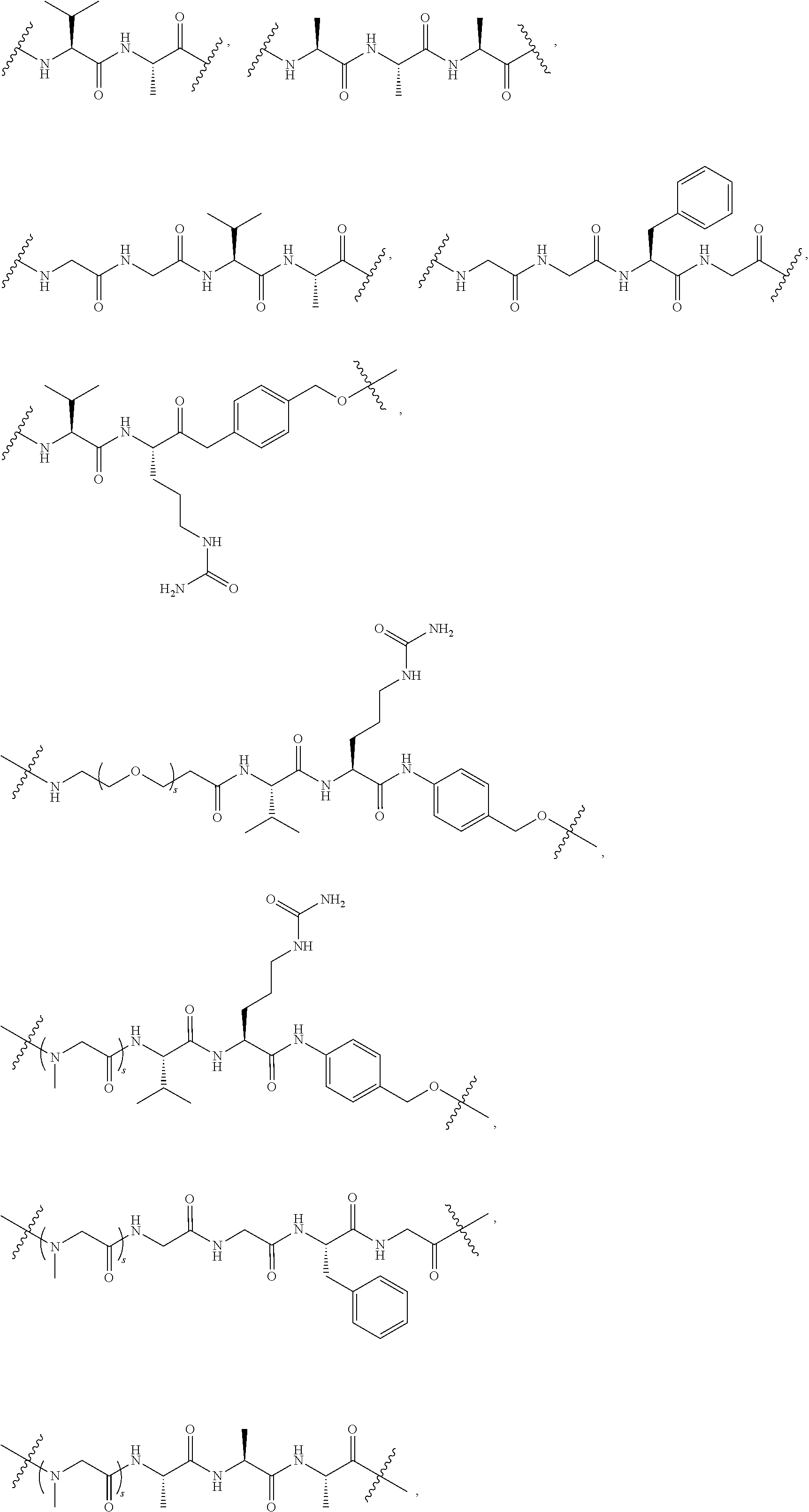
wherein s is an integer selected from 1 to 20, preferably s is an integer selected from 1 to 15, for example, an integer of 1 to 12, 3 to 12, 5 to 10, 8 to 10, for example, s is 1, 2, 3, 4, 5, 6, 7, 8, 9, 10, 11, 12, 13, 14 or 15;
preferably, L is selected from the group consisting of the following substituted or unsubstituted structural fragments: 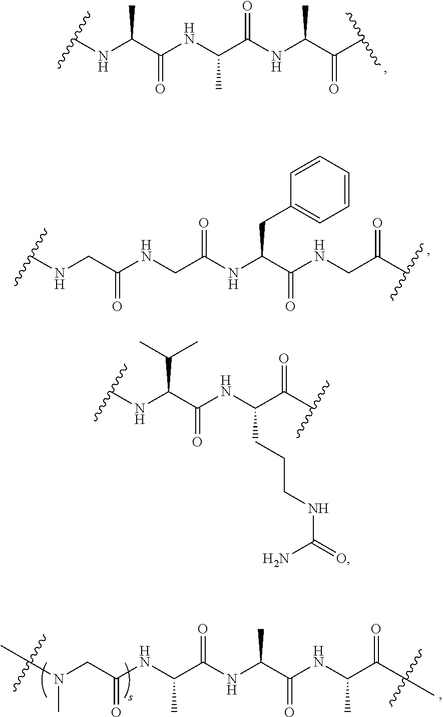
wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

7. The compound or pharmaceutically acceptable salt thereof according to claim 1, wherein E is a single bond, a substituted or unsubstituted —NH—CH₂—, or is selected from the group consisting of the following substituted or unsubstituted structural fragments:

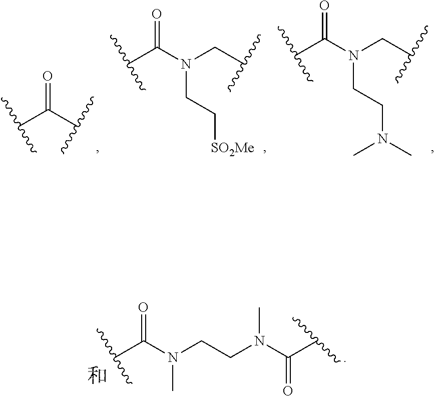

8. The compound or pharmaceutically acceptable salt thereof according to claim 1, wherein, E is a single bond, a substituted or unsubstituted —NH—CH₂—, or 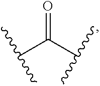 [SMILES: *C(*)=O]
for example, a single bond or

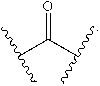 [SMILES: *C(*)=O]

9. The compound or pharmaceutically acceptable salt thereof according to claim 1, wherein, 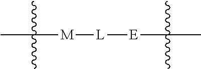
is selected from the group consisting of the following substituted or unsubstituted structures: 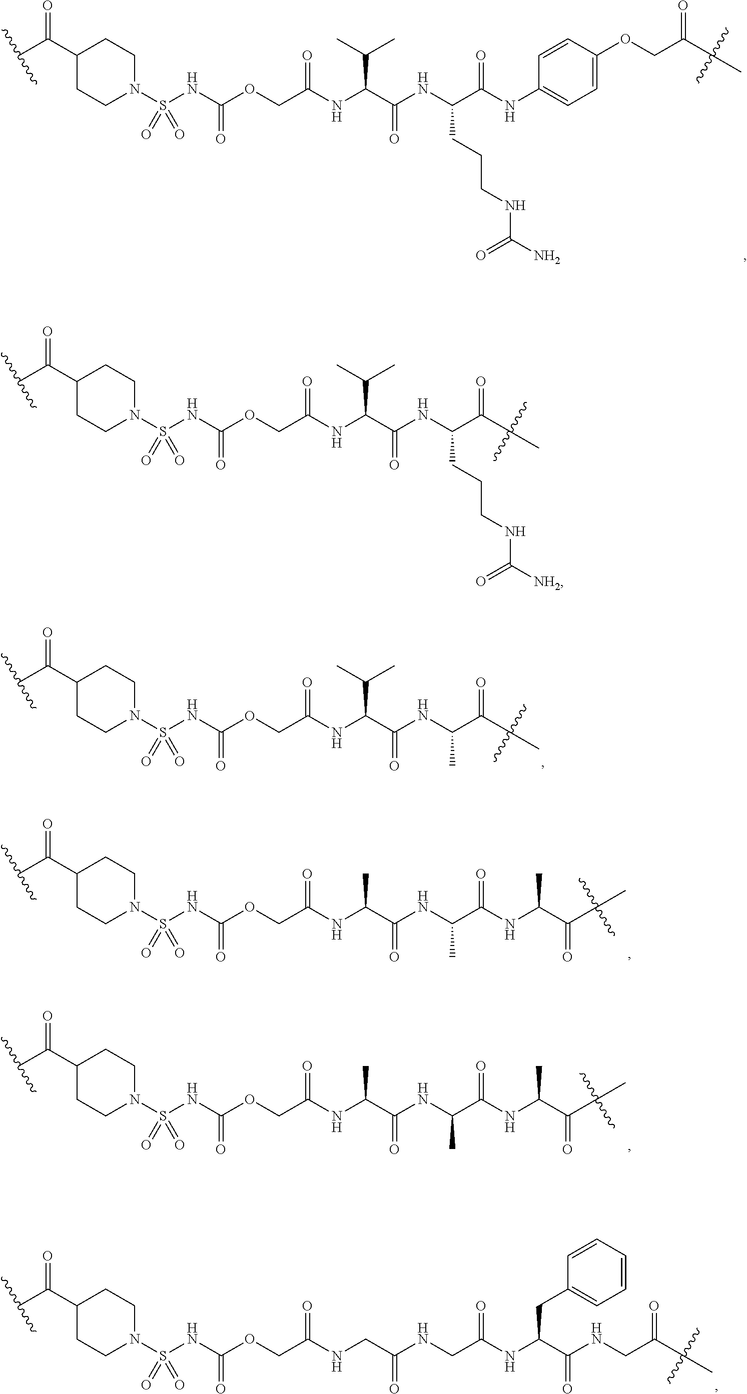 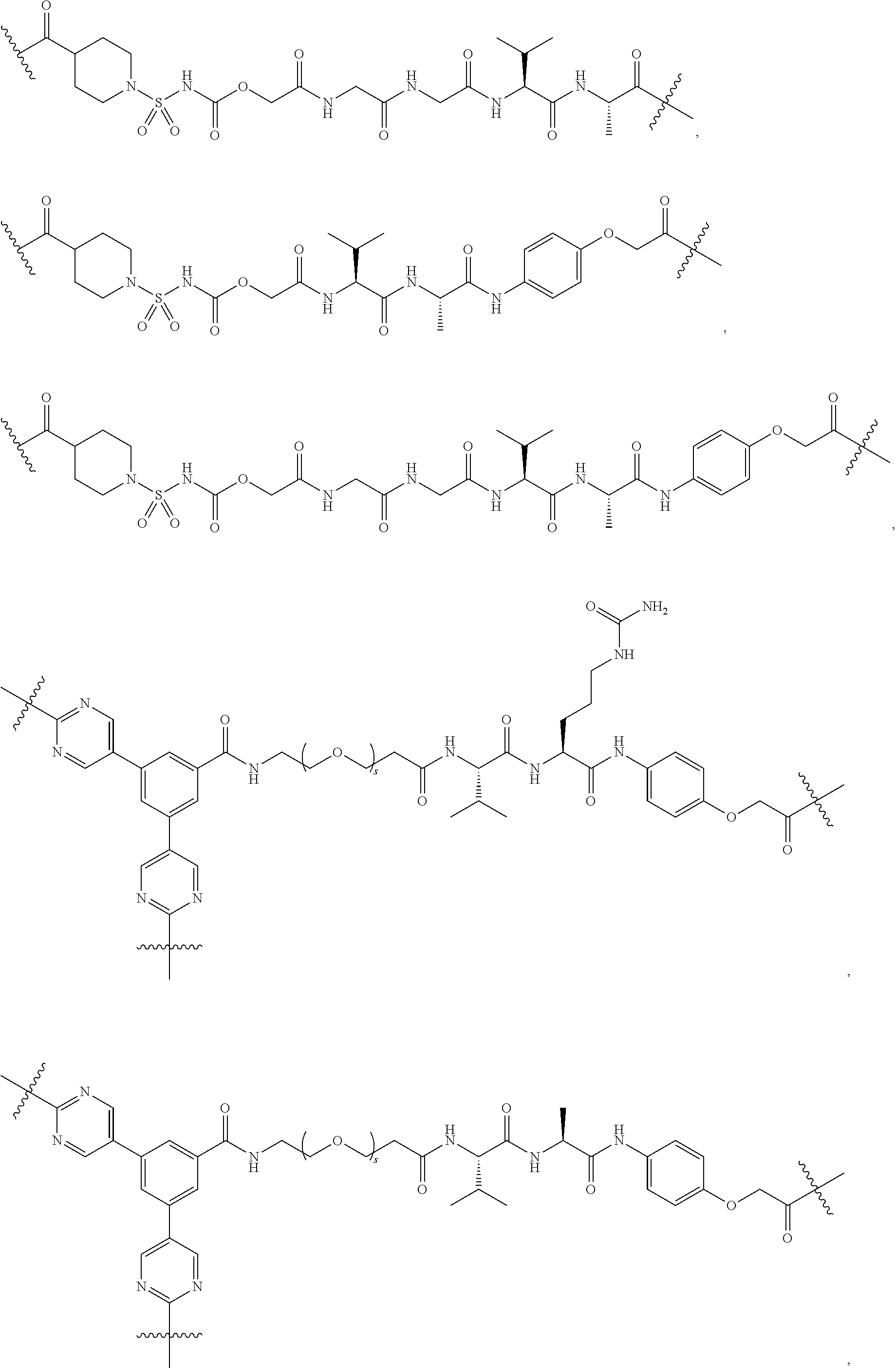 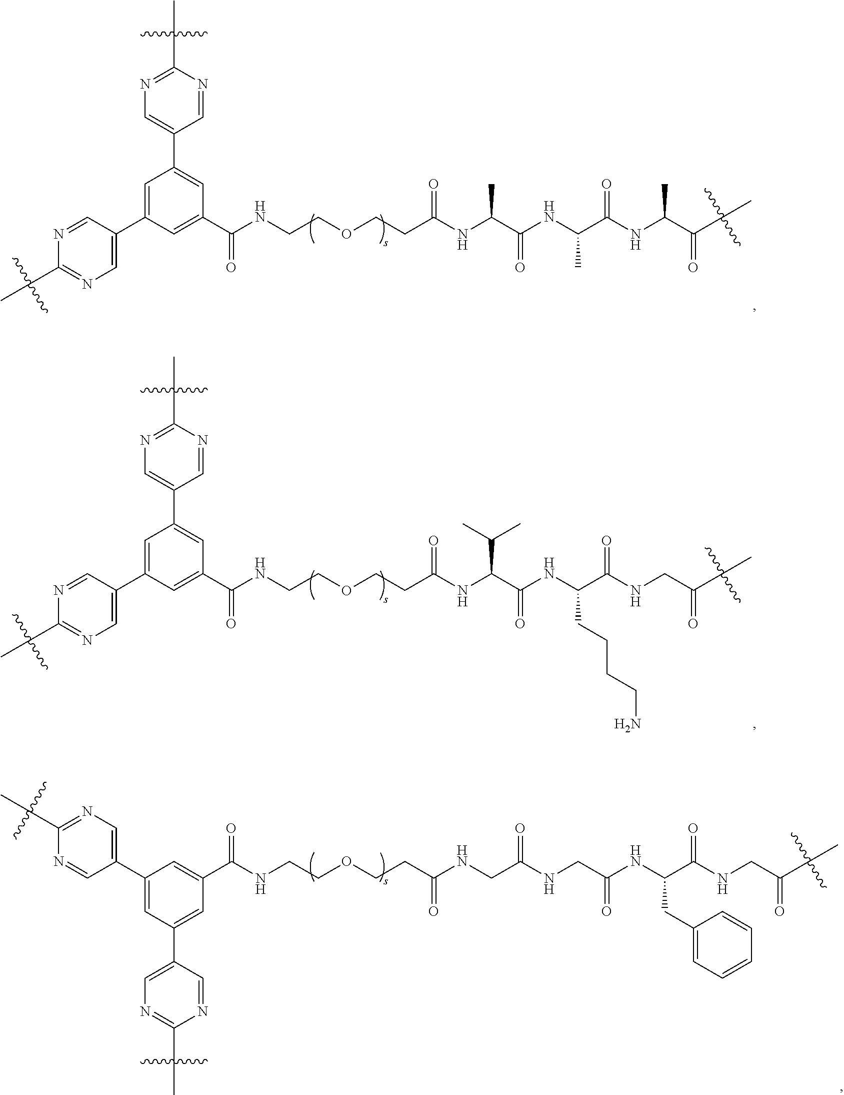 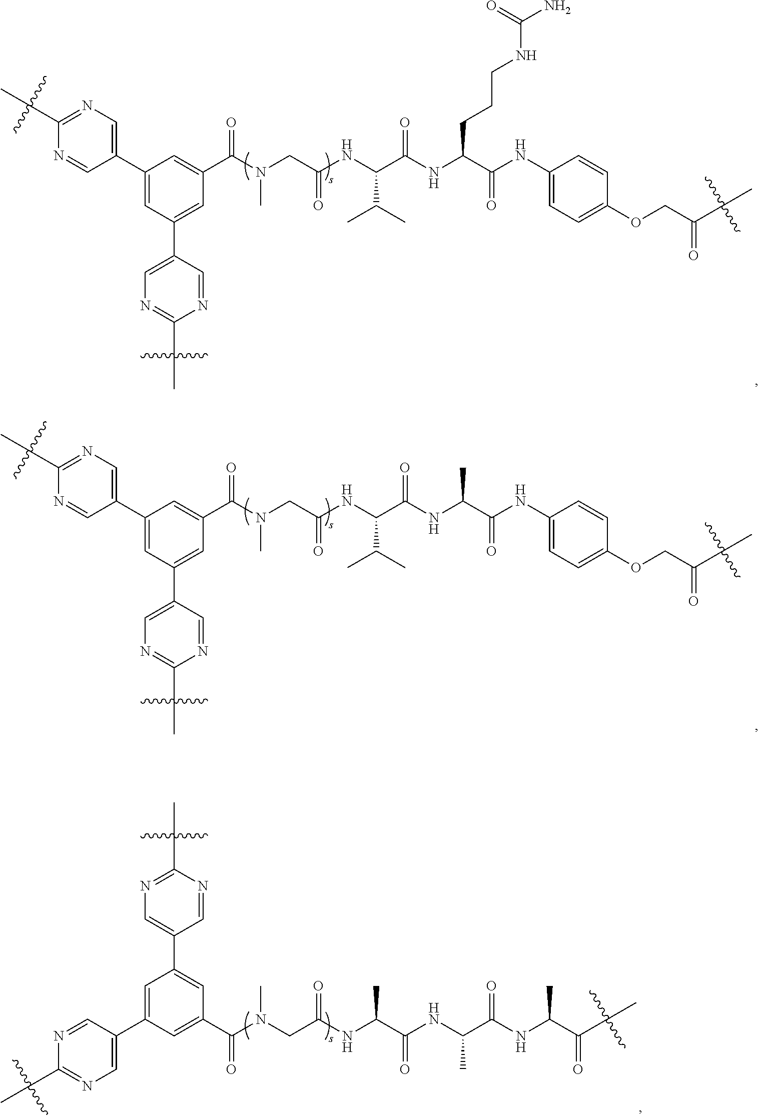 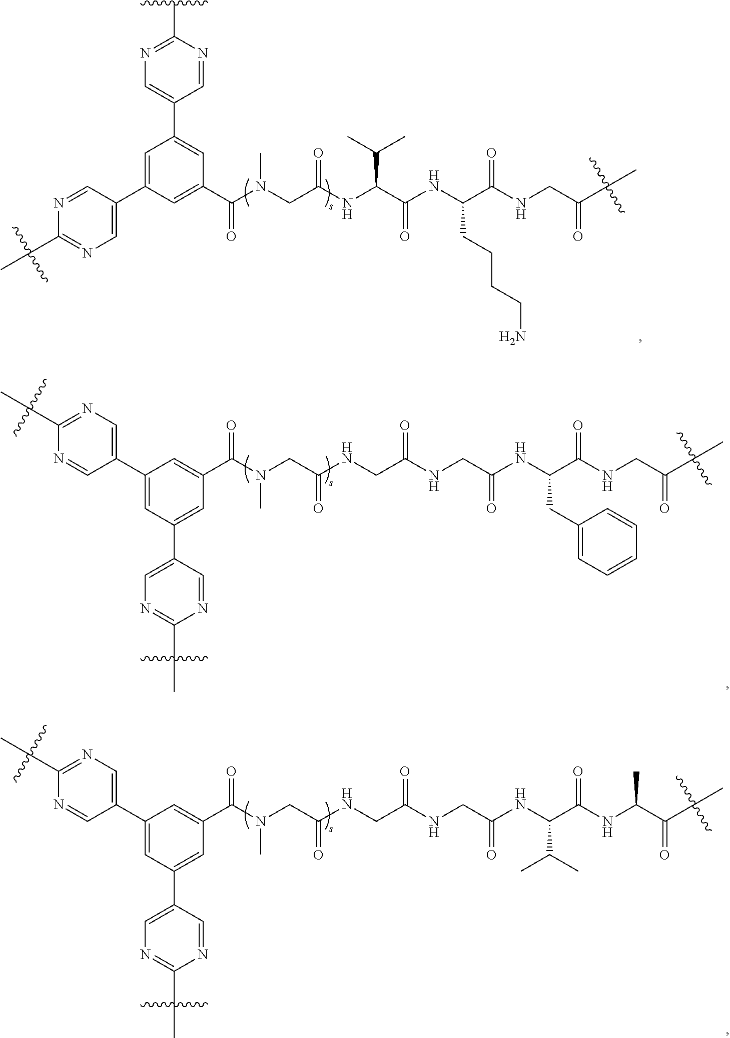 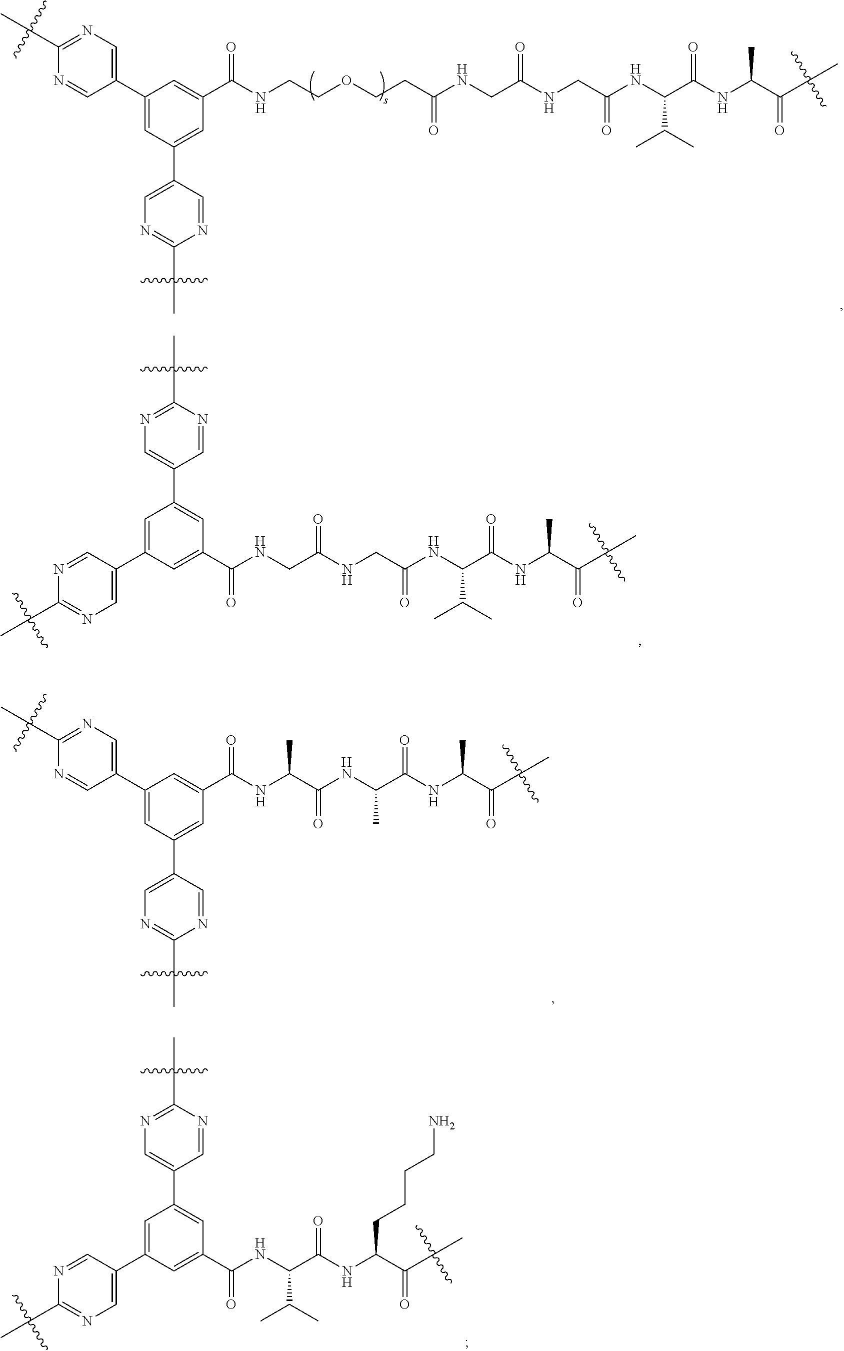
preferably, 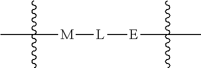
is selected from the group consisting of the following substituted or unsubstituted structures: 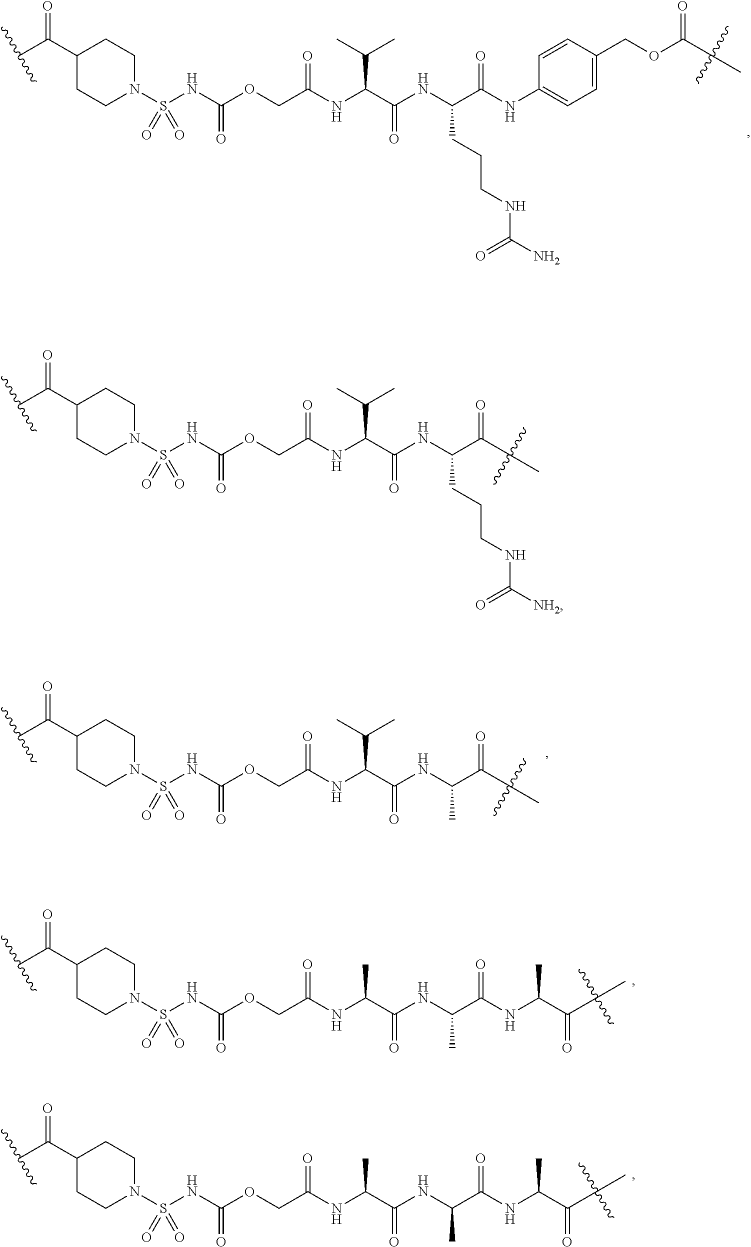 [SMILES: *C(=O)OCc1ccc(NC(=O)[C@H](CCCNC(N)=O)NC(=O)[C@@H](NC(=O)COC(=O)NS(=O)(=O)N2CCC(C(*)=O)CC2)C(C)C)cc1 ; *C(=O)C1CCN(S(=O)(=O)NC(=O)OCC(=O)N[C@H](C(=O)N[C@@H](CCCNC(N)=O)C(*)=O)C(C)C)CC1 ; *C(=O)C1CCN(S(=O)(=O)NC(=O)OCC(=O)N[C@H](C(=O)N[C@@H](C)C(*)=O)C(C)C)CC1 ; *C(=O)C1CCN(S(=O)(=O)NC(=O)OCC(=O)N[C@@H](C)C(=O)N[C@@H](C)C(=O)N[C@@H](C)C(*)=O)CC1 ; *C(=O)C1CCN(S(=O)(=O)NC(=O)OCC(=O)N[C@@H](C)C(=O)N[C@H](C)C(=O)N[C@@H](C)C(*)=O)CC1] 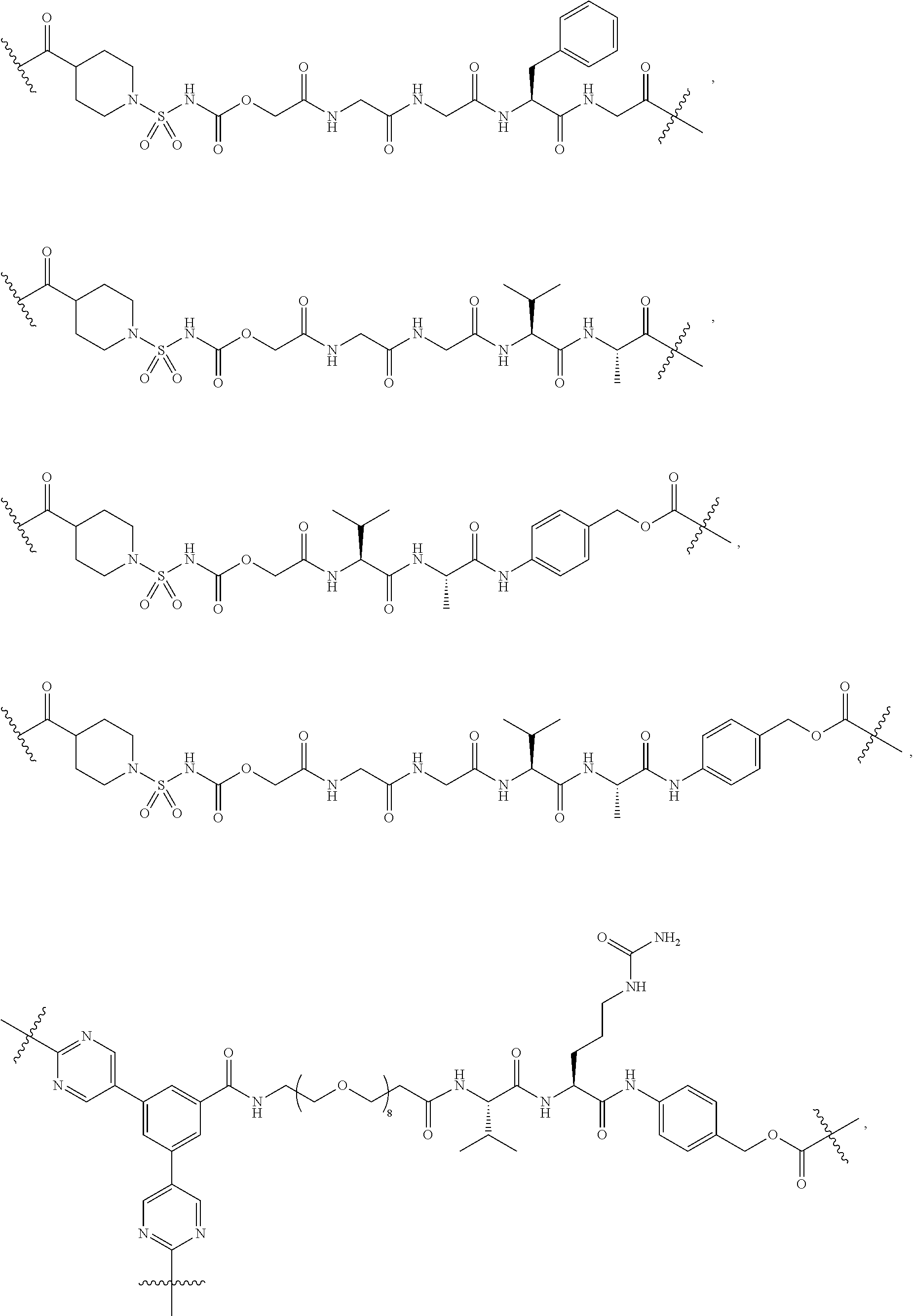 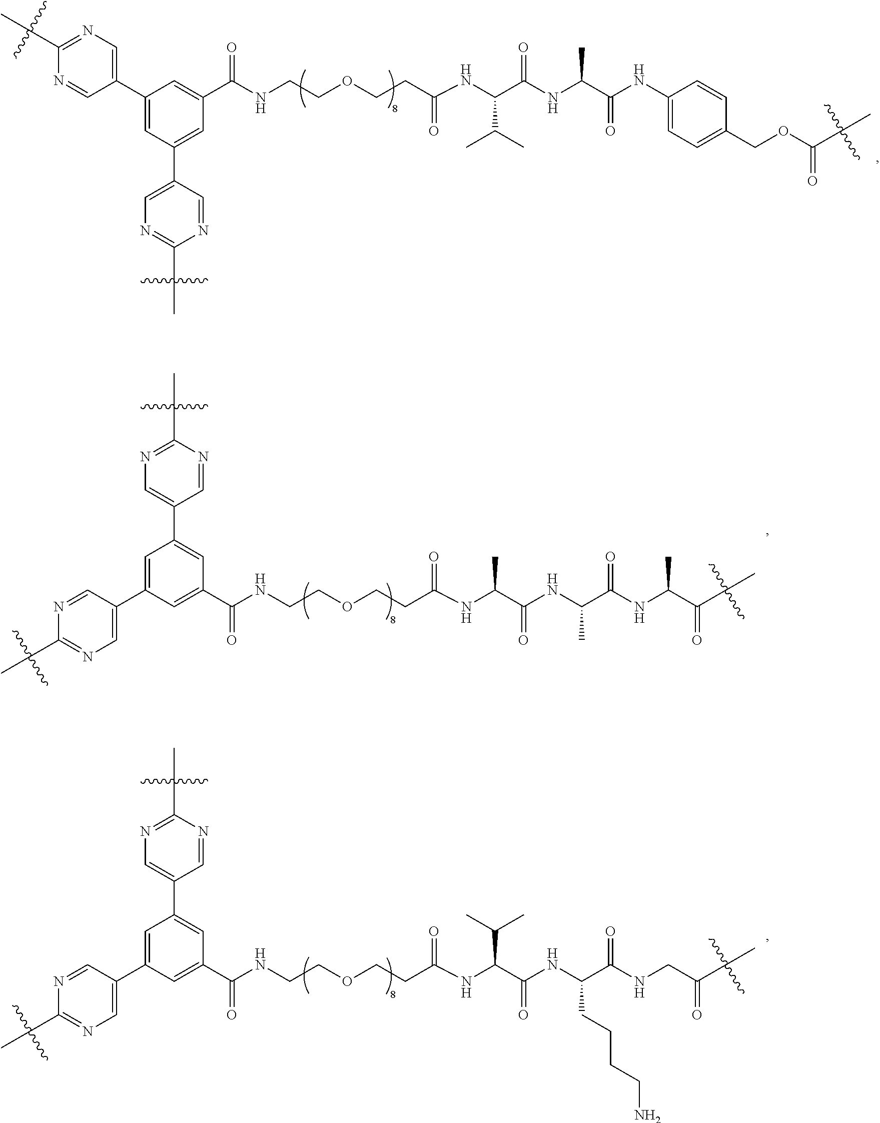 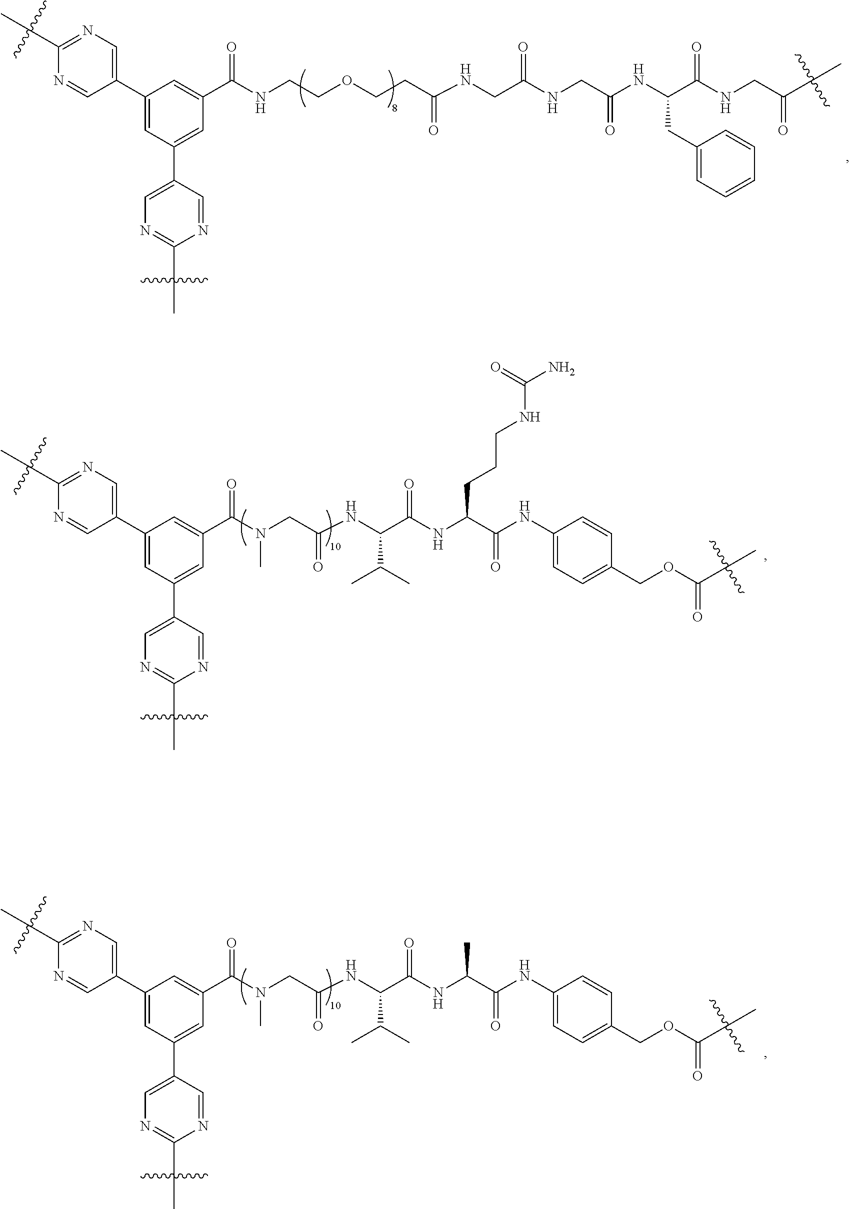 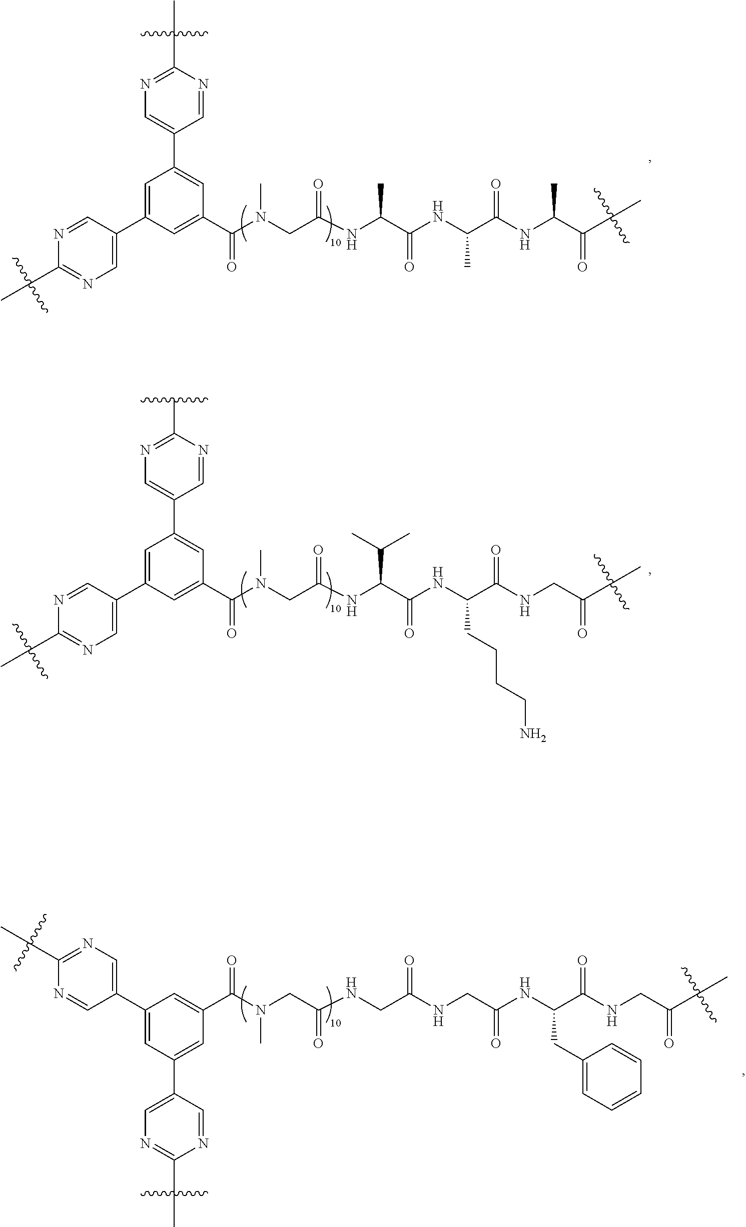 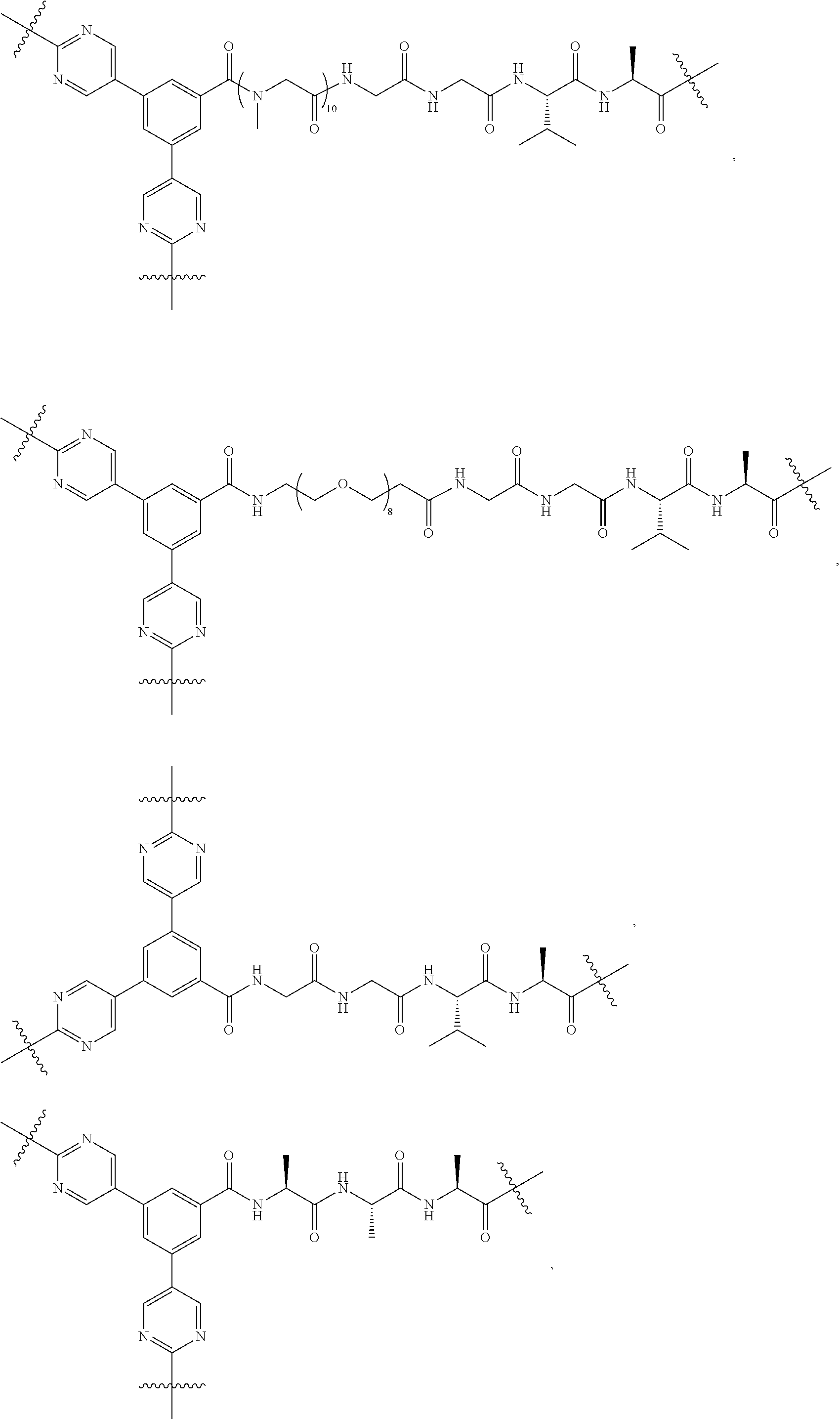 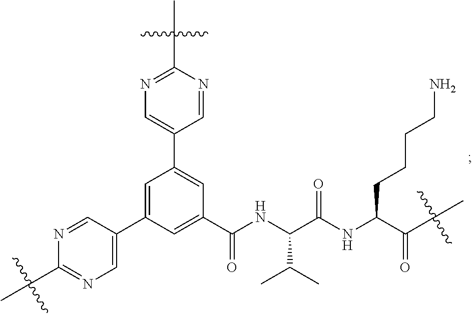 [SMILES: *C(=O)[C@H](CCCCN)NC(=O)[C@@H](NC(=O)c1cc(-c2cnc(*)nc2)cc(-c2cnc(*)nc2)c1)C(C)C]
preferably, 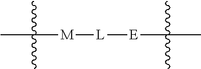
is selected from the group consisting of the following substituted or unsubstituted structures: 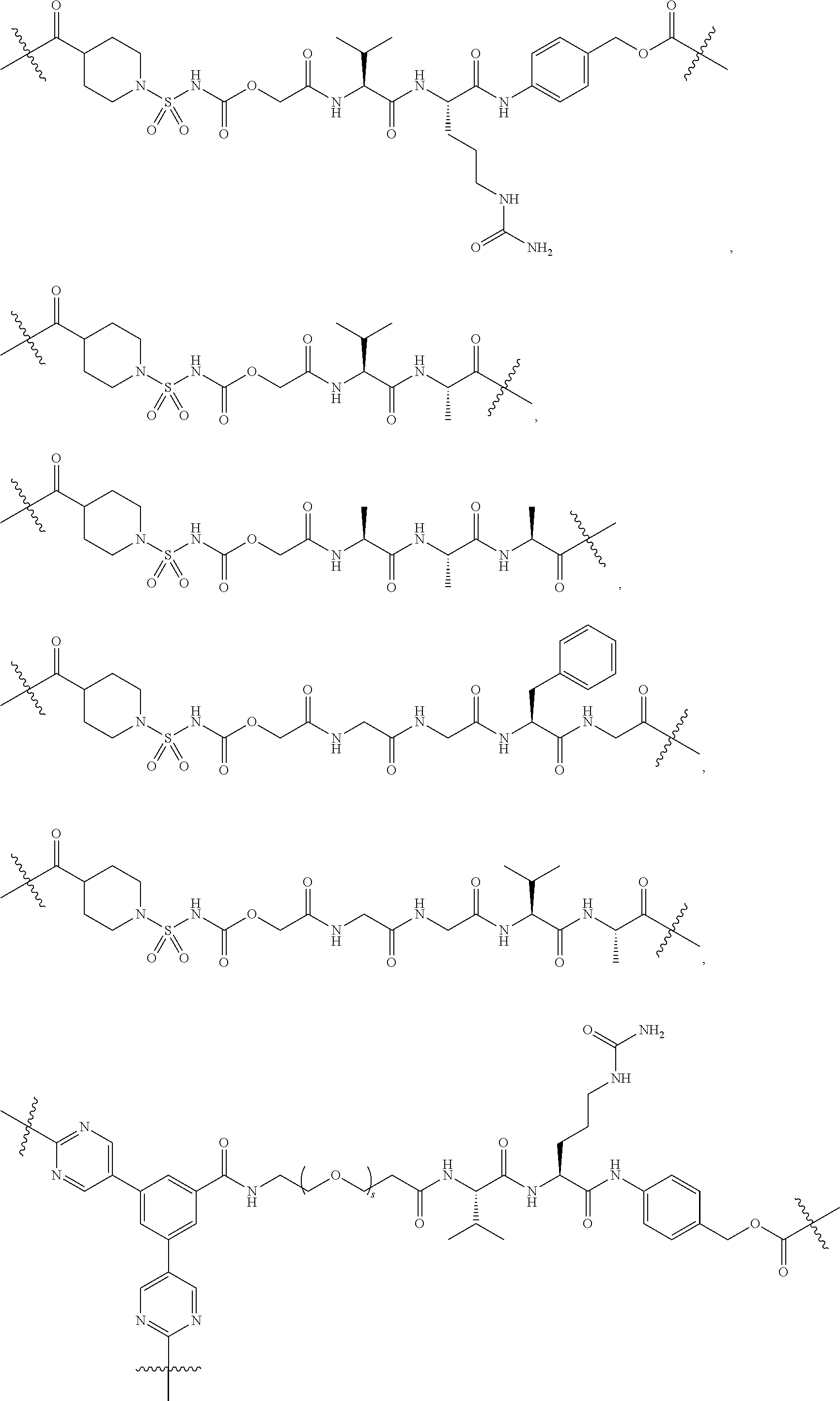 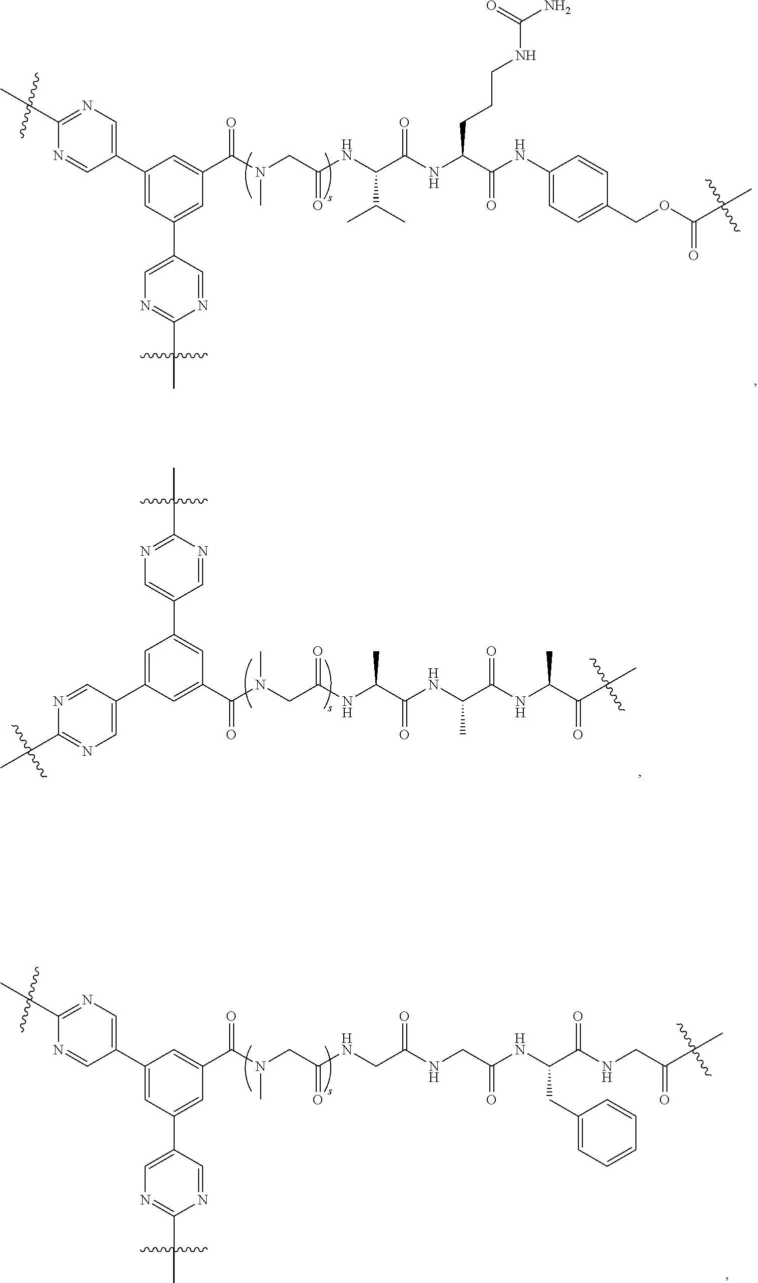
preferably, 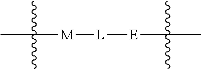
is selected from the group consisting of the following substituted or unsubstituted structures: 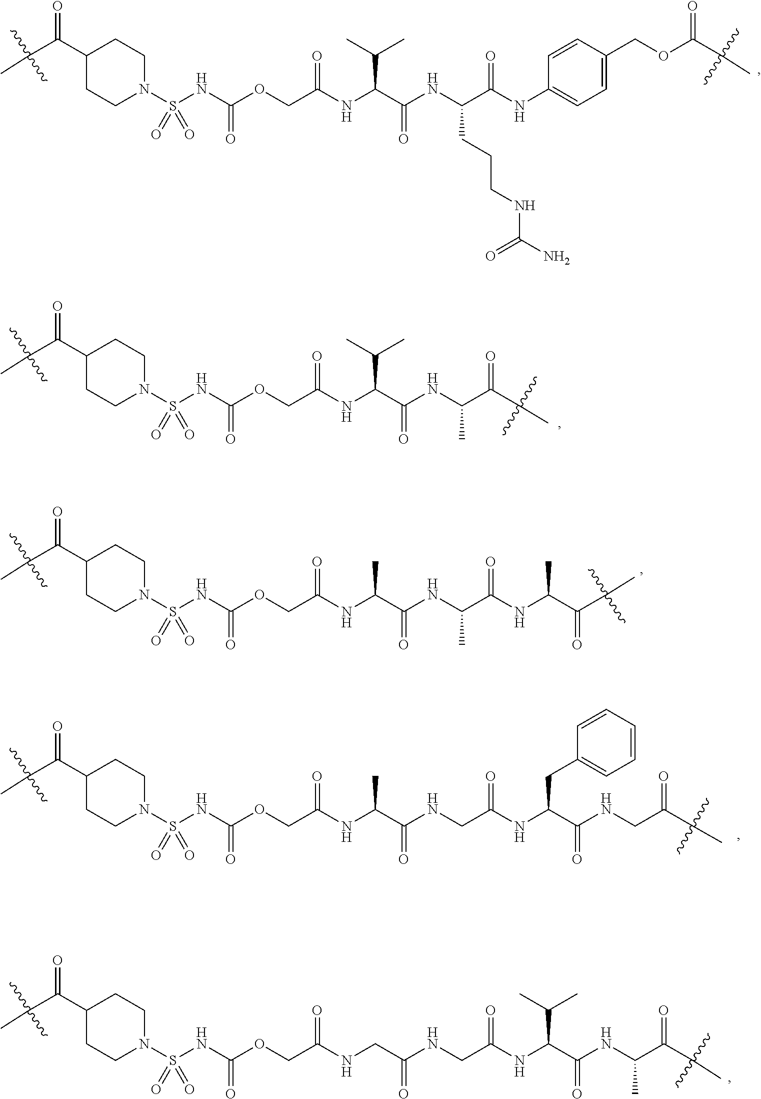 [SMILES: *C(=O)OCc1ccc(NC(=O)[C@H](CCCNC(N)=O)NC(=O)[C@@H](NC(=O)COC(=O)NS(=O)(=O)N2CCC(C(*)=O)CC2)C(C)C)cc1 ; *C(=O)C1CCN(S(=O)(=O)NC(=O)OCC(=O)N[C@H](C(=O)N[C@@H](C)C(*)=O)C(C)C)CC1 ; *C(=O)C1CCN(S(=O)(=O)NC(=O)OCC(=O)N[C@@H](C)C(=O)N[C@@H](C)C(=O)N[C@@H](C)C(*)=O)CC1 ; *C(=O)CNC(=O)[C@H](Cc1ccccc1)NC(=O)CNC(=O)[C@H](C)NC(=O)COC(=O)NS(=O)(=O)N1CCC(C(*)=O)CC1 ; *C(=O)C1CCN(S(=O)(=O)NC(=O)OCC(=O)NCC(=O)NCC(=O)N[C@H](C(=O)N[C@@H](C)C(*)=O)C(C)C)CC1] 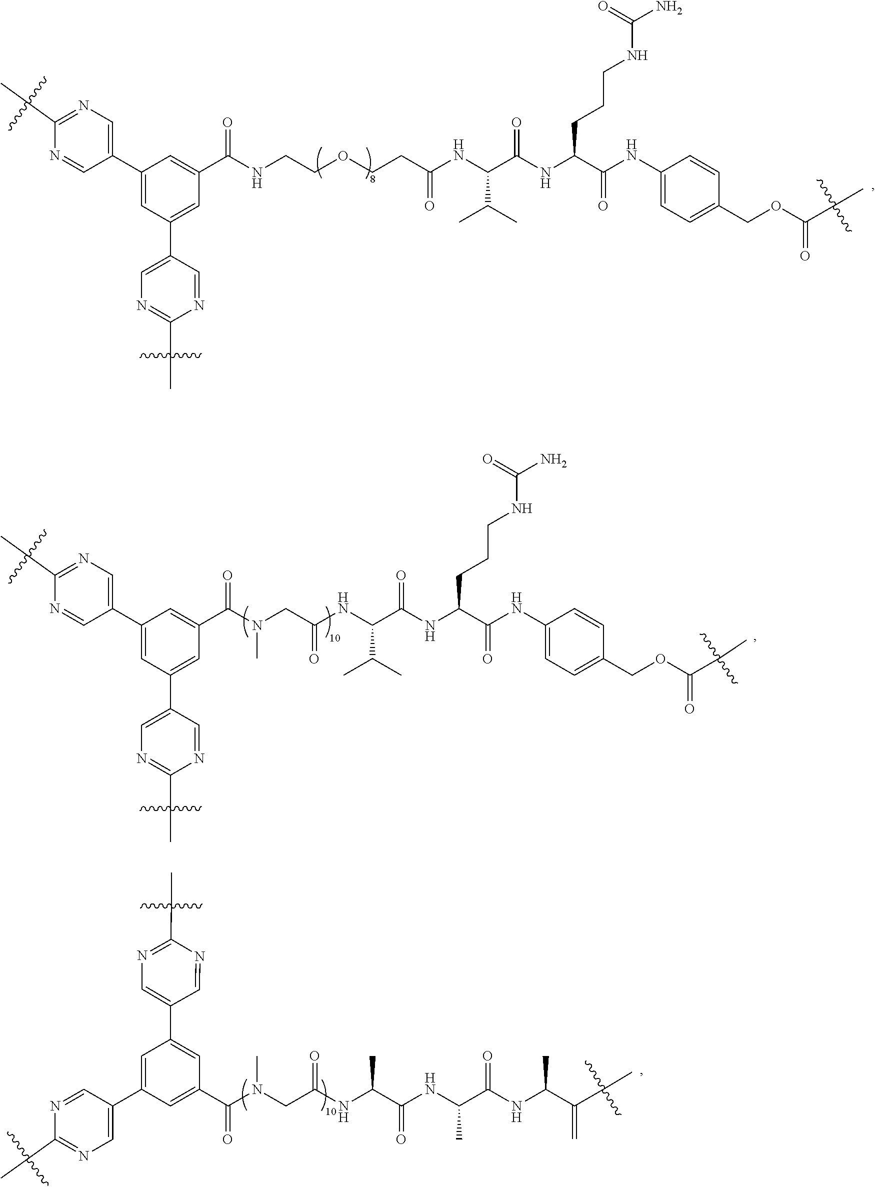 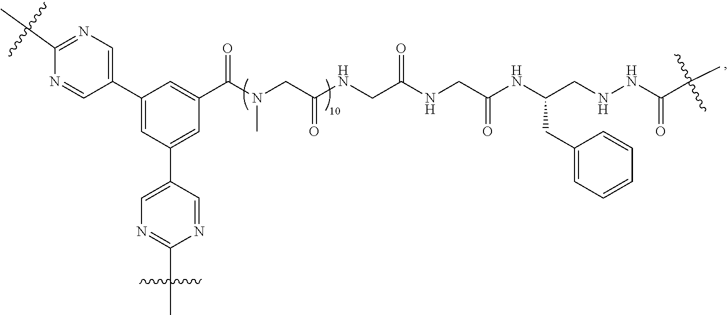
preferably, 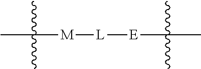
is selected from the group consisting of the following substituted or unsubstituted structures: 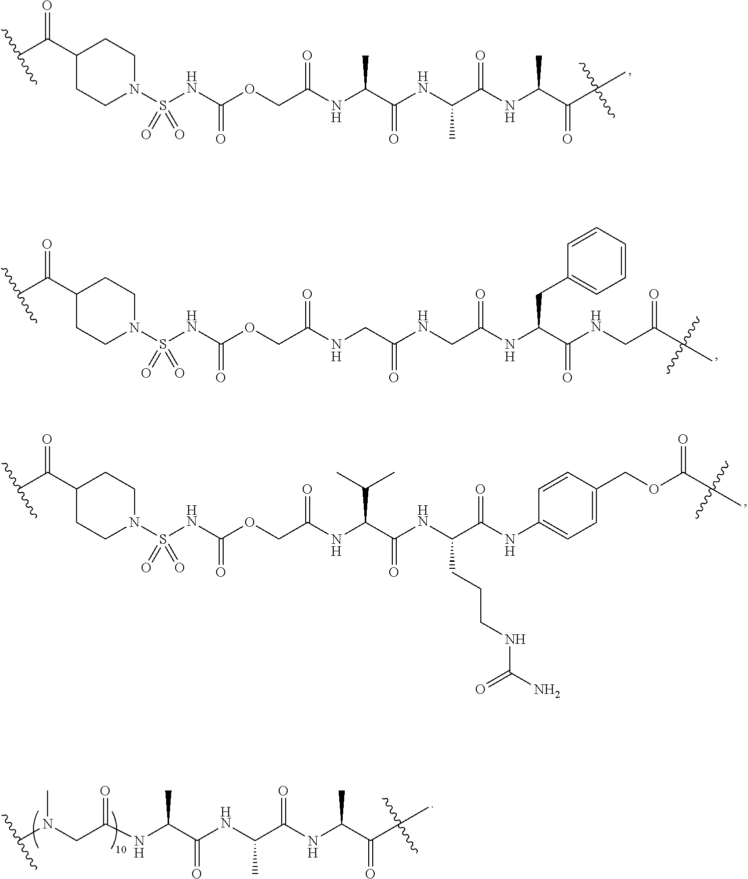
wherein s is an integer selected from 1 to 20, preferably s is an integer selected from 1 to 15, such as an integer of 1 to 12, 3 to 12, 5 to 10, 8 to 10, for example, s is 1, 2, 3, 4, 5, 6, 7, 8, 9, 10, 11, 12, 13, 14 or 15.

10. The compound or pharmaceutically acceptable salt thereof according to claim 1, wherein the cytotoxic drug is an eribulin compound;
preferably, the cytotoxic drug is selected from the group consisting of the following compounds or isotopically labeled compounds thereof: 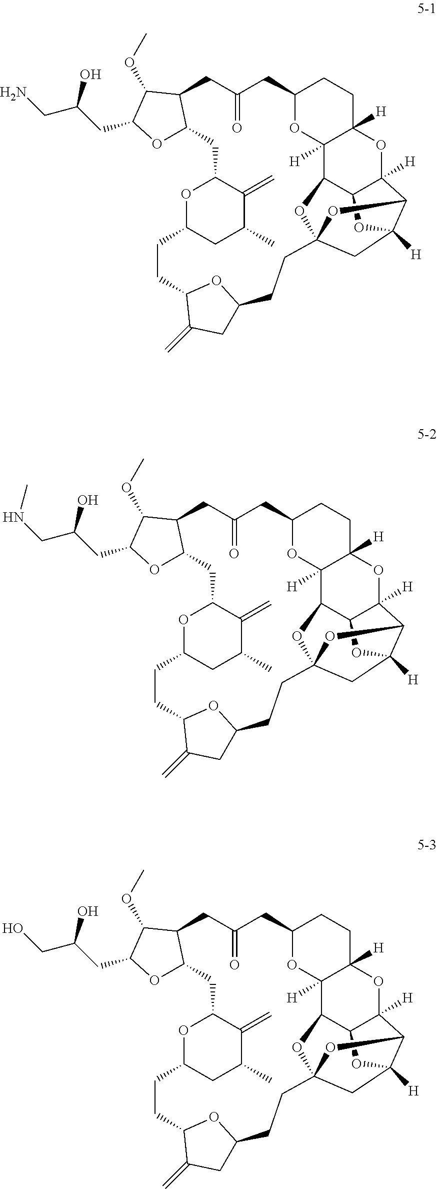
preferably, the cytotoxic drug is connected through the —OH, primary amino group, secondary amine group or tertiary amine group thereon to E in the antibody-drug conjugate.

11. The compound or pharmaceutically acceptable salt thereof according to claim 1, wherein, the compound is selected from the group consisting of: 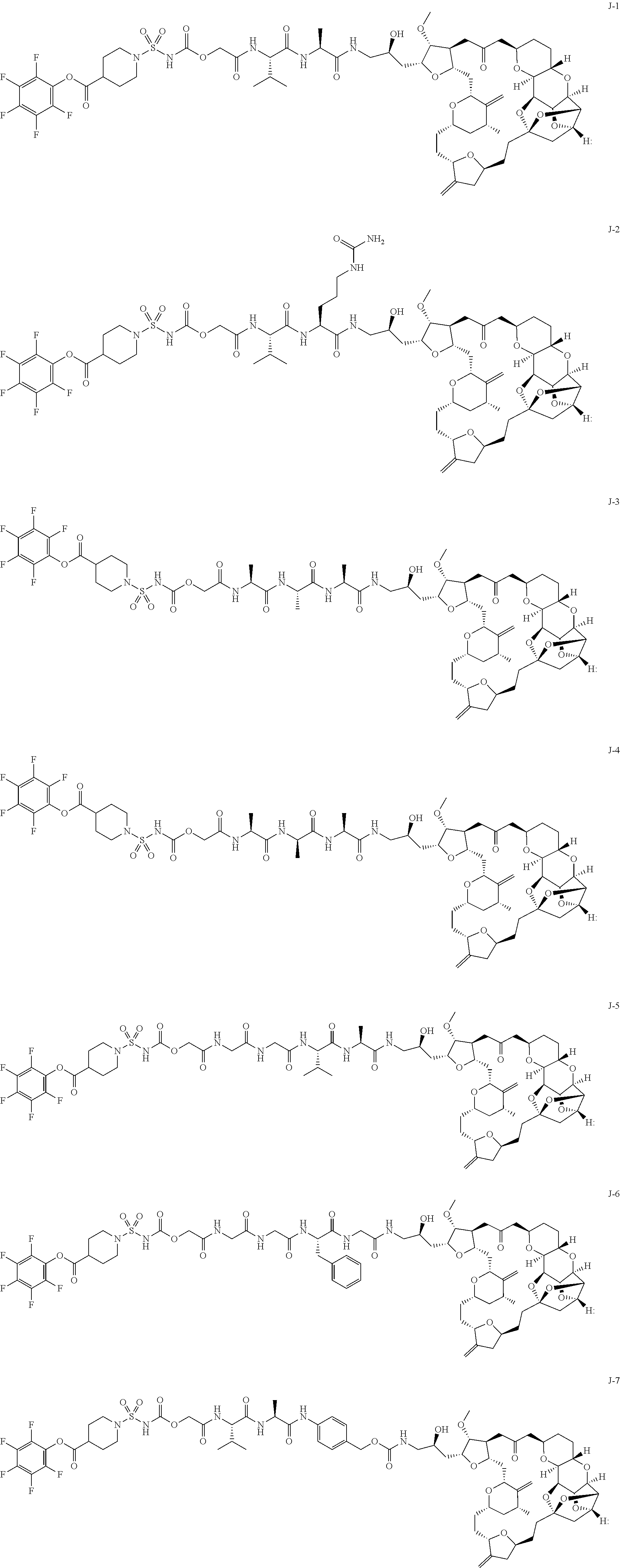 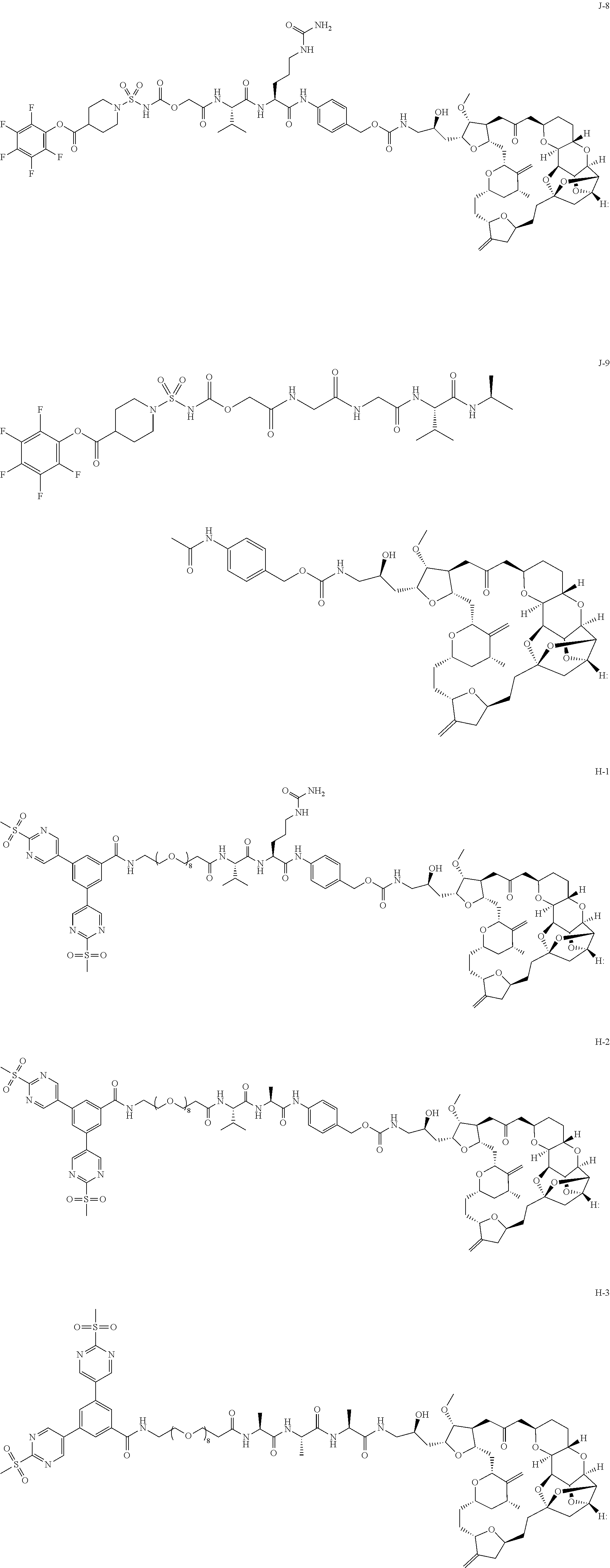 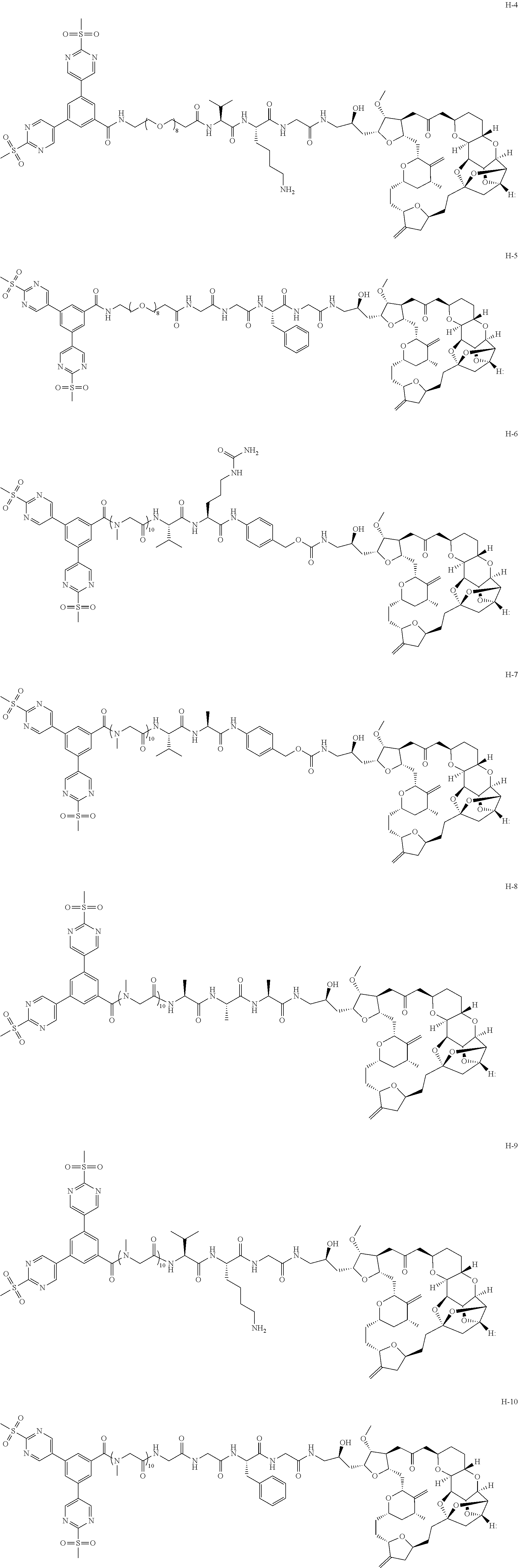 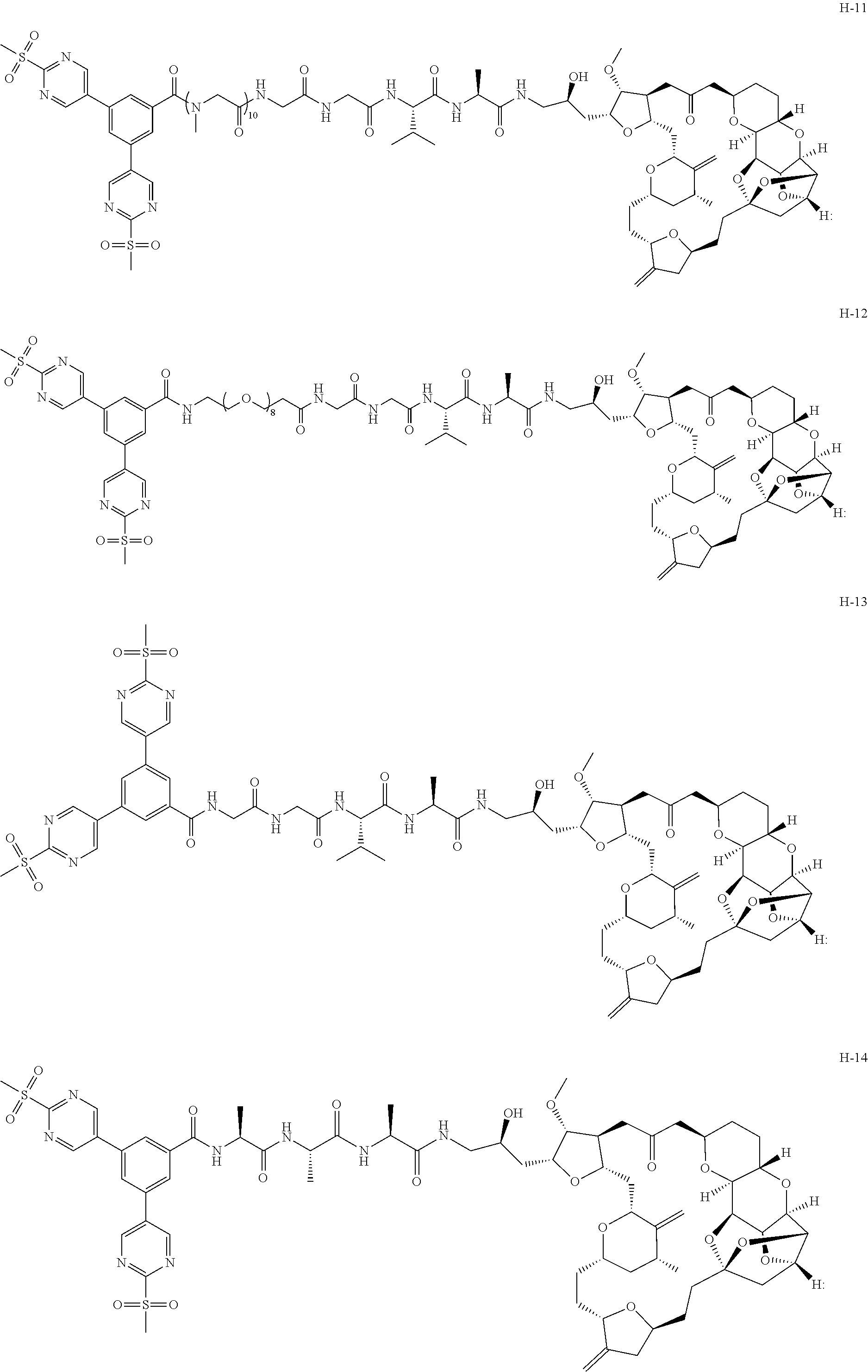 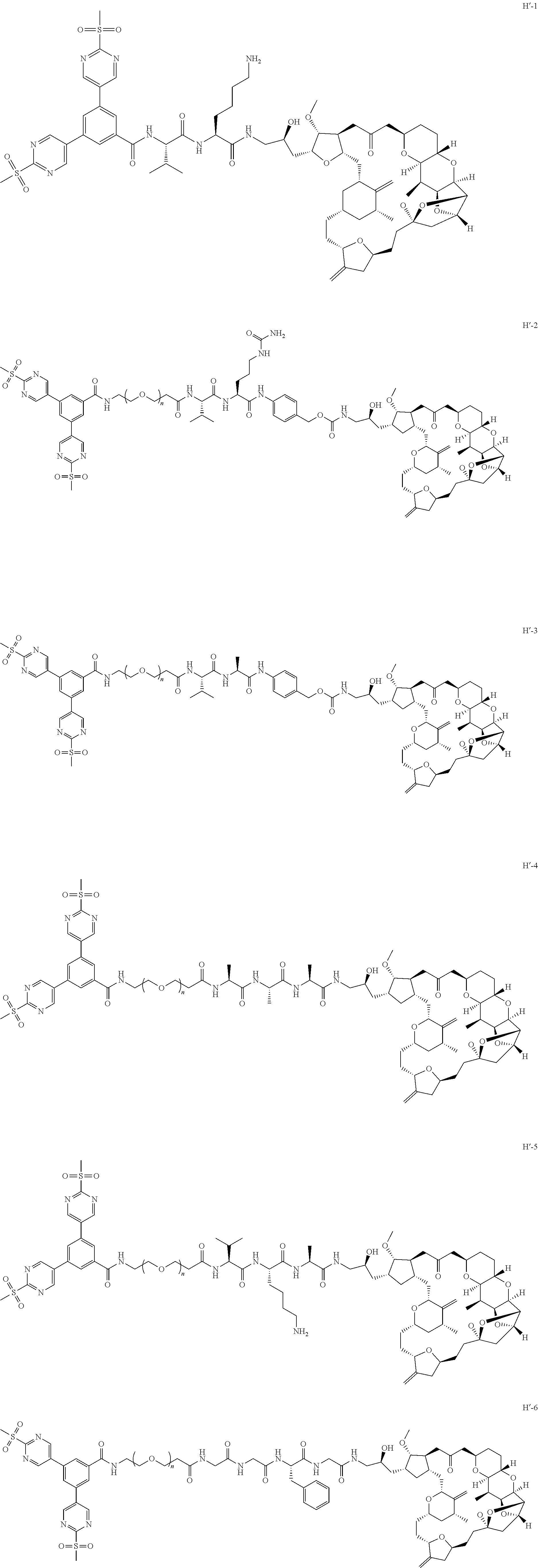 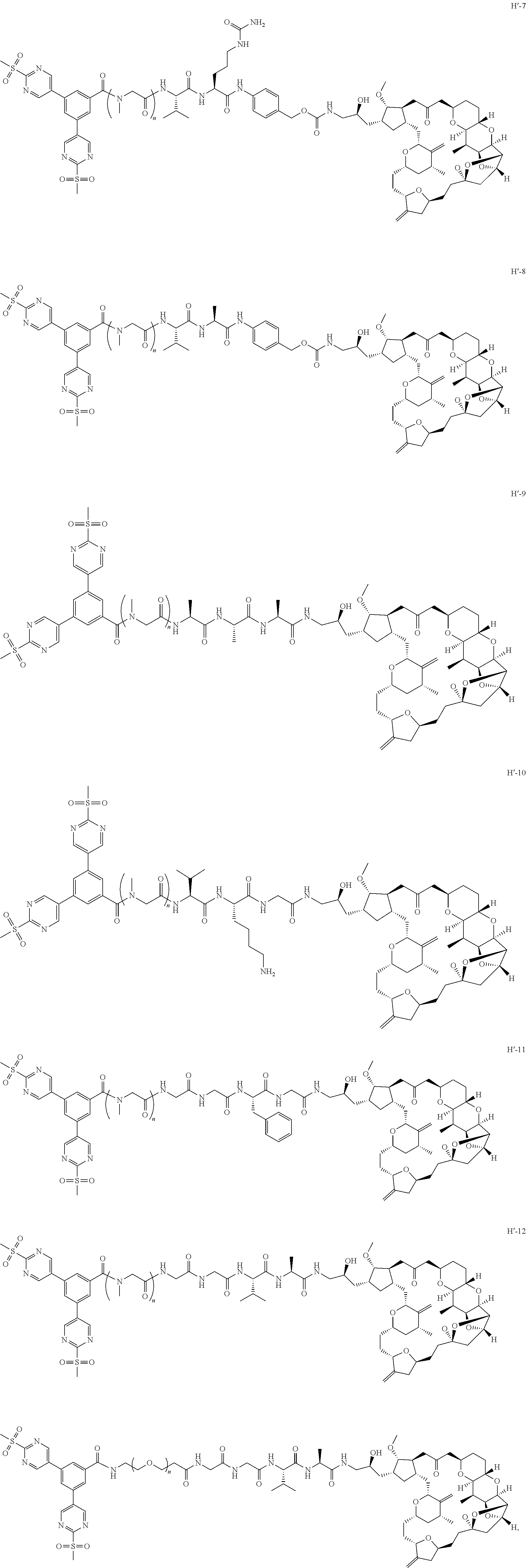
wherein n is an integer selected from 1 to 20, preferably an integer of 1 to 15, such as an integer of 1 to 12, 3 to 12, 5 to 10, 8 to 10, such as 1, 2, 3,4, 5,6, 7,8, 9, 10, 11, 12, 13, 14 or 15, preferably 5, 8, 10;
preferably, the compound is selected from J-1 to J-9, H-1 to H1-15.

12. A conjugate represented by Formula (II): 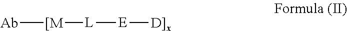
wherein
Ab represents a targeting moiety; M, L, E and D are as described in claim 1;
x is 1 to 10.

13. The conjugate according to claim 12, wherein the Ab represents an antibody or antigen-binding fragment thereof, such as a monoclonal antibody or antigen-binding fragment thereof, wherein the monoclonal antibody or antigen-binding fragment thereof comprises Fab, Fab′, F(ab′)₂, Fd, Fv, dAb, complementary determining region fragment, single chain antibody (e.g., scFv), non-human antibody, humanized antibody, chimeric antibody, fully human antibody, probody, bispecific antibody or multispecific antibody;
preferably, Ab represents an antibody or antigen-binding fragment thereof capable of specifically binding epidermal growth factor receptor 2 (Her2), a member of the ErbB family of receptor tyrosine kinases;
preferably, the antibody or antigen-binding fragment thereof comprises:
(1) the following heavy chain variable region (VH) and/or light chain variable region (VL):
(1a) a heavy chain variable region (VH) comprising the following 3 CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following 3 CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following 3 CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following 3 CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof:
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following 3 CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following 3 CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b), has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following 3 CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following 3 CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following 3 CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following 3 CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
preferably, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following 3 CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following 3 CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following 3 CDRs: a CDR-L1 having a sequence as set forth in SEQ ID NO: 23 or variant thereof, a CDR-L2 having sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following 3 CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following 3 CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following 3 CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following 3 CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL), wherein the CDRs are defined according to the IMGT numbering system:
(4a) a heavy chain variable region (VH) comprising the following 3 CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following 3 CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following 3 CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following 3 CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof:
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
preferably, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following 3 CDRs: a CDR-H1 having a sequence as set forth in SEQ ID NO: 5, a CDR-H2 having a sequence as set forth in SEQ ID NO:
6, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following 3 CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following 3 CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following 3 CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following 3 CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following 3 CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following 3 CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following 3 CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following 3 CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following 3 CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25;
preferably, the antibody or antigen-binding fragment thereof comprises:
(a) a VH as set forth in SEQ ID NO: 1 or variant thereof, and/or, a VL as set forth in SEQ ID NO: 2 or variant thereof; or
(b) a VH as set forth in SEQ ID NO: 3 or variant thereof, and/or, a VL as set forth in SEQ ID NO: 4 or variant thereof,
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution;
preferably, the antibody or antigen-binding fragment thereof comprises:
(a) a VH as set forth in SEQ ID NO: 1, and, a VL as set forth in SEQ ID NO: 2; or
(b) a VH as set forth in SEQ ID NO: 3, and, a VL as set forth in SEQ ID NO: 4;
preferably, the antibody or antigen-binding fragment thereof further comprises:
(a) a human immunoglobulin heavy chain constant region (CH) or variant thereof, wherein the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared with the wild-type sequence from which it is derived; and
(b) a human immunoglobulin light chain constant region (CL) or variant thereof, wherein the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared with the wild-type sequence from which it is derived;
preferably, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region;
preferably, the antibody or antigen-binding fragment thereof comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; such as a conservative substitution of 1, 2, 3, 4 or 5 amino acids) as compared with SEQ ID NO: 35;
preferably, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; such as a conservative substitution of 1, 2, 3, 4 or 5 amino acids) as compared with SEQ ID NO: 36;
preferably, the antibody or antigen-binding fragment thereof comprises a heavy chain constant region (CH) as set forth in SEQ ID NO: 35 and a light chain constant region (CL) as set forth in SEQ ID NO: 36;
preferably, the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and, a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and, a light chain comprising a VL having a sequence as set forth in SEQ ID NO: 4 and a light chain constant region (CL) as set forth in SEQ ID NO: 36;
preferably, Ab is trastuzumab or pertuzumab, or antigen-binding fragment thereof.

14. The conjugate according to claim 12, which is selected from the group consisting of: 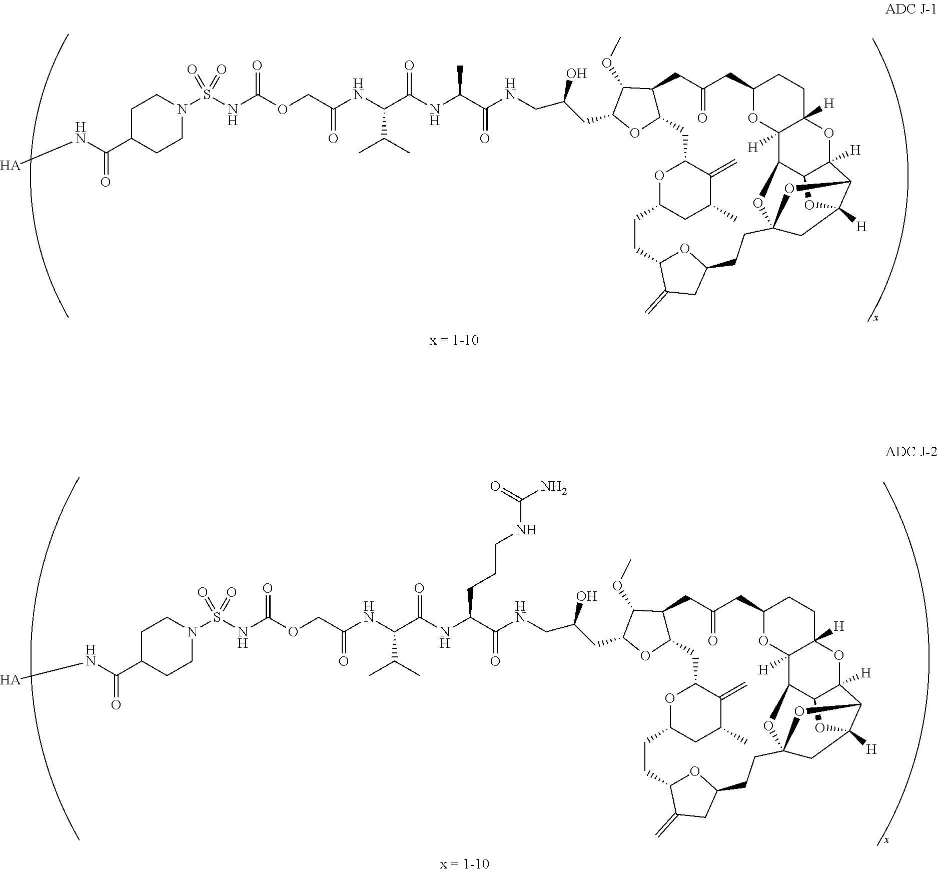 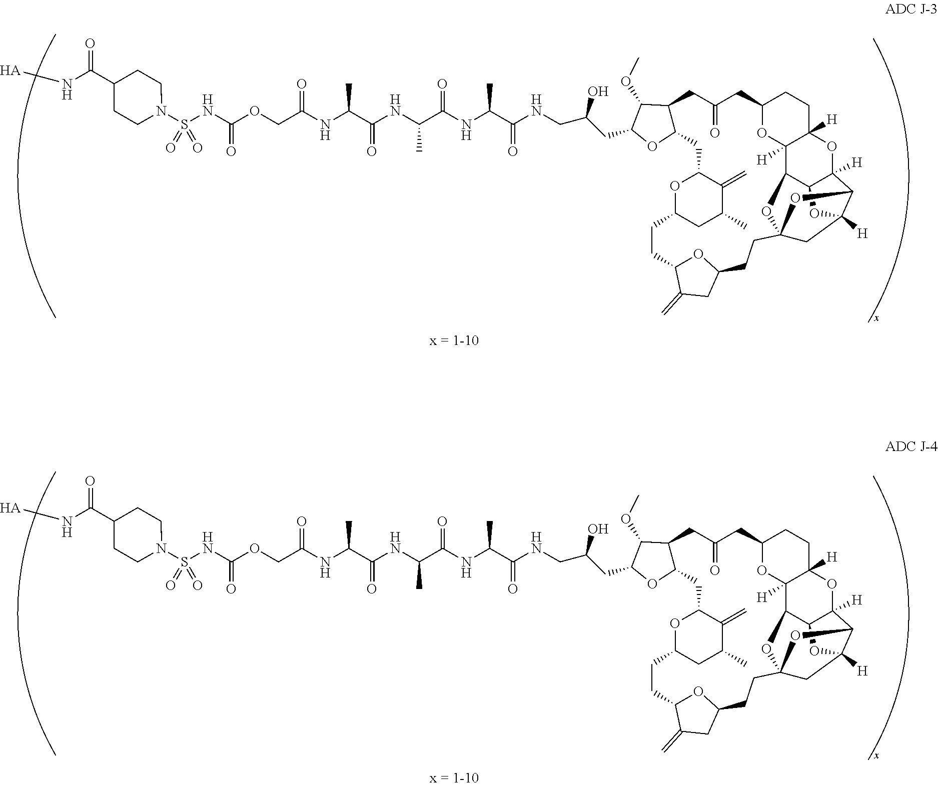 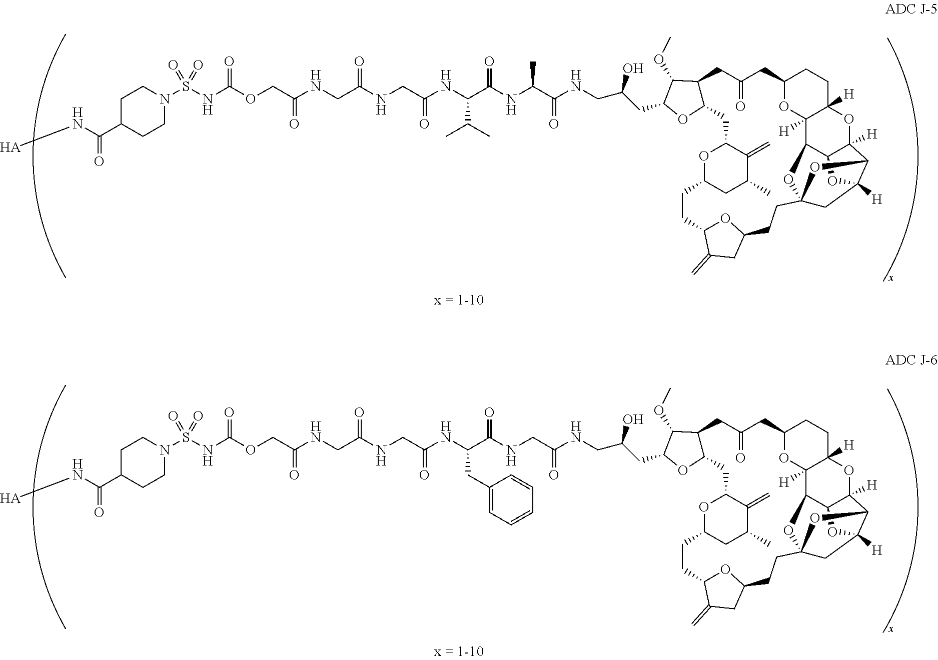 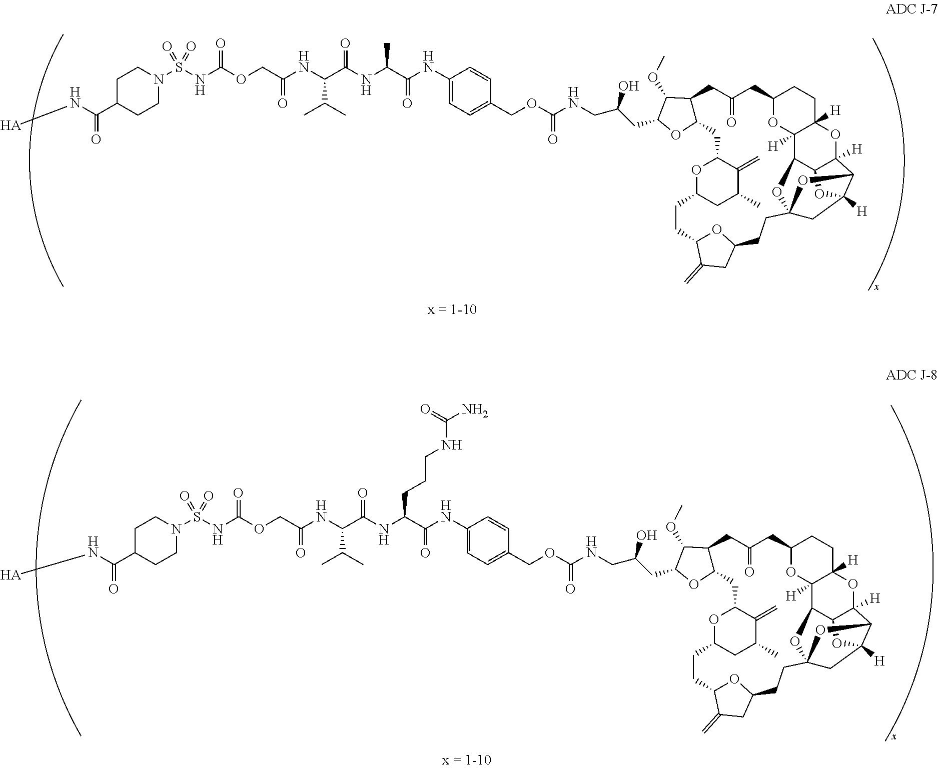 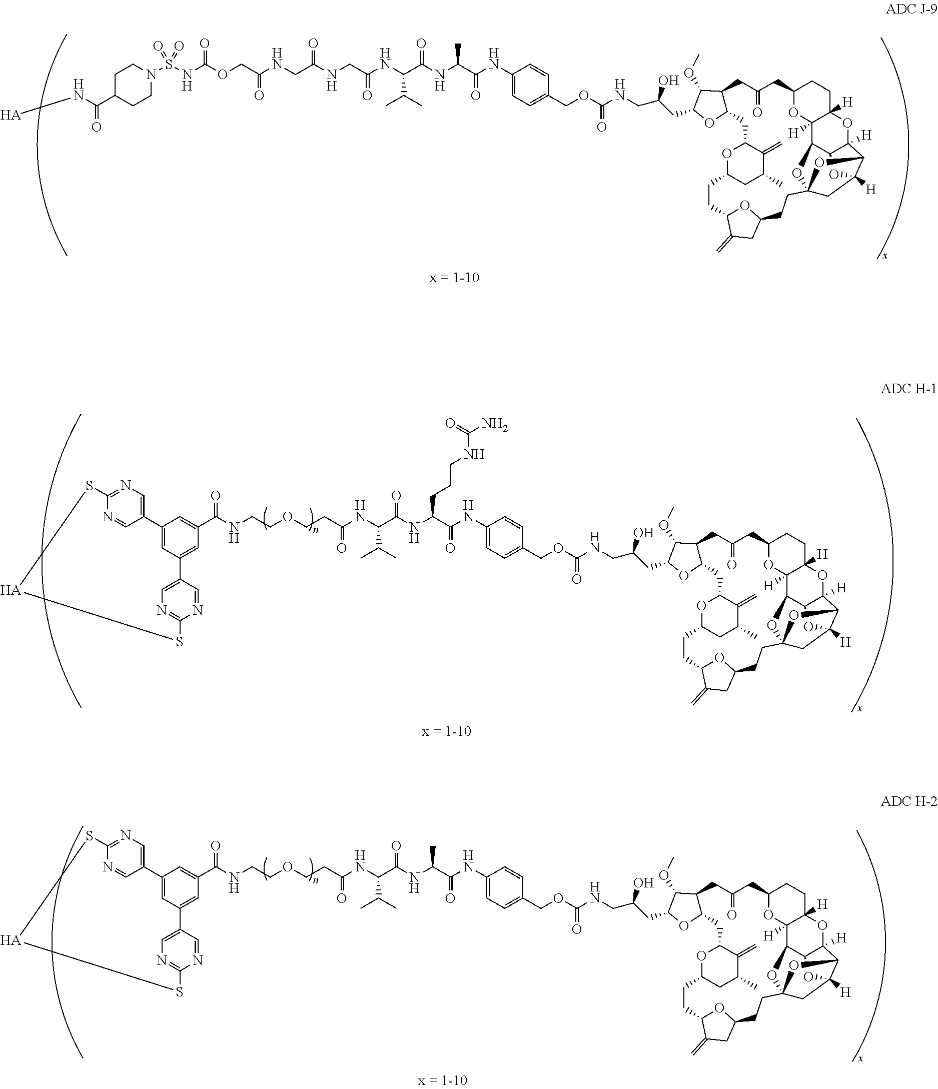 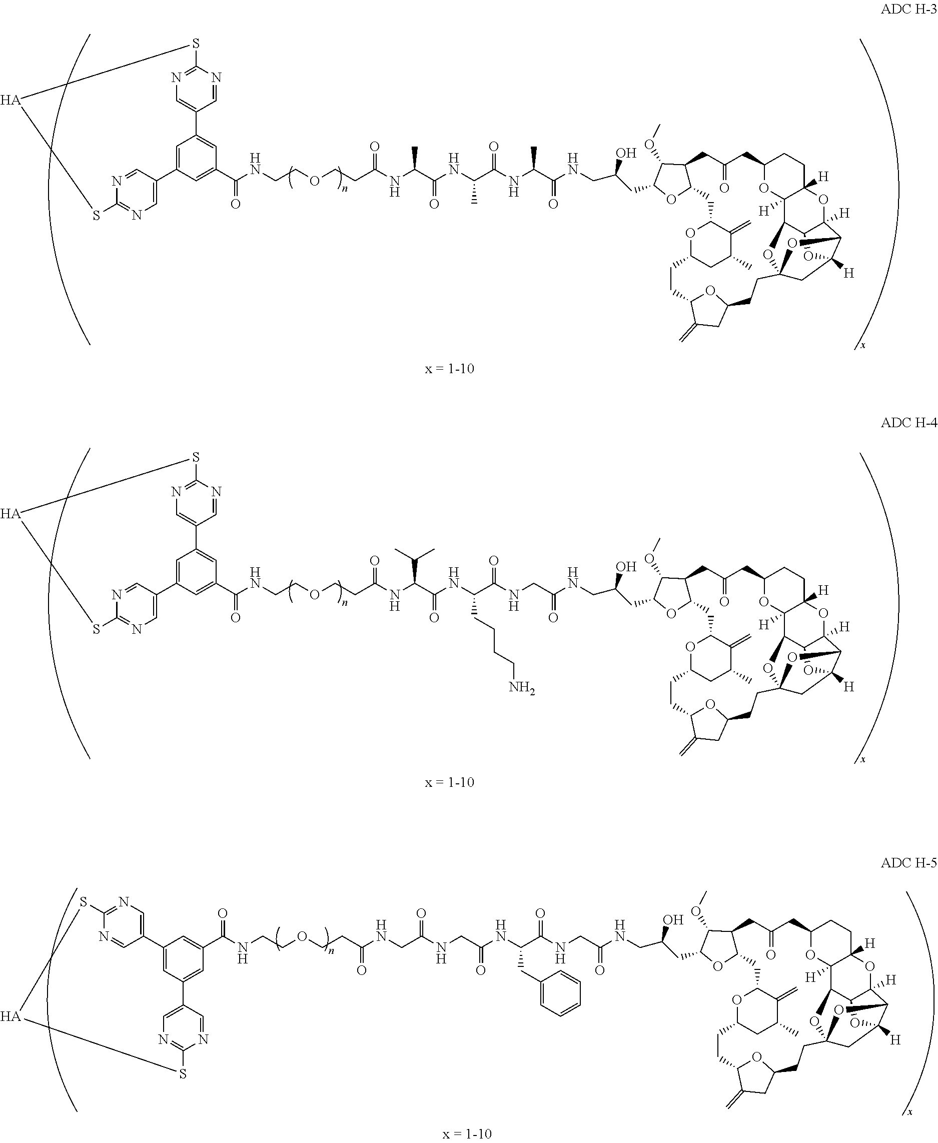 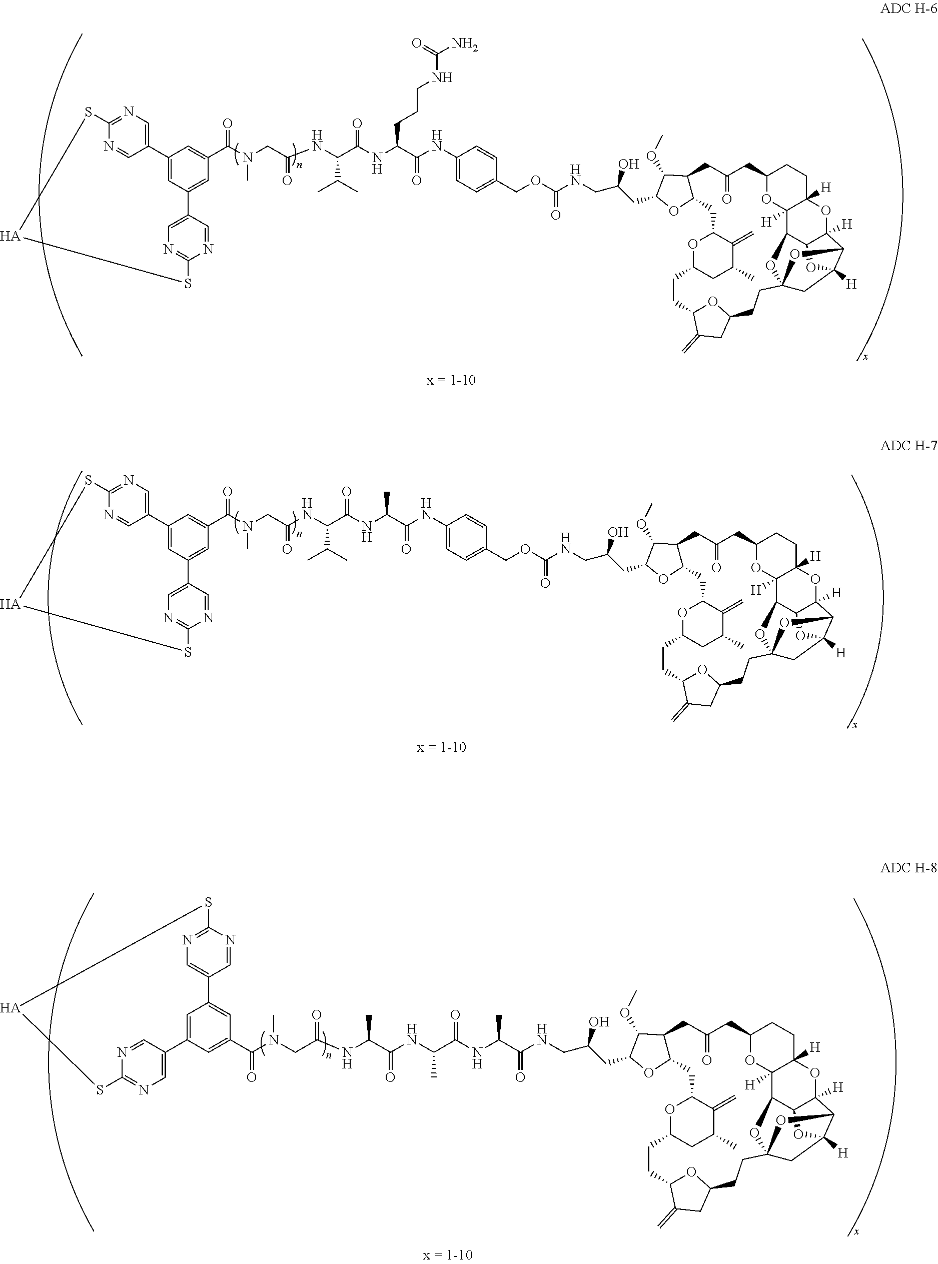 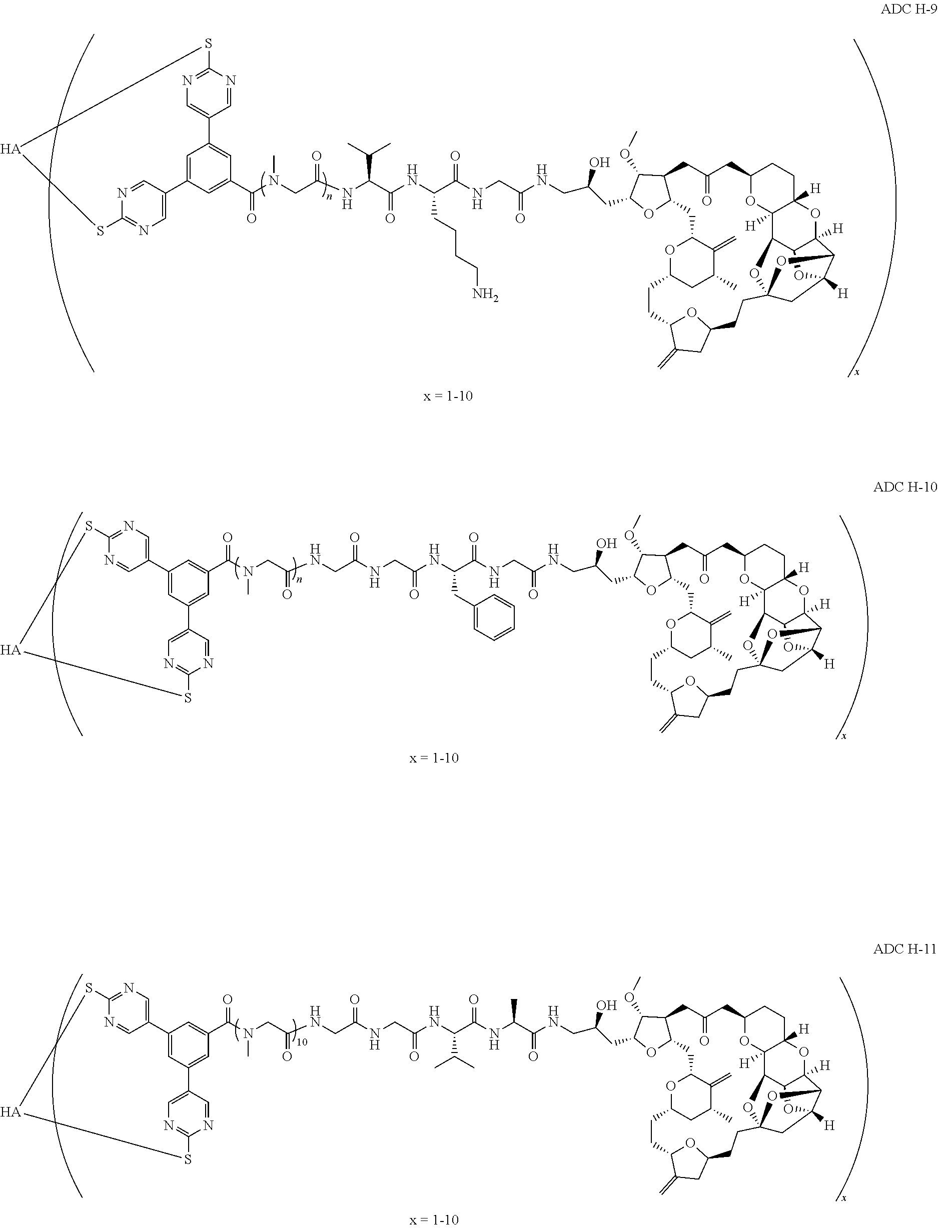 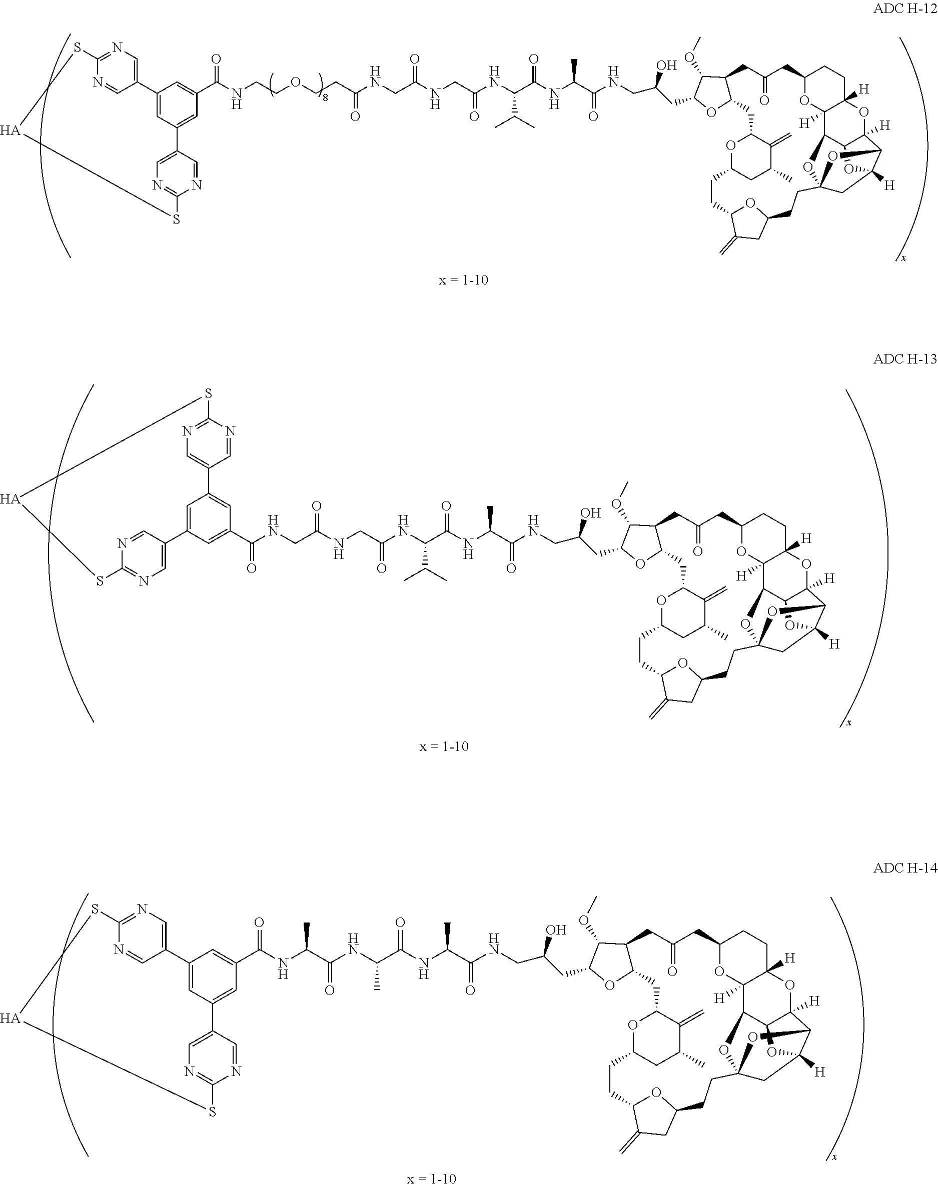 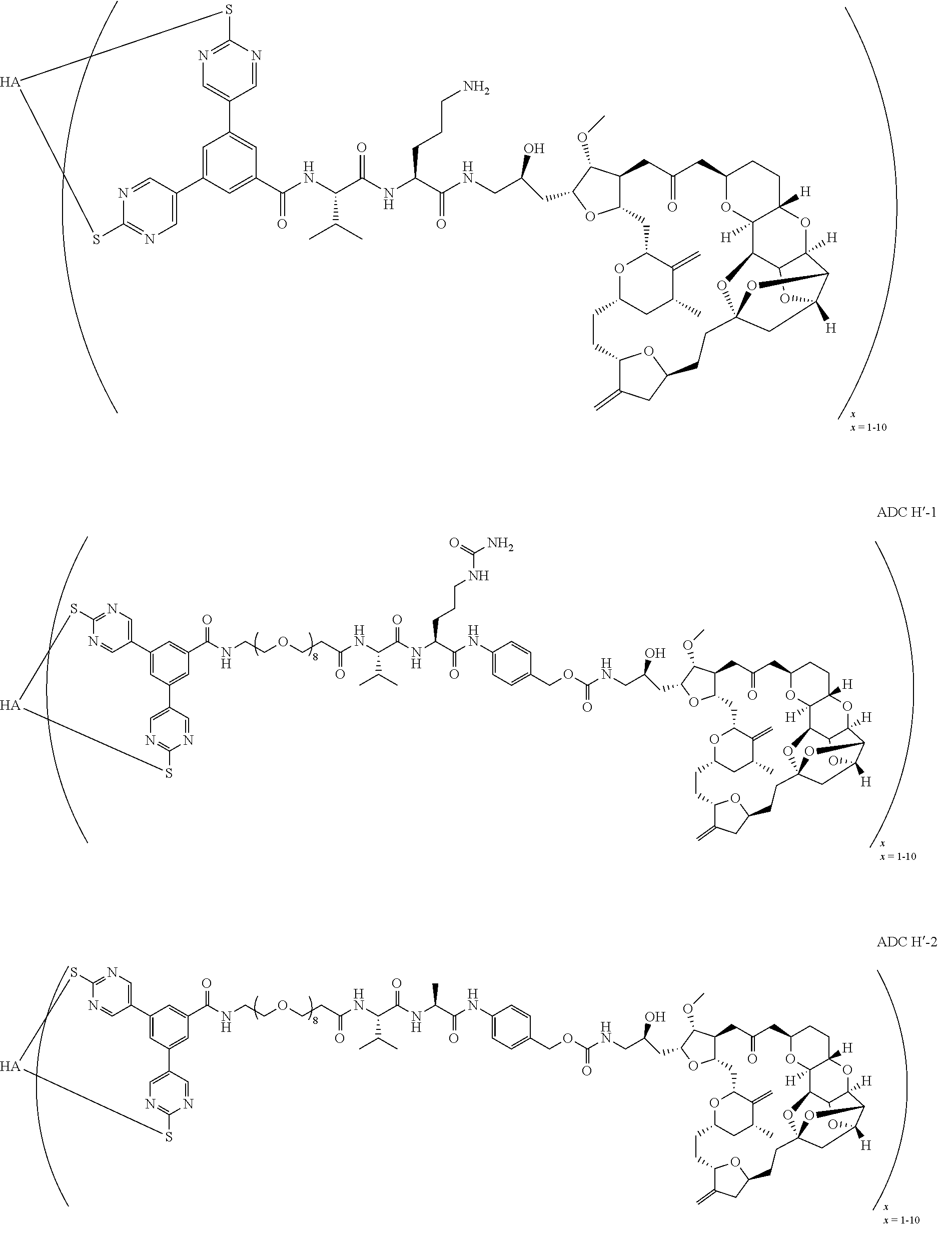 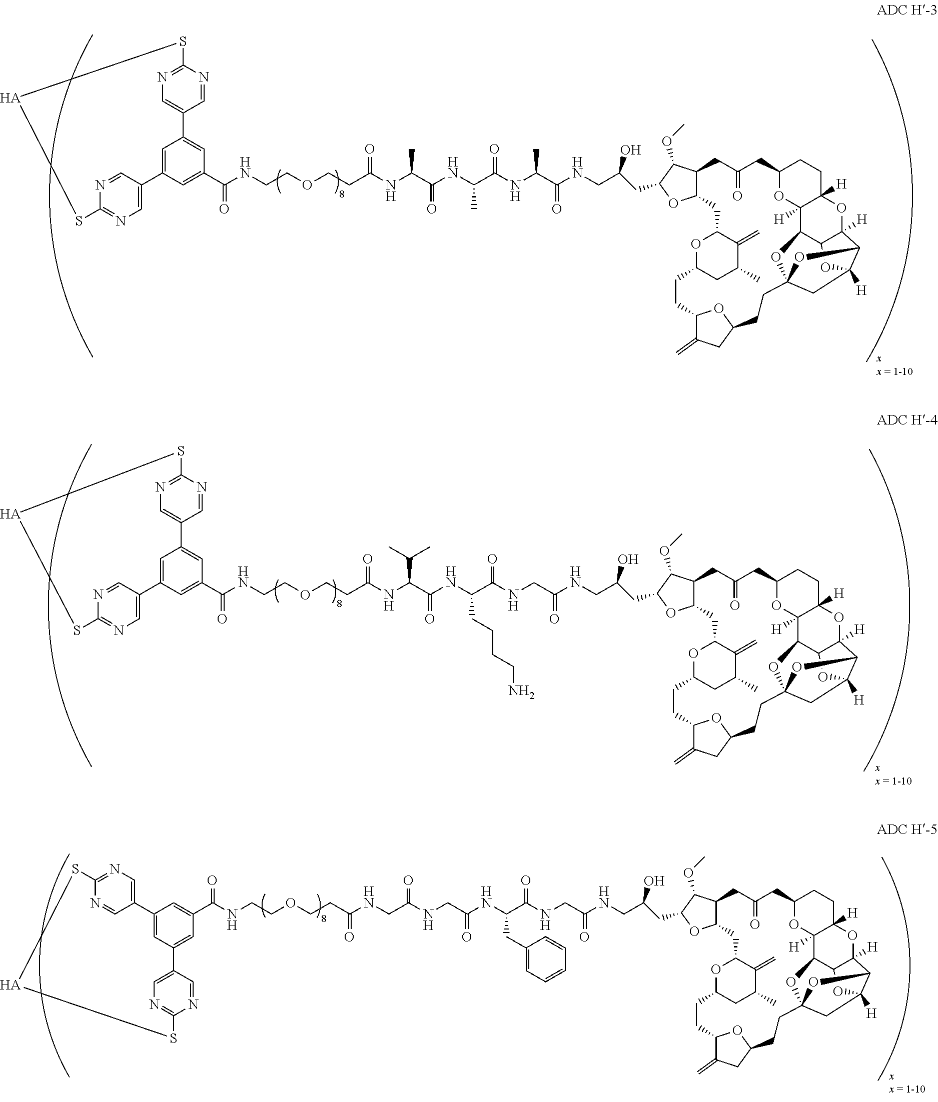 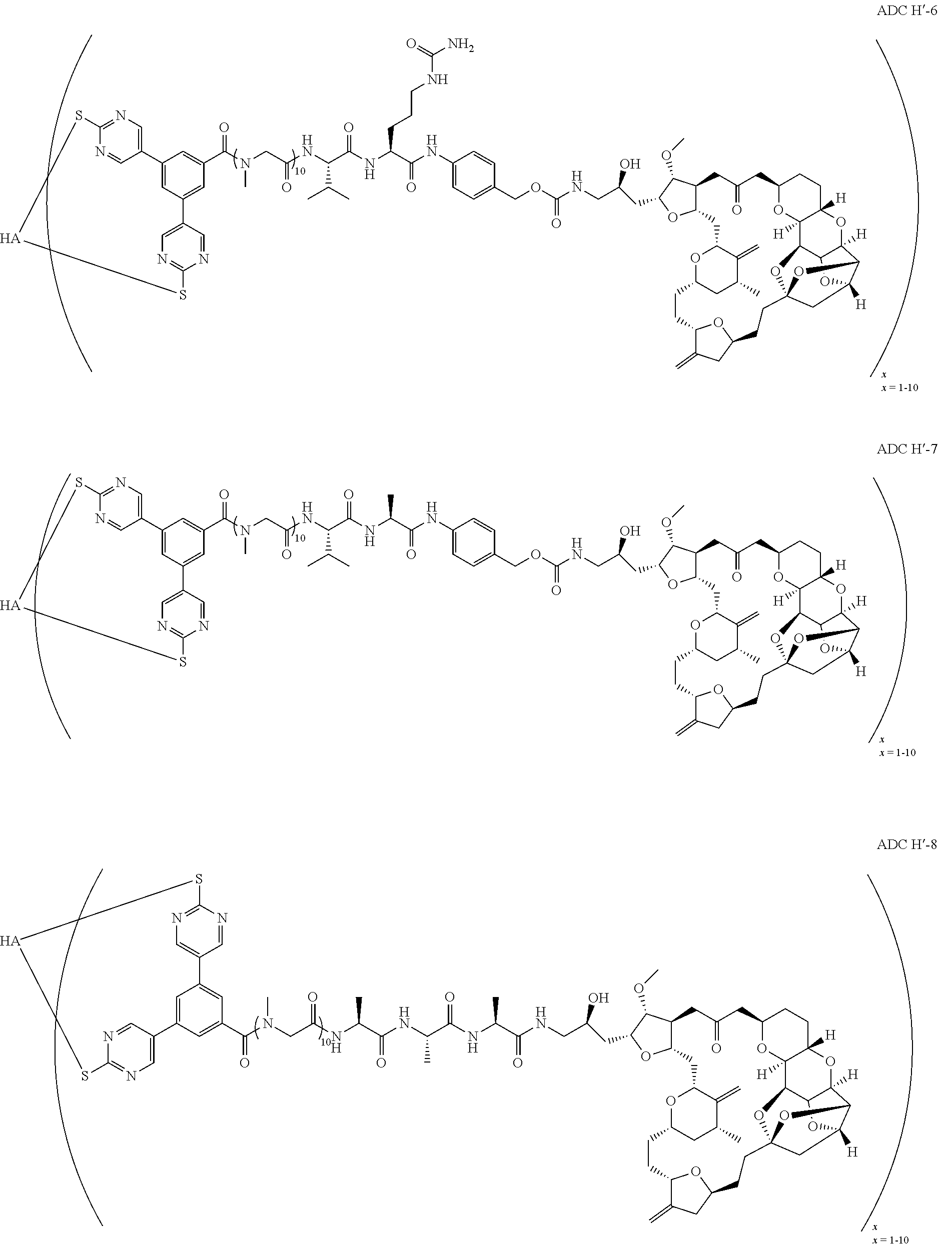 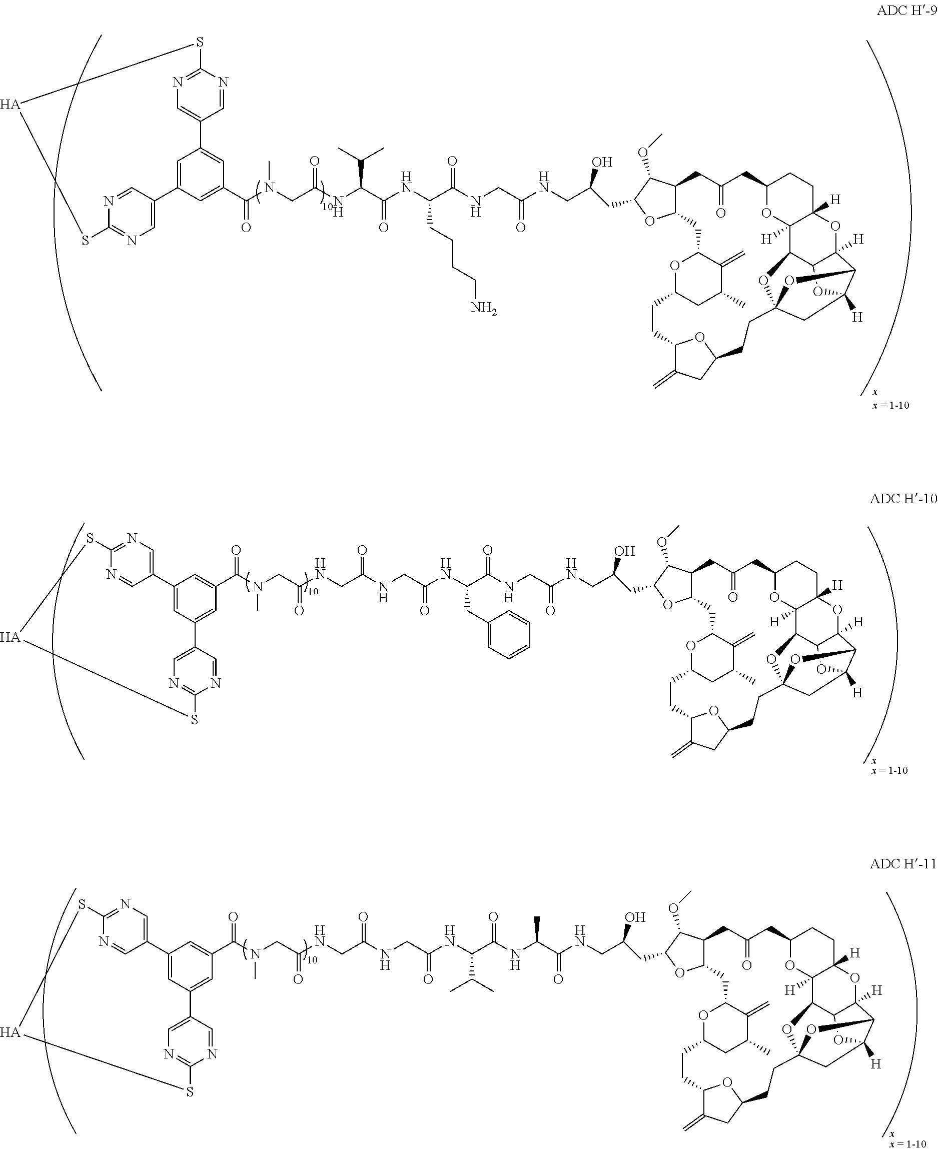 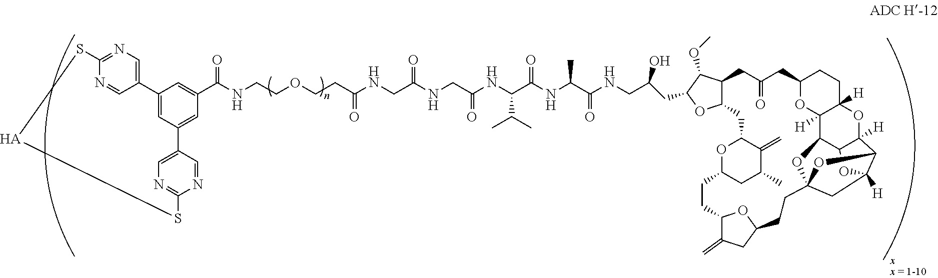
wherein, HA represents an antibody or antigen-binding fragment thereof; n is an integer selected from 1 to 20, preferably an integer of 1 to 15, such as an integer of 1 to 12, 3 to 12, 5 to 10, 8 to 10, such as 1, 2, 3, 4, 5, 6, 7, 8, 9, 10, 11, 12, 13, 14 or 15, preferably 5, 8, or 10; 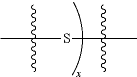 [SMILES: *S*]
represents a specific linkage between a sulfhydryl group in the antibody or antigen-binding fragment thereof and the linker; 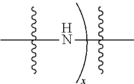 [SMILES: *N*]
represents a specific linkage between an amino group in the antibody or antigen-binding fragment thereof and the linker.

15. A composition, wherein the composition comprises one or more of the conjugates according to claim 12, and the DAR value (drug-antibody ratio) of the composition is 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 8, for example, 3.0 to 3.5, 3.0 to 4.0, 3.0 to 4.5, 3.0 to 5.0, 6.0 to 6.5, 6.0 to 7.0, 6.0 to 7.5, 6.0 to 8.0, 6.0 to 8.5, 6.5 to 7.0, 6.5 to 7.5, 6.5 to 8.0, 6.5 to 8.5, 7.0 to 7.5, 7.0 to 8.0 or 7.5 to 8.0, or, the DAR value of the composition is about 1.0 to 6.0, for example, about 1.0 to 5.5, about 1.0 to 5.0, about 1.5 to 6.0, about 1.5 to about 5.5, about 1.5 to 5.0, 2.0 to 5.5, about 2.0 to about 5.0, for example, about 1.0, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, about 1.2, about 1.21, about 1.22, about 1.23, about 1.24, about 1.25, about 1.26, about 1.27, about 1.28, about 1.29, about 1.3, about 1.31, about 1.32, about 1.33, about 1.34, about 1.35, about 1.36, about 1.37, about 1.38, about 1.39, about 1.4, about 1.41, about 1.42, about 1.43, about 1.44, about 1.45, about 1.46, about 1.47, about 1.48, about 1.49, about 1.5, about 1.51, about 1.52, about 1.53, about 1.54, about 1.55, about 1.56, about 1.57, about 1.58, about 1.59, about 1.6, about 1.61, about 1.62, about 1.63, about 1.64, about 1.65, about 1.66, about 1.67, about 1.68, about 1.69, about 1.7, about 1.71, about 1.72, about 1.73, about 1.74, about 1.75, about 1.76, about 1.77, about 1.78, about 1.79, about 1.8, about 1.81, about 1.82, about 1.83, about 1.84, about 1.85, about 1.86, about 1.87, about 1.88, about 1.89, about 1.9, about 1.91, about 1.92, about 1.93, about 1.94, about 1.95, about 1.96, about 1.97, about 1.98, about 1.99, about 2.0, about 2.01, about 2.02, about 2.03, about 2.04, about 2.05, about 2.06, about 2.07, about 2.08, about 2.9, about 2.1, about 2.11, about 2.12, about 2.13, about 2.14, about 2.15, about 2.16, about 2.17, about 2.18, about 2.19, about 2.2, about 2.21, about 2.22, about 2.23, about 2.24, about 2.25, about 2.26, about 2.27, about 2.28, about 2.29, about 2.3, about 2.31, about 2.32, about 2.33, about 2.34, about 2.35, about 2.36, about 2.37, about 2.38, about 2.39, about 2.4, about 2.41, about 2.42, about 2.43, about 2.44, about 2.45, about 2.46, about 2.47, about 2.48, about 2.49, about 2.5, about 2.51, about 2.52, about 2.53, about 2.54, about 2.55, about 2.56, about 2.57, about 2.58, about 2.59, about 2.6, about 2.61, about 2.62, about 2.63, about 2.64, about 2.65, about 2.66, about 2.67, about 2.68, about 2.69, about 2.7, about 2.71, about 2.72, about 2.73, about 2.74, about 2.75, about 2.76, about 2.77, about 2.78, about 2.79, about 2.8, about 2.81, about 2.82, about 2.83, about 2.84, about 2.85, about 2.86, about 2.87, about 2.88, about 2.89, about 2.9, about 2.91, about 2.92, about 2.93, about 2.94, about 2.95, about 2.96, about 2.97, about 2.98, about 2.99, about 3.0, about 3.01, about 3.02, about 3.03, about 3.04, about 3.05, about 3.06, about 3.07, about 3.08, about 3.09, about 3.1, about 3.11, about 3.12, about 3.13, about 3.14, about 3.15, about 3.16, about 3.17, about 3.18, about 3.19, about 3.2, about 3.21, about 3.22, about 3.23, about 3.24, about 3.25, about 3.26, about 3.27, about 3.28, about 3.29, about 3.3, about 3.31, about 3.32, about 3.33, about 3.34, about 3.35, about 3.36, about 3.37, about 3.38, about 3.39, about 3.4, about 3.41, about 3.42, about 3.43, about 3.44, about 3.45, about 3.46, about 3.47, about 3.48, about 3.49, about 3.5, about 3.51, about 3.52, about 3.53, about 3.54, about 3.55, about 3.56, about 3.57, about 3.58, about3.59, about 3.6, about 3.61, about 3.62, about 3.63, about 3.64, about 3.65, about 3.66, about 3.67, about 3.68, about 3.69, about 3.7, about 3.71, about 3.72, about 3.73, about 3.74, about 3.75, about 3.76, about 3.77, about 3.78, about 3.79, about 3.8, about 3.81, about 3.82, about 3.83, about 3.84, about 3.85, about 3.86, about 3.87, about 3.88, about 3.89, about 3.9, about 3.91, about 3.92, about 3.93, about 3.94, about 3.95, about 3.96, about 3.97, about 3.98, about 3.99, about 4.0, about 4.01, about 4.02, about 4.03, about 4.04, about 4.05, about 4.06, about 4.07, about 4.08, about 4.09, about 4.1, about 4.11, about 4.12, about 4.13, about 4.14, about 4.15, about 4.16, about 4.17, about 4.18, about 4.19, about 4.2, about 4.21, about 4.22, about 4.23, about 4.24, about 4.25, about 4.26, about 4.27, about 4.28, about 4.29, about 4.3, about 4.31, about 4.32, about 4.33, about 4.34, about 4.35, about 4.36, about 4.37, about 4.38, about 4.39, about 4.4, about 4.41, about 4.42, about 4.43, about 4.44, about 4.45, about 4.46, about 4.47, about 4.48, about 4.49, about 4.5, about 4.51, about 4.52, about 4.53, about 4.54, about 4.55, about 4.56, about 4.57, about 4.58, about 4.59, about 4.6, about 4.61, about 4.62, about 4.63, about 4.64, about 4.65, about 4.66, about 4.67, about 4.68, about 4.69, about 4.7, about 4.71, about 4.72, about 4.73, about 4.74, about 4.75, about 4.76, about 4.77, about 4.78, about 4.79, about 4.8, about 4.81, about 4.82, about 4.83, about 4.84, about 4.85, about 4.86, about 4.87, about 4.88, about 4.89, about 4.9, about 4.91, about 4.92, about 4.93, about 4.94, about 4.95, about 4.96, about 4.97, about 4.98, about 4.99, about 5.0.

16. A pharmaceutical composition, which comprises the compound or pharmaceutically acceptable salt thereof according to claim 1, a conjugate represented by Formula (II), or a composition comprising one or more of the conjugates and the DAR value (drug-antibody ratio) of the composition is 1 to 10; and one or more pharmaceutical excipients; 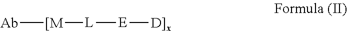
wherein Ab represents a targeting moiety; M, L, E and D are as described in claim 1, and x is 1 to 10.

17.-20. (canceled)

21. A method for preventing or treating a cancer, the method comprising administering to an individual in need thereof a prophylactically or therapeutically effective amount of the compound or pharmaceutically acceptable salt thereof according to claim 1, a conjugate represented by Formula (II), a composition comprising one or more of the conjugates and the DAR value (drug-antibody ratio) of the composition is 1 to 10, or a pharmaceutical composition comprising the compound or pharmaceutically acceptable salt thereof, the conjugate or the composition and one or more pharmaceutical excipients; 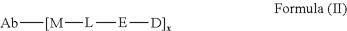
wherein Ab represents a targeting moiety; M, L, E and D are as described in claim 1, and x is 1 to 10.

22. The method according to claim 21, wherein the cancer is solid tumors or hematological malignancies, such as gastric cancer, breast cancer, lung cancer (for example, non-small cell lung cancer, specifically lung adenocarcinoma) and urothelial carcinoma.

23. A compound having the following structure, or a salt, a stereoisomer, a tautomer or an isotopically labeled compound thereof: 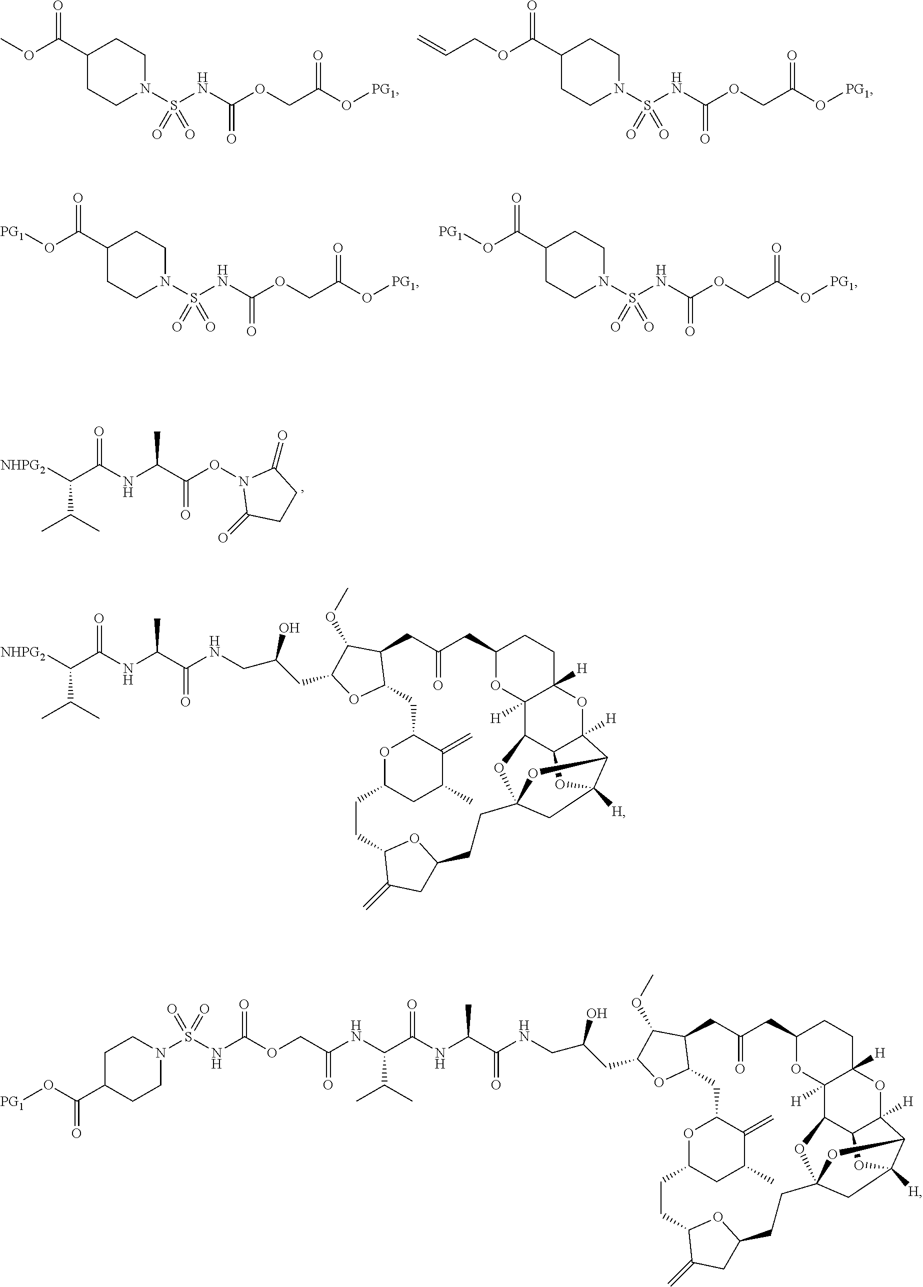 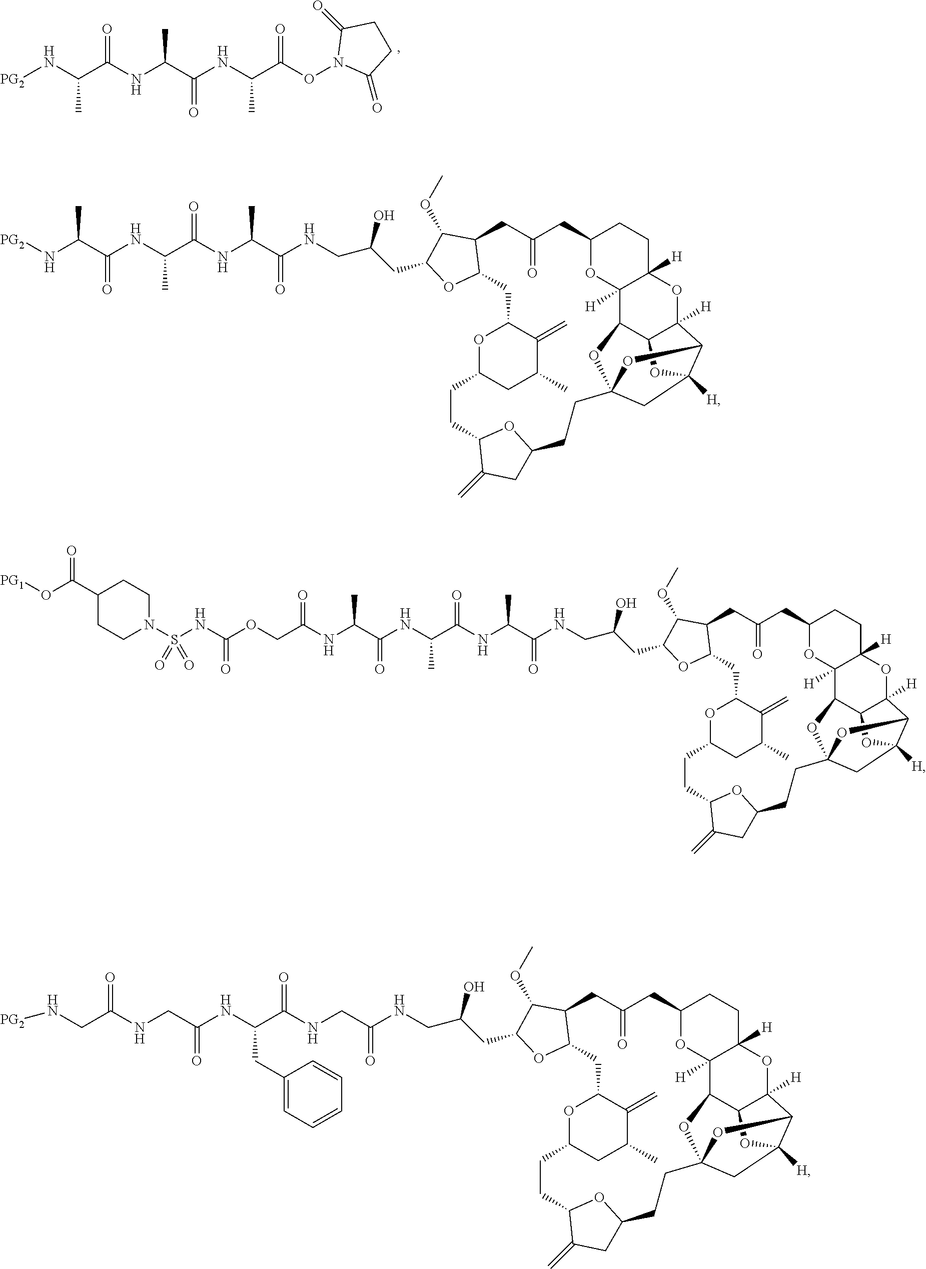 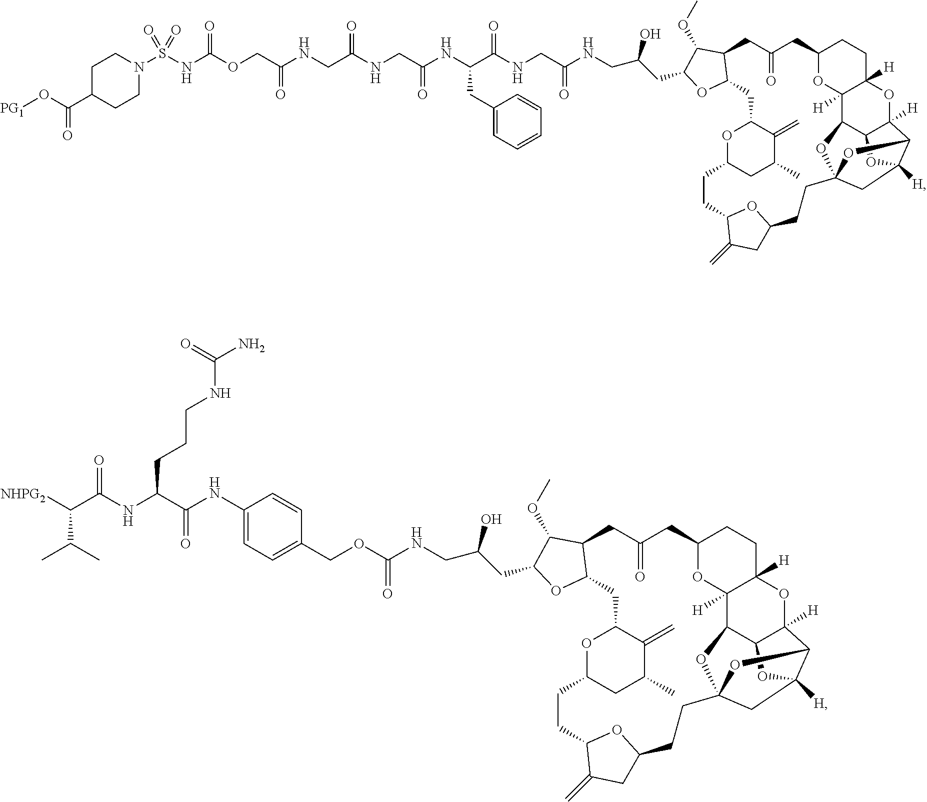 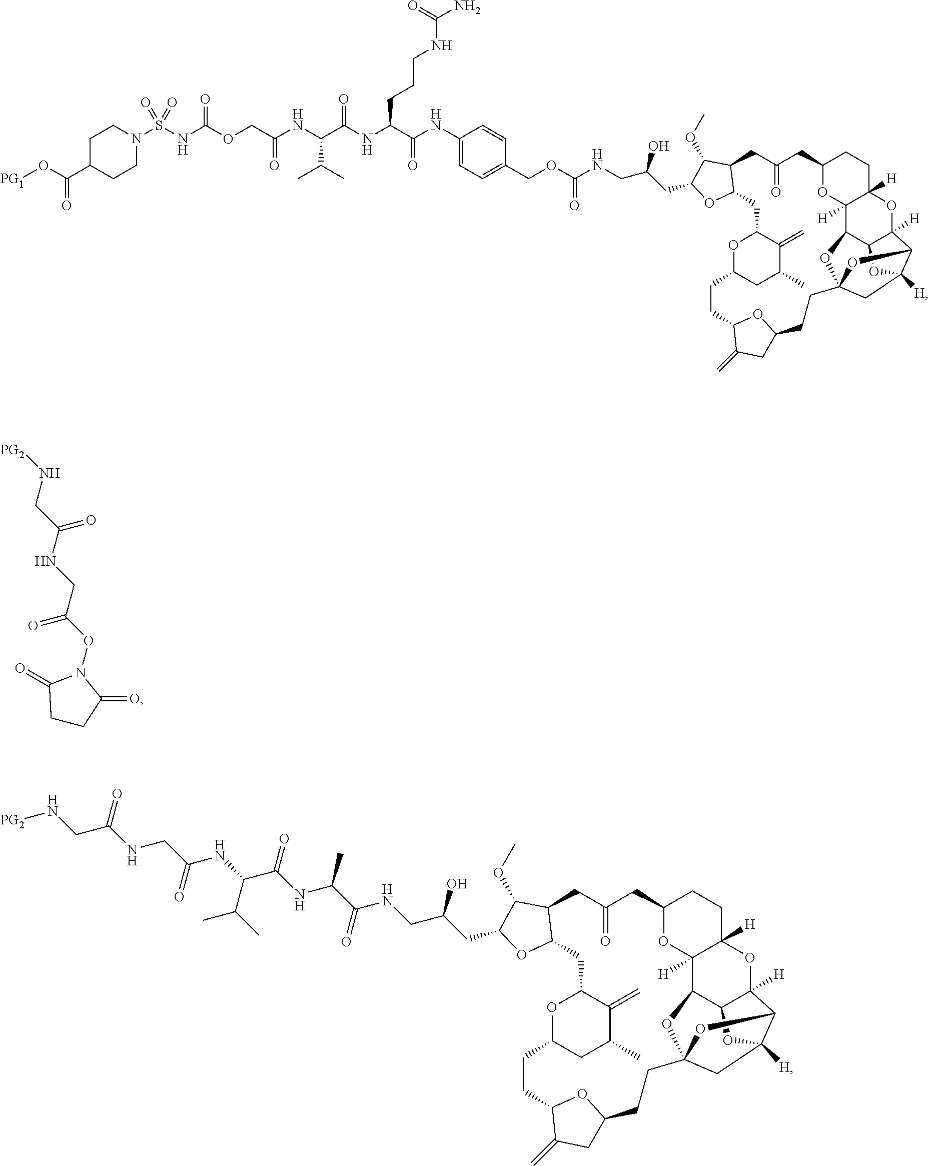 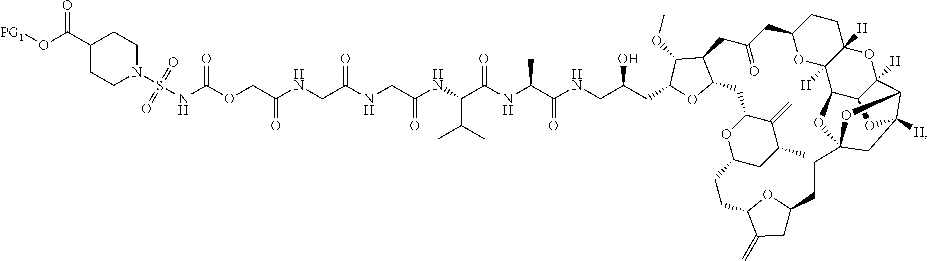
wherein
PG₁ is each independently H or a carboxyl protecting group, such as C₁₋₆ alkyl, allyl, benzyl, 2,4-dimethoxybenzyl, p-methoxybenzyl, methoxyethoxymethyl, pentafluorophenyl, 4-p-methylbenzyloxybenzyl;
PG₂ is each independently H or an amino protecting group, for example, an alkoxycarbonyl amino protecting group, such as benzyloxycarbonyl (Cbz), tert-butyloxycarbonyl (Boc), fluorenylmethoxycarbonyl (Fmoc), allyloxycarbonyl (Alloc), trimethylsilylethoxycarbonyl (Teoc), methyl(or ethyl)oxycarbonyl; an acyl amino protecting group, such as phthaloyl (Pht), p-toluenesulfonyl (Tos), trifluoroacetyl (Tfa), o-(p-)nitrobenzenesulfonyl (Ns), pivaloyl, benzoyl, tert-butyloxycarbonyl, 9-fluorenylmethoxycarbonyl, allyloxycarbonyl, trichloroethoxycarbonyl, trimethylsilylethoxycarbonyl, benzyloxycarbonyl, p-toluenesulfonyl, p-nitrobenzenesulfonyl, trifluoroacetyl, methoxycarbonyl, or ethoxycarbonyl; an alkyl amino protecting group, such as trityl (Trt), 2,4-dimethoxybenzyl (Dmb), 4-methoxybenzyl (PMB), benzyl (Bn).

24. A compound or salt, stereoisomer, tautomer or isotope-labeled compound thereof as shown below: 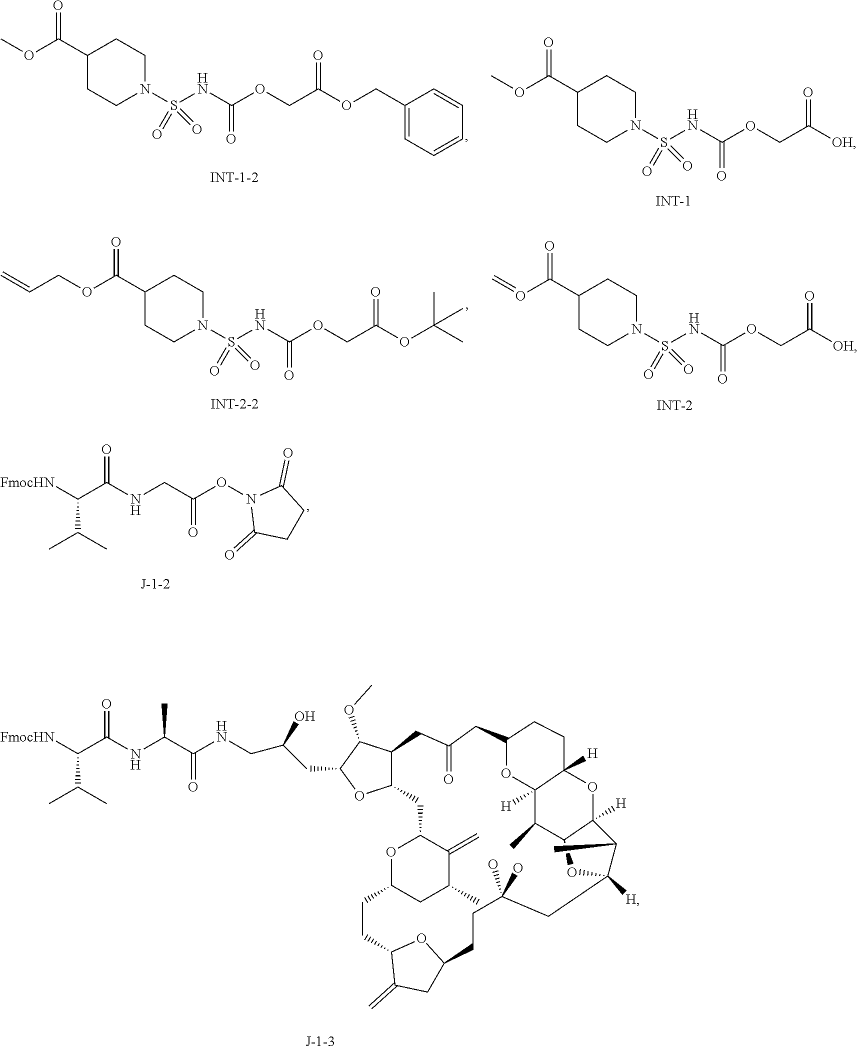 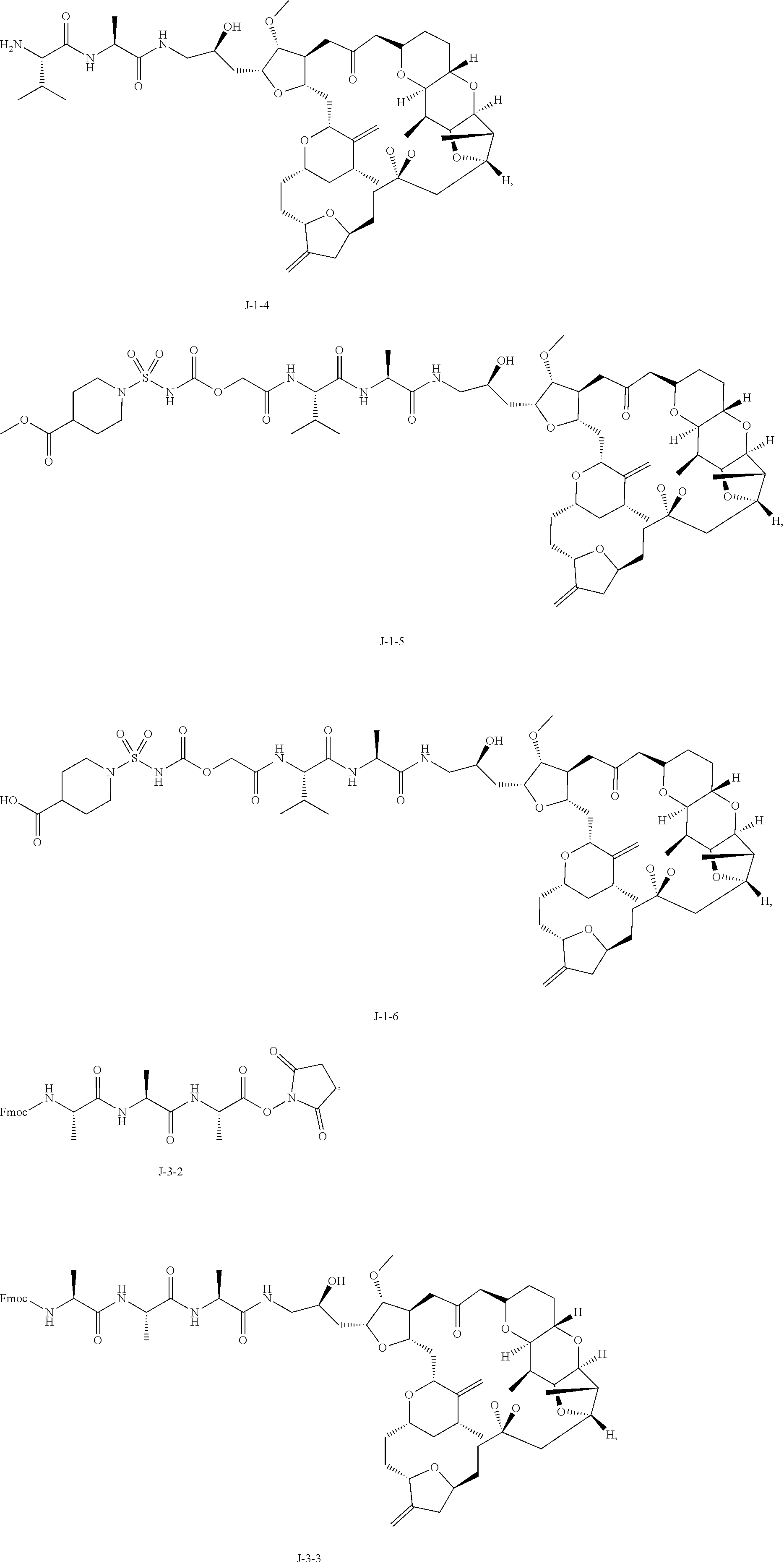 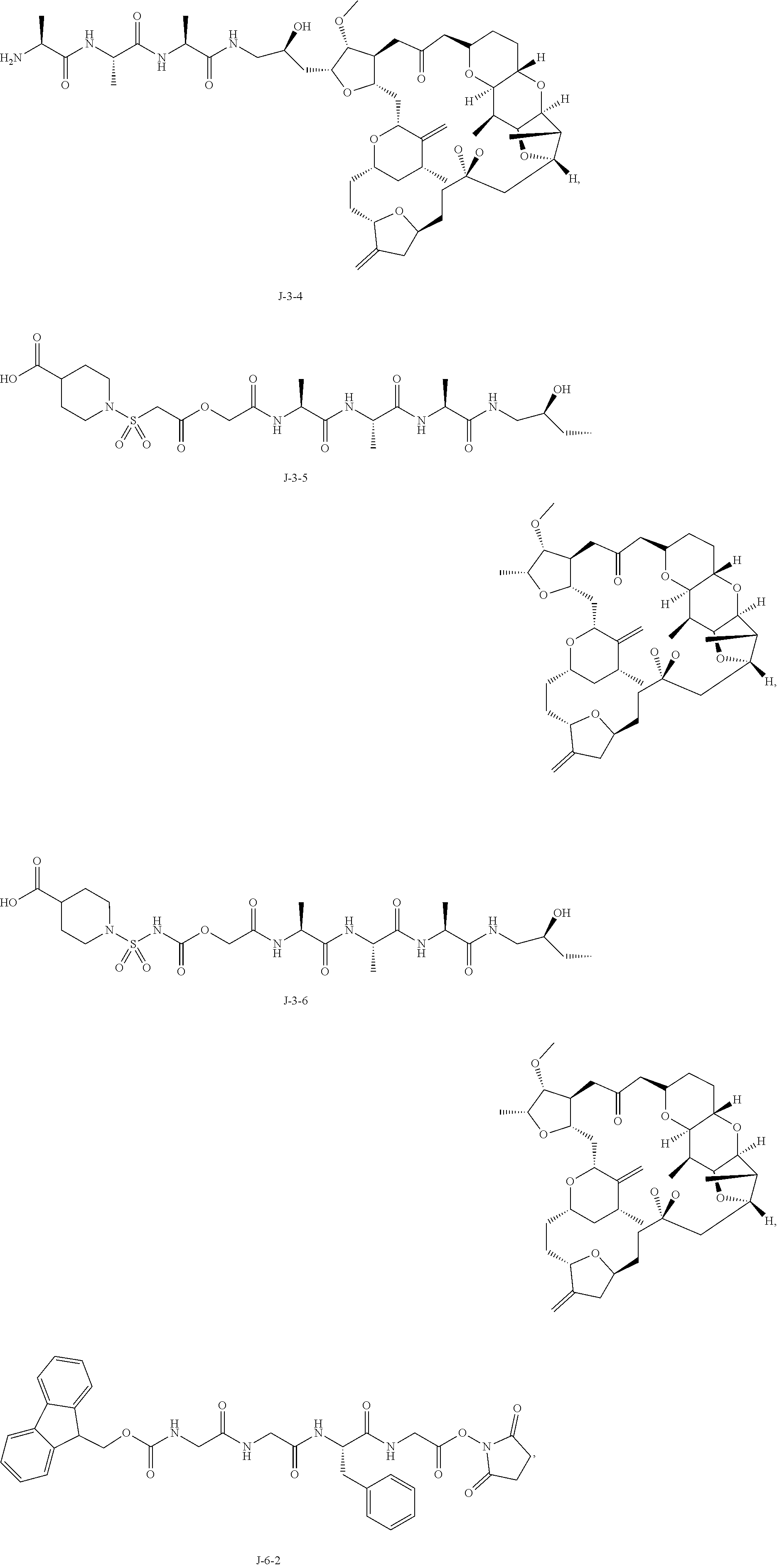 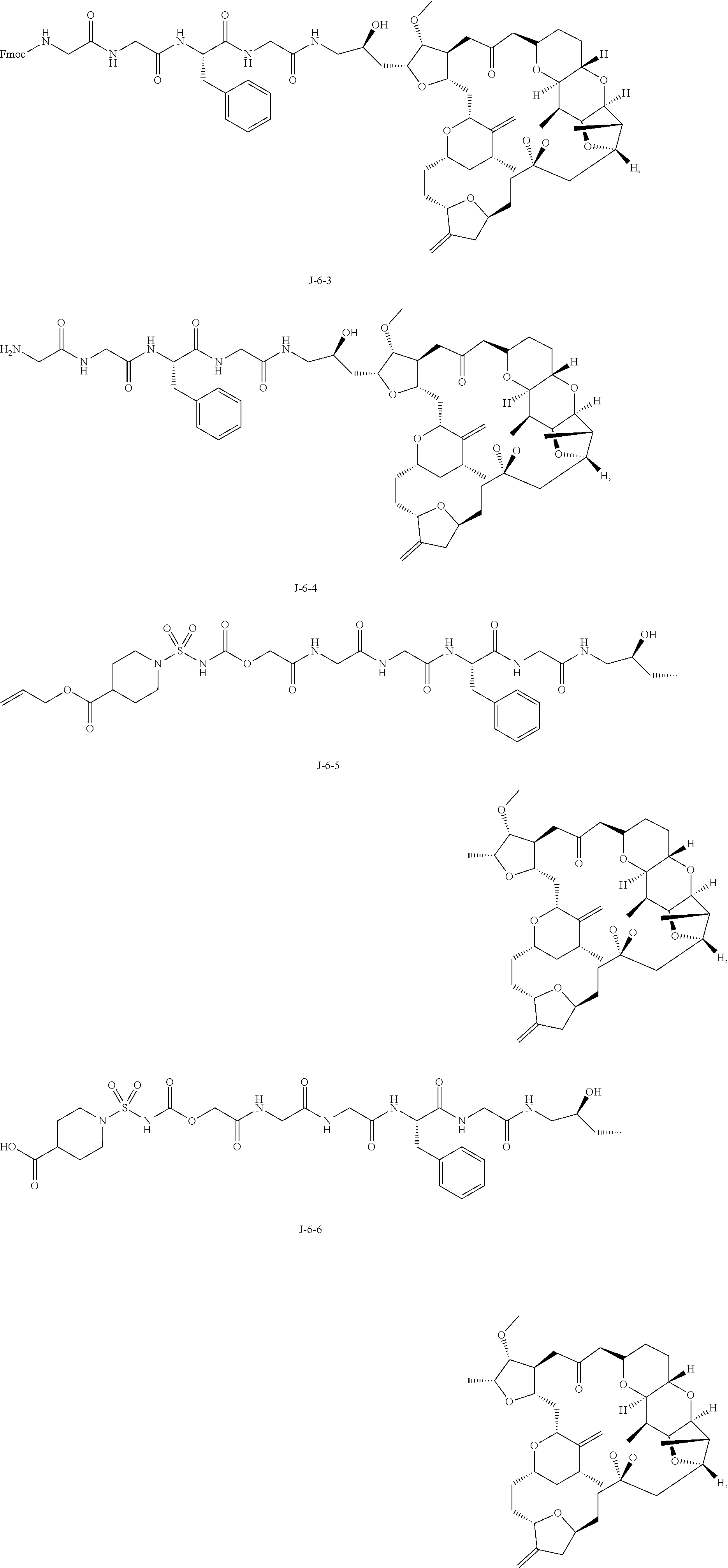 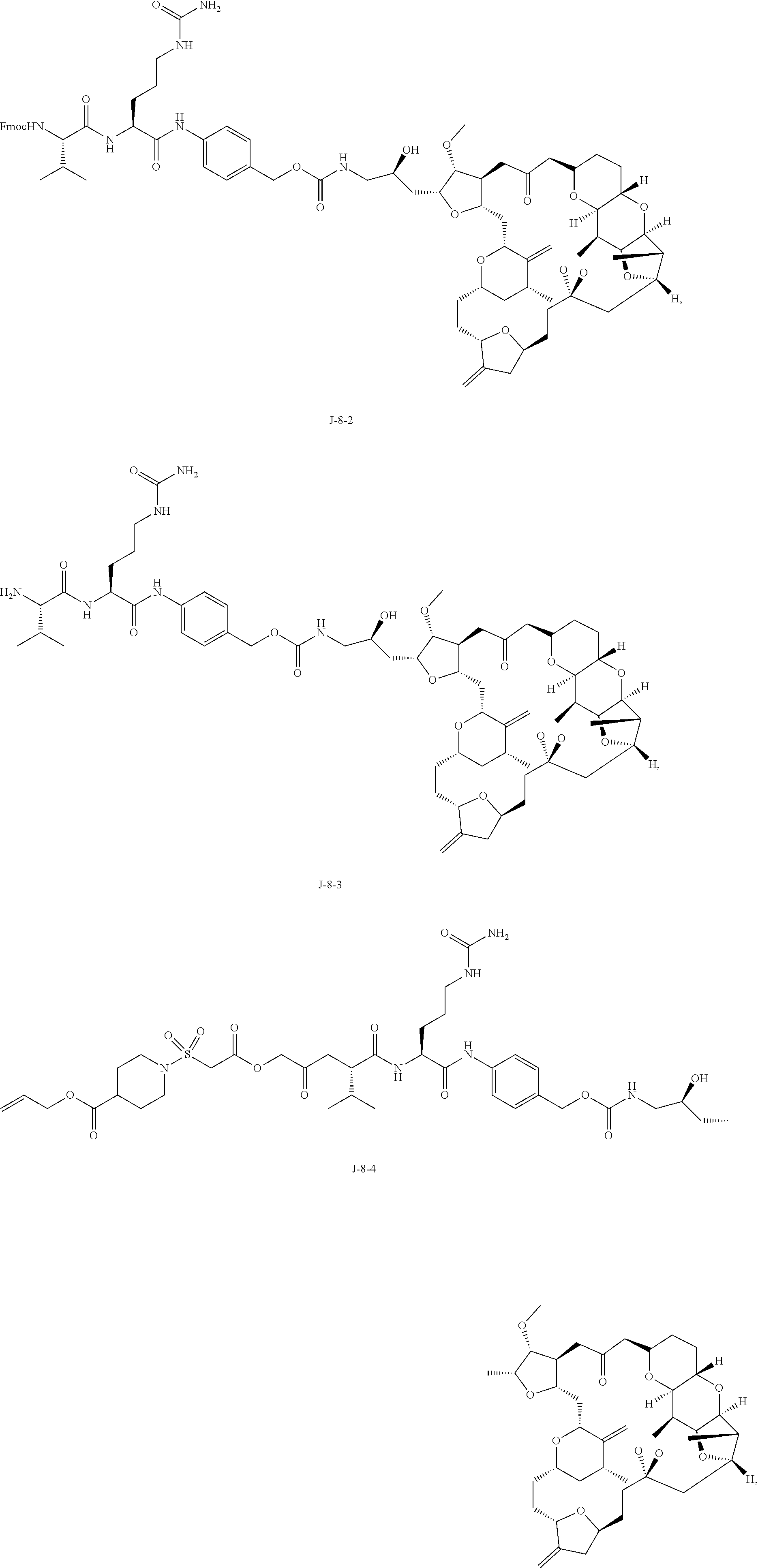 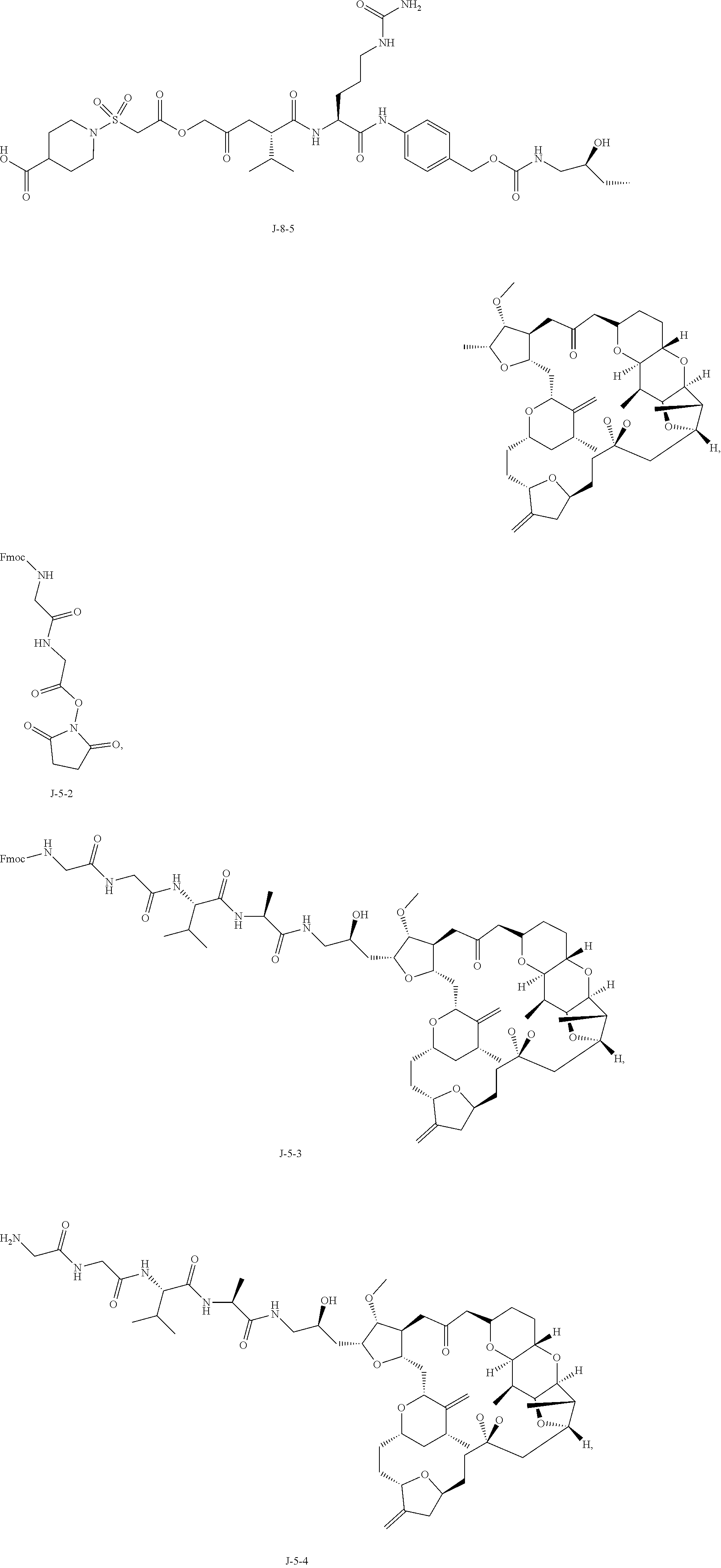 [chemical structure]


SEQUENCE LISTING (47 sequences)

SEQ ID NO: 1 - protein, 120 aa, synthetic construct [sequence omitted]

SEQ ID NO: 2 - protein, 107 aa, synthetic construct [sequence omitted]

SEQ ID NO: 3 - protein, 119 aa, synthetic construct [sequence omitted]

SEQ ID NO: 4 - protein, 107 aa, synthetic construct [sequence omitted]

SEQ ID NO: 5 - protein, 7 aa, synthetic construct
GFNIKDT

SEQ ID NO: 6 - protein, 6 aa, synthetic construct
YPTNGY

SEQ ID NO: 7 - protein, 11 aa, synthetic construct
WGGDGFYAMDY

SEQ ID NO: 8 - protein, 11 aa, synthetic construct
RASQDVNTAVA

SEQ ID NO: 9 - protein, 7 aa, synthetic construct
SASFLYS

SEQ ID NO: 10 - protein, 9 aa, synthetic construct
QQHYTTPPT

SEQ ID NO: 11 - protein, 5 aa, synthetic construct
DTYIH

SEQ ID NO: 12 - protein, 17 aa, synthetic construct
RIYPTNGYTRYADSVKG

SEQ ID NO: 13 - protein, 8 aa, synthetic construct
GFNIKDTY

SEQ ID NO: 14 - protein, 8 aa, synthetic construct
IYPTNGYT

SEQ ID NO: 15 - protein, 13 aa, synthetic construct
SRWGGDGFYAMDY

SEQ ID NO: 16 - protein, 6 aa, synthetic construct
QDVNTA

SEQ ID NO: 17 - skipped

SEQ ID NO: 18 - protein, 10 aa, synthetic construct
GFNIKDTYIH

SEQ ID NO: 19 - protein, 10 aa, synthetic construct
RIYPTNGYTR

SEQ ID NO: 20 - protein, 7 aa, synthetic construct
GFTFTDY

SEQ ID NO: 21 - protein, 6 aa, synthetic construct
NPNSGG

SEQ ID NO: 22 - protein, 10 aa, synthetic construct
NLGPSFYFDY

SEQ ID NO: 23 - protein, 11 aa, synthetic construct
KASQDVSIGVA

SEQ ID NO: 24 - protein, 7 aa, synthetic construct
SASYRYT

SEQ ID NO: 25 - protein, 9 aa, synthetic construct
QQYYIYPYT

SEQ ID NO: 26 - protein, 5 aa, synthetic construct
DYTMD

SEQ ID NO: 27 - protein, 17 aa, synthetic construct
DVNPNSGGSIYNQRFKG

SEQ ID NO: 28 - protein, 8 aa, synthetic construct
GFTFTDYT

SEQ ID NO: 29 - protein, 8 aa, synthetic construct
VNPNSGGS

SEQ ID NO: 30 - protein, 12 aa, synthetic construct
ARNLGPSFYFDY

SEQ ID NO: 31 - protein, 6 aa, synthetic construct
QDVSIG

SEQ ID NO: 32 - skipped

SEQ ID NO: 33 - protein, 10 aa, synthetic construct
GFTFTDYTMD

SEQ ID NO: 34 - protein, 10 aa, synthetic construct
DVNPNSGGSI

SEQ ID NO: 35 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 107 aa, synthetic construct [sequence omitted]

SEQ ID NO: 37 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 449 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 4 aa, synthetic construct
GGFG

SEQ ID NO: 42 - protein, 4 aa, synthetic construct
GGVA

SEQ ID NO: 43 - protein, 4 aa, synthetic construct
GFLG

SEQ ID NO: 44 - protein, 4 aa, synthetic construct
EAAA

SEQ ID NO: 45 - protein, 5 aa, synthetic construct
GGGGG

SEQ ID NO: 46 - protein, 5 aa, synthetic construct
GGGGS

SEQ ID NO: 47 - protein, 20 aa, synthetic construct
GGGGSGGGGSGGGGSGGGGS